SEQUENCE LISTING (262 sequences)

SEQ ID NO: 1 - protein, 119 aa, synthetic construct
QVQLQESGPGLVKPSETLSLTCTVSGGSISSYFWTWIRQPPGKRLEWIGYIHNSGTTNQNPSLKSRVTISIDTSKNHFSLKLSSVTAADTAVYYCARGRGMATLPFDYWGQGTLVTVSS

SEQ ID NO: 2 - protein, 119 aa, synthetic construct
QVQLQESGPGLVKPSETLSLTCTVSGGSISSYFWSWIRQPPGKGLEWIGYIHNSGTTNYNPSLKSRVTISVDTSKNQFSLKLSSVTAADTAVYYCARGRGMATLPFDYWGQGTLVTVSS

SEQ ID NO: 3 - protein, 119 aa, synthetic construct
QVQLQESGPGLVKPSETLSLTCTVSGGSISSYFWTWIRQPPGKGLEWIGYIHNSGTTNYNPSLKSRVTISVDTSKNQFSLKLSSVTAADTAVYYCARGRGMATLPFDYWGQGTLVTVSS

SEQ ID NO: 4 - protein, 119 aa, synthetic construct
QVQLQESGPGLVKPSETLSLTCTVSGGSISSYFWSWIRQPPGKRLEWIGYIHNSGTTNYNPSLKSRVTISVDTSKNQFSLKLSSVTAADTAVYYCARGRGMATLPFDYWGQGTLVTVSS

SEQ ID NO: 5 - protein, 119 aa, synthetic construct
QVQLQESGPGLVKPSETLSLTCTVSGGSISSYFWSWIRQPPGKGLEWIGYIHNSGTTNQNPSLKSRVTISVDTSKNQFSLKLSSVTAADTAVYYCARGRGMATLPFDYWGQGTLVTVSS

SEQ ID NO: 6 - protein, 119 aa, synthetic construct
QVQLQESGPGLVKPSETLSLTCTVSGGSISSYFWSWIRQPPGKGLEWIGYIHNSGTTNYNPSLKSRVTISIDTSKNQFSLKLSSVTAADTAVYYCARGRGMATLPFDYWGQGTLVTVSS

SEQ ID NO: 7 - protein, 119 aa, synthetic construct
QVQLQESGPGLVKPSETLSLTCTVSGGSISSYFWSWIRQPPGKGLEWIGYIHNSGTTNYNPSLKSRVTISVDTSKNHFSLKLSSVTAADTAVYYCARGRGMATLPFDYWGQGTLVTVSS

SEQ ID NO: 8 - protein, 119 aa, synthetic construct
QVQLQESGPGLVKPSETLSLTCTVSGGSISSYFWTWIRQPPGKRLEWIGYIHNSGTTNQNPSLKSRVTISIDKSKNHFSLKLSSVTAADTAVYYCARGRGMATLPFDYWGQGTLVTVSS

SEQ ID NO: 9 - protein, 107 aa, synthetic construct [sequence omitted]

SEQ ID NO: 10 - protein, 107 aa, synthetic construct
DIQMTQSPSSLSASVGDRVTITCQASQDITNYLNWYQQKPGKAPKLLIYDASNLETGVPSRFSGSGSGTDFTFTISSLQPEDIATYYCQQYDNLPYTFGQGTKVEIR

SEQ ID NO: 11 - protein, 108 aa, synthetic construct
EIVLTQSPGTLSLSPGERATLSCRASQSVSSTYLAWYQQKPGQAPRLLIYAASSRATGIPDRFSGSGSGTDFTLTISRLEPEDFAVYYCQQYGSSPWTFGQGTKVEIK

SEQ ID NO: 12 - protein, 113 aa, synthetic construct
DIVMTQSPDSLAVSLGERATINCKSSQSVLYSSKNKNYLAWYQQKPGKAPKLLIYAASSLQSGVPSRFSGSGSGTDFTLTISSLQAEDVAVYYCQQYYSTPWTFGQGTKVEIK

SEQ ID NO: 13 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSYSPTFGQGTKVEIK

SEQ ID NO: 14 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQTEDEAEYYCSSYTSSSTFVFGPGTKVTVL

SEQ ID NO: 15 - protein, 107 aa, synthetic construct
DIQMTQSPSSLSASVGDRVTITCRASQSISNYLNWYQQKPGKAPNLLIYAASSLQSGVPSRFSGSGSGTDFTLTISSLQPEDFATYYCQQSYSTPPTFGPGTKVDIK

SEQ ID NO: 16 - protein, 107 aa, synthetic construct
DIQMTQSPSSLSASVGDRVTITCRASQSISRYLNWYQQKPGKAPKLLIYAASSLQSGVPSRFSGSGSGTDFTLTISSLQPEDFATYYCQQSYTTPLTFGQGTKVEIK

SEQ ID NO: 17 - protein, 107 aa, synthetic construct
DIQMTQSPSSLSASVGDRVTITCRASQTISNYLNWYQQKPGKAPKLLIYAASSLQSGVPSRFSGSGSGTDFTLTISSLQPEDFATYYCQQSYSTPLTFGGGTKVEIK

SEQ ID NO: 18 - protein, 107 aa, synthetic construct
EIVLTQSPGTLSLSPGERVTLSCRASQNIISNYLAWYQQKPGQAPRLLIYGASSRATGIPDRFSGSGSGTDFTLTISSLQPEDFAVYCHQYGSIPLTFGGGTKVEIK

SEQ ID NO: 19 - protein, 110 aa, synthetic construct
QSALTQPPSASGSPGQSVTISCTGTSSDVGGYNYVSWFQQHPGKAPKLIIYEVTKRPSGVPDRFSGSKSGNAASLTVSGLQAEDEADYYCSSYAGSNNLLFGGGTKLTVL

SEQ ID NO: 20 - protein, 8 aa, synthetic construct
GGSISSYF

SEQ ID NO: 21 - protein, 7 aa, synthetic construct
IHNSGTT

SEQ ID NO: 22 - protein, 7 aa, synthetic construct
IHYSGTT

SEQ ID NO: 23 - protein, 13 aa, synthetic construct
ARGRGMATLPFDY

SEQ ID NO: 24 - protein, 6 aa, synthetic construct
QSISSY

SEQ ID NO: 25 - protein, 6 aa, synthetic construct
QDITNY

SEQ ID NO: 26 - protein, 7 aa, synthetic construct
QSVSSTY

SEQ ID NO: 27 - protein, 12 aa, synthetic construct
QSVLYSSKNKNY

SEQ ID NO: 28 - protein, 6 aa, synthetic construct
QSISSW

SEQ ID NO: 29 - protein, 9 aa, synthetic construct
SSDVGGYNY

SEQ ID NO: 30 - protein, 6 aa, synthetic construct
QSISNY

SEQ ID NO: 31 - protein, 6 aa, synthetic construct
QSISRY

SEQ ID NO: 32 - protein, 6 aa, synthetic construct
QTISNY

SEQ ID NO: 33 - protein, 7 aa, synthetic construct
QNIISNY

SEQ ID NO: 34-39 - skipped (6 entries)

SEQ ID NO: 40 - protein, 9 aa, synthetic construct
QQSYSTPLT

SEQ ID NO: 41 - protein, 9 aa, synthetic construct
QQYDNLPYT

SEQ ID NO: 42 - protein, 9 aa, synthetic construct
QQYGSSPWT

SEQ ID NO: 43 - protein, 9 aa, synthetic construct
QQYYSTPWT

SEQ ID NO: 44 - protein, 9 aa, synthetic construct
QQYSSYSPT

SEQ ID NO: 45 - protein, 10 aa, synthetic construct
SSYTSSSTFV

SEQ ID NO: 46 - protein, 9 aa, synthetic construct
QQSYSTPPT

SEQ ID NO: 47 - protein, 9 aa, synthetic construct
QQSYTTPLT

SEQ ID NO: 48 - protein, 9 aa, synthetic construct
HQYGSIPLT

SEQ ID NO: 49 - protein, 10 aa, synthetic construct
SSYAGSNNLL

SEQ ID NO: 50 - protein, 25 aa, synthetic construct
QVQLQESGPGLVKPSETLSLTCTVS

SEQ ID NO: 51 - protein, 17 aa, synthetic construct
WTWIRQPPGKRLEWIGY

SEQ ID NO: 52 - protein, 17 aa, synthetic construct
WSWIRQPPGKGLEWIGY

SEQ ID NO: 53 - protein, 17 aa, synthetic construct
WTWIRQPPGKGLEWIGY

SEQ ID NO: 54 - protein, 17 aa, synthetic construct
WSWIRQPPGKRLEWIGY

SEQ ID NO: 55 - protein, 38 aa, synthetic construct
NQNPSLKSRVTISIDTSKNHFSLKLSSVTAADTAVYYC

SEQ ID NO: 56 - protein, 38 aa, synthetic construct
NYNPSLKSRVTISVDTSKNQFSLKLSSVTAADTAVYYC

SEQ ID NO: 57 - protein, 38 aa, synthetic construct
NQNPSLKSRVTISVDTSKNQFSLKLSSVTAADTAVYYC

SEQ ID NO: 58 - protein, 38 aa, synthetic construct
NYNPSLKSRVTISIDTSKNQFSLKLSSVTAADTAVYYC

SEQ ID NO: 59 - protein, 38 aa, synthetic construct
NYNPSLKSRVTISVDTSKNHFSLKLSSVTAADTAVYYC

SEQ ID NO: 60 - protein, 38 aa, synthetic construct
NQNPSLKSRVTISIDKSKNHFSLKLSSVTAADTAVYYC

SEQ ID NO: 61 - protein, 38 aa, synthetic construct
NQNPSLKSRVTISVDKSKNQFSLKLSSVTAADTAVYYC

SEQ ID NO: 62 - protein, 11 aa, synthetic construct
WGQGTLVTVSS

SEQ ID NO: 63 - protein, 26 aa, synthetic construct [sequence omitted]

SEQ ID NO: 64 - protein, 26 aa, synthetic construct
DIQMTQSPSSLSASVGDRVTITCQAS

SEQ ID NO: 65 - protein, 26 aa, synthetic construct [sequence omitted]

SEQ ID NO: 66 - protein, 26 aa, synthetic construct
DIVMTQSPDSLAVSLGERATINCKSS

SEQ ID NO: 67 - protein, 26 aa, synthetic construct
DIQMTQSPSTLSASVGDRVTITCRAS

SEQ ID NO: 68 - protein, 25 aa, synthetic construct
QSALTQPASVSGSPGQSITISCTGT

SEQ ID NO: 69 - protein, 26 aa, synthetic construct
EIVLTQSPGTLSLSPGERVTLSCRAS

SEQ ID NO: 70 - protein, 25 aa, synthetic construct
QSALTQPPSASGSPGQSVTISCTGT

SEQ ID NO: 71 - protein, 17 aa, synthetic construct
LNWYQQKPGKAPKLLIY

SEQ ID NO: 72 - protein, 17 aa, synthetic construct
LAWYQQKPGQAPRLLIY

SEQ ID NO: 73 - protein, 17 aa, synthetic construct
LAWYQQKPGKAPKLLIY

SEQ ID NO: 74 - protein, 17 aa, synthetic construct
VSWYQQHPGKAPKLMIY

SEQ ID NO: 75 - protein, 17 aa, synthetic construct
LNWYQQKPGKAPNLLIY

SEQ ID NO: 76 - protein, 17 aa, synthetic construct
VSWFQQHPGKAPKLIIY

SEQ ID NO: 77 - protein, 36 aa, synthetic construct
SLQSGVPSRFSGSGSGTDFTLTISSLQPEDFATYYC

SEQ ID NO: 78 - protein, 36 aa, synthetic construct
NLETGVPSRFSGSGSGTDFTFTISSLQPEDIATYYC

SEQ ID NO: 79 - protein, 36 aa, synthetic construct [sequence omitted]

SEQ ID NO: 80 - protein, 36 aa, synthetic construct
SLQSGVPSRFSGSGSGTDFTLTISSLQAEDVAVYYC

SEQ ID NO: 81 - protein, 36 aa, synthetic construct
SLESGVPSRFSGSGSGTEFTLTISSLQPDDFATYYC

SEQ ID NO: 82 - protein, 36 aa, synthetic construct
NRPSGVSNRFSGSKSGNTASLTISGLQTEDEAEYYC

SEQ ID NO: 83 - protein, 35 aa, synthetic construct
SRATGIPDRFSGSGSGTDFTLTISSLQPEDFAVYC

SEQ ID NO: 84 - protein, 36 aa, synthetic construct
KRPSGVPDRFSGSKSGNAASLTVSGLQAEDEADYYC

SEQ ID NO: 85 - protein, 10 aa, synthetic construct
FGGGTKVEIK

SEQ ID NO: 86 - protein, 10 aa, synthetic construct
FGQGTKVEIR

SEQ ID NO: 87 - protein, 10 aa, synthetic construct
FGQGTKVEIK

SEQ ID NO: 88 - protein, 10 aa, synthetic construct
FGPGTKVTVL

SEQ ID NO: 89 - protein, 10 aa, synthetic construct
FGPGTKVDIK

SEQ ID NO: 90 - protein, 10 aa, synthetic construct
FGGGTKLTVL

SEQ ID NO: 91 - protein, 119 aa, synthetic construct
QVQLQESGPGLVKPSETLSLTCTVSGGSISSYFWSWIRQPPGKGLEWIGYIHNSGTTNQNPSLKSRVTISVDKSKNQFSLKLSSVTAADTAVYYCARGRGMATLPFDYWGQGTLVTVSS

SEQ ID NO: 92 - protein, 107 aa, synthetic construct [sequence omitted]

SEQ ID NO: 93 - protein, 119 aa, synthetic construct
QVQLQESGPGLVKPSETLSLTCTVSGGSISSYFWSWIRQPPGKGLEWIGYIHYSGTTNQNPSLKSRVTISVDKSKNQFSLKLSSVTAADTAVYYCARGRGMATLPFDYWGQGTLVTVSS

SEQ ID NO: 94 - protein, 107 aa, synthetic construct [sequence omitted]

SEQ ID NO: 95 - protein, 118 aa, synthetic construct
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 96 - protein, 112 aa, synthetic construct
DVVMTQTPLSLPVSLGDQASISCRSSQSLVNSNGNTYLRWYLQKPGQSPKVLIYKVSNRVSGVPDRFSGSGSGTDFTLKISRVEAEDLGVYFCSQSTHVPWTFGGGTKLEIK

SEQ ID NO: 97 - protein, 362 aa, synthetic construct
GSHHHHHHGSDYKDDDDKGSQDDEFFHELPETFPSDPPEPLPHFLIEPEEAYIVKNKPVNLYCKASPATQIYFKCNSEWVHQKDHVVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPAEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTSWSKWSTCGTECTHWRRRECTAPAPKNGGKDCDGLVLQSKNCTDGLCMQAAPDSDDVALYGS

SEQ ID NO: 98 - protein, 362 aa, synthetic construct
GSHHHHHHGSDYKDDDDKGSQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYGS

SEQ ID NO: 99 - protein, 931 aa, Homo sapiens
MRKGLRATAARCGLGLGYLLQMLVLPALALLSASGTGSAAQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYVGIVIAVIVCLAISVVVALFVYRKNHRDFESDIIDSSALNGGFQPVNIKAARQDLLAVPPDLTSAAAMYRGPVYALHDVSDKIPMTNSPILDPLPNLKIKVYNTSGAVTPQDDLSEFTSKLSPQMTQSLLENEALSLKNQSLARQTDPSCTAFGSFNSLGGHLIVPNSGVSLLIPAGAIPQGRVYEMYVTVHRKETMRPPMDDSQTLLTPVVSCGPPGALLTRPVVLTMHHCADPNTEDWKILLKNQAAQGQWEDVVVVGEENFTTPCYIQLDAEACHILTENLSTYALVGHSTTKAAAKRLKLAIFGPLCCSSLEYSIRVYCLDDTQDALKEILHLERQMGGQLLEEPKALHFKGSTHNLRLSIHDIAHSLWKSKLLAKYQEIPFYHVWSGSQRNLHCTFTLERFSLNTVELVCKLCVRQVEGEGQIFQLNCTVSEEPTGIDLPLLDPANTITTVTGPSAFSIPLPIRQKLCSSLDAPQTRGHDWRMLAHKLNLDRYLNYFATKSSPTGVILDLWEAQNFPDGNLSMLAAVLEEMGRHETVVSLAAEGQY

SEQ ID NO: 100 - protein, 931 aa, Mus musculus
MRKGLRATAARCGLGLGYLLQMLVLPALALLSASGTGSAAQDDEFFHELPETFPSDPPEPLPHFLIEPEEAYIVKNKPVNLYCKASPATQIYFKCNSEWVHQKDHVVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPAEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTSWSKWSTCGTECTHWRRRECTAPAPKNGGKDCDGLVLQSKNCTDGLCMQAAPDSDDVALYVGIVIAVTVCLAITVVVALFVYRKNHRDFESDIIDSSALNGGFQPVNIKAARQDLLAVPPDLTSAAAMYRGPVYALHDVSDKIPMTNSPILDPLPNLKIKVYNSSGAVTPQDDLAEFSSKLSPQMTQSLLENEALNLKNQSLARQTDPSCTAFGTFNSLGGHLIIPNSGVSLLIPAGAIPQGRVYEMYVTVHRKENMRPPMEDSQTLLTPVVSCGPPGALLTRPVILTLHHCADPSTEDWKIQLKNQAVQGQWEDVVVVGEENFTTPCYIQLDAEACHILTENLSTYALVGQSTTKAAAKRLKLAIFGPLCCSSLEYSIRVYCLDDTQDALKEVLQLERQMGGQLLEEPKALHFKGSIHNLRLSIHDIAHSLWKSKLLAKYQEIPFYHIWSGSQRNLHCTFTLERLSLNTVELVCKLCVRQVEGEGQIFQLNCTVSEEPTGIDLPLLDPASTITTVTGPSAFSIPLPIRQKLCSSLDAPQTRGHDWRMLAHKLNLDRYLNYFATKSSPTGVILDLWEAQNFPDGNLSMLAAVLEEMGRHETVVSLAAEGQY

SEQ ID NO: 101 - protein, 340 aa, synthetic construct
QDDEFFHELPETFPSDPPEPLPHFLIEPEEAYIVKNKPVNLYCKASPATQIYFKCNSEWVHQKDHVVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPAEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTSWSKWSTCGTECTHWRRRECTAPAPKNGGKDCDGLVLQSKNCTDGLCMQAAPDSDDVALY

SEQ ID NO: 102 - protein, 340 aa, synthetic construct
QDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALY

SEQ ID NO: 103 - protein, 362 aa, synthetic construct
GSHHHHHHGSDYKDDDDKGSQDDDFFHELPETFPSDPPEPLPHFLIEPEEAYIVKNKPVNLYCKASPATQIYFKCNSEWVHQKDHVVDERVDETSGLIVREVSIEISRQQVEELFGPEDYWCQCVAWSSAGTTKSRKAYVRIAYLRKTFEQEPLGKEVSLEQEVLLQCRPPEGIPMAEVEWLKNEDIIDPVEDRNFYITIDHNLIIKQARLSDTANYTCVAKNIVAKRKSTTATVIVYVNGGWSTWAEWSVCNSRCGRGYQKRTRTCTNPAPLNGGAFCEGQSVQKIACTTLCPVDGRWTSWSKWSTCGTECTHWRRRECTAPAPKNGGKDCDGLVLQSKNCTDGLCMQAAPDSDDVALYGS

SEQ ID NO: 104 - protein, 362 aa, synthetic construct; identical to SEQ ID NO: 98

SEQ ID NO: 105 - protein, 303 aa, synthetic construct
GSHHHHHHGSDYKDDDDKGSQQSATVANPVPGANPDLLPHFLVEPEDVYIVKNKPVLLVCKAVPATQIFFKCNGEWVRQVDHVIERSTDGSSGLPTMEVRINVSRQQVEKVFGLEEYWCQCVAWSSSGTTKSQKAYIRIAYLRKNFEQEPLAKEVSLEQGIVLPCRPPEGIPPAEVEWLRNEDLVDPSLDPNVYITREHSLVVRQARLADTANYTCVAKNIVARRRSASAAVIVYVDGSWSPWSKWSACGLDCTHWRSRECSDPAPRNGGEECQGTDLDTRNCTSDLCVHTASGPEDVALYGS

SEQ ID NO: 106 - protein, 373 aa, synthetic construct
GSHHHHHHGSDYKDDDDKGSGTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQNKKTLSDPNSHLLEASGDAALYGS

SEQ ID NO: 107 - protein, 351 aa, synthetic construct
GSQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYGSKHHHHHH

SEQ ID NO: 108 - protein, 369 aa, synthetic construct
GSHHHHHHGSDYKDDDDKGSARGTDNGEALPESIPSAPGTLPHFIEEPDDAYIIKSNPIALRCKARPAMQIFFKCNGEWVHQNEHVSEETLDESSGLKVREVFINVTRQQVEDFHGPEDYWCQCVAWSHLGTSKSRKASVRIAYLRKNFEQDPQGREVPIEGMIVLHCRPPEGVPAAEVEWLKNEEPIDSEQDENIDTRADHNLIIRQARLSDSGNYTCMAANIVAKRRSLSATVVVYVNGGWSSWTEWSACNVRCGRGWQKRSRTCTNPAPLNGGAFCEGMSVQKITCTSLCPVDGSWEVWSEWSVCSPECEHLRIRECTAPPPRNGGKFCEGLSQESENCTDGLCILDKKPLHEIKPQSIENASDGS

SEQ ID NO: 109 - protein, 1094 aa, synthetic construct
GSHHHHHHGSDYKDDDDKGSHLQVTGFQIKAFTALRFLSEPSDAVTMRGGNVLLDCSAESDRGVPVIKWKKDGIHLALGMDERKQQLSNGSLLIQNILHSRHHKPDEGLYQCEASLGDSGSIISRTAKVAVAGPLRFLSQTESVTAFMGDTVLLKCEVIGEPMPTIHWQKNQQDLTPIPGDSRVVVLPSGALQISRLQPGDIGIYRCSARNPASSRTGNEAEVRILSDPGLHRQLYFLQRPSNVVAIEGKDAVLECCVSGYPPPSFTWLRGEEVIQLRSKKYSLLGGSNLLISNVTDDDSGMYTCVVTYKNENISASAELTVLVPPWFLNHPSNLYAYESMDIEFECTVSGKPVPTVNWMKNGDVVIPSDYFQIVGGSNLRILGVVKSDEGFYQCVAENEAGNAQTSAQLIVPKPAIPSSSVLPSAPRDVVPVLVSSRFVRLSWRPPAEAKGNIQTFTVFFSREGDNRERALNTTQPGSLQLTVGNLKPEAMYTFRVVAYNEWGPGESSQPIKVATQPELQVPGPVENLQAVSTSPTSILITWEPPAYANGPVQGYRLFCTEVSTGKEQNIEVDGLSYKLEGLKKFTEYSLRFLAYNRYGPGVSTDDITVVTLSDVPSAPPQNVSLEVVNSRSIKVSWLPPPSGTQNGFITGYKIRHRKTTRRGEMETLEPNNLWYLFTGLEKGSQYSFQVSAMTVNGTGPPSNWYTAETPENDLDESQVPDQPSSLHVRPQTNCIIMSWTPPLNPNIVVRGYIIGYGVGSPYAETVRVDSKQRYYSIERLESSSHYVISLKAFNNAGEGVPLYESATTRSITDPTDPVDYYPLLDDFPTSVPDLSTPMLPPVGVQAVALTHDAVRVSWADNSVPKNQKTSEVRLYTVRWRTSFSASAKYKSEDTTSLSYTATGLKPNTMYEFSVMVTKNRRSSTWSMTAHATTYEAAPTSAPKDLTVITREGKPRAVIVSWQPPLEANGKITAYILFYTLDKNIPIDDWIMETISGDRLTHQIMDLNLDTMYYFRIQARNSKGVGPLSDPILFRTLKVEHPDKMANDQGRHGDGGYWPVDTNLIDRSTLNEPPIGQMHPPHGSVTPQKNSNGS

SEQ ID NO: 110 - protein, 931 aa, synthetic construct
MRKGLRATAARCGLGLGYLLQMLVLPALALLSASGTGSAAQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYVGIVIAVIVCLAISVVVALFVYRKNHRDFESDIIDSSALNGGFQPVNIKAARQDLLAVPPDLTSAAAMYRGPVYALHDVSDKIPMTNSPILDPLPNLKIKVYNTSGAVTPQDDLSEFTSKLSPQMTQSLLENEALSLKNQSLARQTDPSCTAFGSFNSLGGHLIVPNSGVSLLIPAGAIPQGRVYEMYVTVHRKETMRPPMDDSQTLLTPVVSCGPPGALLTRPVVLTMHHCADPNTEDWKILLKNQAAQGQWEDVVVVGEENFTTPCYIQLDAEACHILTENLSTYALVGHSTTKAAAKRLKLAIFGPLCCSSLEYSIRVYCLDDTQDALKEILHLERQMGGQLLEEPKALHFKGSTHNLRLSIHDIAHSLWKSKLLAKYQEIPFYHVWSGSQRNLHCTFTLERFSLNTVELVCKLCVRQVEGEGQIFQLNCTVSEEPTGIDLPLLDPANTITMVTGPSAFSIPLPIRQKLCSSLDAPQTRGHDWRMLAHKLNLDRYLNYFATKSSPTGVILDLWEAQNFPDGNLSMLAAVLEEMGRHETVVSLAAEGQY

SEQ ID NO: 111 - protein, 214 aa, synthetic construct
GSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVGSHHHHHH

SEQ ID NO: 112 - protein, 119 aa, synthetic construct
QVQLQESGPGLVKPSETLSLTCTVSGGSTGSYYWSWIRQPPGKGLEWIGYTHYSGHTNQNPSLKSRVTMSVDSSKSQLSLKLTSVTAADTAVYYCARGRGMATLPFDSWGQGILVIVSS

SEQ ID NO: 113 - protein, 119 aa, synthetic construct
QVQLQESGPGLVKPSETLSLTCTVSGGSIGSYFWSWIRQTPGKGLEWIGYIHYSGSSNYNPSLKSRVTISVDTFKNQFSLKLTSVTAADTAVYYCARGRGMATLPFDYWGQGTLVTVSS

SEQ ID NO: 114 - protein, 107 aa, synthetic construct [sequence omitted]

SEQ ID NO: 115 - protein, 8 aa, synthetic construct
GGSTGSYY

SEQ ID NO: 116 - protein, 8 aa, synthetic construct
GGSIGSYF

SEQ ID NO: 117 - protein, 7 aa, synthetic construct
THYSGHT

SEQ ID NO: 118 - protein, 7 aa, synthetic construct
IHYSGSS

SEQ ID NO: 119 - protein, 13 aa, synthetic construct
ARGRGMATLPFDS

SEQ ID NO: 120 - protein, 13 aa, synthetic construct
ARGRGMATLPFDY

SEQ ID NO: 121 - protein, 9 aa, synthetic construct
QQSYSTPLT

SEQ ID NO: 122 - protein, 125 aa, synthetic construct
QVQLLETGGGLVQPGGSLRLSCAASGFTFSSYSMNWVRQAPGKGLEWVSSISSSSSYIYYADSVKGRFTISRDNAKNSLYLQMNSLRAEDTAVYYCARDPYSGYEFVSEPYFDYWGQGTLVTVSS

SEQ ID NO: 123 - protein, 119 aa, synthetic construct
EVQLVESGGVVVQPGGSLRLSCAASGFTFDDYAMHWVRQAPGKGLEWVSLISWDGGSTYYADSVKGRFTISRDNSKNSLYLQMNSLRAEDTALYYCAKDSIQLWSFDYWGQGTLVTVSS

SEQ ID NO: 124 - protein, 125 aa, synthetic construct
QVTLKESGAEVKKPGASVKVSCKASGYTFTGYYMHWVRQAPGQGLEWMGRINPNSGGTNYAQKFQGRVTMTRDTSISTAYMELSRLRSDDTAVYYCAREEDCSSTSCYGDAFDIWGQGTMVTVSS

SEQ ID NO: 125 - protein, 116 aa, synthetic construct
QVQLQQSGGGLVKPGGSLRLSCAASGFTFSDYYMSWIRQAPGKGLEWVSYISSSSSYTNYADSVKGRFTISRDNAKNSLYLQMNSLRAEDTAVYYCARGGGGMDVWGQGTMVTVSS

SEQ ID NO: 126 - protein, 124 aa, synthetic construct
QVQLVESGAEVKKPGASVKVSCKASGYTFTGYYMHWVRQAPGQGLEWMGRINPNSGGTNYAQKFQGRVTMTRDTSISTAYMELSRLRSDDTAVYYCAREGDDSSGWLGGAFDIWGQGTMVTVSS

SEQ ID NO: 127 - protein, 125 aa, synthetic construct
EVQLLETGGGLVQPGRSLRLSCAASGFTFDDYAMHWVRQAPGKGLEWVSGISWNSGSIGYADSVKGRFTISRDNAKNSLYLQMNSLRAEDTALYYCAKDSYGDYFRGYYYGMDVWGQGTMVTVSS

SEQ ID NO: 128 - protein, 122 aa, synthetic construct
EVQLLESAGGVVQLGRSLRLSCAASGFTFSNYGMHWVRQAPGKGLEWVAIIYFDGRNKYYAGSVGGRFTISRDNSKNTLYLQMDSLRSDDTAVYYCARPLFPWLRVDGLDVWGHGTMVTVSS

SEQ ID NO: 129 - protein, 121 aa, synthetic construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQRRGSGTRGMDVWGQGTTVTVSS

SEQ ID NO: 130 - protein, 119 aa, synthetic construct
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARGRGGSYYFDYWGQGTLVTVSS

SEQ ID NO: 131 - protein, 126 aa, synthetic construct
QVQLVESGGGLVQPGGSLRLSCSASGFTFINYAMSWVRQAPGKGLEWVSTISGSGASIYYADSVKGRFTISRDNSKNTVYLQMNSLRAEDTAVYYCAKGAYYDSSGYYALDAFDIWGQGTMVTVSS

SEQ ID NO: 132 - protein, 111 aa, synthetic construct
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSNHWVFGGGTKVTVL

SEQ ID NO: 133 - protein, 110 aa, synthetic construct
QSALTQPASVSGSPGQSITISCTGARSDVGGYDYVSWFQQHPGKVPKLMIYDVNNRPSGVSNRFSGSKSGNTASLTISGLQAEDEADYYCSSFTDSSTYVFGTGTKVTVL

SEQ ID NO: 134 - protein, 112 aa, synthetic construct
QSVLTQPHSVSESPGKTVTISCTRSSGSIVSNYVQWYQQRPGSSPTTVIYEDNQRPSGVPDRFSGSIDRSSNSASLTISGLKTEDEGDYYCQSYDKTSRVILFGEGTKVTVL

SEQ ID NO: 135 - protein, 111 aa, synthetic construct
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSNHVVFGGGTKVTVL

SEQ ID NO: 136 - protein, 111 aa, synthetic construct
NFMLTQPHSVLESPGKTVTIPCTRSSGSIASNYVQWYQQRPGSAPTAVIFEDNVRPSGVPDRFSGSVDSSSNSASLTISGLKTEDEGDFYCQSYDSSGHVVFGGGTKVTVL

SEQ ID NO: 137 - protein, 112 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSASYVFGTGTKVTVL

SEQ ID NO: 138 - protein, 111 aa, synthetic construct
QSALTQPASVSGSPGQSITISCTGTSSDVGNYNLVSWYQQYPGKAPKLLIYEVIKRPSGISGRFSGSKSGNTASLTISGLQAEDEADYYCSSYTSSSTDYVFGTGTKVTVL

SEQ ID NO: 139 - protein, 106 aa, synthetic construct
QAGLTQPPSVSVSPGQTASITCSGKKLGDKYTSWYQQKPGQSPLLVIYQDNKRPSGVPGRFSGSNSGNTATLTISGTQPMDEADYYCQAWDSSTVIFGGGTKVTVL

SEQ ID NO: 140 - protein, 110 aa, synthetic construct
QSVLTQPPSASGTPGQRVTISCSGSSSNIGSNTVNWYQQVPGTAPKLLIYSNNQRPSGVPDRFSGSKSGTSASLAISGLQSEDEADYYCAAWDDSLNGWVFGGGTKVTVL

SEQ ID NO: 141 - protein, 105 aa, synthetic construct
QSVLTQPPSVSVSAGQTASITCSGDMLGGKYASWYQHKPGQSPVLVIYQDARRPSGISERFSGSSSGNTATLTISGTQAMDEADYYCQAWGSTAVFGGGTKVTVL

SEQ ID NO: 142 - protein, 8 aa, synthetic construct
GFTFSSYS

SEQ ID NO: 143 - protein, 8 aa, synthetic construct
GFTFDDYA

SEQ ID NO: 144 - protein, 8 aa, synthetic construct
GYTFTGYY

SEQ ID NO: 145 - protein, 8 aa, synthetic construct
GFTFSDYY

SEQ ID NO: 146 - protein, 8 aa, synthetic construct
GFTFSNYG

SEQ ID NO: 147 - protein, 8 aa, synthetic construct
GYSFTSYW

SEQ ID NO: 148 - protein, 9 aa, synthetic construct
GGSISSSNW

SEQ ID NO: 149 - protein, 8 aa, synthetic construct
GFTFINYA

SEQ ID NO: 150 - protein, 8 aa, synthetic construct
ISSSSSYI

SEQ ID NO: 151 - protein, 8 aa, synthetic construct
ISWDGGST

SEQ ID NO: 152 - protein, 8 aa, synthetic construct
INPNSGGT

SEQ ID NO: 153 - protein, 8 aa, synthetic construct
ISSSSSYT

SEQ ID NO: 154 - protein, 8 aa, synthetic construct
ISWNSGSI

SEQ ID NO: 155 - protein, 8 aa, synthetic construct
IYFDGRNK

SEQ ID NO: 156 - protein, 8 aa, synthetic construct
IYPGDSDT

SEQ ID NO: 157 - protein, 7 aa, synthetic construct
IYHSGST

SEQ ID NO: 158 - protein, 8 aa, synthetic construct
ISGSGASI

SEQ ID NO: 159 - protein, 18 aa, synthetic construct
ARDPYSGYEFVSEPYFDY

SEQ ID NO: 160 - protein, 12 aa, synthetic construct
AKDSIQLWSFDY

SEQ ID NO: 161 - protein, 18 aa, synthetic construct
AREEDCSSTSCYGDAFDI

SEQ ID NO: 162 - protein, 9 aa, synthetic construct
ARGGGGMDV

SEQ ID NO: 163 - protein, 17 aa, synthetic construct
AREGDDSSGWLGGAFDI

SEQ ID NO: 164 - protein, 18 aa, synthetic construct
AKDSYGDYFRGYYYGMDV

SEQ ID NO: 165 - protein, 15 aa, synthetic construct
ARPLFPWLRVDGLDV

SEQ ID NO: 166 - protein, 14 aa, synthetic construct
ARQRRGSGTRGMDV

SEQ ID NO: 167 - protein, 12 aa, synthetic construct
ARGRGGSYYFDY

SEQ ID NO: 168 - protein, 19 aa, synthetic construct
AKGAYYDSSGYYALDAFDI

SEQ ID NO: 169 - protein, 8 aa, synthetic construct
SGSIASNY

SEQ ID NO: 170 - protein, 9 aa, synthetic construct
RSDVGGYDY

SEQ ID NO: 171 - protein, 8 aa, synthetic construct
SGSIVSNY

SEQ ID NO: 172 - protein, 9 aa, synthetic construct
SSNIGAGYD

SEQ ID NO: 173 - protein, 9 aa, synthetic construct
SSDVGNYNL

SEQ ID NO: 174 - protein, 6 aa, synthetic construct
KLGDKY

SEQ ID NO: 175 - protein, 8 aa, synthetic construct
SSNIGSNT

SEQ ID NO: 176 - protein, 6 aa, synthetic construct
MLGGKY

SEQ ID NO: 177-183 - skipped (7 entries)

SEQ ID NO: 184 - protein, 10 aa, synthetic construct
QSYDSSNHWV

SEQ ID NO: 185 - protein, 10 aa, synthetic construct
SSFTDSSTYV

SEQ ID NO: 186 - protein, 11 aa, synthetic construct
QSYDKTSRVIL

SEQ ID NO: 187 - protein, 10 aa, synthetic construct
QSYDSSNHVV

SEQ ID NO: 188 - protein, 10 aa, synthetic construct
QSYDSSGHVV

SEQ ID NO: 189 - protein, 12 aa, synthetic construct
QSYDSSLSASYV

SEQ ID NO: 190 - protein, 11 aa, synthetic construct
SSYTSSSTDYV

SEQ ID NO: 191 - protein, 9 aa, synthetic construct
QAWDSSTVI

SEQ ID NO: 192 - protein, 11 aa, synthetic construct
AAWDDSLNGWV

SEQ ID NO: 193 - protein, 8 aa, synthetic construct
QAWGSTAV

SEQ ID NO: 194 - protein, 350 aa, synthetic construct
GSQDDDFFHELPETFPSDPPEPLPHFLIEPEEAYIVKNKPVNLYCKASPATQIYFKCNSEWVHQKDHIVDERVDETSGLIVREVSIEISRQQVEELFGPEDYWCQCVAWSSAGTTKSRKAYVRIAYLRKTFEQEPLGKEVSLEQEVLLQCRPPEGIPVAEVEWLKNEDIIDPVEDRNFYITIDHNLIIKQARLSDTANYTCVAKNIVAKRKSTTATVIVYVNGGWSTWTEWSVCNSRCGRGYQKRTRTCTNPAPLNGGAFCEGQSVQKIACTTLCPVDGRWTPWSKWSTCGTECTHWRRRECTAPAPKNGGKDCDGLVLQSKNCTDGLCMQTAPDSDDVALYGSHHHHHH

SEQ ID NO: 195 - protein, 17 aa, synthetic construct
WSWIRQTPGKGLEWIGY

SEQ ID NO: 196 - protein, 38 aa, synthetic construct
NQNPSLKSRVTMSVDSSKSQLSLKLTSVTAADTAVYYC

SEQ ID NO: 197 - protein, 38 aa, synthetic construct
NYNPSLKSRVTISVDTFKNQFSLKLTSVTAADTAVYYC

SEQ ID NO: 198 - protein, 11 aa, synthetic construct
WGQGILVIVSS

SEQ ID NO: 199 - protein, 25 aa, synthetic construct
QVQLLETGGGLVQPGGSLRLSCAAS

SEQ ID NO: 200 - protein, 17 aa, synthetic construct
MNWVRQAPGKGLEWVSS

SEQ ID NO: 201 - protein, 38 aa, synthetic construct
YYADSVKGRFTISRDNAKNSLYLQMNSLRAEDTAVYYC

SEQ ID NO: 202 - protein, 25 aa, synthetic construct
EVQLVESGGVVVQPGGSLRLSCAAS

SEQ ID NO: 203 - protein, 17 aa, synthetic construct
MHWVRQAPGKGLEWVSL

SEQ ID NO: 204 - protein, 38 aa, synthetic construct
YYADSVKGRFTISRDNSKNSLYLQMNSLRAEDTALYYC

SEQ ID NO: 205 - protein, 25 aa, synthetic construct
QVTLKESGAEVKKPGASVKVSCKAS

SEQ ID NO: 206 - protein, 17 aa, synthetic construct
MHWVRQAPGQGLEWMGR

SEQ ID NO: 207 - protein, 38 aa, synthetic construct
NYAQKFQGRVTMTRDTSISTAYMELSRLRSDDTAVYYC

SEQ ID NO: 208 - protein, 11 aa, synthetic construct
WGQGTMVTVSS

SEQ ID NO: 209 - protein, 25 aa, synthetic construct
QVQLQQSGGGLVKPGGSLRLSCAAS

SEQ ID NO: 210 - protein, 17 aa, synthetic construct
MSWIRQAPGKGLEWVSY

SEQ ID NO: 211 - protein, 38 aa, synthetic construct
NYADSVKGRFTISRDNAKNSLYLQMNSLRAEDTAVYYC

SEQ ID NO: 212 - protein, 25 aa, synthetic construct
QVQLVESGAEVKKPGASVKVSCKAS

SEQ ID NO: 213 - protein, 25 aa, synthetic construct
EVQLLETGGGLVQPGRSLRLSCAAS

SEQ ID NO: 214 - protein, 17 aa, synthetic construct
MHWVRQAPGKGLEWVSG

SEQ ID NO: 215 - protein, 38 aa, synthetic construct
GYADSVKGRFTISRDNAKNSLYLQMNSLRAEDTALYYC

SEQ ID NO: 216 - protein, 25 aa, synthetic construct
EVQLLESAGGVVQLGRSLRLSCAAS

SEQ ID NO: 217 - protein, 17 aa, synthetic construct
MHWVRQAPGKGLEWVAI

SEQ ID NO: 218 - protein, 38 aa, synthetic construct
YYAGSVGGRFTISRDNSKNTLYLQMDSLRSDDTAVYYC

SEQ ID NO: 219 - protein, 11 aa, synthetic construct
WGHGTMVTVSS

SEQ ID NO: 220 - protein, 25 aa, synthetic construct
QVQLVQSGAEVKKPGESLKISCKGS

SEQ ID NO: 221 - protein, 17 aa, synthetic construct
IGWVRQMPGKGLEWMGI

SEQ ID NO: 222 - protein, 38 aa, synthetic construct
RYSPSFQGQVTISADKSISTAYLQWSSLKASDTAMYYC

SEQ ID NO: 223 - protein, 11 aa, synthetic construct
WGQGTTVTVSS

SEQ ID NO: 224 - protein, 25 aa, synthetic construct
QVQLQESGPGLVKPSGTLSLTCAVS

SEQ ID NO: 225 - protein, 17 aa, synthetic construct
WSWVRQPPGKGLEWIGE

SEQ ID NO: 226 - protein, 38 aa, synthetic construct
NYNPSLKSRVTISVDKSKNQFSLKLSSVTAADTAVYYC

SEQ ID NO: 227 - protein, 25 aa, synthetic construct
QVQLVESGGGLVQPGGSLRLSCSAS

SEQ ID NO: 228 - protein, 17 aa, synthetic construct
MSWVRQAPGKGLEWVST

SEQ ID NO: 229 - protein, 38 aa, synthetic construct
YYADSVKGRFTISRDNSKNTVYLQMNSLRAEDTAVYYC

SEQ ID NO: 230 - protein, 25 aa, synthetic construct
NFMLTQPHSVSESPGKTVTISCTGS

SEQ ID NO: 231 - protein, 25 aa, synthetic construct
QSALTQPASVSGSPGQSITISCTGA

SEQ ID NO: 232 - protein, 25 aa, synthetic construct
QSVLTQPHSVSESPGKTVTISCTRS

SEQ ID NO: 233 - protein, 25 aa, synthetic construct
NFMLTQPHSVLESPGKTVTIPCTRS

SEQ ID NO: 234 - protein, 25 aa, synthetic construct
QSVLTQPPSVSGAPGQRVTISCTGS

SEQ ID NO: 235 - protein, 25 aa, synthetic construct
QAGLTQPPSVSVSPGQTASITCSGK

SEQ ID NO: 236 - protein, 25 aa, synthetic construct
QSVLTQPPSASGTPGQRVTISCSGS

SEQ ID NO: 237 - protein, 25 aa, synthetic construct
QSVLTQPPSVSVSAGQTASITCSGD

SEQ ID NO: 238 - protein, 17 aa, synthetic construct
VQWYQQRPGSAPTTVIY

SEQ ID NO: 239 - protein, 17 aa, synthetic construct
VSWFQQHPGKVPKLMIY

SEQ ID NO: 240 - protein, 17 aa, synthetic construct
VQWYQQRPGSSPTTVIY

SEQ ID NO: 241 - protein, 17 aa, synthetic construct
VQWYQQRPGSAPTAVIF

SEQ ID NO: 242 - protein, 17 aa, synthetic construct
VHWYQQLPGTAPKLLIY

SEQ ID NO: 243 - protein, 17 aa, synthetic construct
VSWYQQYPGKAPKLLIY

SEQ ID NO: 244 - protein, 17 aa, synthetic construct
TSWYQQKPGQSPLLVIY

SEQ ID NO: 245 - protein, 17 aa, synthetic construct
VNWYQQVPGTAPKLLIY

SEQ ID NO: 246 - protein, 17 aa, synthetic construct
ASWYQHKPGQSPVLVIY

SEQ ID NO: 247 - protein, 38 aa, synthetic construct
QRPSGVPDRFSGSIDSSSNSASLTISGLKTEDEADYYC

SEQ ID NO: 248 - protein, 36 aa, synthetic construct
NRPSGVSNRFSGSKSGNTASLTISGLQAEDEADYYC

SEQ ID NO: 249 - protein, 38 aa, synthetic construct
QRPSGVPDRFSGSIDRSSNSASLTISGLKTEDEGDYYC

SEQ ID NO: 250 - protein, 38 aa, synthetic construct
VRPSGVPDRFSGSVDSSSNSASLTISGLKTEDEGDFYC

SEQ ID NO: 251 - protein, 36 aa, synthetic construct
NRPSGVPDRFSGSKSGTSASLAITGLQAEDEADYYC

SEQ ID NO: 252 - protein, 36 aa, synthetic construct
KRPSGISGRFSGSKSGNTASLTISGLQAEDEADYYC

SEQ ID NO: 253 - protein, 36 aa, synthetic construct
KRPSGVPGRFSGSNSGNTATLTISGTQPMDEADYYC

SEQ ID NO: 254 - protein, 36 aa, synthetic construct
QRPSGVPDRFSGSKSGTSASLAISGLQSEDEADYYC

SEQ ID NO: 255 - protein, 36 aa, synthetic construct
RRPSGISERFSGSSSGNTATLTISGTQAMDEADYYC

SEQ ID NO: 256 - protein, 10 aa, synthetic construct
FGGGTKVTVL

SEQ ID NO: 257 - protein, 10 aa, synthetic construct
FGTGTKVTVL

SEQ ID NO: 258 - protein, 10 aa, synthetic construct
FGEGTKVTVL

SEQ ID NO: 259 - protein, 41 aa, synthetic construct
YCKASPATQIYFKCNSEWVHQKDHIVDERVDETSGLIVREV

SEQ ID NO: 260 - protein, 41 aa, synthetic construct
RCRAFPATQIYFKCNGEWVSQNDHVTQEGLDEATGLRVREV

SEQ ID NO: 261 - protein, 41 aa, synthetic construct
VCKAVPATQIFFKCNGEWVRQVDHVIERSTDGSSGLPTMEV

SEQ ID NO: 262 - protein, 41 aa, synthetic construct
RCKARPAMQIFFKCNGEWVHQNEHVSEETLDESSGLKVREV

(19) United States
(12) Patent Application Publication
(10) Pub. No.: US 2024/0228604 A1
(43) Pub. Date: Jul. 11, 2024

(54) ANTI-UNC5C ANTIBODIES

(71) Applicant: Alchemab Therapeutics Ltd, London (GB)

(72) Inventors: Iosif Pediaditakis, Brookline, MA (US); Michelle Sidor, Boston, MA (US); James McCarthy, Cambridge (GB); Sandrine Legg, London (GB); Giles Lewis, London (GB); Laura Sophie Mitchell, London (GB); Justin Barton, London (GB); Emma Jenkins, Stansted (GB); Ralph Raymond Minter, London (GB); Donna Finch, London (GB); Jacob Daniel Galson, London (GB); Jane Katharine Osbourn, London (GB); Jorge Norman Dias Do Nascimento, London (GB); Paulina Maria Kolasinska-Zwierz, London (GB); William James Hawthorne, London (GB)

(21) Appl. No.: 18/408,509

(22) Filed: Jan. 9, 2024

(30) Foreign Application Priority Data
Jan. 9, 2023 (GB) 2300277.7

(51) Int. Cl. C07K 16/22; A61K 39/00; A61P 25/28

(52) U.S. Cl. CPC C07K 16/22; A61P 25/28; A61K 2039/505; C07K 2317/565; C07K 2317/567; C07K 2317/73

(57) ABSTRACT

Isolated antibodies which specifically bind to UNC5C protein and mimic and/or compete with Netrin-1 for binding to UNC5C are disclosed. Related products and uses including therapeutic uses in the treatment of neurodegenerative disorders are also disclosed.

### REFERENCE TO AN ELECTRONIC SEQUENCE LISTING

The contents of the electronic sequence listing (A139070004US00-SEQ-EMB.xml; Size: 234,074 bytes; and Date of Creation: Jan. 8, 2024) is herein incorporated by reference in its entirety.

### FIELD OF THE INVENTION

The present invention relates to antibodies and fragments thereof capable of binding to UNC5C, and particularly, although not exclusively to novel therapeutic antibodies. Methods for using anti-UNC5C antibodies in the treatment of neurodegenerative diseases are also described.

### BACKGROUND

The World Health Organisation (WHO) estimates that dementia is currently the seventh leading cause of death among all diseases and one of the major causes of disability and dependency among older people globally.

Alzheimer's disease (AD) is the most common cause of dementia affecting 1 in 14 individuals over 65 years old (Prince, M et al (2014)). AD is a progressive neurodegenerative disorder with brain atrophy leading to progressive cognitive impairment, including short term memory difficulty, impairment in expressive speech, visuospatial processing and executive (mental agility) processing) (Knopman, D. S., et al. (2021)). New therapies to Alzheimer's disease are being actively sought to modify the course of the disease. Current candidates targeting beta-amyloid, Tau, and innate immunity in the brain have in some cases shown pharmacodynamic effects on pathological mechanisms in clinical trials but have yet to demonstrate convincing disease modification in late-stage clinical trials to date (Golde T E (2022)).

Frontotemporal dementia (FTD) is a common form of dementia in those younger than 65 years and is expected to increase in prevalence as the population ages (Rosso S M, et al. 2003). FTD is an umbrella term for a group of neurodegenerative diseases characterised by progressive deficits in behaviour, executive function, and language (Bang J, et al. 2015). Whilst our understanding of the pathophysiology has considerably improved in the past decade, the development of disease-modifying drugs has been suggested to remain years away (Panza, F., et al. (2020)).

AD and FTD are both part of a spectrum of dementias with related aetiology and pathology which also includes Parkinson's disease (PD), vascular dementia, and chronic traumatic encephalopathy (CTE).

Amyotrophic lateral sclerosis (ALS) is subtype of motor neuron disease. It is a neurodegenerative disorder that primarily affects the motor neuron system, leading to muscle weakness and eventual paralysis (Hardiman, O., et al. (2017)). There is currently no effective treatment for ALS. ALS and FTD overlap clinically, radiologically, pathologically, and genetically, and 10 to 15% of ALS patients are additionally diagnosed with FTD (Phukan J, et al. Lancet Neurol. 2007; 6(11):994-100). Due to the mechanistic overlap between FTD and ALS, both diseases are considered two extremes of a disease spectrum, with predominantly cognitive symptoms at one end and motor neuron dysfunction at the other (Burrell J R et al. 2011).

There remains a need for improved disease modifying therapies in neurodegenerative diseases such as AD, FTD, and ALS.

### SUMMARY OF THE INVENTION

The present invention concerns novel antibodies to UNC5C. By studying the immune responses of individuals showing resilience to neurodegeneration despite increased risk of disease, the present inventors identified a convergent heavy chain variable (VH) domain sequence that was present in multiple resilient individuals and therefore likely to have a protective function against neurodegeneration. This VH was paired with a light chain variable (VL) domain and target deconvolution for the resulting antibody, ATL_5262, revealed UNC5C as its target. Further testing of this antibody showed that this antibody competes with netrin 1, UNC5C's ligand, for binding to UNC5C. The present inventors further identified candidate antibodies derived from ATL_5262 by modification of the heavy chain sequence. These modifications were performed to further improve properties, not limited to reduced immunogenicity, improved stability, improved binding potency, and improved pharmacokinetic properties. These novel antibodies with improved properties include antibodies comprising the heavy chain of antibodies referred herein as ATL_0005262, ATL_0005998, ATL_0006001, ATL_0006002, ATL_0006003, ATL_0006036, ATL_0006039, ATL_0006177, ATL_0006178, ATL_0006187, ATL_0006291, ATL_0006530, ATL_0006531, ATL_0006532, ATL_0006533, ATL_0006534, ATL_0006535, ATL_0006536, ATL_0006537, ATL_0006538 and ATL_0006539. The heavy chains of antibodies referred herein as ATL_0005262; ATL_0006002; ATL_0006036; ATL_0006039, ATL_0006177 and ATL_0006178 were shown to be particularly promising. The antibodies described herein are expected to improve or reverse neurodegeneration, for example in neurodegenerative diseases such as FTD, AD and ALS, by binding to UNC5C. In particular, it is relevant to note that the heavy chains described herein and antibodies derived therefrom were identified based on convergence in resilient individuals in cohorts of patients otherwise considered at risk of developing neurodegenerative disease. As such convergence is extremely unlikely by chance, the antibodies are to some extent “biologically validated” as likely to have therapeutic functions.

In a first aspect, the disclosure provides an isolated antibody or antibody fragment thereof which specifically binds to UNC5C protein or a fragment thereof, wherein the antibody mimics and/or competes with Netrin-1 for binding to UNC5C, optionally wherein competition with Netrin-1 is determined by ELISA.

Embodiments of the first aspect may have any one or more of the following optional features.

The isolated antibody may bind human and/or mouse UNC5C, for example the antibody may bind human UNC5C with an EC50 of at most 9.81E-08 M, or at most 1E-08M, or at most 5.5E-09M or at most 9.7E-09M as assessed by ELISA (such as binding of plated rhUNC5C).

The antibody may reduce UNC5C-mediated apoptosis. For example, the antibody may reduce UNC5C-mediated neuronal apoptosis. The antibody may reduce (UNC5C mediated) apoptosis in induced pluripotent stem cell (iPSC)-derived neurons. Apoptosis may be measured by measuring caspase 3/7.

The antibody may selectively bind to UNC5C over other one or more netrin receptors, for example the antibody may selectively bind to UNC5C over one or more (or all of): neogenin, DCC, and DSCAM, UNC5A, UNC5B, UNC5. The antibody may bind to UNC5C in accordance with SEQ ID NO: 99 and to an UNC5C T835M variant in accordance with SEQ ID NO: 110.

The antibody may bind to the extracellular region of UNC5C. In particular, the antibody may bind to an epitope in the N terminal immunoglobulin domain of UNC5C (also referred to as “first immunoglobulin domain). The antibody may bind to one or more residues of UNC5C that are involved in binding of Netrin-1 to UNC5C. The antibody may bind to an epitope in UNC5C comprising one or more of, or all of, the following residues: Thr89, Gln90, Gln102, Lys103, Val107, Asp108, Glu109, Arg110, Val111, Ile118, Arg120 of UNC5C when numbered in accordance with SEQ ID NO: 99, optionally wherein the antibody binds to residues Thr89, Gln90, Gln102, Lys103, Val107, Glu109, Ile118, and Arg120 of UNC5C when numbered in accordance with SEQ ID NO: 99.

The antibody may comprise a heavy chain variable domain (VH) with the following CDRs: CDRH1 comprising amino acid sequence SEQ ID NO:20, CDRH2 comprising amino acid sequence SEQ ID NO:21 or SEQ ID NO:22, and CDRH3 comprising amino acid sequence SEQ ID NO:23, or a set of CDRs which contains one or two amino acid mutations, optionally substitutions, compared with the above set of CDRs. Thus, the antibody may have a VH comprising the CDRH1, CDRH2 and CDRH3 or ATL_5262 or ATL6178, and variants thereof (e.g. ATL_6033, 6034, 6035, 6036, 6037, 6038, 6039).

The antibody may have a heavy chain variable domain (VH) with the following framework sequences: HFWR1 of SEQ ID NO:50, HFWR2 of SEQ ID NO:51, HFWR3 of SEQ ID NO:55, and HFWR4 of SEQ ID NO:62, or framework sequences with one to five mutations, for example substitutions, compared to the framework sequences above. The mutations in the framework sequences may be selected from the following positions in standard IMGT numbering: HFWR2: positions 40, 49, HFWR3: positions 80, 82, 86, and/or wherein the substitutions in the framework sequences are selected from the following positions in standard IMGT numbering: HFWR2: at positions 40: T40S, at position 49: R49G, HFWR3: at position 80: 180V, at position 82: T82K, at position 86: H86Q. The mutations in the framework sequences may not include a mutation at position 67 in standard IMGT numbering.

The antibody may have a heavy chain variable domain (VH) with the following framework sequences: HFWR1 of SEQ ID NO:50; HFWR2 of SEQ ID NO:51, 52, 53 or 54; HFWR3 of SEQ ID NO:55, 56, 57, 58, or 61; HFWR4 of SEQ ID NO:62. For example, the antibody may have a heavy chain variable domain (VH) with the following framework sequences: HFWR1 of SEQ ID NO:50; HFWR2 of SEQ ID NO: 52; HFWR3 of SEQ ID NO: 61; and HFWR4 of SEQ ID NO:62.

The antibody may have a heavy chain variable domain (VH) comprising a sequence selected that has at least 80%, at least 85%, at least 90%, or at least 95% sequence identity with a sequence selected from SEQ ID Nos: 1 (ATL_5262 VH), 5 (ATL_0006036_VH), 8 (ATL_0006039_VH), 91 (ATL_0006177_VH), and 93 (ATL_0006178_VH). For example, the antibody may have a heavy chain variable domain (VH) comprising a sequence that has at least 80%, at least 85%, at least 90%, or a least 95% sequence identity with a sequence selected from: SEQ ID NO: 91 (ATL_6177 VH); and SEQ ID NO:93 (ATL_6178 VH). The antibody may have a heavy chain variable domain (VH) comprising a sequence selected that has at least 80%, at least 85%, at least 90%, or at least 95% sequence identity with a sequence selected from SEQ ID Nos: 1 (ATL_5262 VH), 5 (ATL_0006036_VH), 8 (ATL_0006039_VH), 91 (ATL_0006177_VH), and 93 (ATL_0006178_VH). For example, the antibody may have a heavy chain variable domain (VH) comprising a sequence that has at least 80%, at least 85%, at least 90%, or a least 95% sequence identity with a sequence selected from: SEQ ID NO: 91 (ATL_6177 VH); and SEQ ID NO:93 (ATL_6178 VH).

The antibody may have a heavy chain variable domain (VH) comprising CDRH1, CDRH2, and CDRH3 within a germline framework, provided that position 67 in standard IMGT numbering is Q.

The antibody may comprise a heavy chain variable domain (VH) with the following CDRs: CDRH1 comprising amino acid sequence SEQ ID NO: 115 or 116, CDRH2 comprising amino acid sequence SEQ ID NO: 117 or 118, and CDRH3 comprising amino acid sequence SEQ ID NO: 119 or 120, optionally wherein the antibody comprises a heavy chain variable domain (VH) with the CDRs of ATL_6187 VH (SEQ ID NO: 115, 117 and 119) or the CDRs of ATL_6191 VH (SEQ ID NO: 116, 118 and 120).

The antibody may have a heavy chain variable domain (VH) with the following framework sequences: HFWR1 of SEQ ID NO: 50; HFWR2 of SEQ ID NO: 52 or 195; HFWR3 of SEQ ID NO: 196 or 197; HFWR4 of SEQ ID NO: 198 or 62: or framework sequences comprising 1 to 5 mutations, such as substitutions, compared to these framework sequences. For example, the antibody may comprise a heavy chain variable domain (VH) with the HFWRs of ATL_6187 VH (SEQ ID NO:50, 52, 196, 198) or the HFWRs of ATL_6191 VH (SEQ ID NO: 50, 195, 197, 62). The heavy chain variable domain (VH) may comprise a sequence selected that has at least 80%, at least 85%, at least 90%, or at least 95% sequence identity with a sequence selected from SEQ ID NOs: 112 (ATL_0006187 VH), and 113 (ATL_0006191 VH).

The isolated antibody according to any of the preceding claims, wherein the antibody comprises a light chain variable domain (VL) with the following CDRs: CDRL1 comprising amino acid sequence SEQ ID NO:24, 27, 30, 31 or 32, CDRL2 comprising amino acid sequence SEQ ID NO:34, and CDRL3 comprising amino acid sequence SEQ ID NO:40, 43, 46, 47, or a set of CDRs which contains one, two or three amino acid mutations, for example substitutions compared with the above set of CDRs. Thus, the antibody may have a VL with the CDRs of any of ATL_5262; 6033; 6034; 6035; 6036; 6037; 6038; 6039; 6177; 6178; 5998; 6001; 6002; and 6003, and homologues 6187 and 6191.

The isolated antibody may have a light chain variable domain (VL) with the following framework sequences: LFWR1 of SEQ ID NO:63; 66; LFWR2 of SEQ ID NO:71; 73; 75; LFWR3 of SEQ ID NO:77; 80; and LFWR4 of SEQ ID NO:85; 87; 89; or a set of FWRs which contains one to five amino acid substitutions compared with the above set of FWR. Thus, the antibody may have a VL with the LFWR of any of ATL_5262; 6033; 6034; 6035; 6036; 6037; 6038; 6039; 6177; 6178; 5998; 6001; 6002; and 6003, and homologues ATL6187 and 6191.

The antibody may have a light chain variable domain (VL) comprising a sequence selected that has at least 80%, at least 85%, at least 90%, or a least 95% sequence identity with a sequence selected from SEQ ID Nos: 9 (ATL_5262 VL), 12 (ATL_5998 VL), 15 (ATL_6001 VL), 16 (ATL_6002 VL), 17 (ATL_6003 VL). For example, the antibody may have a light chain variable domain (VL) comprising a sequence that has at least 80%, at least 85%, at least 90%, or at least 95% sequence identity with a sequence selected from: SEQ ID: 9 (ATL_5262 VL), SEQ ID NO: 16 (ATL_6002 VL); and SEQ ID NO:17 (ATL_6003 VL).

One or more of (or all of) VH residues Tyr55, Gly63, Thr64, Thr65, Asn66, Ser74, Arg108, Met110 may interact with one or more residues of UNC5C; and/or one or more of (or all of) VL residues Tyr38, Ser107, Tyr108, Ser109, Thr114, may interact with one or more residues of UNC5C. For example, the antibody may comprise a VH with: (i) at position 55: Tyr, at position 63: Gly, at position 64: Thr, at position 65: Thr, at position 66: Asn, at position 74: Ser, at position 108: Arg, and at position 110: Met, or (ii) a sequence with conservative amino acid substitutions at one or more of these positions. The antibody may comprise a VL with: at position 38: Tyr, at position 107: Ser, at position 108: Tyr, at position 109: Ser, at position 114: Thr, or a sequence with conservative amino acid substitutions at one or more of these positions.

The antibody may have a VH with at least 80% sequence identity with the VH of SEQ ID NO: 1 (ATL_5262 VH), and a VL with at least 80% sequence identity with the VL of SEQ ID: 9 (ATL_5262 VL), wherein: (a) VH residue 66 is Asn, optionally wherein Asn66 interacts with Val 107 of UNC5C; and/or (b) VH residues 55, 66, 108 and 110 are respectively Tyr, Asn, Arg and Met, optionally wherein Tyr55, Asn66, Arg108, and/or Met110 interact with Glu109 of UNC5C; and/or (c) VH residues 108 is Arg and/or residue 110 is Met, optionally wherein Arg108, and/or Met110 interact with Ile118 of UNC5C; and/or (d) VH residues 55, 64, 65 are respectively Tyr, Thr and Thr, optionally wherein Tyr55, Thr64, and/or Thr65 interacts with Arg120 of UNC5C; and/or (e) VH residue 64 is Thr, optionally wherein Thr64 interacts with Thr89 and/or Arg120 of UNC5C; and/or (f) VH residue 63 is Gly, optionally wherein Gly63 interacts with Gln90 of UNC5C; and/or (g) VH residue 65 is Thr, optionally wherein Thr65 interacts with Gln102 and/or Arg120 of UNC5C; and/or (h) VH residue 74 is Ser, optionally wherein Ser74 interacts with Lys103 of UNC5C; and/or (i) VL residue 114 is Thr, optionally wherein Thr114 interacts with Val107 and/or Glu109, and/or Arg110 of UNC5C; and/or (j) VL residue 109 is Ser, optionally wherein Ser109 interacts with Glu109 and/or Arg110 of UNC5C; and/or (k) VL residue 108 is Tyr, optionally wherein Tyr108 interacts with Arg110 and/or Val111 of UNC5C; and/or (l) VL residue 107 is Ser, optionally wherein Ser107 interacts with Val111 of UNC5C; and/or (m) VL residue 38 is Tyr, optionally wherein Tyr38 interacts with Val111 of UNC5C; wherein the UNC5C residues are numbered in accordance with SEQ ID NO: 99.

The antibody may comprise a heavy chain variable domain (VH) with the following CDRs: CDRH1 comprising amino acid sequence SEQ ID NO: 142-149; CDRH2 comprising amino acid sequence SEQ ID NO: 150-158, and_CDRH3 comprising amino acid sequence SEQ ID NO: 159-168. For example, the antibody may comprise a VH with the CDRs of any of the antibodies ATL_6530 (SEQ ID NO:142, 150, 159); ATL_6531 (SEQ ID NO:143, 151, 160); ATL_6532 (SEQ ID NO: 144, 152, 161); ATL_6533 (SEQ ID NO:145, 153, 162); ATL_6534 (SEQ ID NO:144, 152, 163); ATL_6535 (SEQ ID NO: 143, 154, 164); ATL_6536 (SEQ ID NO:146, 155, 165); ATL_6537 (SEQ ID NO:147, 156, 166); ATL_6538 (SEQ ID NO:148, 157, 167); ATL_6539 (SEQ ID NO:149, 158, 168).

The antibody may have a heavy chain variable domain (VH) with the following framework sequences: HFWR1 of SEQ ID NO: 199, 202, 205, 209, 212, 213, 216, 220, 224, 227; HFWR2 of SEQ ID NO: 200, 203, 206, 210, 214, 217, 221, 225, 228; HFWR3 of SEQ ID NO: 201, 204, 207, 211, 215, 218, 222, 226, 229; HFWR4 of SEQ ID NO: 62, 208, 219, 223, For example, the antibody may have a heavy chain variable domain (VH) with the framework sequences of any of the antibodies ATL_6530 (SEQ ID NO:199, 200, 201, 62); ATL_6531 (SEQ ID NO:202, 203, 204, 62); ATL_6532 (SEQ ID NO:205, 206, 207, 208); ATL_6533 (SEQ ID NO:209, 210, 211, 208); ATL_6534 (SEQ ID NO:212, 206, 207, 208); ATL_6535 (SEQ ID NO:213, 214, 215, 208); ATL_6536 (SEQ ID NO:216, 217, 218, 219); ATL_6537 (SEQ ID NO:220, 221, 222, 223); ATL_6538 (SEQ ID NO: 224, 225, 226, 208); ATL_6539 (SEQ ID NO:227, 228, 229, 208), such as a heavy chain variable domain (VH) with the CDRs and/or the framework sequences of ATL_6532 or ATL6533.

The antibody may have a heavy chain variable domain (VH) comprising a sequence selected that has at least 80%, at least 85%, at least 90%, or at least 95% sequence identity with a sequence selected from SEQ ID NOs: 122 (ATL_0006530 VH); 123 (ATL_0006531 VH); 124 (ATL_0006532 VH); 125 (ATL_0006533 VH); 126 (ATL_0006534 VH); 127 (ATL_0006535 VH); 128 (ATL_0006536 VH); 129 (ATL_0006537 VH); 130 (ATL_0006538 VH). For example, the antibody may have a heavy chain variable domain (VH) comprising a sequence that has at least 70%, at least 75%, at least 80%, at least 85%, at least 90%, or at least 95% sequence identity with a sequence selected from: SEQ ID NO: 124 (ATL_6532 VH); SEQ ID NO: 125 (ATL_6533 VH).

The antibody may comprise a light chain variable domain (VL) with the following CDRs: CDRL1 comprising amino acid sequence SEQ ID NO: 169-176; CDRL2 comprising amino acid sequence SEQ ID NO: 177-183; CDRL3 comprising amino acid sequence SEQ ID NO: 184-193. For example, the antibody may comprise a VL with the CDRs of any of the antibodies ATL_6530 (SEQ ID NO:169, 177, 184); ATL_6531 (SEQ ID NO:170, 178, 185); ATL_6532 (SEQ ID NO:171, 177, 186); ATL_6533 (SEQ ID NO:169, 177, 187); ATL_6534 (SEQ ID NO:169, 177, 188); ATL_6535 (SEQ ID NO:172, 179, 189); ATL_6536 (SEQ ID NO:173, 180, 190); ATL_6537 (SEQ ID NO:174, 181, 191); ATL_6538 (SEQ ID NO:175, 182, 192); ATL_6539 (SEQ ID NO:176, 183, 193).

The antibody may further have a light chain variable domain (VL) with the following framework sequences: LFWR1 of SEQ ID NO: 230 to 237 and 68; LFWR2 of SEQ ID NO: 239 to 246; LFWR3 of SEQ ID NO: 247 to 255; and LFWR4 of SEQ ID NO: 256 to 258. For example, the antibody may comprise a VL with the FWRs of any of the antibodies ATL_6530 (SEQ ID NO:230, 238, 247, 256); ATL_6531 (SEQ ID NO:231, 239, 248, 257); ATL_6532 (SEQ ID NO:232, 240, 249, 258); ATL_6533 (SEQ ID NO:230, 241, 247, 256); ATL_6534 (SEQ ID NO:233, 238, 250, 256); ATL_6535 (SEQ ID NO:234, 242, 251, 257); ATL_6536 (SEQ ID NO:68, 243, 252, 257); ATL_6537 (SEQ ID NO:235, 244, 253, 256); ATL_6538 (SEQ ID NO:236, 245, 254, 256); ATL_6539 (SEQ ID NO:237, 246, 255, 256).

The light chain variable domain (VL) may comprise a sequence selected that has at least 80%, at least 85%, at least 90%, or at least 95% sequence identity with a sequence selected from SEQ ID NO: 132-141 (ATL6530-6539 VL). For example, the antibody may have a light chain variable domain (VL) comprising a sequence that has at least 80%, at least 85%, at least 90%, or at least 95% sequence identity with a sequence selected from: SEQ ID NO: 134 (ATL_0006532); or SEQ ID NO: 135 (ATL_0006533).

The antibody may comprise an scFv antibody molecule, a nanobody, an antibody constant region, or a whole antibody.

The antibody may be a monoclonal antibody and/or wherein the antibody is a whole antibody and/or wherein the antibody is an IgG1 or variant thereof, optionally wherein the antibody is an IgG1 variant L234A/L235A (LALA).

Also described according to a second aspect is an isolated VH domain of an antibody according to any embodiment of the first aspect or any antibody as described herein. Thus, the antibody fragment according to any preceding aspect may comprise an isolated VH domain that has the features of any embodiment of the preceding aspects. Also described herein is an isolated antibody VL domain of an antibody as described herein. Thus, the antibody fragment according to any preceding aspect may comprise an isolated VL domain that has the features of any embodiment of an antibody as described herein.

According to a third aspect, the disclosure provides an isolated nucleic acid which comprises a nucleotide sequence encoding an antibody or antibody fragment thereof, including a VH or VL domain, as described herein.

According to a fourth aspect, the disclosure provides a vector or set of vectors comprising the nucleic acid according to the preceding aspect.

According to a fifth aspect, the disclosure provides host cell comprising the vector or set of vectors according to the sixth aspect, or a host cell in vitro transformed with a nucleic acid of the fifth aspect.

According to an sixth aspect, the disclosure provides composition comprising an antibody or fragment thereof, including an antibody VH domain or antibody VL domain, as described herein, and at least one additional component. The composition may comprise a pharmaceutically acceptable excipient, vehicle or carrier.

According to a seventh aspect, the disclosure provides a method of producing an antibody or fragment thereof, the method comprising culturing host cells according to the fifth aspect under conditions for production of said antibody or fragment thereof. The method may further comprise isolating and/or purifying said antibody or fragment thereof. The method may further comprise formulating the antibody or fragment thereof into a composition including at least one additional component.

According to an eighth aspect, the disclosure provides a method of treating a disease or disorder in a subject, comprising administering to the subject an effective amount of an isolated antibody or antibody fragment as described herein.

According to a ninth aspect, the disclosure provides method of reducing apoptosis in a cell expressing UNC5C, the method comprising contacting the cell with an antibody or antibody fragment as described herein. The cell may be a neuron. The contacting may be performed in vitro.

According to a tenth aspect, the disclosure provides the use of an antibody or fragment thereof that binds UNC5C, in the manufacture of a medicament for treatment of a neurodegenerative disease or disorder. The antibody or fragment thereof may be according to any embodiment as described herein.

According to an eleventh aspect, the disclosure provides a method of treatment of a neurodegenerative disease or disorder, the method comprising administering an antibody or fragment thereof that binds UNC5C in a therapeutically effective amount, to a patient with the disease or disorder or at risk of developing the disease or disorder. The antibody or fragment thereof may be an antibody as described herein.

According to a twelfth aspect, the disclosure provides an antibody or fragment thereof that binds UNC5C, for use in the treatment of a neurodegenerative disease or disorder. The antibody or fragment thereof may be an antibody or fragment thereof as described herein. A neurodegenerative disorder according to any aspect such as any of the 12th, 13th or 14th aspects may be selected from: frontotemporal dementia (FTD), Alzheimer's disease (AD), Huntington's Disease (HD), Parkinson's disease (PD), amyotrophic lateral sclerosis (ALS), human immunodeficiency virus (HIV)-induced encephalitis, Chronic traumatic encephalopathy (CTE), vascular dementia, prion diseases, Lewy body disease, Spinal muscular atrophy (SMA), Motor Neuron Disease (MND), such as amyotrophic lateral sclerosis (ALS), progressive supranuclear palsy (PSP), spinocerebellar ataxias (SCA) types 1, 2, 6, 7 and 17, Machado-Joseph disease (MJD/SCA3), dentatorubral pallidoluysian atrophy (DRPLA), spinal bulbar muscular atrophy X-linked type 1 (SMAX1/SBMA), Anderson-Fabry (X-linked Fabry Disease), and DNAJB6 Myopathies. For example, the neurodegenerative disease may be selected from FTD, AD, HD, and PD.

The invention includes the combination of the aspects and preferred features described except where such a combination is clearly impermissible or expressly avoided.

Thus, also described in embodiments of the first aspect, is an isolated antibody or antibody fragment thereof which comprises a heavy chain variable domain (VH) with the following CDRs: CDRH1 comprising amino acid sequence SEQ ID NO:20, CDRH2 comprising amino acid sequence SEQ ID NO:21 or SEQ ID NO:22, and CDRH3 comprising amino acid sequence SEQ ID NO:23. The antibody may comprise a heavy chain variable domain (VH) with CDRs with one or two amino acid substitutions compared with the following CDRs: CDRH1 comprising amino acid sequence SEQ ID NO:20, CDRH2 comprising amino acid sequence SEQ ID NO:21 or SEQ ID NO:22, and CDRH3 comprising amino acid sequence SEQ ID NO:23.

Also described according to a further aspect, is an isolated antibody or antibody fragment thereof which specifically binds to UNC5C protein or a fragment thereof, wherein the antibody comprises a heavy chain variable domain (VH) with the following CDRs: CDRH1 comprising amino acid sequence SEQ ID NO:20, CDRH2 comprising amino acid sequence SEQ ID NO:21 or SEQ ID NO:22, and CDRH3 comprising amino acid sequence SEQ ID NO:23, or a set of CDRs which contains one or two amino acid substitutions compared with the above set of CDRs. Thus, the antibody may have a VH comprising the CDRH1, CDRH2 and CDRH3 or ATL_5262 or ATL6178, and variants thereof (e.g. ATL_6033, 6034, 6035, 6036, 6037, 6038, 6039). The antibody may mimic and/or compete with Netrin-1 for binding to UNC5C. The antibody may have any of the features described above in relation to the first aspect.

Also described according to a further aspect is an isolated antibody or antibody fragment thereof which specifically binds to UNC5C protein or a fragment thereof, wherein the antibody comprises a heavy chain variable domain (VH) with the following CDRs: CDRH1 comprising amino acid sequence SEQ ID NO: 115 or 116; CDRH2 comprising amino acid sequence SEQ ID NO: 117 or 118; and CDRH3 comprising amino acid sequence SEQ ID NO: 119 or 120. The antibody may comprise a heavy chain variable domain (VH) with the CDRs of ATL_6187 VH (SEQ ID NO: 115, 117 and 119) or the CDRs of ATL_6191 VH (SEQ ID NO: 116, 118 and 120). The antibody may mimic and/or compete with Netrin-1 for binding to UNC5C. The antibody may have any of the features described above in relation to the first aspect.

Also described according to a further aspect is an isolated antibody or antibody fragment thereof which specifically binds to UNC5C protein or a fragment thereof, wherein the antibody comprises a heavy chain variable domain (VH) with the following CDRs: CDRH1 comprising amino acid sequence SEQ ID NO: 142-149; CDRH2 comprising amino acid sequence SEQ ID NO: 150-158, and CDRH3 comprising amino acid sequence SEQ ID NO: 174-183. The antibody may comprise a VH with the CDRs of any of the antibodies ATL_6530 (SEQ ID NO:142, 150, 159); ATL_6531 (SEQ ID NO:143, 151, 160); ATL_6532 (SEQ ID NO: 144, 152, 161); ATL_6533 (SEQ ID NO:145, 153, 162); ATL_6534 (SEQ ID NO:144, 152, 163); ATL_6535 (SEQ ID NO: 143, 154, 164); ATL_6536 (SEQ ID NO:146, 155, 165); ATL_6537 (SEQ ID NO:147, 156, 166); ATL_6538 (SEQ ID NO:148, 157, 167); ATL_6539 (SEQ ID NO:149, 158, 168). The antibody may mimic and/or compete with Netrin-1 for binding to UNC5C. The antibody may have any of the features described above in relation to the first aspect.

According to a third aspect, there is provided an isolated antibody or antibody fragment thereof which specifically binds to UNC5C protein or a fragment thereof, for use in the treatment of a neurodegenerative disorder. The antibody may mimic and/or compete with Netrin-1 for binding to UNC5C. The antibody may comprise a heavy chain variable domain (VH) with the following CDRs: CDRH1 comprising amino acid sequence SEQ ID NO:20, CDRH2 comprising amino acid sequence SEQ ID NO:21 or SEQ ID NO:22, and CDRH3 comprising amino acid sequence SEQ ID NO:23. The antibody may comprise a heavy chain variable domain (VH) with CDRs with one or two amino acid substitution compared with the following CDRs: CDRH1 comprising amino acid sequence SEQ ID NO:20, CDRH2 comprising amino acid sequence SEQ ID NO:21 or SEQ ID NO:22, and CDRH3 comprising amino acid sequence SEQ ID NO:23. The antibody may comprise a heavy chain variable domain (VH) with the following CDRs: CDRH1 comprising amino acid sequence SEQ ID NO: 115 or 116; CDRH2 comprising amino acid sequence SEQ ID NO: 117 or 118; and CDRH3 comprising amino acid sequence SEQ ID NO: 119 or 120. antibody may comprise a heavy chain variable domain (VH) with the following CDRs: CDRH1 comprising amino acid sequence SEQ ID NO: 142-149; CDRH2 comprising amino acid sequence SEQ ID NO: 150-158, and CDRH3 comprising amino acid sequence SEQ ID NO: 174-183.

Embodiments of any of the above aspects may have any one or more of the following optional features.

The antibody may bind the same binding site on UNC5C as Netrin-1. The antibody may have an epitope that at least partially overlaps the binding site of Netrin-1. The antibody of any aspect may comprise a heavy chain variable domain (VH) with CDRs that have at least 70%, 75%, 80%, 85% or 90% sequence identity with the following CDRs: CDRH1 comprising amino acid sequence SEQ ID NO:20, CDRH2 comprising amino acid sequence SEQ ID NO:21 or SEQ ID NO:22, and CDRH3 comprising amino acid sequence SEQ ID NO:23. The antibody may have CDRHs that have the same lengths as those CDRHs. The antibody of any aspect may comprise a heavy chain variable domain (VH) with the CDR of the heavy chain of antibodies ATL_5262, ATL_0006036, ATL_0006039, ATL_0006177 or ATL_0006178, or a set of CDRs with one two three amino acid substitutions or at least 70%, 75%, 80%, 85% or 90% sequence identity with these CDRs. The one or two substitutions may comprise or consist of a substitution at the following residue within the CDRs, using the standard numbering of IMGT: 58 in CDRH2. The substitution may be N58Y. The antibody may have a heavy chain variable domain (VH) with the following framework sequences: HFWR1 of SEQ ID NO:50, HFWR2 of SEQ ID NO:51, HFWR3 of SEQ ID NO:55, and HFWR4 of SEQ ID NO:62, or framework sequences with one to five substitutions compared to the framework sequences above. The substitutions in the framework sequences may be selected from the following positions in standard IMGT numbering: HFWR2: positions 40, 49, HFWR3: positions 80, 82, 86. The substitutions in the framework sequences may be selected from the following positions in standard IMGT numbering: HFWR2: at positions 40: T40S, at position 49: R49G, HFWR3: at position 80: 180V, at position 82: T82K, at position 86: H86Q. The substitutions in the framework sequences may not include a substitution at position 67 in standard IMGT numbering. In other words, the Q at position 67 may be maintained. The present inventors have found that maintaining the Q at position 67 in HFWR3 improved binding affinity to UNC5C. The present inventors have found that removing the N at position 58 in the CDRH2 sequence may remove a potential sequence liability (position that may be problematic during production of the antibody). The present inventors have found that removing the T at position 82 in the HFWR3 reduced the risk of aspartate isomerisation. They have further found that replacing this T with K improved binding affinity of the antibody to UNC5C. The present inventors have found that substitutions at positions 40, 49 (in HFWR2), 80, 86 (in HFWR3), particularly to replace them with the residues on the corresponding germline sequence, may reduce immunogenicity, improve antibody expression and stability. The antibody may have a heavy chain variable domain (VH) with the following framework sequences: HFWR1 of SEQ ID NO:50; HFWR2 of SEQ ID NO:51, 52, 53 or 54; HFWR3 of SEQ ID NO:55, 56, 57, 58, or 61; HFWR4 of SEQ ID NO:62. The antibody may have a heavy chain variable domain (VH) with the following framework sequences: HFWR1 of SEQ ID NO:50; HFWR2 of SEQ ID NO: 52; HFWR3 of SEQ ID NO: 61; and HFWR4 of SEQ ID NO:62.

The antibody may have a heavy chain variable domain (VH) comprising a sequence selected that has a least 95% sequence identity with a sequence selected from SEQ ID Nos: 1 (ATL_5262 VH), 5 (ATL_0006036_VH), 8 (ATL_0006039_VH), 91 (ATL_0006177_VH), and 93 (ATL_0006178_VH). The antibody may have a heavy chain variable domain (VH) comprising a sequence that has a least 95% sequence identity with a sequence selected from: SEQ ID: 91 (ATL_6177 VH) and SEQ ID NO:93 (ATL_6178 VH). ATL_6177 and ATL_6178 are expected to have improved stability and binding potency compared with other antibodies described herein, due to the particular sequence of the VH of these antibodies (SEQ ID Nos: 91 and 93 in Table 1, or as described in FIG. 22A). ATL_5262 VH is described in Table 1, or as described in FIG. 21A. As described further below and in FIG. 21A, ATL_5262 VH has the same sequence as ATL_0005996_VH, ATL_0005997_VH, ATL_0005998_VH, ATL_0005999_VH, ATL_0006000_VH, ATL_0006001_VH, ATL_0006002_VH, ATL_0006003_VH, ATL_0006004_VH, and ATL_0006005_VH. The antibody may comprise a heavy chain variable domain that has a sequence selected from SEQ ID Nos: 1 (ATL_5262 VH), 5 (ATL_0006036_VH), 8 (ATL_0006039_VH), 91 (ATL_0006177_VH), and 93 (ATL_0006178_VH). The antibody may have a heavy chain variable domain (VH) comprising CDRH1, CDRH2, and CDRH3 within a germline framework, provided that position 67 in standard IMGT numbering is Q.

The antibody may bind human and/or mouse UNC5C. The antibody may bind human UNC5C with an EC50 of at most 9.81E-08 M, or at most 1E-08M, or at most 5.5E-09M as assessed by ELISA (such as binding of plated rhUNC5C). The antibody may reduce UNC5C-mediated apoptosis. The antibody may reduce UNC5C-mediated neuronal apoptosis. The antibody may reduce apoptosis in induced pluripotent stem cell (iPSC)-derived neurons. Reduction in apoptosis can be verified by comparison to a suitable control (e.g. vehicle only, control antibody that does not bind UNC5C, etc.) in a caspase 3/7 assay as described herein. The antibody may have an effect on synaptic health. For example, the antibody may increase synaptic plasticity. Thus, the antibody may increase one or more measures of synaptic health (including but not limited to e.g. plasticity), such as the number of processes per cell, length of neurite outgrowth and/or branching.

The antibody may selectively bind to UNC5C over other one or more netrin receptors. The antibody may selectively bind to UNC5C over one or more (or all of): neogenin, DCC, and DSCAM, UNC5A, UNC5B, UNC5. Selective binding may refer to preferential binding (e.g. lower EC50) for one species (e.g. UNC5C) over another (e.g. other netrin receptor), or to no detectable binding to comparative targets (e.g. other netrin receptors). Selective binding may be verified by biolayer interferometry analysis as described herein. The antibody may bind to monomeric UNC5C and/or dimeric UNC5C and/or homodimeric UNC5C. The antibody may bind to monomeric UNC5C.

The antibody may comprise an scFv antibody molecule, a nanobody, an antibody constant region, or a whole antibody. The antibody may be a whole antibody. The antibody may be an IgG1 or variant thereof, optionally wherein the antibody is an IgG1 variant L234A/L235A (LALA).

The antibody may further comprise a light chain variable domain (VL) with the following CDRs: CDRL1 comprising amino acid sequence SEQ ID NO:24, 31 or 32, CDRL2 comprising amino acid sequence SEQ ID NO:34, and CDRL3 comprising amino acid sequence SEQ ID NO:40, 47, or a set of CDRs which contains one, two or three amino acid substitutions compared with the above set of CDRs. The antibody of any aspect may comprise a light chain variable domain (VL) with CDRs that have at least 70%, 75%, 80%, 85% or 90% sequence identity with the following CDRs: CDRL1 comprising amino acid sequence SEQ ID NO:24, 31, or 32, CDRL2 comprising amino acid sequence SEQ ID NO:34, and CDRL3 comprising amino acid sequence SEQ ID NO:40 or 47. The antibody may have CDRLs that have the same lengths as those CDRLs. The antibody of any aspect may comprise a light chain variable domain (VL) with the CDR of the light chain of antibodies ATL_5262, ATL_0006002 or ATL_0006003, or a set of CDRs with one two three amino acid substitutions or at least 70%, 75%, 80%, 85% or 90% sequence identity with these CDRs. The antibody of any aspect may comprise a light chain variable domain (VL) with the CDR of the light chain of antibodies ATL_5262 or ATL_0006002, or a set of CDRs with one two three amino acid substitutions or at least 70%, 75%, 80%, 85% or 90% sequence identity with these CDRs. The CDRL1, CDRL2 and CDRL3 of the VL domain may be within a germline framework. The antibody may further comprises a light chain variable domain (VL) with the following framework regions: LFWR1 comprising any of amino acid sequences SEQ ID NO: 63 to 70, LFWR2 comprising any of amino acid sequences SEQ ID NO:71 to 76, LFWR3 comprising any of amino acid sequences SEQ ID NO:77 to 84, and LFWR4 comprising any of amino acid sequences SEQ ID NO:85 to 90, or a set of FWRs which contains one to five amino acid substitution compared with the above set of FWRs. The antibody may have a light chain variable domain (VL) with the following framework sequences: LFWR1 of SEQ ID NO:63; LFWR2 of SEQ ID NO:71; LFWR3 of SEQ ID NO:77; and LFWR4 of SEQ ID NO:85 or 87; or a set of FWRs which contains one to five amino acid substitutions compared with the above set of FWR.

The antibody may have a light chain variable domain (VL) comprising a sequence selected that has a least 95% sequence identity with a sequence selected from SEQ ID Nos: 9 (ATL_5262 VL), or 10 to 19. The antibody may have a light chain variable domain (VL) with the following framework sequences: LFWR1 of SEQ ID NO:63; LFWR2 of SEQ ID NO:71; LFWR3 of SEQ ID NO:77; and LFWR4 of SEQ ID NO:85 or 87; or a set of FWRs which contains one to five amino acid substitutions compared with the above set of FWR. The antibody may have a light chain variable domain (VL) comprising a sequence that has a least 95% sequence identity with a sequence selected from: SEQ ID: 9 (ATL_5262 VL), 16 (ATL_6002 VL) and SEQ ID NO:17 (ATL_6003 VL).

### SUMMARY OF THE FIGURES

Embodiments and experiments illustrating the principles of the invention will now be discussed with reference to the accompanying figures in which:

FIG. 1 shows schematically a process for unbiased antibody discovery.

FIG. 2 shows results of ELISA assays for binding of antibodies identified in FTD resilient individuals to recombinant UNC5C. The pool of antibodies tested consisted of 18 antibodies identified from a FTD cohort, one antibody from a Huntington cohort, and an anti-PDL1 antibody, previously used as a positive control in a Retrogenix target deconvolution assay. The antibody, ATL_5262 binds to UNC5C. The blue bars indicate raw absorbance signals to recombinant UNC5C and the orange bars represent absorbance signals to an irrelevant protein, lysozyme. FIG. 2 shows absorbance at 450 nm. The anti-Fc tag bars show the results for an anti-Fc-HRP treated well which controls that the UNC5C is correctly coated on the assay plate.

FIG. 3 shows the presence of UNC5C antibodies in subjects who are resilient to neurodegeneration in different neurodegenerative disease cohorts. UNC5C antibodies were found in resilient centenarians (>100 year old subjects with no symptoms of neurodegeneration); resilient Alzheimer's disease (AD) patients (a resilient Alzheimer's disease subject showing slow cognitive progression), Frontotemporal dementia (FTD) patients (three subjects with resilience for FTD—carriers of high risk mutations and older age but no progression to FTD), Parkinson's disease (PD) patients (prodromal REM sleep behaviour disorder (RBD) but no Parkinson's diagnosis), and cognitively healthy controls (predominantly individuals over 60 years of age). Repertoires were searched for VH sequences containing the same V gene and J gene as ATL5262 and at most 2 amino acid mismatches across the junction region. Dots represent the number of matching sequences per repertoire and colour indicates the isotypes of sequences identified. The majority of matching sequences are class switched to IgG1 supporting a similar function for these antibodies across individuals. Unclassified PD subject SU_0001278 was initially included as a healthy control for this cohort, but subsequently removed since they have suspected REM sleep behaviour disorder, which is a prodromal marker for PD. Since they have no PD diagnosis they are ‘potentially resilient’.

FIG. 4A shows the results of a Netrin 1 competition assay. ELISA plates were coated with rhUNC5C (recombinant human UNC5C) and preincubated with His-tagged recombinant Netrin 1 prior to adding ATL_5262 or control antibody ATL_5338. Netrin 1 was detected using an anti-His (horseradish peroxidase) HRP antibody.

FIG. 4B shows the results of a Netrin 1 competition assay. ELISA plates were coated with rhUNC5C (recombinant human UNC5C) and preincubated with His-tagged recombinant Netrin 1 prior to adding ATL_5262 or control antibody ATL_5338. Antibody (ATL_5262 or control) was detected by anti-F(ab)′2-HRP detection. Assays were set up in duplicate whereby one replicate had Netrin 1 binding detected with anti-His-HRP (A) and the second replicate was used to determine ATL_0005262 antibody binding using the anti-Fab′2-HRP detection. FIG. 4B shows absorbance at 450 nm.

FIG. 5 shows the results of a concentration-dependent Netrin 1 competition assay. ELISA plates were coated with rhUNC5C and preincubated with a 12-point dilution series of His-tagged recombinant Netrin 1 (starting at 3 nM) prior to adding ATL_5262. Netrin 1 was detected using anti-His HRP detection and is shown in FIG. 5 as “Netrin 1 signal (+ATL_5262)”. ATL_5262 was detected using anti-F(ab)′2-HRP detection and is shown in FIG. 5 as “ATL_0005262 signal (+Netrin1)”. Control wells contained Netrin 1 alone. Data represented as percentage of maximum absorbance signal.

FIG. 6 shows the results of a Netrin 1 competition assay. ELISA plates were coated with rhUNC5C and preincubated with ATL_5262 or control antibody ATL_5338 prior to adding His-tagged recombinant Netrin 1. Netrin 1 was detected using anti-His HRP detection and is shown in FIG. 6 as “Netrin signal (+ATL_0005262)” or “Netrin signal (+ATL_00005338)”. ATL_5262 was detected using anti-F(ab)′2-HRP detection. FIG. 6 shows absorbance at 450 nm.

FIG. 7A shows binding of ATL_5262 to recombinant human (rh) UNC5C using ELISA. FIG. 7A shows absorbance at 450 nm.

FIG. 7B shows binding of ATL_5262 to recombinant mouse (rm) UNC5C, using ELISA. FIG. 7B shows absorbance at 450 nm.

FIG. 7C shows binding of ATL_5262 to lysozyme, using ELISA. FIG. 7C shows absorbance at 450 nm.

FIG. 8A shows a VH alignment of ATL_5262 and ATL_5262 homologues identified across neurodegeneration cohorts in resilient individuals. FIG. 8A shows IMGT numbering. FIG. 8A shows sequences of antibodies described herein (in particular, VH of ATL_0005262, SEQ ID NO: 1; VH of ATL_0006187, SEQ ID NO: 112; VH of ATL_0006191, SEQ ID NO: 113), with variable positions in the heavy chain highlighted.

FIG. 8B shows the heavy chain sequences (VH FW1 and CDR1) of the antibodies in FIG. 8A as well as ATL_6178 (VH of ATL_6178, SEQ ID NO: 93), aligned by IMGT position numbering. ATL_5262 and ATL_6178 have the same VL. Homologues were identified based on VH alone so only VH sequences are shown.

FIG. 8C shows the heavy chain sequences (VH FW2 and CDR2) of the antibodies in FIG. 8A as well as ATL_6178 (VH of ATL_6178, SEQ ID NO: 93), aligned by IMGT position numbering. ATL_5262 and ATL_6178 have the same VL. Homologues were identified based on VH alone so only VH sequences are shown.

FIG. 8D shows the heavy chain sequences (VH FW3) of the antibodies in FIG. 8A as well as ATL_6178 (VH of ATL_6178, SEQ ID NO: 93), aligned by IMGT position numbering. ATL_5262 and ATL_6178 have the same VL. Homologues were identified based on VH alone so only VH sequences are shown.

FIG. 8E shows the heavy chain sequences (VH CDR3 and FW4) of the antibodies in FIG. 8A as well as ATL_6178 (VH of ATL_6178, SEQ ID NO: 93), aligned by IMGT position numbering. ATL_5262 and ATL_6178 have the same VL. Homologues were identified based on VH alone so only VH sequences are shown.

FIG. 9 illustrates a workflow for a 2-week and 4-week stability study and a freeze-thaw (3× FT) stability study for ATL_5262. Quality control (QC) analysis was performed using size exclusion high performance liquid chromatography (SEC-HPLC), sodium dodecyl sulfate-polyacrylamide gel electrophoresis (SDS—PAGE); Capillary electrophoresis sodium dodecyl sulfate (CE-SDS) capillary isoelectric focusing (cIEF); dynamic light scattering (DLS); and binding ELISA.

FIG. 10 shows the results of a thermal shift assay for ATL_5262 at 1 (left) and 5 mg/ml (right) (ratio of unfolded-to-folded protein as a function of temperature).

FIG. 11A shows electropherograms of the charge variants of ATL_5262 tested by cIEF two weeks after storage at the indicated temperatures (−80° C., 4° C., and 40° C.).

FIG. 11B shows an electropherogram of the charge variants of ATL_5262 tested by cIEF 4 weeks after storage at the indicated temperatures (−80° C., 4° C., and 40° C.).

FIG. 11C shows electropherograms of the charge variants of ATL_5262 tested by cIEF two weeks (left) and 4 weeks (right) after storage at the indicated temperatures (−80° C., 4° C., and 40° C.). The figure shows a zoomed-in version of FIG. 11A and FIG. 11B showing the acidic species on the left-hand side of the graphs.

FIG. 12A shows a SEC-HPLC chromatogram for ATL_5262 after 2 weeks at the indicated temperatures (−80° C., 4° C., and 40° C.).

FIG. 12B shows a SEC-HPLC chromatogram for ATL_5262 after 4 weeks at the indicated temperatures (−80° C., 4° C., and 40° C.).

FIG. 13A shows the results of an SDS-PAGE for ATL_5262 after 2 weeks at the indicated temperatures (−80° C., 4° C., and 40° C.) FT=Freeze Thaw; NIST=NIST mAb control; R=Reduced; NR=Non-Reduced.

FIG. 13B shows the results of an SDS-PAGE for ATL_5262 after 4 weeks at the indicated temperatures (−80° C., 4° C., and 40° C.) or after a 3× FT study. FT=Freeze Thaw; NIST=NIST mAb control; R=Reduced; NR=Non-Reduced.

FIG. 14A shows the results of CE-SDS for ATL_5262 after 2 weeks at the indicated temperatures (−80, +4 and +40 C).

FIG. 14B shows the results of CE-SDS for ATL_5262 after 4 weeks (non-reduced samples) at the indicated temperatures (−80, +4 and +40 C).

FIG. 14C shows the results of CE-SDS for ATL_5262 after 2 weeks (reduced samples) at the indicated temperatures (−80, +4 and +40 C).

FIG. 14D shows the results of CE-SDS for ATL_5262 after 4 weeks (reduced samples) at the indicated temperatures (−80, +4 and +40 C).

FIG. 15A shows the results of an ELISA testing the binding of ATL_5262 to rhUNC5C after 2 weeks (−80° C., 4° C., and 40° C.).

FIG. 15B shows the results of an ELISA testing the binding of ATL_5262 to rhUNC5C after 4 weeks at the indicated temperatures (−80° C., 4° C., and 40° C.) or after a 3× FT study.

FIG. 16 shows the results of an ELISA testing the binding of different VH variants of ATL_5262 to rhUNC5C. ATL_5338 was included as a negative control. FIG. 16 shows absorbance at 450 nm.

FIG. 17A shows the results of an ELISA testing the binding of different VH variants of ATL_5262 to rhUNC5C. ATL_5338 was included as a negative control. ATL_6002 and ATL_6003 (VL pairing variants) were included for comparison. FIG. 17A shows absorbance at 450 nm.

FIG. 17B shows the results of an ELISA testing the binding of different VH variants of ATL_5262 to rmUNC5C. ATL_5338 was included as a negative control. ATL_6002 and ATL_6003 (VL pairing variants) were included for comparison. FIG. 17B shows absorbance at 450 nm.

FIG. 17C shows the results of an ELISA testing the binding of different VH variants of ATL_5262 to lysozyme. ATL_5338 was included as a negative control. ATL_6002 and ATL_6003 (VL pairing variants) were included for comparison. FIG. 17C shows absorbance at 450 nm.

FIG. 18A shows the results of an ELISA testing the binding of different VH variants of ATL_5262 to Fc tagged rhUNC5C. ATL_5338 was included as a negative control. ATL_6002 and ATL_6003 (VL pairing variants) were included for comparison. FIG. 18A shows absorbance at 450 nm.

FIG. 18B shows the results of an ELISA testing the binding of different VH variants of ATL_5262 to His-tagged rhUNC5C. ATL_5338 was included as a negative control. ATL_6002 and ATL_6003 (VL pairing variants) were included for comparison. FIG. 18B shows absorbance at 450 nm.

FIG. 19A shows the results of an ELISA testing the binding of different VH variants of ATL_5262 to rhUNC5C. ATL_5338 was included as a negative control. FIG. 19A shows absorbance at 450 nm.

FIG. 19B shows the results of an ELISA testing the binding of different VH variants of ATL_5262 to rmUNC5C. ATL_5338 was included as a negative control. FIG. 19B shows absorbance at 450 nm.

FIG. 19C shows the results of an ELISA testing the binding of different VH variants of ATL_5262 to lysozyme. ATL_5338 was included as a negative control. FIG. 19C shows absorbance at 450 nm.

FIG. 20A shows the results of an in vivo mouse pharmacokinetic study assessing antibody levels in serum following administration of escalation doses of ATL_5262 or a control antibody, ATL_5338.

FIG. 20B shows the results of an in vivo mouse pharmacokinetic study assessing antibody levels in CSF following administration of escalation doses of ATL_5262 or a control antibody, ATL_5338.

FIG. 21A shows sequences of antibodies described herein, with variable positions in the heavy chain highlighted. Heavy chains FW1 sequences, aligned by IMGT position numbering. VH of ATL_0005262, 5996, 5997, 5998, 5999, 6000, 6001, 6002, 6003, 6004, 6005: SEQ ID NO: 1; VH of ATL_0006033: SEQ ID NO: 2; VH of ATL_6034: SEQ ID NO: 3, VH of ATL_0006035: SEQ ID NO: 4, VH of ATL_0006036: SEQ ID NO: 5; VH of ATL_0006037: SEQ ID NO: 6; VH of ATL_0006038: SEQ ID NO: 7; VH of ATL_0006039: SEQ ID NO: 8; VH of ATL_0006177: SEQ ID NO: 91; VH of ATL_0006178: SEQ ID NO: 93.

FIG. 21B shows the CDR1 sequences of the heavy chains of the antibodies described in relation to FIG. 21A.

FIG. 21C shows the FW2 and CDR2 sequences of the heavy chains of the antibodies described in relation to FIG. 21A.

FIG. 21D shows the FW3 sequences of the heavy chains of the antibodies described in relation to FIG. 21A.

FIG. 21E shows the CDR3 and FW4 sequences of the heavy chains of the antibodies described in relation to FIG. 21A.

FIG. 21F shows sequences of antibodies described herein: Light chains FW1 sequences, aligned by IMGT position numbering. VL of ATL_0005262: SEQ ID NO: 9; VL of ATL_0005996: SEQ ID NO: 10; VL of ATL_0005997: SEQ ID NO: 11; VL of ATL_0005998: SEQ ID NO: 12; VL of ATL_0005999: SEQ ID NO: 13; VL of ATL_0006000: SEQ ID NO: 14; VL of ATL_0006001: SEQ ID NO: 15; VL of ATL_0006002: SEQ ID NO: 16; VL of ATL_0006003: SEQ ID NO: 17; VL of ATL_0006004: SEQ ID NO: 18; VL of ATL_0006005: SEQ ID NO: 19; VL of ATL_0006033, 6034, 6035, 6036, 6037, 6038, 6039, 6177: SEQ ID NO: 92; VL of ATL_0006178: SEQ ID NO: 94.

FIG. 21G shows the CDR1 sequences of the light chains of the antibodies described in relation to FIG. 21F.

FIG. 21H shows the FW2 and CDR2 sequences of the light chains of the antibodies described in relation to FIG. 21F.

FIG. 21I shows the FW3 sequences of the light chains of the antibodies described in relation to FIG. 21F.

FIG. 21J shows the CDR3 and FW4 sequences of the light chains of the antibodies described in relation to FIG. 21F.

FIG. 22 shows the results of a bio-layer interferometry assay to assess binding of ATL_5262 and ATL_6178 to different Netrin 1 receptors and related proteins. For each antigen-antibody-pair, aligned, the graph shows reference-corrected sensor response at the end of the measured association step (average of 25-30 seconds signal) of a BLI kinetic assay. Antigen was loaded as ligand and dipped into antibody as analyte during association, and assay buffer during dissociation.

FIG. 23A shows the results of a netrin-1 competition assay demonstrating that binding of ATLX-1282 and ATL_5262 to mouse brain tissue is blocked via Netrin-1. Graph shows effect of Netrin-1 in competition with ATL_5262 in tissues with high UNC5C expression (“High expression region”=mouse brain tissue) or control tissues with no or low UNC5C expression (“Liver ctrl”=mouse liver tissue).

FIG. 23B shows the results of a netrin-1 competition assay demonstrating that binding of ATLX-1282 and ATL_5262 to mouse brain tissue is blocked via Netrin-1. Fluorescent and Transmitted light tissue section images (20×), showing binding of ATL_5262 to mouse brain or control liver tissue under various blocking conditions (no netrin block; netrin-1 block; clusterin block (control)).

FIG. 23C shows the results of a netrin-1 competition assay demonstrating that binding of ATLX-1282 and ATL_5262 to mouse brain tissue is blocked via Netrin-1. Effect of Netrin-1 and Netrin-4 blocking in competition with ATLX-1282 in naïve (i.e., untreated) mouse brain sections, or liver control tissue (“Liver ctrl”).

FIG. 23D shows the results of a netrin-1 competition assay demonstrating that binding of ATLX-1282 and ATL_5262 to mouse brain tissue is blocked via Netrin-1. Representative Fluorescent and Transmitted light tissue section images showing blocking effect of Netrin-4 and Netrin-1 of ATLX-1282 binding (resulting in reduced staining) in naïve mouse brain sections.

FIG. 24A shows the results of a flow cytometry assay showing binding of ATL5262 and ATLX-1282 to UNC5C and the UNC5C T835M variant (AD variant) overexpressed in HEK-293 cells. Quantification of UNC5C binding as measured by the % of IgG+ cells (detecting the antibody).

FIG. 24B shows the results of a flow cytometry assay showing binding of ATL5262 and ATLX-1282 to UNC5C and the UNC5C T835M variant (AD variant) overexpressed in HEK-293 cells. Quantification of UNC5C binding as measured by the Mean fluorescence intensity (MFI) of IgG.

FIG. 25A shows the X-ray crystal structure of an ATL_5262 Fab fragment bound to human UNC5C Ig domains. FIG. 25A shows a cartoon view of the full Fab-UNC5C complex.

FIG. 25B shows the X-ray crystal structure of an ATL_5262 Fab fragment bound to human UNC5C Ig domains. The figure shows a first close-up view of the specific interactions between the ATL_5262 and UNC5C as shown in FIG. 25A.

FIG. 25C shows the X-ray crystal structure of an ATL_5262 Fab fragment bound to human UNC5C Ig domains. The figure shows a second close-up view of the specific interactions between the ATL_5262 and UNC5C as shown in FIG. 25A.

FIG. 25D shows the X-ray crystal structure of an ATL_5262 Fab fragment bound to human UNC5C Ig domains. The figure shows a third close-up view of the specific interactions between the ATL_5262 and UNC5C as shown in FIG. 25A.

FIG. 25E shows the X-ray crystal structure of an ATL_5262 Fab fragment bound to human UNC5C Ig domains. The figure shows a fourth close-up view of the specific interactions between the ATL_5262 and UNC5C as shown in FIG. 25A.

FIG. 25F shows the X-ray crystal structure of an ATL_5262 Fab fragment bound to human UNC5C Ig domains. The figure shows a fifth close-up view of the specific interactions between the ATL_5262 and UNC5C as shown in FIG. 25A.

FIG. 25G shows the X-ray crystal structure of an ATL_5262 Fab fragment bound to human UNC5C Ig domains. The figure shows a protein alignment of human UNC5 family members B (SEQ ID NO: 260), A (SEQ ID NO; 261) and D (SEQ ID NO: 262) to region of human UNC5C Ig-like domain 1 that interacts with ATL_5262 (SEQ ID NO: 259).

FIG. 26A shows sequences of antibodies described herein, with variable positions in the heavy chain highlighted. Heavy chains FW1 and CDR1 sequences, aligned by IMGT position numbering. VH of ATL_0005262: SEQ ID NO: 1; VH of ATL_0006178: SEQ ID NO: 93; VH of ATL_0006530: SEQ ID NO: 122; VH of ATL_0006531: SEQ ID NO: 123; VH of ATL_0006532: SEQ ID NO:124; VH of ATL_0006533: SEQ ID NO:125; VH of ATL_0006534: SEQ ID NO:126; VH of ATL_0006535: SEQ ID NO:127; VH of ATL_0006536: SEQ ID NO:128; VH of ATL_0006537: SEQ ID NO:129; VH of ATL_0006538: SEQ ID NO:130; VH of ATL_0006539: SEQ ID NO:131.

FIG. 26B shows the FW2 and CDR2 sequences of the heavy chains described in relation to FIG. 26A.

FIG. 26C shows the FW3 sequences of the heavy chains described in relation to FIG. 26A.

FIG. 26D shows the CDR3 and FW4 sequences of the heavy chains described in relation to FIG. 26A.

FIG. 26E. Light chains FW1 and CDR1, aligned by IMGT position numbering. VL of ATL_0005262: SEQ ID NO: 9; VL of ATL_0006178: SEQ ID NO: 92; VL of ATL_0006530: SEQ ID NO: 132; VL of ATL_0006531: SEQ ID NO: 133; VL of ATL_0006532: SEQ ID NO:134; VL of ATL_0006533: SEQ ID NO:135; VL of ATL_0006534: SEQ ID NO:136; VL of ATL_0006535: SEQ ID NO:137; VL of ATL_0006536: SEQ ID NO:138; VL of ATL_0006537: SEQ ID NO:139; VL of ATL_0006538: SEQ ID NO:140; VL of ATL_0006539: SEQ ID NO:141.

FIG. 26F shows the FW2 and CDR2 sequences of the light chains described in relation to FIG. 26E.

FIG. 26G shows the FW3 sequences of the light chains described in relation to FIG. 26E.

FIG. 26H shows the CDR3 and FW4 sequences of the light chains described in relation to FIG. 26E.

### DETAILED DESCRIPTION OF THE INVENTION

Aspects and embodiments of the present invention will now be discussed with reference to the accompanying figures. Further aspects and embodiments will be apparent to those skilled in the art.

Disclosed herein are antibodies and fragments thereof that are capable of specifically binding to UNC5C protein or a fragment thereof. As used herein, an antibody capable of “specific binding” or “specifically binding” a target is one able to bind through the association of the epitope recognition site with an epitope within the target. It is distinct from non-specific binding, for example Fc-mediated binding, ionic and/or hydrophobic interactions. In other words, an antibody which specifically binds a target recognises and binds to a specific protein structure within it rather than to proteins generally.

The present disclosure refers to antibodies described herein using references specified as “ATL_000 xxxx”, “ATL_xxxx” or “xxxx”, where “xxxx” is a four digits reference number specific to an antibody described herein. All of the above notations are used interchangeably to refer to the same antibody or a portion thereof (e.g. a VH, VL or part thereof, of the antibody). For example, antibody ATL_0005262 is interchangeably referred to herein as ATL_5262 and 5262.

### Binding to UNC5C

Unc-5 netrin receptor C (UNC5C) belongs to the UNC5 family of cell membrane netrin receptors, which are part of the immunoglobulin superfamily (Leonardo, E., et al. (1997)). UNC5 family members have two extracellular Ig domains and two thrombospondin type 1 (TSP-1) domains. The intracellular c terminus consists of a zona occludens 5 (ZU5) domain, a DCC-binding domain and a death domain involved in apoptosis regulation (Ackerman S L, Knowles B B. 1998). Four subtypes of UNC5 have been identified, namely UNC5 A-D (referred to as UNC5 H1-4 in other mammalian species), which are widely expressed in the central nervous system (CNS) (Rajasekharan, S., Kennedy, T. E. (2009)). Additional netrin receptors include Down's syndrome cell adhesion molecule (DSCAM), neogenin, Deleted in Colorectal Cancer (DCC) and CD146 (also known as Melanoma Cell Adhesion Molecule (MCAM or Mel-CAM). These receptors differ in the number of n-terminal Ig domains, inclusion of fibronectin type Ill domains and intracellular domains. For an overview of cell membrane netrin receptors, see Dun et al., 2017. The antibodies described herein are capable of specifically binding UNC5C over other netrin receptors such as neogenin, DCC, DSCAM, UNC5A, UNC5B, and UNC5D. Binding of an antibody to a candidate protein can be verified for example by biolayer interferometry analysis as described herein.

UNC5C is a dependence receptor responsible for the regulation of neuronal apoptosis, and whether UNC5C promotes or inhibits apoptosis depends on its binding to netrin-1 (Lambi et al (2001); Poliak S, et al. 2015). UNC5 receptors have intracellular death domains. This domain can interact with death-associated protein kinase (DAPK1) to induce apoptosis in the absence of ligand binding, whilst the receptors remain in monomeric form. The T835M mutation in the death domain of UNC5C further increases apoptosis. This increased signal allowed the determination of the involvement of death-associated protein kinase 1/protein kinase D/apoptosis signal-regulating kinase 1 (ASK1)/JNK/NADPH oxidase/caspases in this signalling cascade (Hashimoto et al 2016 Signal Transduction 291(23), 12282-12293)). UNC5-induced cell death is increased by caspase cleavage in its intracellular death domain which may lead to a feed-forward loop once apoptosis is initiated. All Unc5 receptors are cleaved by caspase-3 in vitro (Llambi et al 2001) Cleavage of UNC5C by LGMN has also been proposed to increase UNC5C mediated neuronal apoptosis relevant to Alzheimer's disease (Chen et al 2021). However, in the presence of netrin-1, UNC5C forms a dimer and promotes neuronal survival, migration, and differentiation, i.e. it has anti-apoptotic role. Multimerisation of UNC5 receptors is sufficient to prevent the apoptotic signal pathway activation (Mille et al 2009 Cell Death and Differentiation 16, 1344-1351) and netrin-1 directly inhibits apoptosis in neurones (Llambi et al 2001). UNC5C has also been shown to form heterodimers with DCC or DSCAM (Boyer and Gupton, 2018). Antibodies described herein may bind monomeric UNC5C and dimeric UNC5C. Antibodies described herein may bind homodimeric UNC5C.

UNC5C has been implicated in Alzheimer's disease where truncated UNC5C resulting in enhanced neuronal apoptosis was found to be associated with accelerated AD pathology in mice (Chen et al., 2021). Further, several SNPs in UNC5C are linked to the development of Alzheimer's disease (AD). For example, the SNP T835M predisposes to the AD and increases neuronal apoptosis (Li Q, et al. 2018). UNC5C has also been implicated in Parkinson's Disease (PD) where cleavage of UNC5C was shown to cause neuronal loss in a mouse model of PD (Chen et al. 2022).

Netrin-1 is one of several netrins, all members of the laminin superfamily. Netrins are secreted proteins that direct axon extension and cell migration during neural development and regulate cell adhesion, the maturation of cell morphology, cell survival and tumorigenesis (Rajasekharan, et al. (2009)). They are bifunctional proteins that act as attractants for some cell types and as repellents for others. The UNC-5 family of receptors mediate the repellent response to netrin. Netrin 1 consists of a laminin-like domain in the N-terminal (LN, also known as domain VI), followed by three epidermal growth factor (EGF) repeats (EGF1, EGF2 and EGF3, also known as domain V), and a C-terminal Netrin-like domain (NTR) that shares homology with domains found in complement and other proteins (Dun et al. 2017).

In embodiments, antibodies described herein specifically bind to UNC5C and mimics and/or competes with Netrin-1 for binding with UNC5C. Competition with Netrin-1 binding may be assessed by ELISA assays as described herein. Thus, antibodies described herein may compete for the same binding site on UNC5C as Netrin-1. Antibodies described herein may bind the same binding site on UNC5C as Netrin-1. An antibody mimicking netrin-1 may refer to the antibody interacting with UNC5C in a way that mimics the effect of netrin-1 on UNC5C structure and/or activity. An antibody competing with netrin-1 for binding to UNC5C may refer to the binding of the antibody to UNC5C being dependent on the presence or absence of Netrin-1, and/or the binding of Netrin-1 to UNC5C being dependent on the presence or absence of the antibody. An antibody competing with netrin-1 for binding to UNC5C may refer to the binding of the antibody to UNC5C being inhibited by Netrin-1. For example, an antibody competing with netrin-1 for binding to UNC5C may refer to the binding of the antibody to UNC5C being at least partially inhibited in the presence of Netrin-1. This may be assessed for example using an ELISA assay, such as described herein. For example, UNC5C may be pre-incubated with Netrin-1 prior to incubation with the antibody, and the amount of bound antibody may be compared to a control condition without Netrin-1 pre-incubation. A reduction in the amount of bound antibody may indicate that the antibody competes with Netrin-1 for binding to UNC5C. In embodiments, a signal indicative of the presence of the antibody bound to UNC5C may be reduced by at least 50%, at least 60%, at least 70%, at least 80% or at least 90% in the presence of Netrin-1 (such as e.g. following pre-incubation with 200 microg/l Netrin-1). In embodiments, binding of the antibody to UNC5C may be reduced by at least 50%, at least 60%, at least 70%, at least 80% or at least 90% in the presence of Netrin-1 (such as e.g. following pre-incubation with 200 microg/l Netrin-1).

Antibodies as described herein may be such that binding of the antibody to UNC5C is inhibited by Netrin-1 in a concentration dependent manner. This can be assessed for example using an ELISA assay as described herein, or any other binding assay in combination with incubation with a plurality of different concentrations of Netrin-1 (e.g. a Netrin-1 dilution series). A reduction in binding of the antibody to UNC5C in the presence of Netrin-1 (e.g. when the antibody is incubated with UNC5C following pre-incubation with Netrin-1), which is dependent on the concentration of Netrin-1, may be considered to be indicative of Netrin-1 concentration-dependent inhibition of binding of the antibody to UNC5C. In embodiments, a signal indicative of the presence of the antibody bound to UNC5C may be reduced by at least 50% in the presence of at least 10⁻⁸ M of Netrin-1 (such as e.g. following pre-incubation with 10⁻⁸ M of Netrin-1). In embodiments, binding of the antibody to UNC5C may be reduced by at least 50% in the presence at least 10⁻⁸ M of Netrin-1 (such as e.g. following pre-incubation with 10⁻⁸ M of Netrin-1). In embodiments, a signal indicative of the presence of the antibody bound to UNC5C may be reduced by at least 80% in the presence of at least 10⁻⁷ M of Netrin-1 (such as e.g. following pre-incubation with 10⁻⁷ M of Netrin-1). In embodiments, binding of the antibody to UNC5C may be reduced by at least 80% in the presence at least 10⁻⁷ M of Netrin-1 (such as e.g. following pre-incubation with 10⁻⁷ M of Netrin-1). In embodiments, a signal indicative of the presence of the antibody bound to UNC5C may be reduced by at least 90% or at least 95% in the presence of at least 5×10⁻⁷ or 1×10⁻⁶ M of Netrin-1 (such as e.g. following pre-incubation with 5×10⁻⁷ or 1×10⁻⁶ M of Netrin-1). In embodiments, binding of the antibody to UNC5C may be reduced by at least 90% or at least 95% in the presence at least 5×10⁻⁷ or 1×10⁻⁶ M of Netrin-1 (such as e.g. following pre-incubation with 5×10⁻⁷ or 1×10⁻⁶ M of Netrin-1). Any pre-incubation may be for a suitable amount of time, such as e.g. around 30 minutes.

Antibodies described herein may have a binding site that at least partially overlaps with the netrin-1 binding site. Antibodies described herein may cause the dimerization of UNC5C.

The human gene encoding UNC5C (Gene ID: 8633) is present on 4q22.3 and consists of 20 exons. Reference non-human UNC5C amino acid and coding sequences are available in public databases.

The sequence of human UNC5C is available under Uniprot identifier 095185. It comprises 931 amino acids. Amino acids 62-159 form an Ig-like domain, amino acids 161-256 form an Ig-like C2-type domain, amino acids 260-314 and 316-368 form TSP type-1 1 and TSP type-1 2 domains, amino acids 530-673 form a ZU5 domain and amino acids 850-929 form a death domain. The protein is cleaved at amino acids 415-416 by caspase-3. A reference human UNC5C amino acid sequence is provided as SEQ ID NO: 99.

The sequence of mouse UNC5C is available under Uniprot identifier 008747. It comprises 931 amino acids. Amino acids 62-159 form an Ig-like domain (also referred to herein as the first immunoglobulin domain or N terminal immunoglobulin domain), amino acids 161-256 form an Ig-like C2-type domain (also referred to herein as the second immunoglobulin domain), amino acids 260-314 and 316-368 form TSP type-1 1 and TSP type-1 2 domains, amino acids 530-673 form a ZU5 domain and amino acids 850-929 form a death domain. The protein is cleaved at amino acids 415-416 by caspase-3. A reference mouse UNC5C amino acid sequence is provided as SEQ ID NO: 100. However, as used herein, the term “UNC5C” encompasses truncations, derivatives, and variants of the sequence of UNC5C provided herein, and may refer to any protein with at least 80%, at least 90%, or at least 95% sequence identity. The antibodies described herein are capable of specifically binding a peptide or protein having or comprising the amino acid sequence of SEQ ID NO: 99 (including a full length UNC5C protein comprising said sequence, or a fragment of said protein comprising said sequence), or a fragment thereof, such as e.g. a fragment comprising or consisting of amino acids 41-380 of human or murine (mouse) UNC5C (such as e.g. the amino acid sequence of SEQ ID NO: 101 or 102) or a corresponding homologous sequence. The antibodies described herein are capable of specifically binding a peptide or protein comprising the extracellular domain of UNC5C or a part thereof, such as e.g. a part comprising amino acids 41 to 380 of human or murine UNC5C, or a corresponding homologous sequence. Antibodies described herein are capable of specifically binding human UNC5C and human mutant UNC5C T835M. Antibodies described herein may bind to mutant forms of human UNC5C that comprise one or more mutations that are not in the extracellular region of the protein. The antibody may bind to an epitope located in the extracellular region of UNC5C. In particular, the antibody may bind to the N terminal immunoglobulin domain of UNC5C (also referred to as “first immunoglobulin domain). The antibody may bind to one or more residues of UNC5C that are involved in binding of Netrin-1 to UNC5C. Thus, the antibody may bind to one or more residues of UNC5C located in the first immunoglobulin domain of UNC5C.

Antibodies described herein bind UNC5C (e.g. mouse and/or human) with high affinity. High affinity binding may be assessed by measuring the EC50 value, the concentration at which the antibody produces a half-maximal binding, for example via ELISA as described herein. For example, affinity of the antibody may be assessed by binding to plate-bound recombinant human UNC5C as described herein. High affinity as referred to herein means an EC50 value of at most 1E-06 M (i.e. 1000 nM), at most 2E-06 M, at most 3E-06M, at most 4E-06, at most 5E-06 M, at most 6E-06 M, at most 7E-06, at most 8E-06 M, at most 9E-06 M, at most 1E-07 M (i.e. 100 nM), at most 5E-07 M, at most 1 E-08 M, at most 2 E-08 M, at most 3 E-08 M, at most 4 E-08 M, at most 5 E-08 M, at most 6 E-08 M, at most 7 E-08 M, at most 8 E-08 M, at most 9 E-08 M, at most 1 E-09 M, at most 2 E-09 M, at most 3 E-09 M, at most 4 E-09 M, at most 5E-09 M, at most 6 E-09 M, at most 7 E-09 M; at most 8 E-09 M, at most 9 E-09 M, at most 1E-10 M, at most 2 E-10 M, at most 3 E-10 M, at most 4 E-10 M, at most 5E-10 M, at most 6 E-10 M, at most 7 E-10 M. at most 8 E-10 M, at most 9 E-10 M, at most 1E-11M, at most 2 E-11M, at most 3 E-11M, at most 4E-11M, at most 5 E-11M, at most 6E-11M, at most 7 E-11M, at most 8 E-11M, at most 9 E-11M, at most 1E-12M, at most 2 E-12M, at most 3 E-12M, at most 4 E-12M, 5E-12M, at most 6 E-12M, at most 7 E-12M, at most 8 E-12M, at most 9 E-12M or 1E-13M. In embodiments, the antibodies described herein bind UNC5C with an EC50 of at most 100 nM, as assessed via ELISA (e.g. as described herein).

In some embodiments, the antibody binds human UNC5C with an EC50 of at most 1E-08M, at most 1.1E-08M, at most 2.24E-08, at most 2.40E-08, at most 9.81E-08 M, at most 4.52E-09, at most 5.5E-09M, at most 9.62E-09M, at most 5.50E-10, at most 7.10E-10, at most 1.00E-11, at most 1.30E-11, at most 3.90E-11, at most 2.80E-12, or at most 9.20E-12.

In some embodiments, the antibody binds human UNC5C with an EC50 of about 1.1E-08M, about 2.24E-08, about 2.40E-08, about 9.81E-08 M, about 4.52E-09, about 5.5E-09M, about 8.00E-09, about 9.62E-09M, about, 5.50E-10, about 7.10E-10, about 1.00E-11, about 1.30E-11, about 3.90E-11, about 2.80E-12, about 9.20E-12.

In some embodiments, the EC50 value is between 1E-07 M and 1E-13M, 5E-07 M and 1E-13M; 1E-08 M and 1E-13M; 5E-08 M and 1E-13M; 1E-09 M and 1E-13M; 5E-09 M and 1E-13M; 1E-10 M and 1E-13M; 5E-10 M and 1E-13M; 1E-11 M and 1E-13M; 5E-11 M and 1E-13M; 1E-12 M and 1E-13M; 5E-12 M and 1E-13M; between 1E-07 M and 1E-12M, 5E-07 M and 1E-12M; 1E-08 M and 1E-12M; 5E-08 M and 1E-12M; 1E-09 M and 1E-12M; 5E-09 M and 1E-12M; 1E-10 M and 1E-12M; 5E-10 M and 1E-12M; 1E-11 M and 1E-12M; 5E-11 M and 1E-12M; 1E-07 M and 1E-11M, 5E-07 M and 1E-11M; 1E-08 M and 1E-11M; 5E-08 M and 1E-11M; 1E-09 M and 1E-11M; 5E-09 M and 1E-11M; 1E-10 M and 1E-11M; 5E-10 M and 1E-1M; 1E-07 M and 1E-10M, 5E-07 M and 1E-10M; 1E-08 M and 1E-10M; 5E-08 M and 1E-10M; 1E-09 M and 1E-10M; 5E-09 M and 1E-10M; 1E-07 M and 1E-09M, 5E-07 M and 1E-09M; 1E-08 M and 1E-09M; 5E-08 M and 1E-09M; 1E-07 M and 1E-08M, 5E-07 M and 1E-08M.

In some embodiments, antibodies described herein are capable of specifically binding a UNC5C protein or protein fragments comprising or consisting of a UNC5C variant amino acid sequence. In some embodiments, the UNC5C variant protein or fragment comprises an amino acid sequence that has at least 70%, at least 80%, at least 85%, at least 90%, at least 95%, or at least 99% identity with SEQ ID NO: 99. In some embodiments, the antibodies disclosed herein are capable of specifically binding a UNC5C fragment comprising at least 20%, 30%, 40%, 50%, 60%, 70%, 80%, 90%, 95% or more of the UNC5C amino acid sequence, or a UNC5C variant sequence.

UNC5C may be human UNC5C or mouse UNC5C. Suitably, UNC5C may be human UNC5C. UNC5C may refer to human UNC5C unless context indicates otherwise. In other embodiments, for example when the individual to be treated is a non-human mammal, UNC5C may be non-human UNC5C.

### Structural Properties

Antibodies of the present disclosure may specifically target a single epitope on the UNC5C protein. The term epitope, also known as antigenic determinant, as used herein refers to any protein determinant capable of specific binding to an immunoglobulin or fragment thereof. Epitopic, or antigenic, determinants usually consist of chemically active surface groupings of molecules such as amino acids or sugar side chains and usually have specific three-dimensional structural characteristics, as well as specific charge characteristics.

Antibodies described herein may bind to a discontinuous epitope of UNC5C. Antibodies described herein may bind to an epitope that comprises one or more of, or all of, the following residues of UNC5C provided as SEQ ID NO: 99: Thr89, Gln90, Gln102, Lys103, Val107, Asp108, Glu109, Arg110, Val111, Ile118, Arg120 or corresponding residues in a homologous sequence. In some embodiments, e.g. in instances where the antibody is a heavy-chain only antibody, the antibody binds to residues Thr89, Gln90, Gln102, Lys103, Val107, Glu109, Ile118, and Arg120.

In embodiments, one or more of (or all of) of VH residues 55, 63, 64, 65, 66, 74, 108, 110 in IMGT numbering interact with one or more residues in the epitope. In embodiments, one or more of (or all of) of VH residues 55 (Tyr), 63 (Gly), 64 (T, H or S, more specifically Thr), 65 (T or S, more specifically Thr), 66 (Asn), 74 (Ser), 108 (Arg), 110 (Met) in IMGT numbering interact with one or more residues in the epitope. All of these residues are conserved across the VH of antibodies 5262, 5996, 5997, 5998, 5999, 6000, 6001, 6002, 6003, 6004, 6005, 6033, 6034, 6035, 6036, 6037, 6038, 6039, 6177, 6178. All of these residues apart from 64 (Thr) and/or 65 (Thr) are conserved across the VH of antibodies 6187 (where position 64 is H and position 65 is T), 6191 (where position 64 is S and position 65 is S). This indicates that substitutions at these positions would be possible while maintaining binding to the epitope. Thus, in embodiments, one or more of (or all of) of VH residues 55 (Tyr), 63 (Gly), 64 (Thr or conservative substitution e.g. H or S), 65 (Thr or conservative substitution e.g. S), 66 (Asn), 74 (Ser), 108 (Arg), 110 (Met) in IMGT numbering interact with one or more residues in the epitope. In embodiments, one or more of (or all of) VL residues 38, 107, 108, 109, and 114 in IMGT numbering interact with one or more residues in the epitope. In embodiments, one or more of (or all of) VL residues 38 (Y, D, L or T, such as Tyr in specific embodiments), 107 (S, Y, F or W, such as Ser in specific embodiments), 108 (Y, D, T, or G, such as Tyr in specific embodiments), 109 (S, D, K or absent, such as Ser in specific embodiments), 114 (T, N, S, R, G, or A, such as Thr in specific embodiments) in IMGT numbering interact with one or more residues in the epitope. Position 38 is Tyr in antibodies 5262, 6178, 6530, 6531, 6533, 6534, 6537, 6539, D in antibody 6335, L in antibody 6336 and T in antibody 6538. Position 107 is S in antibodies 5262 and 6178 (same VL), Y in antibodies 6530, 6532, 6533, 6534, 6535 and 6536, F in 6531, and W in antibodies 6537, 6538 and 6539. Position 108 is Y in antibodies 5262 and 6178, D in antibodies 6530, 6532, 6533, 6534, 6535, 6537 and 6538, T in antibodies 6531 and 6536, and G in antibody 6539. Position 109 is S in antibodies 5262, 6178, 6530, 6533, 6534, 6535, 6536 and 6537, D in antibodies 6531 and 6538, K in antibody 6532 and absent in antibody 6539. Position 114 is T in antibodies 5262, 6178 and 6536, N in antibodies 6530, 6533 and 6538, S in antibodies 6531 and 6537, R in antibody 6532, G in antibody 6534, A in antibody 6535. Thus, in embodiments, the VH of an antibody as described herein has: at residue 55: Tyr, at residue 63: Gly, at residue 64: T, H or S, more specifically Thr, at residue 65: T or S, more specifically Thr, at residue 66: Asn, at residue 74: Ser, at residue 108: Arg, at residue 110: Met, where all positions are in IMGT numbering. Further, in embodiments, the VL of an antibody as described herein has: at residue 38: Y, D, L or T, such as Tyr in specific embodiments, at residue 107: S, Y, F or W, such as Ser in specific embodiments, at residue 108: Y, D, T, or G, such as Tyr in specific embodiments, at residue 109: S, D, K or absent (deletion), such as Ser in specific embodiments, at residue 114: T, N, S, R, G, or A, such as Thr in specific embodiments, where all positions are in IMGT numbering. Any of the amino acids at any of these positions can be combined with each other, and all resulting combinations are explicitly envisaged.

In some instances, (a) VH residue 64 (e.g. Thr) of antibodies described herein interacts with Thr 89 of the discontinuous epitope; and/or (b) VH residue 63 (e.g. Gly) of antibodies described herein interacts with Gln 90 of the discontinuous epitope; and/or (c) VH residue 65 (e.g. Thr) of antibodies described herein interacts with Gln 102 of the discontinuous epitope; and/or (d) VH residue 74 (e.g. Ser) of antibodies described herein interacts with Lys103 of the discontinuous epitope; and/or (e) VH residue 66 (Asn) of antibodies described herein interacts with Val 107 of the discontinuous epitope; and/or (f) VH residues 55 (e.g. Tyr), 66 (e.g. Asn), 108 (e.g. Arg), and/or 110 (e.g. Met) of antibodies described herein interact with Glu109 of the discontinuous epitope; and/or (g) VH residues 108 (e.g. Arg), and/or 110 (e.g. Met) of antibodies described herein interact with Ile118 of the discontinuous epitope; and/or (h) VH residue 55 (e.g. Tyr), 64 (e.g. Thr), and/or 65 (e.g. Thr) of antibodies described herein interacts with Arg120 of the discontinuous epitope. In some instances, (i) VL residue 114 (e.g. Thr) of antibodies described herein interacts with Val107 and/or Asp108, and/or Glu109 of the discontinuous epitope; and/or (j) VL residue 109 (e.g. Ser) of antibodies described herein interacts with Glu109 and/or Arg110 of the discontinuous epitope; and/or (k) VL residue 108 (e.g. Tyr) of antibodies described herein interacts with Arg110 and/or Val111 of the discontinuous epitope; wherein the UNC5C residues are those at the specified positions in SEQ ID NO: 99 or corresponding positions in a homologous sequence. The skilled person will be familiar with methods for mapping epitopes, including but not limited to X-ray co-crystallography as described herein; cryogenic electron microscopy as described, for example, in Renaud et al., 2018; mutagenesis strategies, such as alanine scanning mutagenesis as described, for example, in Cunningham et al., 1989.

An “antigen binding domain” describes the part of a molecule that binds to all or part of the target antigen. An antibody generally comprises six complementarity-determining regions (CDRs); three in the VH region: HCDR1, HCDR2 and HCDR3, and three in the VL region: LCDR1, LCDR2, and LCDR3. The six CDRs together define the paratope of the antigen binding domain, which is the part of the antigen binding domain which binds to the target antigen. The paratope of an antibody as described herein may include the following VH residues: 55 (e.g. Tyr), 63 (e.g. Gly), 64 (e.g. Thr, His or Ser), 65 (e.g. Thr or Ser), 66 (e.g. Asn), 74 (e.g. Ser), 108 (e.g. Arg), 110 (e.g. Met) in IMGT numbering. The paratope of an antibody as described herein may include the following VL residues: 38 (e.g. Tyr, D, L or T), 107 (e.g. Ser, Y, F, or W), 108 (e.g. Tyr, D, T or G), 109 (e.g. Ser, D, K or − (deletion)), 114 (e.g. Thr, N, S, R, G or A) in IMGT numbering. Thus, an antibody according to any embodiment of the present disclosure may comprise a VH with the following residues: at position 55: Tyr, at position 63: Gly, at position 64: Thr, at position 65: Thr, at position 66: Asn, at position 74: Ser, at position 108: Arg, at position 110: Met in IMGT numbering.

Further, an antibody according to any embodiment of the present disclosure may comprise a VL with the following residues: at position 38: Tyr, at position 107: Ser, at position 108: Tyr, at position 109: Ser, at position 114: Thr in IMGT numbering. The present disclosure relates primarily to antibody molecules, whether whole antibody (e.g. IgG, such as IgG1) or antibody fragments (e.g. single-chain variable fragment (scFv), Antibody fragments (Fab) or bivalent antibody fragments (F(ab′)2), single-domain antibody (sdAb). Antibody antigen binding regions (also referred to as “antigen binding portions”) are provided, as are antibody heavy chain variable (VH) and light chain variable (VL) domains. Within VH and VL domains are provided complementarity determining regions (CDRs), which may be provided within different framework regions (FRs), to form VH or VL domains, as the case may be. An antigen binding site may consist of an antibody VH domain and/or a VL domain. Thus, an antibody according to the present disclosure may be an scFv antibody molecule, a nanobody, or a whole antibody. The antibody may comprise an antibody constant region. The antibody constant region may be a human constant region, such as a human IgG1 constant region. The antibody may be a whole antibody. The antibody may be an IgG, such as an IgG1 or variant thereof. The antibody may be an IgG1 variant L234A/L235A (LALA).

Antibodies according to the present disclosure may be provided in isolated form. The term “antibody” encompasses a fragment or derivative thereof, or a synthetic antibody or synthetic antibody fragment.

The antigen-binding portion may be a part of an antibody (e.g. a Fab fragment) or a synthetic antibody fragment (e.g. an scFV). Suitable antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 25 792-5 799).

An antibody or fragment thereof may be a monoclonal antibody (mAb). mAbs are homogenous populations of antibodies specifically targeting a single epitope on an antigen.

Fragments of antibodies, such as Fab and F(ab′)2 fragments, may also be provided, as can genetically engineered antibodies and antibody fragments. The VH and VL domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parent antibody (Morrison et al (1984) Proc. Natl. Acad. Sd. USA 81, 6851-6855).

That antigenic specificity is conferred by variable domains and is independent of the constant domains as known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are linked via a flexible oligopeptide (Bird et al (1988) Science 20 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The term “ScFv molecules” refers to molecules wherein the VH and VL partner domains are covalently linked, e.g. by a flexible oligopeptide. Fab, Fv, ScFv and sdAb antibody fragments can all be expressed in and secreted from E. coli, thus allowing the facile production of large amounts of the said fragments.

Whole antibodies, and F(ab′)2 fragments are “bivalent”. The term “bivalent” means that the said antibodies and F(ab′)2 fragments have two antigen combining sites. In contrast, Fab, Fv, ScFv and dAb fragments are monovalent, having only one antigen combining site.

Antibodies according to the present disclosure may be detectably labelled or, at least, capable of detection. For example, the antibody may be labelled with a radioactive atom or a coloured molecule or a fluorescent molecule or a molecule which can be readily detected in any other way. Suitable detectable molecules include fluorescent proteins, luciferase, enzyme substrates, and radiolabels. The binding moiety (antibody or fragment thereof) may be directly labelled with a detectable label or it may be indirectly labelled. For example, the binding moiety may be an unlabelled antibody which can be detected by another antibody which is itself labelled. Alternatively, the second antibody may have bound to it biotin and binding of labelled streptavidin to the biotin is used to indirectly label the first antibody.

A “fragment” of an antibody may comprise any number of residues of a “parental” antibody, whilst retaining target binding ability. A fragment may lack effector function, for example may be entirely unable to bind or show diminished binding to the Fc receptor, relative to the parent. A fragment is typically smaller than the parental antibody. A fragment may comprise 50%, 60%, 70%, 80%, 90%, 95% or more of the contiguous or non-contiguous amino acids of the parental antibody. A fragment may comprise 50, 100, 150, 200, 250, 300 or more contiguous or non-contiguous amino acids of the parental antibody. A fragment may comprise deletions in the Fc region, or of the Fc region. A fragment may retain the CDRs and/or the variable domains of the parental antibody, unaltered. In some embodiments, a fragment is an Fab fragment or an F(ab′)2 fragment.

CDR sequences are described herein using the IMGT numbering (Lefranc, M.-P., Immunology Today, 18, 509 (1997)).

Antibodies according to the present disclosure may have a heavy chain variable domain (VH) with the CDRs of the heavy chain variable domain (VH) of antibody ATL_5262, ATL_6036, ATL_6039, ATL_6177, or ATL_6178. Antibodies according to the present disclosure may have the following heavy chain CDRs: CDRH1: SEQ ID NO:20; CDRH2: SEQ ID NO:21 or SEQ ID NO:22; and CDRH3: SEQ ID NO:23.

The disclosure further provides antibodies comprising a heavy chain variable domain (VH) with the CDRs of the VH of antibodies ATL_6187 or ATL_6191: CDRH1 comprising amino acid sequence SEQ ID NO: 115 or SEQ ID NO: 116, CDRH2 comprising amino acid sequence SEQ ID NO: 117 or SEQ ID NO: 118, and CDRH3 comprising amino acid sequence SEQ ID NO: 119 or SEQ ID NO: 120. In particular, antibodies according to the present disclosure may have a heavy chain variable domain (VH) with the CDRs of the heavy chain variable domain (VH) of antibody ATL_6187, i.e. CDRH1 comprising amino acid sequence SEQ ID NO: 115, CDRH2 comprising amino acid sequence SEQ ID NO: 117, and CDRH3 comprising amino acid sequence SEQ ID NO: 119. Further, antibodies according to the present disclosure may have a heavy chain variable domain (VH) with the CDRs of the heavy chain variable domain (VH) of antibody ATL_6191, i.e. CDRH1 comprising amino acid sequence SEQ ID NO: 116, CDRH2 comprising amino acid sequence SEQ ID NO: 118, and CDRH3 comprising amino acid sequence SEQ ID NO: 120. In an antibody according to the present disclosure at least one of the VH CDR 1-3 sequences may vary. For example, a variant may have VH CDRs that have up to 3 amino acid mutations over the above VH CDRs.

The disclosure further provides antibodies comprising a heavy chain variable domain (VH) with the CDRs of the VH of the antibodies ATL_6530; ATL_6531; ATL_6532; ATL_6533; ATL_6534; ATL_6535; ATL_6536; ATL_6537; ATL_6538; ATL_6539: CDRH1 comprising amino acid sequence SEQ ID NO: 142-149, CDRH2 comprising amino acid sequence SEQ ID NO: 150-158, and CDRH3 comprising amino acid sequence SEQ ID NO: 159-168. In particular, antibodies according to the present disclosure may have a heavy chain variable domain (VH) with the CDRs of the heavy chain variable domain (VH) of antibody ATL_6530, i.e. CDRH1 comprising amino acid sequence SEQ ID NO: 142, CDRH2 comprising amino acid sequence SEQ ID NO: 150, and CDRH3 comprising amino acid sequence SEQ ID NO: 159. In particular, antibodies according to the present disclosure may have a heavy chain variable domain (VH) with the CDRs of the heavy chain variable domain (VH) of antibody ATL_6531, i.e. CDRH1 comprising amino acid sequence SEQ ID NO: 143, CDRH2 comprising amino acid sequence SEQ ID NO: 151, and CDRH3 comprising amino acid sequence SEQ ID NO: 160. In particular, antibodies according to the present disclosure may have a heavy chain variable domain (VH) with the CDRs of the heavy chain variable domain (VH) of antibody ATL_6532, i.e. CDRH1 comprising amino acid sequence SEQ ID NO: 144, CDRH2 comprising amino acid sequence SEQ ID NO: 152, and CDRH3 comprising amino acid sequence SEQ ID NO: 161. In particular, antibodies according to the present disclosure may have a heavy chain variable domain (VH) with the CDRs of the heavy chain variable domain (VH) of antibody ATL_6533, i.e. CDRH1 comprising amino acid sequence SEQ ID NO: 145, CDRH2 comprising amino acid sequence SEQ ID NO: 153, and CDRH3 comprising amino acid sequence SEQ ID NO: 162. In particular, antibodies according to the present disclosure may have a heavy chain variable domain (VH) with the CDRs of the heavy chain variable domain (VH) of antibody ATL_6534, i.e. CDRH1 comprising amino acid sequence SEQ ID NO: 144, CDRH2 comprising amino acid sequence SEQ ID NO: 152, and CDRH3 comprising amino acid sequence SEQ ID NO: 163. In particular, antibodies according to the present disclosure may have a heavy chain variable domain (VH) with the CDRs of the heavy chain variable domain (VH) of antibody ATL_6535, i.e. CDRH1 comprising amino acid sequence SEQ ID NO: 143, CDRH2 comprising amino acid sequence SEQ ID NO: 154, and CDRH3 comprising amino acid sequence SEQ ID NO: 164. In particular, antibodies according to the present disclosure may have a heavy chain variable domain (VH) with the CDRs of the heavy chain variable domain (VH) of antibody ATL_6536, i.e. CDRH1 comprising amino acid sequence SEQ ID NO: 146, CDRH2 comprising amino acid sequence SEQ ID NO: 155, and CDRH3 comprising amino acid sequence SEQ ID NO: 165. In particular, antibodies according to the present disclosure may have a heavy chain variable domain (VH) with the CDRs of the heavy chain variable domain (VH) of antibody ATL_6537, i.e. CDRH1 comprising amino acid sequence SEQ ID NO: 147, CDRH2 comprising amino acid sequence SEQ ID NO: 156, and CDRH3 comprising amino acid sequence SEQ ID NO: 166.

In particular, antibodies according to the present disclosure may have a heavy chain variable domain (VH) with the CDRs of the heavy chain variable domain (VH) of antibody ATL_6538, i.e. CDRH1 comprising amino acid sequence SEQ ID NO: 148, CDRH2 comprising amino acid sequence SEQ ID NO: 157, and CDRH3 comprising amino acid sequence SEQ ID NO: 167. In particular, antibodies according to the present disclosure may have a heavy chain variable domain (VH) with the CDRs of the heavy chain variable domain (VH) of antibody ATL_6539, i.e. CDRH1 comprising amino acid sequence SEQ ID NO: 149, CDRH2 comprising amino acid sequence SEQ ID NO: 158, and CDRH3 comprising amino acid sequence SEQ ID NO: 168. In an antibody according to the present disclosure at least one of the VH CDR 1-3 sequences may vary. For example, a variant may have VH CDRs that have up to 3 amino acid mutations over the above VH CDRs.

In an antibody according to the present disclosure at least one of the VH CDR 1-3 sequences may vary. That is, the above set of CDRs may comprise one, two, or three mutations. Mutations as used herein refers to amino acid substitutions, insertions, or deletions. Mutations may be introduced by standard techniques, such as site-directed mutagenesis and PCR-mediated mutagenesis. A variant may have one or two amino acid mutations, such as substitutions compared with the set of VH CDR1-3 described above. In embodiments, an antibody according to the disclosure comprises CDRs with sequences that have one or two mutations, such as substitutions compared to the VH CDR sequences of any antibody described herein. For example, an antibody according to the disclosure may comprise VH CDRs with the sequences of any antibody above, except that one or two of the CDRHs comprise a substitution, where the total number of substitutions across the CDRHs does not exceed 2. In embodiments, a variant may have one, two or three, preferably at most one or two substitutions in each of one or more of the VH CDR1-3 described above. CDRH1 regions of any antibodies or fragments described herein may have a length of 8 amino acids. CDRH2 regions of any antibodies or fragments described herein may have a length of 7 amino acids. CDRH3 regions of any antibodies or fragments described herein may have a length of 13 amino acids. In embodiments, a variant may have VH CDRs that have at least 70%, at least 80% or at least 90% sequence identity with any set of VH CDRs described herein. For example, a variant with a combined CDRH 1-3 length of 28 amino acids may have up to 8, up to 5, or up to 2 amino acid substitutions compared to a set of VH CDRs described herein. In embodiments, a variant may have one or two substitutions comprising or consisting of a substitution at position 58 in CDRH2. The substitution may be N58Y.

An antibody according to the present disclosure may have a VH with the following CDRHs: CDRH1 comprising amino acid sequence SEQ ID NO:20, CDRH2 comprising amino acid sequence SEQ ID NO:21 and CDRH3 comprising amino acid sequence SEQ ID NO:23. These are the VH CDRs of ATL_5262 and all variants thereof except ATL_6178 (i.e. ATL_6033, 6034, 6035, 6036, 6037, 6038, 6039, 6177).

An antibody according to the present disclosure may have a VH with the following CDRHs: CDRH1 comprising amino acid sequence SEQ ID NO:20, CDRH2 comprising amino acid sequence SEQ ID NO:22 and CDRH3 comprising amino acid sequence SEQ ID NO:23. These are the VH CDRs of ATL_6178

An antibody according to the present disclosure may have a VH with the following CDRHs: CDRH1 comprising amino acid sequence SEQ ID NO:144, CDRH2 comprising amino acid sequence SEQ ID NO:152 and CDRH3 comprising amino acid sequence SEQ ID NO:161. These are the VH CDRs or ATL_6532. These are the CDRs of ATL_6533. Antibodies according to the present disclosure may have a light chain variable domain (VL) with the CDRs of the light chain variable domain (VL) of antibody ATL_5262 (identical to those of ATL_6178), ATL_6002, or ATL_6003, or ATL6187, or more specifically ATL_5262, or ATL_6002. Antibodies according to the present disclosure may have the following light chain CDRs:
CDRL1: SEQ ID NO: 24, 31 or 32,
CDRL2: SEQ ID NO:34 and
CDRL3: SEQ ID NO:40 or 47 or 121.

In an antibody according to the present disclosure at least one of the VL CDR 1-3 sequences may vary. A variant may have 1, 2, or 3 amino acid mutations, such as substitutions compared with the set of VL CDR1-3 described above. In embodiments, an antibody according to the disclosure comprises CDRs with sequences that have between 1 and 3 mutations, such as substitutions compared to the VL CDR sequences of any antibody described herein. For example, an antibody according to the disclosure may comprise VL CDRs with the sequences of any antibody above, except that one to 3 of the CDRLs comprise a substitution, where the total number of substitutions across the CDRLs does not exceed 3. In embodiments, a variant may have one, two or three, preferably at most one or two substitutions in each of one or more of the VL CDR1-3 described above. CDRL1 regions of any antibodies or fragments described herein may have a length of between 6 and 12 amino acids, preferably 6 amino acids. CDRL2 regions of any antibodies or fragments described herein may have a length of 3 amino acids. CDRL3 regions of any antibodies or fragments described herein may have a length of 9 or 10 amino acids, preferably 9. In embodiments, a variant may have VL CDRs that have at least 70%, at least 80% or at least 90% sequence identity with any set of VL CDRs described herein. For example, a variant with a combined CDRL 1-3 length of 18 amino acids (such as e.g. a variant of the CDRLs ATL_0005262 or ATL0006003) may have up to 5, up to 3, or up to 1 amino acid substitutions compared to a set of VL CDRs described herein.

Antibodies according to the present disclosure may have a VL with the following CDRLs: CDRL1: SEQ ID NO: 24, CDRL2: SEQ ID NO:34, CDRL3: SEQ ID NO:40. These are the VL CDRs of antibodies ATL_5262; 6033; 6034; 6035; 6036; 6037; 6038; 6039; 6177; 6178, 6187 and 6191.

Antibodies according to the present disclosure may have a VL comprising:
(a) the framework sequences of ATL_5262 (and 6002, 6003, 6187, 6191): LFWR1 of SEQ ID NO: 63; LFWR2 of SEQ ID NO: 71; LFWR3 of SEQ ID NO: 77; and LFWR4 of SEQ ID NO: 85;
(b) the framework sequences of ATL_5996: LFWR1 of SEQ ID NO: 64; LFWR2 of SEQ ID NO: 71; LFWR3 of SEQ ID NO: 78; and LFWR4 of SEQ ID NO: 86;
(c) the framework sequences of ATL_5997: LFWR1 of SEQ ID NO: 65; LFWR2 of SEQ ID NO: 72; LFWR3 of SEQ ID NO: 79; and LFWR4 of SEQ ID NO: 87;
(d) the framework sequences of ATL_5998: LFWR1 of SEQ ID NO: 66; LFWR2 of SEQ ID NO: 73; LFWR3 of SEQ ID NO: 80; and LFWR4 of SEQ ID NO: 87;
(e) the framework sequences of ATL_5999: LFWR1 of SEQ ID NO: 67; LFWR2 of SEQ ID NO: 73; LFWR3 of SEQ ID NO: 81; and LFWR4 of SEQ ID NO: 87;
(f) the framework sequences of ATL_6000: LFWR1 of SEQ ID NO: 68; LFWR2 of SEQ ID NO: 74; LFWR3 of SEQ ID NO: 82; and LFWR4 of SEQ ID NO: 88;
(g) the framework sequences of ATL_6001: LFWR1 of SEQ ID NO: 63; LFWR2 of SEQ ID NO: 75; LFWR3 of SEQ ID NO: 77; and LFWR4 of SEQ ID NO: 89;
(h) the framework sequences of ATL_6004: LFWR1 of SEQ ID NO: 69; LFWR2 of SEQ ID NO: 72; LFWR3 of SEQ ID NO: 83; and LFWR4 of SEQ ID NO: 85;
(i) the framework sequences of ATL_6005: LFWR1 of SEQ ID NO: 70; LFWR2 of SEQ ID NO: 76; LFWR3 of SEQ ID NO: 84; and LFWR4 of SEQ ID NO: 90;
(j) or framework sequences comprising up to 5 or up to 10 mutations, such as substitutions, compared to the above sequences.

Antibodies according to the present disclosure may have a VL comprising the framework sequences of any of ATL_6530, ATL_6531, ATL_6532, ATL_6533, ATL_6534, ATL_6535, ATL_6536, ATL_6537, ATL_6538, ATL_6539.

The VH CDRs 1-3 and optionally VL CDRs 1-3 of any of the antibodies described above may also be particularly useful in conjunction with a number of different framework regions. Accordingly, light and/or heavy chains having CDRs 1-3 as described above may possess an alternative framework region. Suitable framework regions are known in the art and are described for example in M. Lefranc & G. Le Franc (2001) “The Immunoglobulin Facts Book”, Academic Press.

An antibody of the disclosure may have the CDRH1, CDRH2 and CDRH3 of the VH domain within a germline framework. In some embodiments, the antibody of the disclosure has a heavy chain variable domain (VH) comprising CDRH1, CDRH2, and CDRH3 within a germline framework, provided that position 67 in standard IMGT numbering is Q.

An antibody of the disclosure may have a heavy chain variable domain (VH) with the framework sequences FWRH1, FWRH2, FWRH3 and FWRH4 of any of the ATL_5262 variants (i.e. antibodies ATL_0005996, ATL_0005997, ATL_0005998, ATL_0005999, ATL_0006000, ATL_0006001, ATL_0006002, ATL_0006003, ATL_0006004, ATL_0006005, ATL_0006036, ATL_0006039, ATL_0006177, ATL_0006178).

An antibody according to the disclosure may have a VH with the following framework sequences: HFWR1 of SEQ ID NO: 50; HFWR2 of SEQ ID NO: 51; HFWR3 of SEQ ID NO: 55; and HFWR4 of SEQ ID NO: 62; or framework sequences comprising one to five mutations, such as substitutions, compared to these framework sequences.

Mutations in the FWRH sequences may include one or more of, or all of: (i) HFWR2: positions 40, 49, (ii) HFWR3: positions 80, 82, 86. In embodiments, the mutations in the framework sequences are substitutions and are selected from the following positions in standard IMGT numbering: (i) HFWR2: at positions 40: T40S, at position 49: R49G, (ii) HFWR3: at position 80: 180V, at position 82: T82K, at position 86: H86Q; in standard IMGT numbering.

Mutations in the FWRH sequences may not include a substitution at position 67 in standard IMGT numbering.

An antibody according to the disclosure may have a VH with the following framework sequences: HFWR1 of SEQ ID NO: 50; HFWR2 of SEQ ID NO: 51-54; HFWR3 of SEQ ID NO: 55-61; and HFWR4 of SEQ ID NO: 62.

The antibody according to the present disclosure may have a heavy chain variable domain (VH) comprising the following framework sequences:
HFWR1 of ATL_6187 or ATL_6191: QVQLQESGPGLVKPSETLSLTCTVS (SEQ ID NO: 50);
HFWR2 of ATL_6187 or ATL_6191: WSWIRQPPGKGLEWIGY (SEQ ID NO: 52) or WSWIRQTPGKGLEWIGY (SEQ ID NO: 195);
HFWR3 of ATL_6187 or ATL_6191: NQNPSLKSRVTMSVDSSKSQLSLKLTSVTAADTAVYYC (SEQ ID NO: 196) or NYNPSLKSRVTISVDTFKNQFSLKLTSVTAADTAVYYC (SEQ ID NO: 197);
HFWR4 of ATL_6187 or ATL_6191: WGQGILVIVSS (SEQ ID NO: 198) or WGQGTLVTVSS (SEQ ID NO: 62), or
framework sequences comprising one to ten or one to 5 mutations, such as substitutions, compared to these framework sequences.

Mutations in the FWRH may include one or more, or all, of:
In HFWR1: position 25, optionally wherein the mutation is the substitution V25Q;
In HFWR2: position 43, optionally wherein the mutation is the substitution 142V; position 49 optionally wherein the mutation is the substitution R49A;
In HFWR3: position 68, optionally wherein the mutation is the substitution N68S; position 78, optionally wherein the mutation is the substitution 178M; position 81, optionally wherein the mutation is the substitution D81Y; position 83, optionally wherein the mutation is the substitution S83F; position 84, optionally wherein the mutation is the substitution K84T; position 87, optionally wherein the mutation is the substitution F87L;
In HFWR4: position 122, optionally wherein the mutation is the substitution 1122K; position 125, optionally wherein the mutation is the substitution T1251.

The antibody according to the present disclosure may have a heavy chain variable domain (VH) comprising the framework sequences of any of antibodies ATL_6530, 6531, 6532, 6533, 6534, 6535, 656, 6537, 6538 or 6539, or framework sequences comprising one to five mutations, such as substitutions, compared to these framework sequences. Thus, antibodies according to the present disclosure may have a VH comprising
(a) HFWR1 of QVQLLETGGGLVQPGGSLRLSCAAS SEQ ID NO: 199; HFWR2 of MNWVRQAPGKGLEWVSS SEQ ID NO: 200; HFWR3 of YYADSVKGRFTISRDNAKNSLYLQMNSLRAEDTAVYYC SEQ ID NO: 201; HFWR4 of WGQGTLVTVSS SEQ ID NO: 62, (framework sequences of ATL_6530);
(b) HFWR1 of EVOLVESGGVVVQPGGSLRLSCAAS SEQ ID NO: 202; HFWR2 of MHWVRQAPGKGLEWVSL SEQ ID NO: 203; HFWR3 of YYADSVKGRFTISRDNSKNSLYLQMNSLRAEDTALYYC SEQ ID NO: 204; HFWR4 of WGQGTLVTVSS SEQ ID NO: 62, (framework sequences of ATL_6531)
(c) HFWR1 of QVTLKESGAEVKKPGASVKVSCKASSEQ ID NO: 205; HFWR2 of MHWVRQAPGQGLEWMGR SEQ ID NO: 206; HFWR3 of NYAQKFQGRVTMTRDTSISTAYMELSRLRSDDTAVYYC SEQ ID NO: 207; HFWR4 of WGQGTMVTVSS SEQ ID NO: 208, (framework sequences of ATL_6532)
(d) HFWR1 of QVQLQQSGGGLVKPGGSLRLSCAAS SEQ ID NO: 209; HFWR2 of MSWIRQAPGKGLEWVSY SEQ ID NO: 210; HFWR3 of NYADSVKGRFTISRDNAKNSLYLQMNSLRAEDTAVYYC SEQ ID NO: 211; HFWR4 of WGQGTMVTVSS SEQ ID NO: 208, (framework sequences of ATL_6533)
(e) HFWR1 of QVQLVESGAEVKKPGASVKVSCKAS SEQ ID NO: 212; HFWR2 of MHWVRQAPGQGLEWMGR SEQ ID NO: 206; HFWR3 of NYAQKFQGRVTMTRDTSISTAYMELSRLRSDDTAVYYC SEQ ID NO: 207; HFWR4 of WGQGTMVTVSS SEQ ID NO: 208, (framework sequences of ATL_6534)
(f) HFWR1 of EVQLLETGGGLVQPGRSLRLSCAAS SEQ ID NO: 13; HFWR2 of MHWVRQAPGKGLEWVSG SEQ ID NO: 14; HFWR3 of GYADSVKGRFTISRDNAKNSLYLQMNSLRAEDTALYYC SEQ ID NO: 15; HFWR4 of WGQGTMVTVSS SEQ ID NO: 208, (framework sequences of ATL_6535)
(g) HFWR1 of EVQLLESAGGVVQLGRSLRLSCAAS SEQ ID NO: 216; HFWR2 of MHWVRQAPGKGLEWVAI SEQ ID NO: 217; HFWR3 of YYAGSVGGRFTISRDNSKNTLYLQMDSLRSDDTAVYYC SEQ ID NO: 218; HFWR4 of WGHGTMVTVSS SEQ ID NO: 219, (framework sequences of ATL_6536)
(h) HFWR1 of QVQLVQSGAEVKKPGESLKISCKGS SEQ ID NO: 220; HFWR2 of IGWVRQMPGKGLEWMGI SEQ ID NO: 221; HFWR3 of RYSPSFQGQVTISADKSISTAYLOWSSLKASDTAMYYC SEQ ID NO: 222; HFWR4 of WGQGTTVTVSS SEQ ID NO: 223, (framework sequences of ATL_6537)
(i) HFWR1 of QVQLQESGPGLVKPSGTLSLTCAVS SEQ ID NO: 224; HFWR2 of WSWVRQPPGKGLEWIGE SEQ ID NO: 225; HFWR3 of NYNPSLKSRVTISVDKSKNQFSLKLSSVTAADTAVYYC SEQ ID NO: 226; HFWR4 of WGQGTLVTVSS SEQ ID NO: 62, (framework sequences of ATL_6538)
(j) HFWR1 of QVQLVESGGGLVQPGGSLRLSCSAS SEQ ID NO: 227; HFWR2 of MSWVRQAPGKGLEWVST SEQ ID NO: 228; HFWR3 of YYADSVKGRFTISRDNSKNTVYLQMNSLRAEDTAVYYC SEQ ID NO: 229; HFWR4 of WGQGTMVTVSS SEQ ID NO: 208, (framework sequences of ATL_6539)
(k) or framework sequences comprising one to five mutations, such as substitutions, compared to these framework sequences.

Mutations in the FWRH may include mutations at one or more, or all, of positions:
In HFWR1: 1, optionally wherein the mutation is the substitution Q1E; 1, optionally wherein the mutation is the substitution Q3T; 6, optionally wherein the mutation is the substitution E6Q; 7, optionally wherein the mutation is the substitution S7T; 12, optionally wherein the mutation is the substitution L12V; 13, optionally wherein the mutation is the substitution V13K; 15, optionally wherein the mutation is the substitution P15L; 16, optionally wherein the mutation is the substitution S16G; 19, optionally wherein the mutation is the substitution L19V; 21, optionally wherein the mutation is the substitution L21V; 22, optionally wherein the mutation is the substitution S22T;
In HFWR2: 48, optionally wherein the mutation is the substitution K48Q;
In HFWR3: 74, optionally wherein the mutation is the substitution S74G; 75, optionally wherein the mutation is the substitution R75Q; 76, optionally wherein the mutation is the substitution V76F; 78, optionally wherein the mutation is the substitution 178M; 79, optionally wherein the mutation is the substitution S79T; 83, optionally wherein the mutation is the substitution S83A; 84, optionally wherein the mutation is the substitution K841; 85, optionally wherein the mutation is the substitution N85S; 89, optionally wherein the mutation is the substitution L89M; 93, optionally wherein the mutation is the substitution S93R; 96, optionally wherein the mutation is the substitution A96S;
In HFWR4: 120, optionally wherein the mutation is the substitution Q120H.

In this specification, antibodies may have VH (and optionally VL) regions comprising an amino acid sequence that has a high percentage sequence identity to the VH and/or VL amino acid sequences described above.

For example, antibodies according to the present disclosure include antibodies that bind UNC5C and have a VH regions that comprises an amino acid sequence having at least 70%, more preferably one of at least 75%, 80%, 85%, 90%, 95%, 96%, 97%, 98%, 99% or 100% sequence identity to the VH region amino acid sequence of any antibody described herein, such as ATL_5262, ATL_6036, ATL_6039, ATL_6177, ATL_6178, ATL_6187, ATL_6191, ATL_6530-6539 (SEQ ID NOs: 1, 5, 8, 91, 93, 112, 113, 122-131). Note that any sequence obtained by applying conservative one or more conservative amino acid substitutions to a sequence as described herein is also encompassed, provided that the overall sequence identity is within the ranges provided and that the resulting antibody still binds UNC5C and competes with/mimic netrin-1 binding to UNC5C.

An antibody of the disclosure may have the CDRL1, CDRL2 and CDRL3 of the VL domain within a germline framework. An antibody of the disclosure may have a light chain variable domain (VL) with the framework sequences LFWR1, LFWR2, LFWR3 and LFWR4 of any of ATL_5262 variants, including ATL_5262, ATL_6036, ATL_6039, ATL_6177 or ATL_6178.

Accordingly, the antibody of the disclosure may have a light chain variable domain (VL) with the following framework sequences: LFWR1 of SEQ ID NO:63; LFWR2 of SEQ ID NO:71; LFWR3 of SEQ ID NO:77; and LFWR4 of SEQ ID NO:85 or 87; or a set of FWRs which contains one to five amino acid mutations, such as substitutions, compared with the above set of FWR.

Alternatively or additionally, antibodies of the disclosure may have a VL region that comprises an amino acid sequence having at least 70%, more preferably one of at least 75%, 80%, 85%, 90%, 95%, 96%, 97%, 98%, 99% or 100% sequence identity to the VL region amino acid sequence of any antibody described herein, such as ATL_5262, ATL_5998, ATL_6001, ATL_6002, ATL_6003, ATL_6177, ATL_6178, ATL_6187, ATL_6191, ATL6530-6539 (SEQ ID Nos: 9, 12, 15, 16, 17, 92, 94, 114, 132-141). For example, antibodies of the disclosure may have a VL region that comprises an amino acid sequence having at least 70%, more preferably one of at least 75%, 80%, 85%, 90%, 95%, 96%, 97%, 98%, 99% or 100% sequence identity to the VL region amino acid sequence of antibody ATL_5262 or ATL_6002 (SEQ ID Nos: 9, 16). For example, antibodies of the disclosure may have a VL region that comprises an amino acid sequence having at least 70%, more preferably one of at least 75%, 80%, 85%, 90%, 95%, 96%, 97%, 98%, 99% or 100% sequence identity to the VL region amino acid sequence of antibody ATL_5262, or ATL_6178 (SEQ ID Nos: 9, 94).

Antibodies of the disclosure may have a type L or type K VL region.

The antibodies described herein may comprise: a heavy chain variable domain (VH) comprising a sequence that has at least 70%, at least 75%, at least 80%, at least 85%, at least 90%, or at least 95%, 96%, 97%, 98%, 99% or 100% sequence identity to sequence identity to SEQ ID NO: 1 and a and a light chain variable domain (VL) comprising a sequence that has at least 70%, at least 75%, at least 80%, at least 85%, at least 90%, or at least 95%, 96%, 97%, 98%, 99% or 100% sequence identity to sequence identity to SEQ ID NO: 9.

The antibodies described herein may comprise: a heavy chain variable domain (VH) comprising a sequence that has at least 70%, at least 75%, at least 80%, at least 85%, at least 90%, or at least 95%, 96%, 97%, 98%, 99% or 100% sequence identity to sequence identity to SEQ ID NO: 93 and a and a light chain variable domain (VL) comprising a sequence that has at least 70%, at least 75%, at least 80%, at least 85%, at least 90%, or at least 95%, 96%, 97%, 98%, 99% or 100% sequence identity to sequence identity to SEQ ID NO: 94.

Antibodies described herein include antibodies that have a heavy chain variable domain (VH) comprising a sequence of the VH of any antibody described herein and a light chain variable domain (VL) comprising a sequence of the VH of any antibody described herein. In particular, antibodies described herein include antibodies that have: (i) a heavy chain variable domain (VH) comprising a sequence of the VH of ATL_5262, ATL_6177, ATL_6178, ATL_6033, ATL_6034, ATL6035, ATL_6036, ATL_6037, ATL_6038, ATL_6039, ATL_5998, ATL_6001, ATL_6002, ATL_6003, ATL_6187, ATL_6191, ATL_6530, ATL_6531, ATL_6532, ATL_6533, ATL_6534, ATL_6535, ATL_6536, ATL_6537, ATL_6538, ATL_6539, and (ii) a light chain variable domain (VL) comprising a sequence of the VL of ATL_5262, ATL_6177, ATL_6178, ATL_6033, ATL_6034, ATL6035, ATL_6036, ATL_6037, ATL_6038, ATL_6039, ATL_5998, ATL_6001, ATL_6002, ATL_6003, ATL_6187, ATL_6191, ATL_6530, ATL_6531, ATL_6532, ATL_6533, ATL_6534, ATL_6535, ATL_6536, ATL_6537, ATL_6538, ATL_6539. In particular, also described are antibodies that have: (i) a heavy chain variable domain (VH) comprising a sequence of the VH of ATL_5262, ATL_6177, ATL_6178, ATL_6033, ATL_6034, ATL6035, ATL_6036, ATL_6037, ATL_6038, ATL_6039, ATL_5998, ATL_6001, ATL_6002, ATL_6003, ATL_6187, ATL_6191, and (ii) a light chain variable domain (VL) comprising a sequence of the VL of ATL_5262, ATL_6177, ATL_6178, ATL_6033, ATL_6034, ATL6035, ATL_6036, ATL_6037, ATL_6038, ATL_6039, ATL_5998, ATL_6001, ATL_6002, ATL_6003, ATL_6187, ATL_6191. Further, also described are antibodies that have: (i) a heavy chain variable domain (VH) comprising a sequence of the VH of ATL_6530, ATL_6531, ATL_6532, ATL_6533, ATL_6534, ATL_6535, ATL_6536, ATL_6537, ATL_6538, ATL_6539, and (ii) a light chain variable domain (VL) comprising a sequence of the VL of ATL_6530, ATL_6531, ATL_6532, ATL_6533, ATL_6534, ATL_6535, ATL_6536, ATL_6537, ATL_6538, ATL_6539.

The antibodies of the disclosure are derived from human antibodies and may therefore be referred to as human antibodies. Antibodies of the disclosure were obtained by pairing a human VH domain with a VL domain using a transformer-based model. Thus, antibodies of the disclosure may differ from naturally occurring antibodies at least by their VH-VL pairing. Antibodies of the disclosure may comprise human VH and/or VL sequences. Antibodies of the disclosure may be formulated as human IgG1 antibodies or variants thereof. Antibodies of the disclosure may have a human framework sequence and/or a human Fc domain.

Overall percentage identity of a variable region or full length heavy/light chain sequence may be combined with specific CDR sequences from the same antibody.

Percentage (%) sequence identity is defined as the percentage of amino acid residues in a candidate sequence that are identical with residues in a comparative sequence after aligning the sequences and introducing gaps if necessary, to achieve the maximum sequence identity, and not considering any conservative substitutions as part of the sequence identity. Sequence identity is preferably calculated over the entire length of the respective sequences. Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence. Sequence identity may be defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters may be used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), SSEARCH (Smith and Waterman (1981) J. Mol Biol. 147: 195-197), HMMER3 (Johnson L S et al BMC Bioinformatics. 2010 Aug. 18; 11( ):431) or the TBLASTN program, of Altschul et al. (1990) supra, generally employing default parameters (see for example Pearson Curr Prot Bioinformatics (2013) Chapt 3 Uniy 3.1 doi:10.1002/0471250953.bi0301s42). In particular, the psi-Blast algorithm may be used (Altschul et al. Nucl. Acids Res. (1997) 25 3389-3402). Sequence identity and similarity may also be determined using Genomequest™ software (Gene-IT, Worcester MA USA). Sequence comparisons are preferably made over the full-length of the relevant sequences to be compared.

The antibodies of the present disclosure may comprise one or more substitutions within the framework of the VH and/or VL region. As used herein, “substitution” refers to the exchange of one amino acid for another at a specific position, relative to the same position in a baseline molecule. In some embodiments, the baseline molecules are exemplified antibodies herein, for example ATL_5262, ATL_6177, ATL_6178, ATL_6002 or ATL_6003 (VH sequences of SEQ ID Nos: 1, 91, 93; VL sequences of SEQ ID Nos: 9, 16, 17).

In some embodiments, antibodies or fragments thereof according to the present disclosure are able to cross the blood-brain barrier. In some embodiments, the antibodies of the present disclosure are bispecific antibodies. For example, antibodies according to the present disclosure may have one scFV chain that binds a receptor in the brain, such as the transferrin receptor (Yu et al., Sci Transl Med. 2014 Nov. 5; 6(261):261ra154), as well as a scFv chain that binds UNC5C as described herein. In some embodiments, antibodies according to the disclosure comprise an antibody or fragment thereof that binds UNC5C as described herein (such as e.g. an antibody, scFV, sdAb, etc.), and a further binding moiety that binds to another target. The other target may be a receptor in the brain, such as the transferring receptor. The further binding moiety may be an antibody, a scFv, a nanobody, or an aptamer. The two binding moieties of such a bispecific molecule may form a fusion protein.

Also described herein are single domain antibodies (sdAbs), otherwise known as nanobodies, comprising the heavy chain CDRs and/or the VH sequence of any antibody described herein. Thus, also described herein are antibodies or fusion molecules comprising a nanobody that binds UNC5C as described herein, and a nanobody that binds a receptor in the brain. Also described herein are antibodies of fusion molecules comprising a scFV chain or nanobody that binds UNC5C as described herein, and an aptamer that binds a receptor in the brain.

Isolated nucleic acids encoding an antibody, antigen binding fragment, or polypeptide as described herein are provided. Also provided is a vector comprising a nucleic acid described herein, and a host cell comprising the vector. For example, the host cell may be a eukaryotic, or mammalian, e.g. Chinese Hamster Ovary (CHO), cell or may be a prokaryotic cell, e.g. E. coli. In some embodiments, the vector is a viral vector, for example a bacteriophage.

Further provided are methods for making an antibody, or antibody fragment as described herein, the method comprising culturing a host cell as described herein under conditions suitable for the expression of a vector encoding the antibody, or antibody fragment, and isolating and/or purifying the antibody, or antibody fragment. The method further comprises formulating the antibody or antibody fragment into a composition including at least an additional component.

### Therapeutic Uses and In Vitro/In Vivo Effects

Antibodies described herein may reduce UNC5C-mediated apoptosis. For example, antibodies described herein may reduce UNC5C-mediated apoptosis in neurons, e.g. induced pluripotent stem cell (iPSC)-derived neurons; and/or in neuroblastoma cells, such as SH-SY5Y cells. The skilled person will be familiar with methods to measure apoptosis, such as by measuring caspase 3/7 production as described herein, or measuring cell viability, e.g. by staining the cell with a live/dead cell marker, such as DAPI (4′,6-diamidino-2-phenylindole).

As described above, antibodies of the disclosure have been found to mimic/compete with binding of netrin-1 to UNC5C. Netrin 1 levels correlate with dementia severity and netrin 1 is decreased in mild cognitive impairment and AD patients (Ju T, et al. 2022).

Netrin-1 has been shown to protect against β-amyloid-induced neurotoxicity through NF-κB/Nrf2 dependent mechanism (Zamani et al., 2020).

Netrin-1 has been shown to be involved in synaptic plasticity, i.e. the ability of neurons to bring about changes in the connections between neuronal networks in response to stimuli, in adults. UNC5C is a netrin 1 receptor which plays a role in axonal outgrowth and guidance. Whilst this has been most studied in development, it is a mechanism that could be important in preventing neuronal degeneration. Thus, antibodies and fragments thereof as described herein may be effective and/or may be used in promoting synaptic health, e.g. by promoting synaptic plasticity, which may be assessed by quantifying the effect of the antibody on the number of processes per cell, the length of neurite outgrowth, and branching. For example, methods as described herein and in Spiijkers et al., 2021 may be used.

The antibodies and fragments thereof described may find use in therapy.

A subject to be treated or diagnosed may be any animal or human. The subject is preferably mammalian, more preferably human. The subject may be male or female. The subject may be a patient. Therapeutic uses may be in human or animals (veterinary use).

Medicaments and pharmaceutical compositions according to aspects of the present invention may be formulated for administration by a number of routes, including but not limited to, parenteral, intravenous, intra-arterial, intramuscular, oral and nasal. The medicaments and compositions may be formulated for injection.

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 5 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans. The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. the anti-CFH antibodies used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Conditions treatable in accordance with the present disclosure include any in which UNC5C plays a role, including neurodegenerative disorders, and in particular those characterised by increased neuronal apoptosis, decreased synaptic health and/or altered synaptic plasticity (e.g. reduced or abnormal synaptic plasticity). The antibodies of the present disclosure were originally identified through analysis of resilient frontotemporal dementia (FTD) patients. These antibodies were subsequently found in other resilient and control individuals in Alzheimer's disease (AD); Parkinson's disease (PD); and in resilient centenarians indicating relevance beyond FTD to other neurodegenerative diseases. Thus, also described herein are antibodies of the disclosure for use as a medicament. Also described are antibodies of the disclosure for use in the treatment or prevention of a neurodegenerative disease. Also described herein are antibodies of the disclosure for use in the manufacture of a medicament, such as a medicament for the treatment or prevention of a neurodegenerative disease or disorder. Also described herein a method of treating a subject who has been diagnosed as having or being at risk of having a neurodegenerative disorder, the method comprising administering an antibody as described herein in a therapeutically effective amount to the subject. A neurodegenerative disease or disorder can comprise one or more of the following: FTD, AD, HD, Parkinson's disease (PD), human immunodeficiency virus (HIV)-induced encephalitis, Chronic traumatic encephalopathy (CTE), vascular dementia, prion diseases, Lewy body disease, Spinal muscular atrophy (SMA); Motor Neuron Disease (MND), such as amyotrophic lateral sclerosis (ALS), progressive supranuclear palsy (PSP), spinocerebellar ataxias (SCA) types 1, 2, 6, 7 and 17, Machado-Joseph disease (MJD/SCA3), dentatorubral pallidoluysian atrophy (DRPLA), spinal bulbar muscular atrophy X-linked type 1 (SMAX1/SBMA), Anderson-Fabry (X-linked Fabry Disease), and DNAJB6 Myopathies. In embodiments, the neurodegenerative disease is selected from FTD, AD, HD, MND, such as ALS, and PD. In embodiments, the neurodegenerative disease is a MND, such as ALS. In embodiments, the neurodegenerative disease is FTD, AD, HD or PD.

The antibodies of the disclosure may be used in therapy with further therapeutic agents. As used herein, a “further therapeutic agent” is an additional compound, protein, vector, antibody, cell or entity with a therapeutic effect. The antibodies may be co-administered with a further therapeutic agent. The antibodies may be co-formulated with a further therapeutic agent. The antibodies may be sequentially administered before or after a further therapeutic agent.

The antibodies described herein may be used as biomarkers indicating that a subject is likely to respond to therapy using an antibody or antibody fragment as described herein. A method of determining whether a subject is likely to respond to treatment with an antibody or fragment thereof as described herein, the method comprising obtaining BCR sequence data from the subject and determining, using said sequence data, whether the subject's BCR repertoire comprises one or more antibodies that are likely to bind to UNC5C (e.g. antibodies as described herein, such as e.g. antibodies having at least 70%, at least 80%, at least 85%, at least 90%, at least 95% or at least 99% homology to any specific antibody or antibody fragment described herein), wherein a subject whose BCR repertoire does not comprise one or more antibodies that are likely to bind to UNC5C is likely to respond to treatment with an antibody or antibody fragment thereof as described herein.

Thus, also described herein is a method of treating a subject who has been diagnosed as having or likely to have a disease in which UNC5C plays a role (e.g. a neurodegenerative disorder), the method comprising: obtaining BCR sequence data from the subject; determining, using said sequence data, whether the subject's BCR repertoire comprises one or more antibodies that are likely to bind to UNC5C; and administering to a subject whose BCR repertoire does not comprise one or more antibodies that are likely to bind to UNC5C a therapeutically effective amount of an antibody or antibody fragment thereof as described herein.

Some methods of the present disclosure involve a sample containing cells. The sample may be a culture of cells grown in vitro. For example, the culture may comprise a suspension of cells or cells cultured in a culture plate or dish.

Methods according to the present disclosure may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

According to some aspects of the present disclosure a kit of parts is provided comprising an antibody according to the present invention. In some embodiments, the kit comprises an antibody according to the present invention and one or more of: reagents for use in immunochemistry; the antibodies immobilised to a solid support; means for labelling the antibodies; means for linking the antibodies to a cytotoxic moiety; a further therapeutic agent.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

All references cited herein are incorporated herein by reference in their entirety. For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### Sequences

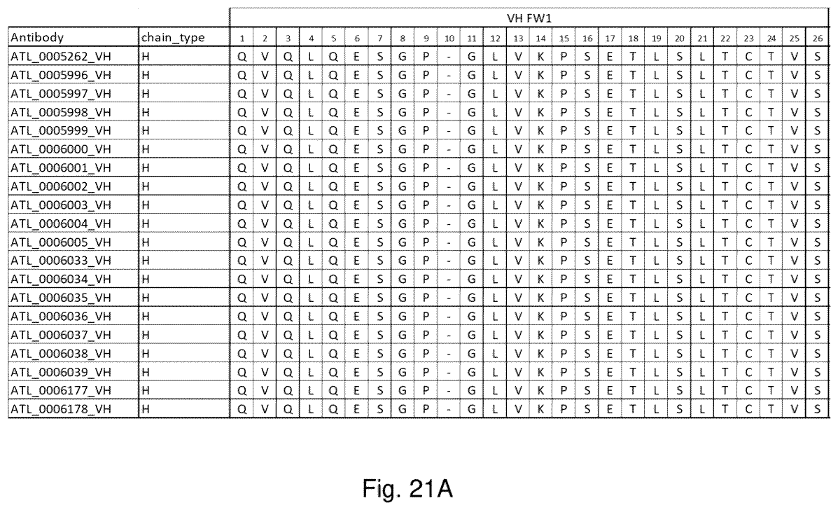

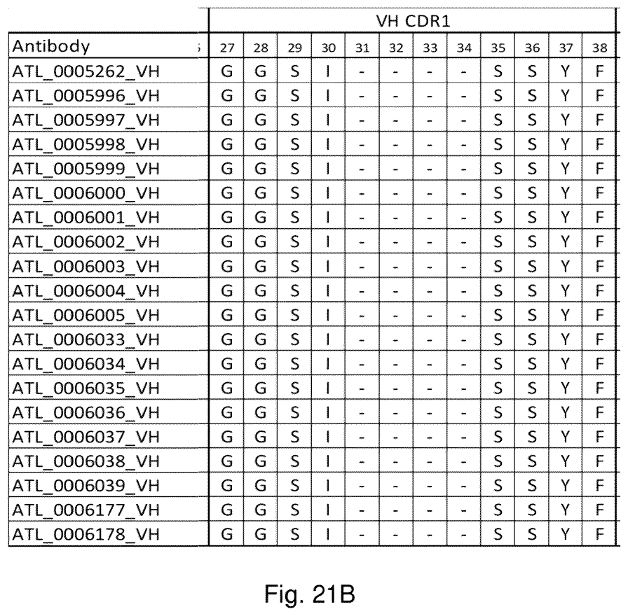

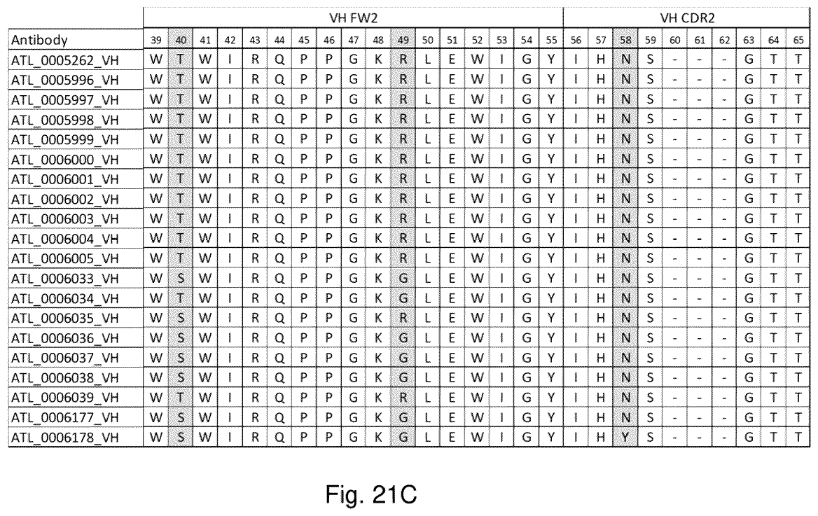

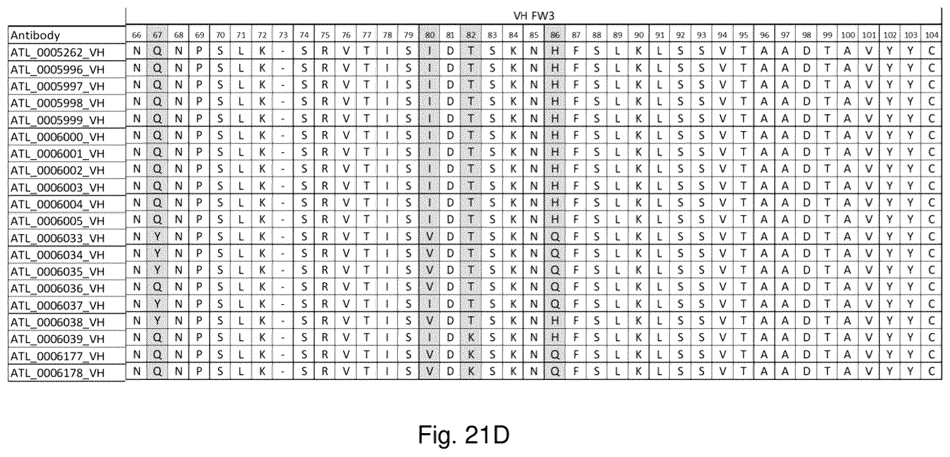

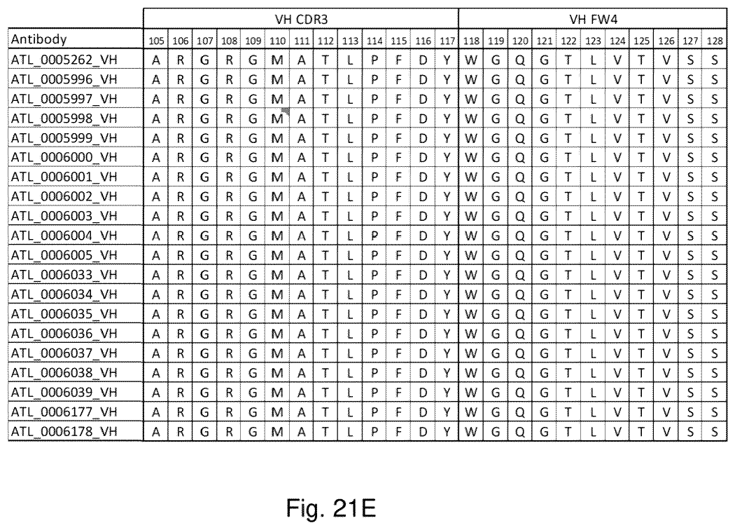

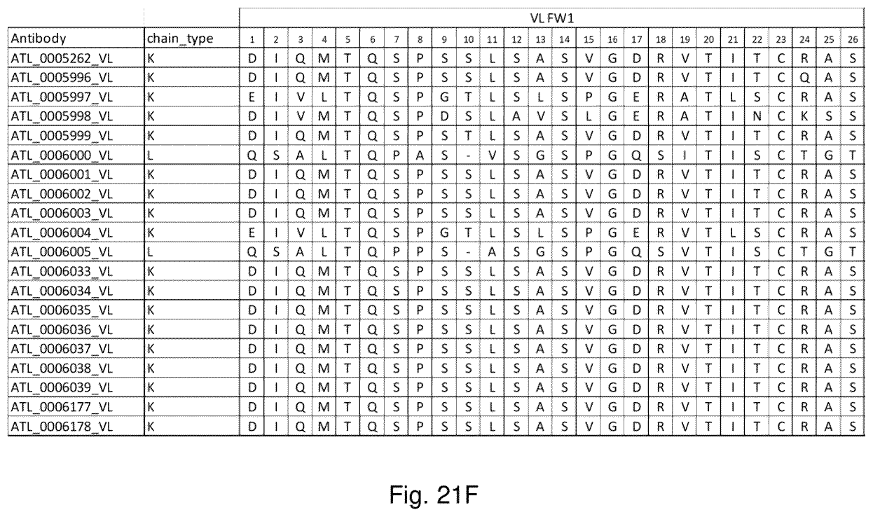

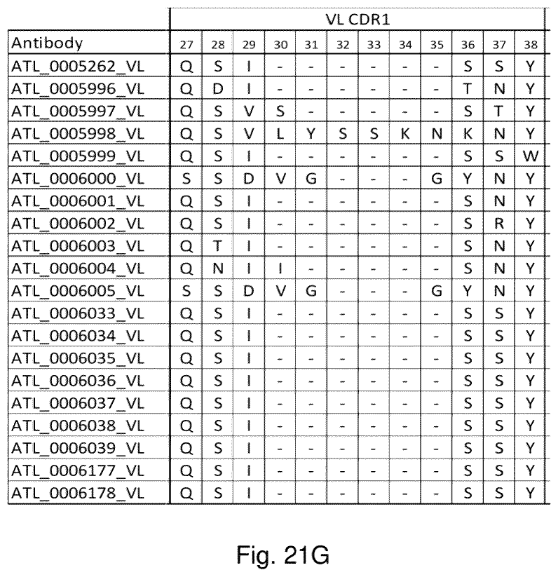

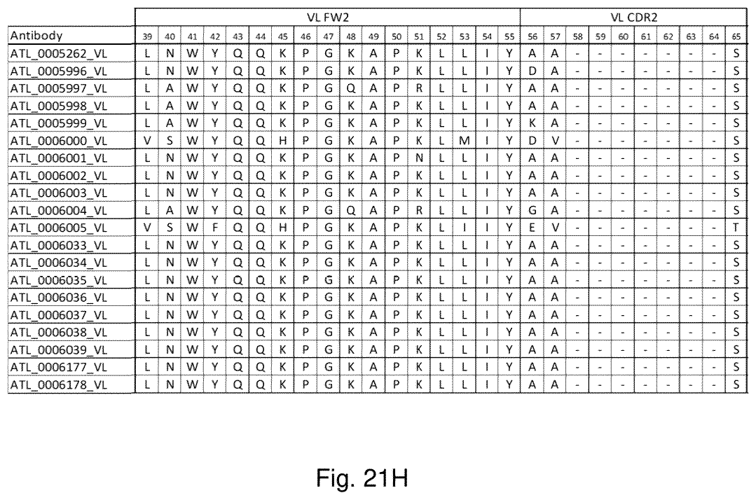

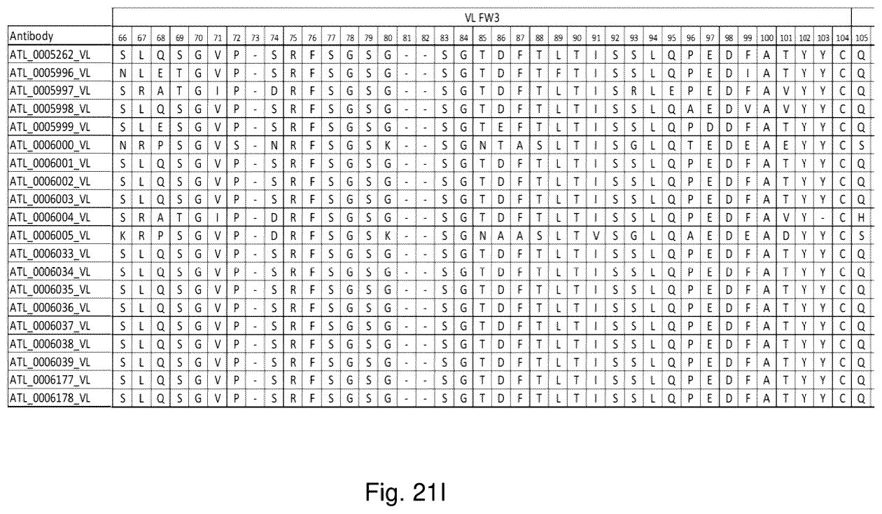

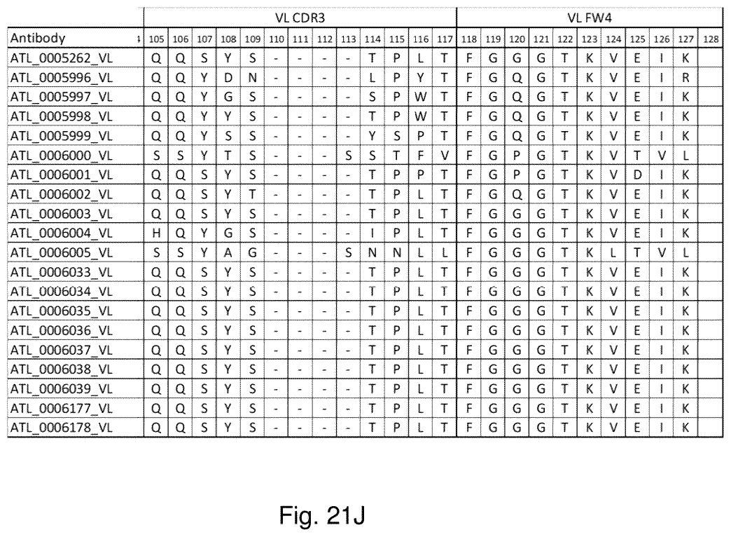

Example sequence of antibodies described herein are provided in FIG. 21, 8B, 32 (aligned by IMGT position numbering) and in the table below.

| SEQ | | |
|---|---|---|
| ID NO | Description | Sequence |
| 1 | ATL_5262 VH | QVQLQESGPGLVKPSETLSLTCTVSGGSISSYFWTWIRQPPGKRL |
| | | EWIGYIHNSGTTNQNPSLKSRVTISIDTSKNHFSLKLSSVTAADTA |
| | | VYYCARGRGMATLPFDYWGQGTLVTVSS |
| 2 | ATL_6033 VH | QVQLQESGPGLVKPSETLSLTCTVSGGSISSYFWSWIRQPPGKG |
| | | LEWIGYIHNSGTTNYNPSLKSRVTISVDTSKNQFSLKLSSVTAADT |
| | | AVYYCARGRGMATLPFDYWGQGTLVTVSS |
| 3 | ATL_6034 VH | QVQLQESGPGLVKPSETLSLTCTVSGGSISSYFWTWIRQPPGKG |
| | | LEWIGYIHNSGTTNYNPSLKSRVTISVDTSKNQFSLKLSSVTAADT |
| | | AVYYCARGRGMATLPFDYWGQGTLVTVSS |
| 4 | ATL_6035 VH | QVQLQESGPGLVKPSETLSLTCTVSGGSISSYFWSWIRQPPGKR |
| | | LEWIGYIHNSGTTNYNPSLKSRVTISVDTSKNQFSLKLSSVTAADT |
| | | AVYYCARGRGMATLPFDYWGQGTLVTVSS |
| 5 | ATL_6036 VH | QVQLQESGPGLVKPSETLSLTCTVSGGSISSYFWSWIRQPPGKG |
| | | LEWIGYIHNSGTTNQNPSLKSRVTISVDTSKNQFSLKLSSVTAADT |
| | | AVYYCARGRGMATLPFDYWGQGTLVTVSS |
| 6 | ATL_6037 VH | QVQLQESGPGLVKPSETLSLTCTVSGGSISSYFWSWIRQPPGKG |
| | | LEWIGYIHNSGTTNYNPSLKSRVTISIDTSKNQFSLKLSSVTAADTA |
| | | VYYCARGRGMATLPFDYWGQGTLVTVSS |
| 7 | ATL_6038 VH | QVQLQESGPGLVKPSETLSLTCTVSGGSISSYFWSWIRQPPGKG |
| | | LEWIGYIHNSGTTNYNPSLKSRVTISVDTSKNHFSLKLSSVTAADT |
| | | AVYYCARGRGMATLPFDYWGQGTLVTVSS |
| 8 | ATL_6039 VH | QVQLQESGPGLVKPSETLSLTCTVSGGSISSYFWTWIRQPPGKRL |
| | | EWIGYIHNSGTTNQNPSLKSRVTISIDKSKNHFSLKLSSVTAADTA |
| | | VYYCARGRGMATLPFDYWGQGTLVTVSS |
| 9 | ATL_5262 VL | DIQMTQSPSSLSASVGDRVTITCRASQSISSYLNWYQQKPGKAPK |
| | | LLIYAASSLQSGVPSRFSGSGSGTDFTLTISSLQPEDFATYYCQQS |
| | | YSTPLTFGGGTKVEIK |
| 10 | ATL_5996 VL | DIQMTQSPSSLSASVGDRVTITCQASQDITNYLNWYQQKPGKAPK |
| | | LLIYDASNLETGVPSRFSGSGSGTDFTFTISSLQPEDIATYYCQQY |
| | | DNLPYTFGQGTKVEIR |
| 11 | ATL_5997 VL | EIVLTQSPGTLSLSPGERATLSCRASQSVSSTYLAWYQQKPGQA |
| | | PRLLIYAASSRATGIPDRFSGSGSGTDFTLTISRLEPEDFAVYYCQ |
| | | QYGSSPWTFGQGTKVEIK |
| 12 | ATL_5998 VL | DIVMTQSPDSLAVSLGERATINCKSSQSVLYSSKNKNYLAWYQQK |
| | | PGKAPKLLIYAASSLQSGVPSRFSGSGSGTDFTLTISSLQAEDVAV |
| | | YYCQQYYSTPWTFGQGTKVEIK |
| 13 | ATL_5999 VL | DIQMTQSPSTLSASVGDRVTITCRASQSISSWLAWYQQKPGKAP |
| | | KLLIYKASSLESGVPSRFSGSGSGTEFTLTISSLQPDDFATYYCQQ |
| | | YSSYSPTFGQGTKVEIK |
| 14 | ATL_6000 VL | QSALTQPASVSGSPGQSITISCTGTSSDVGGYNYVSWYQQHPGK |
| | | APKLMIYDVSNRPSGVSNRFSGSKSGNTASLTISGLQTEDEAEYY |
| | | CSSYTSSSTFVFGPGTKVTVL |
| 15 | ATL_6001 VL | DIQMTQSPSSLSASVGDRVTITCRASQSISNYLNWYQQKPGKAPN |
| | | LLIYAASSLQSGVPSRFSGSGSGTDFTLTISSLQPEDFATYYCQQS |
| | | YSTPPTFGPGTKVDIK |
| 16 | ATL_6002 VL | DIQMTQSPSSLSASVGDRVTITCRASQSISRYLNWYQQKPGKAPK |
| | | LLIYAASSLQSGVPSRFSGSGSGTDFTLTISSLQPEDFATYYCQQS |
| | | YTTPLTFGQGTKVEIK |
| 17 | ATL__6003 VL | DIQMTQSPSSLSASVGDRVTITCRASQTISNYLNWYQQKPGKAPK |
| | | LLIYAASSLQSGVPSRFSGSGSGTDFTLTISSLQPEDFATYYCQQS |
| | | YSTPLTFGGGTKVEIK |
| 18 | ATL_6004 VL | EIVLTQSPGTLSLSPGERVTLSCRASQNIISNYLAWYQQKPGQAP |
| | | RLLIYGASSRATGIPDRFSGSGSGTDFTLTISSLQPEDFAVYCHQY |
| | | GSIPLTFGGGTKVEIK |
| 19 | ATL_6005 VL | QSALTQPPSASGSPGQSVTISCTGTSSDVGGYNYVSWFQQHPG |
| | | KAPKLIIYEVTKRPSGVPDRFSGSKSGNAASLTVSGLQAEDEADY |
| | | YCSSYAGSNNLLFGGGTKLTVL |
| 20 | CDRH1 (all ATL_5262 | GGSISSYF |
| | variants) | |
| 21 | CDRH2 (all ATL_5262 | IHNSGTT |
| | variants except | |
| | ATL_6178) | |
| 22 | CDRH2 ATL_6178 | IHYSGTT |
| 23 | CDRH3 (all ATL_5262 | ARGRGMATLPFDY |
| | variants) | |
| 24 | CDRL1 (ATL_5262; | QSISSY |
| | 6033; 6034; 6035; 6036; | |
| | 6037; 6038; 6039; 6177; | |
| | 6178) | |
| 25 | CDRL1 (ATL_5996) | QDITNY |
| 26 | CDRL1 (ATL_5997) | QSVSSTY |
| 27 | CDRL1 (ATL_5998) | QSVLYSSKNKNY |
| 28 | CDRL1 (ATL_5999) | QSISSW |
| 29 | CDRL1 (ATL_6000; | SSDVGGYNY |
| | ATL_6005) | |
| 30 | CDRL1 (ATL_6001) | QSISNY |
| 31 | CDRL1 (ATL_6002) | QSISRY |
| 32 | CDRL1 (ATL_6003) | QTISNY |
| 33 | CDRL1 (ATL_6004) | QNIISNY |
| 34 | CDRL2 (ATL_ 5262; | AAS |
| | 5997; 5998; 6001; 6002; | |
| | 6003; 6033; 6034; 6035; | |
| | 6036; 6037; 6038; 6039; | |
| | 6177; 6178) | |
| 35 | CDRL2 (ATL_5996) | DAS |
| 36 | CDRL2 (ATL_5999) | KAS |
| 37 | CDRL2 (ATL_6000) | DVS |
| 38 | CDRL2 (ATL_6004) | GAS |
| 39 | CDRL2 (ATL_6005) | EVT |
| 40 | CDRL3 (ATL_5262; | QQSYSTPLT |
| | 6003;6033; 6034; 6035; | |
| | 6036; 6037; 6038;6039; | |
| | 6177; 6178) | |
| 41 | CDR3L (ATL_5996) | QQYDNLPYT |
| 42 | CDR3L (ATL_5997) | QQYGSSPWT |
| 43 | CDR3L (ATL_5998) | QQYYSTPWT |
| 44 | CDR3L (ATL_5999) | QQYSSYSPT |
| 45 | CDR3L (ATL_6000) | SSYTSSSTFV |
| 46 | CDR3L (ATL_6001) | QQSYSTPPT |
| 47 | CDR3L (ATL_6002) | QQSYTTPLT |
| 48 | CDR3L (ATL_ 6004) | HQYGSIPLT |
| 49 | CDR3L (ATL_6005) | SSYAGSNNLL |
| 50 | HFWR1 (all ATL_5262 | QVQLQESGPGLVKPSETLSLTCTVS |
| | variants and | |
| | homologues 6187 and | |
| | 6191) | |
| 51 | HFWR2 (ATL_5262; | WTWIRQPPGKRLEWIGY |
| | 5996; 5997; 5998; 5999; | |
| | 6000; 6001; 6002; 6003; | |
| | 6004; 6005; 6039) | |
| 52 | HFWR2 (ATL_6033; | WSWIRQPPGKGLEWIGY |
| | 6036; 6037; 6038; 6177; | |
| | 6178; 6187) | |
| 53 | HFWR2 (ATL_6034 | WTWIRQPPGKGLEWIGY |
| 54 | HFWR2 (ATL_6035) | WSWIRQPPGKRLEWIGY |
| 55 | HFWR3 (ATL_5262; | NQNPSLKSRVTISIDTSKNHFSLKLSSVTAADTAVYYC |
| | 5996; 5997; 5998; 5999; | |
| | 6000; 6001; 6002; 6003; | |
| | 6004; 6005; ) | |
| 56 | HFWR3 (ATL_6033; | NYNPSLKSRVTISVDTSKNQFSLKLSSVTAADTAVYYC |
| | 6034; 6035) | |
| 57 | HFWR3 (ATL_6036) | NQNPSLKSRVTISVDTSKNQFSLKLSSVTAADTAVYYC |
| 58 | HFWR3 (ATL_6037) | NYNPSLKSRVTISIDTSKNQFSLKLSSVTAADTAVYYC |
| 59 | HFWR3 (ATL_6038) | NYNPSLKSRVTISVDTSKNHFSLKLSSVTAADTAVYYC |
| 60 | HFWR3 (ATL_6039) | NQNPSLKSRVTISIDKSKNHFSLKLSSVTAADTAVYYC |
| 61 | HFWR3 (ATL_6177; | NQNPSLKSRVTISVDKSKNQFSLKLSSVTAADTAVYYC |
| | 6178) | |
| 62 | HFWR4 (all ATL_5262 | WGQGTLVTVSS |
| | variants, and | |
| | homologue | |
| | ATL_0006191, phage | |
| | display antibodies 6530, | |
| | 6531, 6538) | |
| 63 | LFWR1 (ATL_5262; | DIQMTQSPSSLSASVGDRVTITCRAS |
| | 6001; 6002; 6003; 6033; | |
| | 6034; 6035; 6036; 6037; | |
| | 6038; 6039; 6177; 6178) | |
| 64 | LFWR1 (ATL_5996) | DIQMTQSPSSLSASVGDRVTITCQAS |
| 65 | LFWR1 (ATL_5997) | EIVLTQSPGTLSLSPGERATLSCRAS |
| 66 | LFWR1 (ATL_5998) | DIVMTQSPDSLAVSLGERATINCKSS |
| 67 | LFWR1 (ATL_5999) | DIQMTQSPSTLSASVGDRVTITCRAS |
| 68 | LFWR1 (ATL_6000, 6536) | QSALTQPASVSGSPGQSITISCTGT |
| 69 | LFWR1 (ATL_6004) | EIVLTQSPGTLSLSPGERVTLSCRAS |
| 70 | LFWR1 (ATL_6005) | QSALTQPPSASGSPGQSVTISCTGT |
| 71 | LFWR2 (ATL_5262; | LNWYQQKPGKAPKLLIY |
| | 5996; 6002; 6003; 6033; | |
| | 6034; 6035; 6036; 6037; | |
| | 6038; 6039; 6177; 6178) | |
| 72 | LFWR2 (ATL_5997, 6004) | LAWYQQKPGQAPRLLIY |
| 73 | LFWR2 (ATL_5998; 5999) | LAWYQQKPGKAPKLLIY |
| 74 | LFWR2 (ATL_6000) | VSWYQQHPGKAPKLMIY |
| 75 | LFWR2 (ATL_6001) | LNWYQQKPGKAPNLLIY |
| 76 | LFWR2 (ATL_6005) | VSWFQQHPGKAPKLIIY |
| 77 | LFWR3 (ATL_5262; | SLQSGVPSRFSGSGSGTDFTLTISSLQPEDFATYYC |
| | 6001; 6002; 6003; 6033; | |
| | 6034; 6035; 6036; 6037; | |
| | 6038; 6039; 6177; 6178) | |
| 78 | LFWR3 (ATL_5996) | NLETGVPSRFSGSGSGTDFTFTISSLQPEDIATYYC |
| 79 | LFWR3 (ATL_5997) | SRATGIPDRFSGSGSGTDFTLTISRLEPEDFAVYYC |
| 80 | LFWR3 (ATL_5998) | SLQSGVPSRFSGSGSGTDFTLTISSLQAEDVAVYYC |
| 81 | LFWR3 (ATL_5999) | SLESGVPSRFSGSGSGTEFTLTISSLQPDDFATYYC |
| 82 | LFWR3 (ATL_6000) | NRPSGVSNRFSGSKSGNTASLTISGLQTEDEAEYYC |
| 83 | LFWR3 (ATL_6004) | SRATGIPDRFSGSGSGTDFTLTISSLQPEDFAVYC |
| 84 | LFWR3 (ATL_6005) | KRPSGVPDRFSGSKSGNAASLTVSGLQAEDEADYYC |
| 85 | LFWR4 (ATL_5262; | FGGGTKVEIK |
| | 6003; 6004; 6033; 6034; | |
| | 6035; 6036; 6037; 6038; | |
| | 6039; 6177; 6178) | |
| 86 | LFWR4 (ATL_5996) | FGQGTKVEIR |
| 87 | LFWR4 (ATL_5997; | FGQGTKVEIK |
| | 5998; 5999; 6002) | |
| 88 | LFWR4 (ATL_6000) | FGPGTKVTVL |
| 89 | LFWR4 (ATL_6001) | FGPGTKVDIK |
| 90 | LFWR4 (ATL_6005) | FGGGTKLTVL |
| 91 | ATL_6177 VH | QVQLQESGPGLVKPSETLSLTCTVSGGSISSYFWSWIRQPPGKG |
| | | LEWIGYIHNSGTTNQNPSLKSRVTISVDKSKNQFSLKLSSVTAADT |
| | | AVYYCARGRGMATLPFDYWGQGTLVTVSS |
| 92 | ATL_6177 VL | DIQMTQSPSSLSASVGDRVTITCRASQSISSYLNWYQQKPGKAPK |
| | | LLIYAASSLQSGVPSRFSGSGSGTDFTLTISSLQPEDFATYYCQQS |
| | | YSTPLTFGGGTKVEIK |
| 93 | ATL_6178 VH | QVQLQESGPGLVKPSETLSLTCTVSGGSISSYFWSWIRQPPGKG |
| | | LEWIGYIHYSGTTNQNPSLKSRVTISVDKSKNQFSLKLSSVTAADT |
| | | AVYYCARGRGMATLPFDYWGQGTLVTVSS |
| 94 | ATL_6178 VL | DIQMTQSPSSLSASVGDRVTITCRASQSISSYLNWYQQKPGKAPK |
| | | LLIYAASSLQSGVPSRFSGSGSGTDFTLTISSLQPEDFATYYCQQS |
| | | YSTPLTFGGGTKVEIK |
| 95 | ATL_5338 VH | EVKLDETGGGLVQPGRPMKLSCVASGFTFSDYWMNWVRQSPEK |
| | | GLEWVAQIRNKPYNYETYYSDSVKGRFTISRDDSKSSVYLQMNN |
| | | LRVEDMGIYYCTGSYYGMDYWGQGTSVTVSS |
| 96 | ATL_5338 VL | DVVMTQTPLSLPVSLGDQASISCRSSQSLVNSNGNTYLRWYLQK |
| | | PGQSPKVLIYKVSNRVSGVPDRFSGSGSGTDFTLKISRVEAEDLG |
| | | VYFCSQSTHVPWTFGGGTKLEIK |
| 97 | rmUNC5C (N-terminal | GSHHHHHHGSDYKDDDDKGSQDDEFFHELPETFPSDPPEPLPH |
| | His and FLAG tag + | FLIEPEEAYIVKNKPVNLYCKASPATQIYFKCNSEWVHQKDHVVDE |
| | Amino acids 41-380 of | RVDETSGLIVREVSIEISRQQVEELFGPEDYWCQCVAWSSAGTTK |
| | the murine UNC5C | SRKAYVRIAYLRKTFEQEPLGKEVSLEQEVLLQCRPPEGIPVAEVE |
| | sequence, extracellular | WLKNEDIIDPAEDRNFYITIDHNLIIKQARLSDTANYTCVAKNIVAKR |
| | domain). In-house | KSTTATVIVYVNGGWSTWTEWSVCNSRCGRGYQKRTRTCTNPA |
| | production from | PLNGGAFCEGQSVQKIACTTLCPVDGRWTSWSKWSTCGTECTH |
| | HEK293F cells | WRRRECTAPAPKNGGKDCDGLVLQSKNCTDGLCMQAAPDSDD |
| | | VALYGS |
| 98 | rhUNC5C (N-terminal | GSHHHHHHGSDYKDDDDKGSQDDDFFHELPETFPSDPPEPLPH |
| | His and FLAG tag + | FLIEPEEAYIVKNKPVNLYCKASPATQIYFKCNSEWVHQKDHIVDE |
| | Amino acids 41- 380 of | RVDETSGLIVREVSIEISRQQVEELFGPEDYWCQCVAWSSAGTTK |
| | the human UNC5C | SRKAYVRIAYLRKTFEQEPLGKEVSLEQEVLLQCRPPEGIPVAEVE |
| | sequence, extracellular | WLKNEDIIDPVEDRNFYITIDHNLIIKQARLSDTANYTCVAKNIVAKR |
| | domain). In-house | KSTTATVIVYVNGGWSTWTEWSVCNSRCGRGYQKRTRTCTNPA |
| | production from | PLNGGAFCEGQSVQKIACTTLCPVDGRWTPWSKWSTCGTECTH |
| | HEK293F cells. | WRRRECTAPAPKNGGKDCDGLVLQSKNCTDGLCMQTAPDSDDV |
| | | ALYGS |
| 99 | >sp\|095185\|UNC5C_HUMAN | MRKGLRATAARCGLGLGYLLQMLVLPALALLSASGTGSAAQDDD |
| | etrin receptor | FFHELPETFPSDPPEPLPHFLIEPEEAYIVKNKPVNLYCKASPATQI |
| | UNC5C OS = Homo | YFKCNSEWVHQKDHIVDERVDETSGLIVREVSIEISRQQVEELFGP |
| | sapiens OX = 9606 | EDYWCQCVAWSSAGTTKSRKAYVRIAYLRKTFEQEPLGKEVSLE |
| | GN = UNC5C PE = 1 | QEVLLQCRPPEGIPVAEVEWLKNEDIIDPVEDRNFYITIDHNLIIKQA |
| | SV = 2 | RLSDTANYTCVAKNIVAKRKSTTATVIVYVNGGWSTWTEWSVCN |
| | | SRCGRGYQKRTRTCTNPAPLNGGAFCEGQSVQKIACTTLCPVDG |
| | | RWTPWSKWSTCGTECTHWRRRECTAPAPKNGGKDCDGLVLQS |
| | | KNCTDGLCMQTAPDSDDVALYVGIVIAVIVCLAISVVVALFVYRKN |
| | | HRDFESDIIDSSALNGGFQPVNIKAARQDLLAVPPDLTSAAAMYR |
| | | GPVYALHDVSDKIPMTNSPILDPLPNLKIKVYNTSGAVTPQDDLSE |
| | | FTSKLSPQMTQSLLENEALSLKNQSLARQTDPSCTAFGSFNSLGG |
| | | HLIVPNSGVSLLIPAGAIPQGRVYEMYVTVHRKETMRPPMDDSQT |
| | | LLTPVVSCGPPGALLTRPVVLTMHHCADPNTEDWKILLKNQAAQG |
| | | QWEDVVVVGEENFTTPCYIQLDAEACHILTENLSTYALVGHSTTK |
| | | AAAKRLKLAIFGPLCCSSLEYSIRVYCLDDTQDALKEILHLERQMG |
| | | GQLLEEPKALHFKGSTHNLRLSIHDIAHSLWKSKLLAKYQEIPFYH |
| | | VWSGSQRNLHCTFTLERFSLNTVELVCKLCVRQVEGEGQIFQLN |
| | | CTVSEEPTGIDLPLLDPANTITTVTGPSAFSIPLPIRQKLCSSLDAP |
| | | QTRGHDWRMLAHKLNLDRYLNYFATKSSPTGVILDLWEAQNFPD |
| | | GNLSMLAAVLEEMGRHETVVSLAAEGQY |
| 100 | >sp\|008747\|UNC5C_ | MRKGLRATAARCGLGLGYLLQMLVLPALALLSASGTGSAAQDDE |
| | MOUSE Netrin receptor | FFHELPETFPSDPPEPLPHFLIEPEEAYIVKNKPVNLYCKASPATQI |
| | UNC5C OS = Mus | YFKCNSEWVHQKDHVVDERVDETSGLIVREVSIEISRQQVEELFG |
| | musculus OX = 10090 | PEDYWCQCVAWSSAGTTKSRKAYVRIAYLRKTFEQEPLGKEVSL |
| | GN = Unc5c PE = 1 SV = 1 | EQEVLLQCRPPEGIPVAEVEWLKNEDIIDPAEDRNFYITIDHNLIIKQ |
| | | ARLSDTANYTCVAKNIVAKRKSTTATVIVYVNGGWSTWTEWSVC |
| | | NSRCGRGYQKRTRTCTNPAPLNGGAFCEGQSVQKIACTTLCPVD |
| | | GRWTSWSKWSTCGTECTHWRRRECTAPAPKNGGKDCDGLVLQ |
| | | SKNCTDGLCMQAAPDSDDVALYVGIVIAVTVCLAITVVVALFVYRK |
| | | NHRDFESDIIDSSALNGGFQPVNIKAARQDLLAVPPDLTSAAAMY |
| | | RGPVYALHDVSDKIPMTNSPILDPLPNLKIKVYNSSGAVTPQDDLA |
| | | EFSSKLSPQMTQSLLENEALNLKNQSLARQTDPSCTAFGTFNSLG |
| | | GHLIIPNSGVSLLIPAGAIPQGRVYEMYVTVHRKENMRPPMEDSQ |
| | | TLLTPVVSCGPPGALLTRPVILTLHHCADPSTEDWKIQLKNQAVQ |
| | | GQWEDVVVVGEENFTTPCYIQLDAEACHILTENLSTYALVGQSTT |
| | | KAAAKRLKLAIFGPLCCSSLEYSIRVYCLDDTQDALKEVLQLERQM |
| | | GGQLLEEPKALHFKGSIHNLRLSIHDIAHSLWKSKLLAKYQEIPFYH |
| | | IWSGSQRNLHCTFTLERLSLNTVELVCKLCVRQVEGEGQIFQLNC |
| | | TVSEEPTGIDLPLLDPASTITTVTGPSAFSIPLPIRQKLCSSLDAPQT |
| | | RGHDWRMLAHKLNLDRYLNYFATKSSPTGVILDLWEAQNFPDGN |
| | | LSMLAAVLEEMGRHETVVSLAAEGQY |
| 101 | rmUNC5C (Amino acids | QDDEFFHELPETFPSDPPEPLPHFLIEPEEAYIVKNKPVNLYCKAS |
| | 41-380 of the murine | PATQIYFKCNSEWVHQKDHVVDERVDETSGLIVREVSIEISRQQV |
| | UNC5C sequence, | EELFGPEDYWCQCVAWSSAGTTKSRKAYVRIAYLRKTFEQEPLG |
| | corresponding to the | KEVSLEQEVLLQCRPPEGIPVAEVEWLKNEDIIDPAEDRNFYITIDH |
| | extracellular domain). | NLIIKQARLSDTANYTCVAKNIVAKRKSTTATVIVYVNGGWSTWTE |
| | In-house production | WSVCNSRCGRGYQKRTRTCTNPAPLNGGAFCEGQSVQKIACTT |
| | from HEK293F cells. | LCPVDGRWTSWSKWSTCGTECTHWRRRECTAPAPKNGGKDCD |
| | | GLVLQSKNCTDGLCMQAAPDSDDVALY |
| 102 | rhUNC5C (Amino acids | QDDDFFHELPETFPSDPPEPLPHFLIEPEEAYIVKNKPVNLYCKAS |
| | 41- 380 of the human | PATQIYFKCNSEWVHQKDHIVDERVDETSGLIVREVSIEISRQQVE |
| | UNC5C sequence, | ELFGPEDYWCQCVAWSSAGTTKSRKAYVRIAYLRKTFEQEPLGK |
| | corresponding to the | EVSLEQEVLLQCRPPEGIPVAEVEWLKNEDIIDPVEDRNFYITIDHN |
| | extracellular domain). | LIIKQARLSDTANYTCVAKNIVAKRKSTTATVIVYVNGGWSTWTEW |
| | In-house production | SVCNSRCGRGYQKRTRTCTNPAPLNGGAFCEGQSVQKIACTTLC |
| | from HEK293F cells. | PVDGRWTPWSKWSTCGTECTHWRRRECTAPAPKNGGKDCDGL |
| | | VLQSKNCTDGLCMQTAPDSDDVALY |
| 103 | Recombinant rat | GSHHHHHHGSDYKDDDDKGSQDDDFFHELPETFPSDPPEPLPH |
| | UNC5C (N-terminal His | FLIEPEEAYIVKNKPVNLYCKASPATQIYFKCNSEWVHQKDHVVDE |
| | and FLAG tag) In-house | RVDETSGLIVREVSIEISRQQVEELFGPEDYWCQCVAWSSAGTTK |
| | production from | SRKAYVRIAYLRKTFEQEPLGKEVSLEQEVLLQCRPPEGIPMAEV |
| | HEK293F cells. | EWLKNEDIIDPVEDRNFYITIDHNLIIKQARLSDTANYTCVAKNIVAK |
| | | RKSTTATVIVYVNGGWSTWAEWSVCNSRCGRGYQKRTRTCTNP |
| | | APLNGGAFCEGQSVQKIACTTLCPVDGRWTSWSKWSTCGTECT |
| | | HWRRRECTAPAPKNGGKDCDGLVLQSKNCTDGLCMQAAPDSD |
| | | DVALYGS |
| 104 | Recombinant cyno | GSHHHHHHGSDYKDDDDKGSQDDDFFHELPETFPSDPPEPLPH |
| | UNC5C (N-terminal His | FLIEPEEAYIVKNKPVNLYCKASPATQIYFKCNSEWVHQKDHIVDE |
| | and FLAG tag) In-house | RVDETSGLIVREVSIEISRQQVEELFGPEDYWCQCVAWSSAGTTK |
| | production from | SRKAYVRIAYLRKTFEQEPLGKEVSLEQEVLLQCRPPEGIPVAEVE |
| | HEK293F cells. | WLKNEDIIDPVEDRNFYITIDHNLIIKQARLSDTANYTCVAKNIVAKR |
| | | KSTTATVIVYVNGGWSTWTEWSVCNSRCGRGYQKRTRTCTNPA |
| | | PLNGGAFCEGQSVQKIACTTLCPVDGRWTPWSKWSTCGTECTH |
| | | WRRRECTAPAPKNGGKDCDGLVLQSKNCTDGLCMQTAPDSDDV |
| | | ALYGS |
| 105 | Human recombinant | GSHHHHHHGSDYKDDDDKGSQQSATVANPVPGANPDLLPHFLV |
| | UNC5A | EPEDVYIVKNKPVLLVCKAVPATQIFFKCNGEWVRQVDHVIERST |
| | | DGSSGLPTMEVRINVSRQQVEKVFGLEEYWCQCVAWSSSGTTK |
| | | SQKAYIRIAYLRKNFEQEPLAKEVSLEQGIVLPCRPPEGIPPAEVE |
| | | WLRNEDLVDPSLDPNVYITREHSLVVRQARLADTANYTCVAKNIV |
| | | ARRRSASAAVIVYVDGSWSPWSKWSACGLDCTHWRSRECSDPA |
| | | PRNGGEECQGTDLDTRNCTSDLCVHTASGPEDVALYGS |
| 106 | Human recombinant | GSHHHHHHGSDYKDDDDKGSGTDSGSEVLPDSFPSAPAEPLPY |
| | UNC5B | FLQEPQDAYIVKNKPVELRCRAFPATQIYFKCNGEWVSQNDHVT |
| | | QEGLDEATGLRVREVQIEVSRQQVEELFGLEDYWCQCVAWSSA |
| | | GTTKSRRAYVRIAYLRKNFDQEPLGKEVPLDHEVLLQCRPPEGVP |
| | | VAEVEWLKNEDVIDPTQDTNFLLTIDHNLIIRQARLSDTANYTCVAK |
| | | NIVAKRRSTTATVIVYVNGGWSSWAEWSPCSNRCGRGWQKRTR |
| | | TCTNPAPLNGGAFCEGQAFQKTACTTICPVDGAWTEWSKWSAC |
| | | STECAHWRSRECMAPPPQNGGRDCSGTLLDSKNCTDGLCMQN |
| | | KKTLSDPNSHLLEASGDAALYGS |
| 107 | Human recombinant | GSQDDDFFHELPETFPSDPPEPLPHFLIEPEEAYIVKNKPVNLYCK |
| | UNC5C version 1 | ASPATQIYFKCNSEWVHQKDHIVDERVDETSGLIVREVSIEISRQQ |
| | | VEELFGPEDYWCQCVAWSSAGTTKSRKAYVRIAYLRKTFEQEPL |
| | | GKEVSLEQEVLLQCRPPEGIPVAEVEWLKNEDIIDPVEDRNFYITID |
| | | HNLIIKQARLSDTANYTCVAKNIVAKRKSTTATVIVYVNGGWSTWT |
| | | EWSVCNSRCGRGYQKRTRTCTNPAPLNGGAFCEGQSVQKIACT |
| | | TLCPVDGRWTPWSKWSTCGTECTHWRRRECTAPAPKNGGKDC |
| | | DGLVLQSKNCTDGLCMQTAPDSDDVALYGSKHHHHHH |
| 108 | Human recombinant | GSHHHHHHGSDYKDDDDKGSARGTDNGEALPESIPSAPGTLPHF |
| | UNC5D | IEEPDDAYIIKSNPIALRCKARPAMQIFFKCNGEWVHQNEHVSEET |
| | | LDESSGLKVREVFINVTRQQVEDFHGPEDYWCQCVAWSHLGTS |
| | | KSRKASVRIAYLRKNFEQDPQGREVPIEGMIVLHCRPPEGVPAAE |
| | | VEWLKNEEPIDSEQDENIDTRADHNLIIRQARLSDSGNYTCMAANI |
| | | VAKRRSLSATVVVYVNGGWSSWTEWSACNVRCGRGWQKRSRT |
| | | CTNPAPLNGGAFCEGMSVQKITCTSLCPVDGSWEVWSEWSVCS |
| | | PECEHLRIRECTAPPPRNGGKFCEGLSQESENCTDGLCILDKKPL |
| | | HEIKPQSIENASDGS |
| 109 | Human recombinant | GSHHHHHHGSDYKDDDDKGSHLQVTGFQIKAFTALRFLSEPSDA |
| | DCC | VTMRGGNVLLDCSAESDRGVPVIKWKKDGIHLALGMDERKQQLS |
| | | NGSLLIQNILHSRHHKPDEGLYQCEASLGDSGSIISRTAKVAVAGP |
| | | LRFLSQTESVTAFMGDTVLLKCEVIGEPMPTIHWQKNQQDLTPIP |
| | | GDSRVVVLPSGALQISRLQPGDIGIYRCSARNPASSRTGNEAEVRI |
| | | LSDPGLHRQLYFLQRPSNVVAIEGKDAVLECCVSGYPPPSFTWLR |
| | | GEEVIQLRSKKYSLLGGSNLLISNVTDDDSGMYTCVVTYKNENISA |
| | | SAELTVLVPPWFLNHPSNLYAYESMDIEFECTVSGKPVPTVNWM |
| | | KNGDVVIPSDYFQIVGGSNLRILGVVKSDEGFYQCVAENEAGNAQ |
| | | TSAQLIVPKPAIPSSSVLPSAPRDVVPVLVSSRFVRLSWRPPAEAK |
| | | GNIQTFTVFFSREGDNRERALNTTQPGSLQLTVGNLKPEAMYTFR |
| | | VVAYNEWGPGESSQPIKVATQPELQVPGPVENLQAVSTSPTSILIT |
| | | WEPPAYANGPVQGYRLFCTEVSTGKEQNIEVDGLSYKLEGLKKF |
| | | TEYSLRFLAYNRYGPGVSTDDITVVTLSDVPSAPPQNVSLEVVNS |
| | | RSIKVSWLPPPSGTQNGFITGYKIRHRKTTRRGEMETLEPNNLWY |
| | | LFTGLEKGSQYSFQVSAMTVNGTGPPSNWYTAETPENDLDESQV |
| | | PDQPSSLHVRPQTNCIIMSWTPPLNPNIVVRGYIIGYGVGSPYAET |
| | | VRVDSKQRYYSIERLESSSHYVISLKAFNNAGEGVPLYESATTRSI |
| | | TDPTDPVDYYPLLDDFPTSVPDLSTPMLPPVGVQAVALTHDAVRV |
| | | SWADNSVPKNQKTSEVRLYTVRWRTSFSASAKYKSEDTTSLSYT |
| | | ATGLKPNTMYEFSVMVTKNRRSSTWSMTAHATTYEAAPTSAPKD |
| | | LTVITREGKPRAVIVSWQPPLEANGKITAYILFYTLDKNIPIDDWIME |
| | | TISGDRLTHQIMDLNLDTMYYFRIQARNSKGVGPLSDPILFRTLKV |
| | | EHPDKMANDQGRHGDGGYWPVDTNLIDRSTLNEPPIGQMHPPH |
| | | GSVTPQKNSNGS |
| 110 | Human UNC5C T835M | MRKGLRATAARCGLGLGYLLQMLVLPALALLSASGTGSAAQDDD |
| | variant | FFHELPETFPSDPPEPLPHFLIEPEEAYIVKNKPVNLYCKASPATQI |
| | | YFKCNSEWVHQKDHIVDERVDETSGLIVREVSIEISRQQVEELFGP |
| | | EDYWCQCVAWSSAGTTKSRKAYVRIAYLRKTFEQEPLGKEVSLE |
| | | QEVLLQCRPPEGIPVAEVEWLKNEDIIDPVEDRNFYITIDHNLIIKQA |
| | | RLSDTANYTCVAKNIVAKRKSTTATVIVYVNGGWSTWTEWSVCN |
| | | SRCGRGYQKRTRTCTNPAPLNGGAFCEGQSVQKIACTTLCPVDG |
| | | RWTPWSKWSTCGTECTHWRRRECTAPAPKNGGKDCDGLVLQS |
| | | KNCTDGLCMQTAPDSDDVALYVGIVIAVIVCLAISVVVALFVYRKN |
| | | HRDFESDIIDSSALNGGFQPVNIKAARQDLLAVPPDLTSAAAMYR |
| | | GPVYALHDVSDKIPMTNSPILDPLPNLKIKVYNTSGAVTPQDDLSE |
| | | FTSKLSPQMTQSLLENEALSLKNQSLARQTDPSCTAFGSFNSLGG |
| | | HLIVPNSGVSLLIPAGAIPQGRVYEMYVTVHRKETMRPPMDDSQT |
| | | LLTPVVSCGPPGALLTRPVVLTMHHCADPNTEDWKILLKNQAAQG |
| | | QWEDVVVVGEENFTTPCYIQLDAEACHILTENLSTYALVGHSTTK |
| | | AAAKRLKLAIFGPLCCSSLEYSIRVYCLDDTQDALKEILHLERQMG |
| | | GQLLEEPKALHFKGSTHNLRLSIHDIAHSLWKSKLLAKYQEIPFYH |
| | | VWSGSQRNLHCTFTLERFSLNTVELVCKLCVRQVEGEGQIFQLN |
| | | CTVSEEPTGIDLPLLDPANTITMVTGPSAFSIPLPIRQKLCSSLDAP |
| | | QTRGHDWRMLAHKLNLDRYLNYFATKSSPTGVILDLWEAQNFPD |
| | | GNLSMLAAVLEEMGRHETVVSLAAEGQY |
| 111 | Human UNC5C | GSDPPEPLPHFLIEPEEAYIVKNKPVNLYCKASPATQIYFKCNSEW |
| | fragment ACB_429 | VHQKDHIVDERVDETSGLIVREVSIEISRQQVEELFGPEDYWCQC |
| | | VAWSSAGTTKSRKAYVRIAYLRKTFEQEPLGKEVSLEQEVLLQCR |
| | | PPEGIPVAEVEWLKNEDIIDPVEDRNFYITIDHNLIIKQARLSDTANY |
| | | TCVAKNIVAKRKSTTATVIVYVGSHHHHHH |
| 112 | ATL_0006187 VH | QVQLQESGPGLVKPSETLSLTCTVSGGSTGSYYWSWIRQPPGK |
| | | GLEWIGYTHYSGHTNQNPSLKSRVTMSVDSSKSQLSLKLTSVTAA |
| | | DTAVYYCARGRGMATLPFDSWGQGILVIVSS |
| 113 | ATL_0006191 VH | QVQLQESGPGLVKPSETLSLTCTVSGGSIGSYFWSWIRQTPGKG |
| | | LEWIGYIHYSGSSNYNPSLKSRVTISVDTFKNQFSLKLTSVTAADT |
| | | AVYYCARGRGMATLPFDYWGQGTLVTVSS |
| 114 | ATL_0006187 | DIQMTQSPSSLSASVGDRVTITCRASQSISSYLNWYQQKPGKAPK |
| | VL ;; ATL_0006191 VL | LLIYAASSLQSGVPSRFSGSGSGTDFTLTISSLQPEDFATYYCQQS |
| | (duplicate sequence, | YSTPLTFGGGTKVEIK |
| | SEQ ID NO: 9, 92, 94) | |
| 115 | HCDR1_ATL_0006187 | GGSTGSYY |
| 116 | HCDR1_ATL_0006191 | GGSIGSYF |
| 117 | HCDR2_ATL_0006187 | THYSGHT |
| 118 | HCDR2_ATL_0006191 | IHYSGSS |
| 119 | HCDR3_ATL_0006187 | ARGRGMATLPFDS |
| 120 | HCDR3_ATL_0006191 | ARGRGMATLPFDY |
| 24 | LCDR1_ATL_0006187; | QSISSY |
| | LCDR1_ATL_0006191 | |
| 34 | LCDR2_ATL_0006187; | AAS |
| | LCDR2_ATL_0006191 | |
| 121 | LCDR3_ATL_0006187; | QQSYSTPLT |
| | LCDR3_ATL_0006191( | |
| | duplicate sequence | |
| | SEQ ID : 40) | |
| 122 | ATL_0006530 VH | QVQLLETGGGLVQPGGSLRLSCAASGFTFSSYSMNWVRQAPGK |
| | | GLEWVSSISSSSSYIYYADSVKGRFTISRDNAKNSLYLQMNSLRA |
| | | EDTAVYYCARDPYSGYEFVSEPYFDYWGQGTLVTVSS |
| 123 | ATL_0006531 VH | EVQLVESGGVVVQPGGSLRLSCAASGFTFDDYAMHWVRQAPGK |
| | | GLEWVSLISWDGGSTYYADSVKGRFTISRDNSKNSLYLQMNSLR |
| | | AEDTALYYCAKDSIQLWSFDYWGQGTLVTVSS |
| 124 | ATL_0006532 VH | QVTLKESGAEVKKPGASVKVSCKASGYTFTGYYMHWVRQAPGQ |
| | | GLEWMGRINPNSGGTNYAQKFQGRVTMTRDTSISTAYMELSRLR |
| | | SDDTAVYYCAREEDCSSTSCYGDAFDIWGQGTMVTVSS |
| 125 | ATL_0006533 VH | QVQLQQSGGGLVKPGGSLRLSCAASGFTFSDYYMSWIRQAPGK |
| | | GLEWVSYISSSSSYTNYADSVKGRFTISRDNAKNSLYLQMNSLRA |
| | | EDTAVYYCARGGGGMDVWGQGTMVTVSS |
| 126 | ATL_0006534 VH | QVQLVESGAEVKKPGASVKVSCKASGYTFTGYYMHWVRQAPGQ |
| | | GLEWMGRINPNSGGTNYAQKFQGRVTMTRDTSISTAYMELSRLR |
| | | SDDTAVYYCAREGDDSSGWLGGAFDIWGQGTMVTVSS |
| 127 | ATL_0006535 VH | EVQLLETGGGLVQPGRSLRLSCAASGFTFDDYAMHWVRQAPGK |
| | | GLEWVSGISWNSGSIGYADSVKGRFTISRDNAKNSLYLQMNSLR |
| | | AEDTALYYCAKDSYGDYFRGYYYGMDVWGQGTMVTVSS |
| 128 | ATL_0006536 VH | EVQLLESAGGVVQLGRSLRLSCAASGFTFSNYGMHWVRQAPGK |
| | | GLEWVAIIYFDGRNKYYAGSVGGRFTISRDNSKNTLYLQMDSLRS |
| | | DDTAVYYCARPLFPWLRVDGLDVWGHGTMVTVSS |
| 129 | ATL_0006537 VH | QVQLVQSGAEVKKPGESLKISCKGSGYSFTSYWIGWVRQMPGK |
| | | GLEWMGIIYPGDSDTRYSPSFQGQVTISADKSISTAYLQWSSLKA |
| | | SDTAMYYCARQRRGSGTRGMDVWGQGTTVTVSS |
| 130 | ATL_0006538 VH | QVQLQESGPGLVKPSGTLSLTCAVSGGSISSSNWWSWVRQPPG |
| | | KGLEWIGEIYHSGSTNYNPSLKSRVTISVDKSKNQFSLKLSSVTAA |
| | | DTAVYYCARGRGGSYYFDYWGQGTLVTVSS |
| 131 | ATL_0006539 VH | QVQLVESGGGLVQPGGSLRLSCSASGFTFINYAMSWVRQAPGK |
| | | GLEWVSTISGSGASIYYADSVKGRFTISRDNSKNTVYLQMNSLRA |
| | | EDTAVYYCAKGAYYDSSGYYALDAFDIWGQGTMVTVSS |
| 132 | ATL_0006530 VL | NFMLTQPHSVSESPGKTVTISCTGSSGSIASNYVQWYQQRPGSA |
| | | PTTVIYEDNQRPSGVPDRFSGSIDSSSNSASLTISGLKTEDEADYY |
| | | CQSYDSSNHWVFGGGTKVTVL |
| 133 | ATL_0006531 VL | QSALTQPASVSGSPGQSITISCTGARSDVGGYDYVSWFQQHPGK |
| | | VPKLMIYDVNNRPSGVSNRFSGSKSGNTASLTISGLQAEDEADYY |
| | | CSSFTDSSTYVFGTGTKVTVL |
| 134 | ATL_0006532 VL | QSVLTQPHSVSESPGKTVTISCTRSSGSIVSNYVQWYQQRPGSS |
| | | PTTVIYEDNQRPSGVPDRFSGSIDRSSNSASLTISGLKTEDEGDYY |
| | | CQSYDKTSRVILFGEGTKVTVL |
| 135 | ATL_0006533 VL | NFMLTQPHSVSESPGKTVTISCTGSSGSIASNYVQWYQQRPGSA |
| | | PTTVIYEDNQRPSGVPDRFSGSIDSSSNSASLTISGLKTEDEADYY |
| | | CQSYDSSNHVVFGGGTKVTVL |
| 136 | ATL_0006534 VL | NFMLTQPHSVLESPGKTVTIPCTRSSGSIASNYVQWYQQRPGSA |
| | | PTAVIFEDNVRPSGVPDRFSGSVDSSSNSASLTISGLKTEDEGDF |
| | | YCQSYDSSGHVVFGGGTKVTVL |
| 137 | ATL_0006535 VL | QSVLTQPPSVSGAPGQRVTISCTGSSSNIGAGYDVHWYQQLPGT |
| | | APKLLIYGNSNRPSGVPDRFSGSKSGTSASLAITGLQAEDEADYY |
| | | CQSYDSSLSASYVFGTGTKVTVL |
| 138 | ATL_0006536 VL | QSALTQPASVSGSPGQSITISCTGTSSDVGNYNLVSWYQQYPGK |
| | | APKLLIYEVIKRPSGISGRFSGSKSGNTASLTISGLQAEDEADYYC |
| | | SSYTSSSTDYVFGTGTKVTVL |
| 139 | ATL_0006537 VL | QAGLTQPPSVSVSPGQTASITCSGKKLGDKYTSWYQQKPGQSPL |
| | | LVIYQDNKRPSGVPGRFSGSNSGNTATLTISGTQPMDEADYYCQ |
| | | AWDSSTVIFGGGTKVTVL |
| 140 | ATL_0006538 VL | QSVLTQPPSASGTPGQRVTISCSGSSSNIGSNTVNWYQQVPGTA |
| | | PKLLIYSNNQRPSGVPDRFSGSKSGTSASLAISGLQSEDEADYYC |
| | | AAWDDSLNGWVFGGGTKVTVL |
| 141 | ATL_0006539 VL | QSVLTQPPSVSVSAGQTASITCSGDMLGGKYASWYQHKPGQSP |
| | | VLVIYQDARRPSGISERFSGSSSGNTATLTISGTQAMDEADYYCQ |
| | | AWGSTAVFGGGTKVTVL |
| 142 | HCDR1_ATL_0006530 | GFTFSSYS |
| 143 | HCDR1_ATL_0006531; | GFTFDDYA |
| | HCDR1_ATL_0006535 | |
| 144 | HCDR1_ATL_0006532; | GYTFTGYY |
| | HCDR1_ATL_0006534 | |
| 145 | HCDR1_ATL_0006533 | GFTFSDYY |
| 146 | HCDR1_ATL_0006536 | GFTFSNYG |
| 147 | HCDR1_ATL_0006537 | GYSFTSYW |
| 148 | HCDR1_ATL_0006538 | GGSISSSNW |
| 149 | HCDR1_ATL_0006539 | GFTFINYA |
| 150 | HCDR2_ATL_0006530 | ISSSSSYI |
| 151 | HCDR2_ATL_0006531 | ISWDGGST |
| 152 | HCDR2_ATL_0006532; | INPNSGGT |
| | HCDR2_ATL_0006534 | |
| 153 | HCDR2_ATL_0006533 | ISSSSSYT |
| 154 | HCDR2_ATL_0006535 | ISWNSGSI |
| 155 | HCDR2_ATL_0006536 | IYFDGRNK |
| 156 | HCDR2_ATL_0006537 | IYPGDSDT |
| 157 | HCDR2_ATL_0006538 | IYHSGST |
| 158 | HCDR2_ATL_0006539 | ISGSGASI |
| 159 | HCDR3_ATL_0006530 | ARDPYSGYEFVSEPYFDY |
| 160 | HCDR3_ATL_0006531 | AKDSIQLWSFDY |
| 161 | HCDR3_ATL_0006532 | AREEDCSSTSCYGDAFDI |
| 162 | HCDR3_ATL_0006533 | ARGGGGMDV |
| 163 | HCDR3_ATL_0006534 | AREGDDSSGWLGGAFDI |
| 164 | HCDR3_ATL_0006535 | AKDSYGDYFRGYYYGMDV |
| 165 | HCDR3_ATL_0006536 | ARPLFPWLRVDGLDV |
| 166 | HCDR3_ATL_0006537 | ARQRRGSGTRGMDV |
| 167 | HCDR3_ATL_0006538 | ARGRGGSYYFDY |
| 168 | HCDR3_ATL_0006539 | AKGAYYDSSGYYALDAFDI |
| 169 | LCDR1_ATL_0006530; | SGSIASNY |
| | LCDR1_ATL_0006533; | |
| | LCDR1_ATL_0006534 | |
| 170 | LCDR1_ATL_0006531 | RSDVGGYDY |
| 171 | LCDR1_ATL_0006532 | SGSIVSNY |
| 172 | LCDR1 ATL_0006535 | SSNIGAGYD |
| 173 | LCDR1_ATL_0006536 | SSDVGNYNL |
| 174 | LCDR1_ATL_0006537 | KLGDKY |
| 175 | LCDR1_ATL_0006538 | SSNIGSNT |
| 176 | LCDR1_ATL_0006539 | MLGGKY |
| 177 | LCDR2_ATL_0006530; | EDN |
| | LCDR2_ATL_0006532; | |
| | LCDR2_ATL_0006533; | |
| | LCDR2_ATL_0006534 | |
| 178 | LCDR2_ATL_0006531 | DVN |
| 179 | LCDR2_ATL_0006535 | GNS |
| 180 | LCDR2_ATL_0006536 | EVI |
| 181 | LCDR2_ATL_0006537 | QDN |
| 182 | LCDR2_ATL_0006538 | SNN |
| 183 | LCDR2_ATL_0006539 | QDA |
| 184 | LCDR3_ATL_0006530 | QSYDSSNHWV |
| 185 | LCDR3_ATL_0006531 | SSFTDSSTYV |
| 186 | LCDR3_ATL_0006532 | QSYDKTSRVIL |
| 187 | LCDR3_ATL_0006533 | QSYDSSNHVV |
| 188 | LCDR3_ATL_0006534 | QSYDSSGHVV |
| 189 | LCDR3_ATL_0006535 | QSYDSSLSASYV |
| 190 | LCDR3_ATL_0006536 | SSYTSSSTDYV |
| 191 | LCDR3_ATL_0006537 | QAWDSSTVI |
| 192 | LCDR3_ATL_0006538 | AAWDDSLNGWV |
| 193 | LCDR3_ATL_0006539 | QAWGSTAV |
| 194 | human UNC5C version | GSQDDDFFHELPETFPSDPPEPLPHFLIEPEEAYIVKNKPVNLYCK |
| | 2 | ASPATQIYFKCNSEWVHQKDHIVDERVDETSGLIVREVSIEISRQQ |
| | | VEELFGPEDYWCQCVAWSSAGTTKSRKAYVRIAYLRKTFEQEPL |
| | | GKEVSLEQEVLLQCRPPEGIPVAEVEWLKNEDIIDPVEDRNFYITID |
| | | HNLIIKQARLSDTANYTCVAKNIVAKRKSTTATVIVYVNGGWSTWT |
| | | EWSVCNSRCGRGYQKRTRTCTNPAPLNGGAFCEGQSVQKIACT |
| | | TLCPVDGRWTPWSKWSTCGTECTHWRRRECTAPAPKNGGKDC |
| | | DGLVLQSKNCTDGLCMQTAPDSDDVALYGSHHHHHH |
| 195 | HFWR2_ATL_0006191 | WSWIRQTPGKGLEWIGY |
| 196 | HFWR3_ATL0006187 | NQNPSLKSRVTMSVDSSKSQLSLKLTSVTAADTAVYYC |
| 197 | HFWR3_ATL0006191 | NYNPSLKSRVTISVDTFKNQFSLKLTSVTAADTAVYYC |
| 198 | HFWR4_ATL0006187 | WGQGILVIVSS |
| 199 | HFWR1_ATL_0006530 | QVQLLETGGGLVQPGGSLRLSCAAS |
| 200 | HFWR2_ATL_0006530 | MNWVRQAPGKGLEWVSS |
| 201 | HFWR3_ATL_0006530 | YYADSVKGRFTISRDNAKNSLYLQMNSLRAEDTAVYYC |
| 202 | HFWR1_ATL_0006531 | EVQLVESGGVVVQPGGSLRLSCAAS |
| 203 | HFWR2_ATL_0006531 | MHWVRQAPGKGLEWVSL |
| 204 | HFWR3_ATL_0006531 | YYADSVKGRFTISRDNSKNSLYLQMNSLRAEDTALYYC |
| 205 | HFWR1_ATL_0006532 | QVTLKESGAEVKKPGASVKVSCKAS |
| 206 | HFWR2_ATL_0006532, | MHWVRQAPGQGLEWMGR |
| | 6534 | |
| 207 | HFWR3_ATL_0006532, 6534 | NYAQKFQGRVTMTRDTSISTAYMELSRLRSDDTAVYYC |
| 208 | HFWR4_ATL_0006532, | WGQGTMVTVSS |
| | 6533, 6534, 6535, 6539 | |
| 209 | HFWR1_ATL_0006533 | QVQLQQSGGGLVKPGGSLRLSCAAS |
| 210 | HFWR2_ATL_0006533 | MSWIRQAPGKGLEWVSY |
| 211 | HFWR3_ATL_0006533 | NYADSVKGRFTISRDNAKNSLYLQMNSLRAEDTAVYYC |
| 212 | HFWR1_ATL_0006534 | QVQLVESGAEVKKPGASVKVSCKAS |
| 213 | HFWR1 ATL_0006535 | EVQLLETGGGLVQPGRSLRLSCAAS |
| 214 | HFWR2_ATL_0006535 | MHWVRQAPGKGLEWVSG |
| 215 | HFWR3_ATL_0006535 | GYADSVKGRFTISRDNAKNSLYLQMNSLRAEDTALYYC |
| 216 | HFWR1_ATL_0006536 | EVQLLESAGGVVQLGRSLRLSCAAS |
| 217 | HFWR2_ATL_0006536 | MHWVRQAPGKGLEWVAI |
| 218 | HFWR3_ATL_0006536 | YYAGSVGGRFTISRDNSKNTLYLQMDSLRSDDTAVYYC |
| 219 | HFWR4_ATL_0006536 | WGHGTMVTVSS |
| 220 | HFWR1_ATL_0006537 | QVQLVQSGAEVKKPGESLKISCKGS |
| 221 | HFWR2_ATL_0006537 | IGWVRQMPGKGLEWMGI |
| 222 | HFWR3_ATL_0006537 | RYSPSFQGQVTISADKSISTAYLQWSSLKASDTAMYYC |
| 223 | HFWR4_ATL_0006537 | WGQGTTVTVSS |
| 224 | HFWR1_ATL_0006538 | QVQLQESGPGLVKPSGTLSLTCAVS |
| 225 | HFWR2_ATL_0006538 | WSWVRQPPGKGLEWIGE |
| 226 | HFWR3_ATL_0006538 | NYNPSLKSRVTISVDKSKNQFSLKLSSVTAADTAVYYC |
| 227 | HFWR1_ATL_0006539 | QVQLVESGGGLVQPGGSLRLSCSAS |
| 228 | HFWR2_ATL_0006539 | MSWVRQAPGKGLEWVST |
| 229 | HFWR3_ATL_0006539 | YYADSVKGRFTISRDNSKNTVYLQMNSLRAEDTAVYYC |
| 230 | LFWR1_ATL_0006530, 6533 | NFMLTQPHSVSESPGKTVTISCTGS |
| 231 | LFWR1_ATL_0006531 | QSALTQPASVSGSPGQSITISCTGA |
| 232 | LFWR1_ATL_0006532 | QSVLTQPHSVSESPGKTVTISCTRS |
| 233 | LFWR1_ATL_0006534 | NFMLTQPHSVLESPGKTVTIPCTRS |
| 234 | LFWR1_ATL_0006535 | QSVLTQPPSVSGAPGQRVTISCTGS |
| 235 | LFWR1_ATL_0006537 | QAGLTQPPSVSVSPGQTASITCSGK |
| 236 | LFWR1 ATL_0006538 | QSVLTQPPSASGTPGQRVTISCSGS |
| 237 | LFWR1_ATL_0006539 | QSVLTQPPSVSVSAGQTASITCSGD |
| 238 | LFWR2_ATL_0006530, 6533 | VQWYQQRPGSAPTTVIY |
| 239 | LFWR2_ATL_0006531 | VSWFQQHPGKVPKLMIY |
| 240 | LFWR2_ATL_0006532 | VQWYQQRPGSSPTTVIY |
| 241 | LFWR2_ATL_0006534 | VQWYQQRPGSAPTAVIF |
| 242 | LFWR2_ATL_0006535 | VHWYQQLPGTAPKLLIY |
| 243 | LFWR2_ATL_0006536 | VSWYQQYPGKAPKLLIY |
| 244 | LFWR2_ATL_0006537 | TSWYQQKPGQSPLLVIY |
| 245 | LFWR2_ATL_0006538 | VNWYQQVPGTAPKLLIY |
| 246 | LFWR2_ATL_0006539 | ASWYQHKPGQSPVLVIY |
| 247 | LFWR3_ATL_0006530, 6533 | QRPSGVPDRFSGSIDSSSNSASLTISGLKTEDEADYYC |
| 248 | LFWR3_ATL_0006531 | NRPSGVSNRFSGSKSGNTASLTISGLQAEDEADYYC |
| 249 | LFWR3_ATL_0006532 | QRPSGVPDRFSGSIDRSSNSASLTISGLKTEDEGDYYC |
| 250 | LFWR3_ATL_0006534 | VRPSGVPDRFSGSVDSSSNSASLTISGLKTEDEGDFYC |
| 251 | LFWR3_ATL_0006535 | NRPSGVPDRFSGSKSGTSASLAITGLQAEDEADYYC |
| 252 | LFWR3_ATL_0006536 | KRPSGISGRFSGSKSGNTASLTISGLQAEDEADYYC |
| 253 | LFWR3_ATL_0006537 | KRPSGVPGRFSGSNSGNTATLTISGTQPMDEADYYC |
| 254 | LFWR3_ATL_0006538 | QRPSGVPDRFSGSKSGTSASLAISGLQSEDEADYYC |
| 255 | LFWR3_ATL_0006539 | RRPSGISERFSGSSSGNTATLTISGTQAMDEADYYC |
| 256 | LFWR4_ATL_0006530, | FGGGTKVTVL |
| | 6533, 6534, 6537, 6538, | |
| | 6539 | |
| 257 | LFWR4_ATL_0006531, | FGTGTKVTVL |
| | 6535, 6536 | |
| 258 | LFWR4_ATL_0006532 | FGEGTKVTVL |
| 259 | Region of UNC5C_HS- | YCKASPATQIYFKCNSEWVHQKDHIVDERVDETSGLIVREV |
| | 095185-1 that interacts | |
| | with ATL_5262 | |
| 260 | Region of UNC5B_Hs- | RCRAFPATQIYFKCNGEWVSQNDHVTQEGLDEATGLRVREV |
| | Q8IZJ1-1 that is | |
| | homologous to SEQ ID | |
| | NO: 259 | |
| 261 | Region of UNC5A_Hs- | VCKAVPATQIFFKCNGEWVRQVDHVIERSTDGSSGLPTMEV |
| | Q6ZN44-1 that is | |
| | homologous to SEQ ID | |
| | NO: 259 | |
| 262 | Region of UNC5D_Hs- | RCKARPAMQIFFKCNGEWVHQNEHVSEETLDESSGLKVREV |
| | Q6UXZ4-1 that is | |
| | homologous to SEQ ID | |
| | NO: 259 | |

Table 1. Sequences referred to in the present disclosure. VH = Heavy chain variable domain;
VL = Light chain variable domain; CDR = Complementarity-Determining Region, CDRL refers to
CDRs in the light chain variable domain (numbered 1, 2 or 3 from N-ter); CDRH refers to
CDRs in the heavy chain variable domain ((numbered 1, 2 or 3 from N-ter)); FWR = Framework
region; LFWR refers to an FWR in the light chain (numbered 1, 2, 3 or 4 from N-ter) whereas
HFWR refers to an FWR in the heavy chain (numbered 1, 2, 3 or 4 from N-ter). “ATL_” is used
as an antibody identifier.

### EXAMPLES

These examples demonstrate the identification of potential therapeutic antibodies from a cohort of subjects that have or at considered at risk of developing frontotemporal dementia or Parkinson's disease (PD) (Example 1), as well as their in vitro and in vivo characterisation (Examples 2-5, 7-19) and optimisation (Example 6). Further antibodies were subsequently identified that display similar properties to those of Example 1 and 6 (Example 20).

### Materials and Methods

### Human and Mouse UNC5C ELISA

UNC5C recombinant human antigen (Fc-tagged R&D Systems, #1005-UN; or in-house production from HEK293F cells with N-terminal His or FLAG tag (SEQ ID NO: 98)—in-house human antigen was used to confirm results were not driven by Fc tag) or mouse antigen (in-house production from HEK393F cells with N-terminal His or FLAG tag (SEQ ID NO:97—the expressed murine UNC5C extracellular domain comprises AA 41-380 of the murine UNC5C sequence); rat antigen (SEQ ID NO: 103); or cyno antigen (SEQ ID NO: 104) were directly absorbed to an ELISA plate at 3 μg/ml (50 μl per well) and incubated overnight at 4° C. The plate was washed with PBS. Plates were blocked with 200 μl/well of blocking solution (1% BSA w/v in PBS) for 1 hour at room temperature. Following this, the blocking solution was removed and antibodies to be assessed were diluted in dilution series (1 μM to 0.05 nM) in blocking solution (1% BSA w/v in PBS) and applied to the plate. Plates were incubated at room temperature for 1 hour. The plate was washed with PBS/0.1% Tween. Anti-Human IgG HRP (Jackson ImmunoResearch; #109-035-097) was added to plate and incubated for 1 hour at room temperature to detect antibody binding. The plate was washed with PBS/0.1% Tween and TMB solution (Life Technology; #002023) added. Plates were incubated for 5 minutes at room temperature prior to the addition of stopping solution (0.5M sulphuric acid). Absorbance was read on Molecular Devices FilterMaxF5 plate reader at 450 nm. Analysis was performed using a non-linear fit curve fitting algorithm on GraphPad prism and EC50 calculated.

### Target Deconvolution Using Retrogenix Proteomics

The pool of 20 antibodies used in this screen were 18 antibodies identified from FTD sample cohort analysis, one antibody from a Huntington cohort and one positive control anti-PDL1 antibody. The antibodies were mixed in equal proportions (concentrations) to create the pool. The antibody pool was screened at 4 ug/ml, so each individual antibody within the pool was screened at 0.2 ug/ml. The pool of antibodies was screened for binding against fixed HEK293 cells/slides expressing the “Retrogenix library” comprising duplicate 6019 human plasma membrane proteins, secreted and cell surface tethered human secreted proteins and 397 human heterodimers. Screens were performed in duplicate. Antibody binding is detected using an AlexaFluor647-labelled secondary antibody and scanning slides. UNC5C was identified as a weak binder in one replicate and a very weak binder in the second replicate.

Following this result, each antibody from the pool was tested individually for binding to rhUNC5C protein by ELISA.

### Netrin-1 Competition Assay

UNC5C recombinant human antigen (R&D Systems; #1005-UN) or mouse antigen (in-house production) were directly absorbed to ELISA plate at 3 μg/ml (50 μl per well) and incubated overnight at 4 C. The plate was washed with PBS. Plates were blocked with 200 μl/well of blocking solution (1% BSA w/v in PBS) for 1 hour at room temperature. Following this, recombinant human Netrin 1 with C-terminal His tag (R&D Systems; #6419-N1) or vehicle was added to the plate for a pre-incubation step at room temperature for 30 minutes. Netrin 1 concentration was 200 μg/ml for single point competition assay and in a dilution range from 200 μg/ml to 0.003 μg/ml. The assay was set up in replicate wells to be able to detect both ATL_0005262 antibody binding and Netrin 1 binding. ATL_0005262 (at 4 μg/ml) was applied to the wells containing Netrin 1 and was incubated at room temperature for 1 hour. The plate was then washed with PBS/0.1% Tween. Anti-Human IgG HRP was added to plate (Jackson ImmunoResearch; #109-035-097) and incubated for 1 hour at room temperature to detect antibody binding to UNC5C. Anti-His-HRP (Invitrogen #MA1-21315-HRP) was added to wells to detect Netrin 1 binding to UNC5C. The plate was washed with PBS/0.1% Tween and TMB solution (LifeTechnology; #002023) added. Plates were incubated for 5 minutes at room temperature prior to the addition of stopping solution (0.5M sulphuric acid). Absorbance read on Molecular Devices FilterMaxF5 plate reader at 450 nm.

For the reverse experiment to see if Netrin-1 could displace ATL_0005252, UNC5C recombinant human antigen (R&D Systems; #1005-UN) was directly absorbed to ELISA plate at 3 ug/ml (50 μl per well) and incubated overnight at 4 C. The plate was washed with PBS. Plates were blocked with 200 ul/well of blocking solution (1% BSA w/v in PBS) for 1 hour at room temperature. Following this, ATL_0005262 or negative control antibody ATL_0005338 were added to the plates in a 12 point 3-fold dilution curve starting from 666 nM. Plates were incubated for 30 minutes at room temperature. Netrin 1 was then added to the wells at a final concentration of 75 nM, and incubated at room temperature for 1 hour. The assay was set up in replicate wells to be able to detect both ATL_0005262 antibody binding and Netrin 1 binding. The plate was then washed with PBS/0.1% Tween. Anti-Human IgG HRP was added to plate (Jackson ImmunoResearch; #109-035-097) and incubated for 1 hour at room temperature to detect antibody binding to UNC5C. Anti-His-HRP (Invitrogen #MA1-21315-HRP) was added to wells to detect Netrin 1 binding to UNC5C. The plate was washed with PBS/0.1% Tween and TMB solution (Life Technology; #002023) added. Plates were incubated for 5 minutes at room temperature prior to the addition of stopping solution (0.5M sulphuric acid). Absorbance read on Molecular Devices FilterMaxF5 plate reader at 450 nm.

### Biolayer Interferometry Assay

Binding interaction of ATL_5262 or ATL_6178 with different netrin-1 receptors was assessed by bio-layer interferometry (BLI) using an Octet Red 96e instrument. Purified recombinant antigens were purchased from a commercial supplier or prepared and purified in-house and details are shown in Table 2. Recombinant human antigens detailed in the table below were captured via his tag on Sartorius Octet HIS1K biosensors in assay buffer (Cytiva HBS-EP+ kinetics buffer. Loaded sensors were equilibrated to assay buffer and then dipped in antibody analyte (ATLX-1282, ATL5262 hIgG1-LALA) and response (association) was measured. Sensors were then dipped in binding buffer and response (dissociation) was measured.

Biosensors were hydrated and conditioned prior to use by immersion in assay buffer and regeneration buffer (10 mM glycine pH 1.5, Cytiva), and brought to equilibrium in assay buffer prior to loading.

The binding signal of a control antibody dipped into the same sample was subtracted from the raw data. Reference data was collected from loaded sensors dipped in a concentration-matched control antibody analyte.

TABLE 2
Recombinant proteins used for BLI.

| Antigen | | Uniprot | Gene | |
|---|---|---|---|---|
| name | Species | accession | name | Supplier |
| UNC5A | Human | Q6ZN44 | UNC5A | R&D Systems |
| | | | | catalogue number |
| | | | | 6767-UN or in house |
| | | | | SEQ ID NO: 105 |
| UNC5B | Human | Q8IZJ1 | UNC5B | R&D Systems |
| | | | | catalogue number |
| | | | | 8869-UN or in house |
| | | | | SEQ ID NO: 106 |
| UNC5C | Human | O95185 | UNC5C | R&D Systems |
| | | | | catalogue number |
| | | | | 1005-UN or in house |
| | | | | SEQ ID NO: 107 |
| UNC5D | Human | Q6UXZ4 | UNC5D | R&D Systems |
| | | | | catalogue number |
| | | | | 1429-UN or in house |
| | | | | SEQ ID NO: 108 |
| Neogenin | Human | Q92859 | NEO1 | R&D Systems |
| | | | | catalogue |
| | | | | number8607-NE |
| Netrin | Human | P43146 | DCC | R&D Systems |
| receptor DCC | | | | Catalogue number |
| | | | | 8637-DC or in house |
| | | | | SEQ ID NO: 109 |
| Cell adhesion | Human | O60469 | DSCAM | R&D Systems |
| molecule | | | | Catalogue number |
| DSCAM | | | | 3666-DS |

Capillary Isoelectric Focusing (cIEF)

Charge variant analysis was performed by preparing a master mix to dilute antibody samples to run on a cIEF cartridge on a Maurice instrument (Protein Simple). The master mix had a final concentration of methyl cellulose 0.35% (Protein Simple, 101876), pharmalyte pH 3-10 4% (Protein Simple, 17-0456-01), 10 mM arginine (Protein Simple, 042-691), pl markers 4.05 and 9.99 0.01% (Protein Simple, 046-029 and 046-034). Samples were diluted at 0.15-0.25 mg/ml in the master mix and were run for 1 minute at 1500 volts followed by 4.5 minutes at 3000 volts. A system suitability standard (Protein Simple, 046-044) was also run at the beginning of the run. Stability data generated at 2 and 4 weeks was overlaid to compare the charge species profile.

Capillary isoelectric focusing (CIEF) for ATL_6187 was performed on a Maurice instrument (ProteinSimple). 20 ug of sample was added to a master mix containing carrier ampholyte, pl markers pl 7 and 10, methyl cellulose and urea and loaded into a cIEF cartridge (ProteinSimple), separation was performed between the pH range pl 7.0 to 10.0 and detection performed at 280 nm.

### Size-Exclusion Chromatography (SEC-HPLC)

Antibody samples were diluted to 1.2 mg/ml in 20 mM histidine acetate, 150 mM NaCl pH 5.5 to runon a Zorbax GF-250 SEC-HPLC column (Agilent) on a Vanquish Flex (Thermo Scientific). Samples were separated by size in mobile phase 20 mM Sodium phosphate, 300 mM Sodium sulfate and 100 mM Arginine at a flow rate of 0.75 ml/minute at +25 C for 25 minutes per sample. Chromeleon software (Thermo Scientific) was used to integrate the chromatograms.

### SDS-PAGE

2 μg of antibody samples were prepared by diluting in water and 2× Laemmli Sample Buffer (Bio-Rad). Samples were analysed at both reduced and non-reduced conditions; reduced samples had the addition of 50 mM DTT (Sigma-Aldrich). All samples were heated to 95° C. for 5 mins and loaded into wells of Bolt 4-12% Bis-Tris Plus Protein gels (Thermo Scientific) alongside 5 μl of a molecular weight marker was loaded on each gel, Precision Plus Protein Dual Colour Standards (Bio-Rad, 1610737) and run in Bolt MES SDS Running Buffer (Thermo Scientific). Samples were run at a constant 200 Volts for 23 minutes followed by staining with InstantBlue Coomassie Stain for a minimum of 1 hour, gels were destained with water washes and an image taken by scanning using the GelDoc Go Imaging system (Bio-Rad).

### Capillary Electrophoresis Sodium Dodecyl Sulfate (CE-SDS)

CE-SDS was performed on a Maurice instrument separating samples on a CE-SDS Plus Cartridge (Protein Simple). Antibody samples were prepared by mixing 5 μL of sample at 12.6 mg/ml with 55 μL of 1× Sample Buffer (Protein Simple), for a 1 mg/mL test solution concentration. For reduced samples 2.5 μL of 2-Mercaptoethanol (Sigma-Aldrich ref: M6250) was added to the mix for non-reduced samples 2.5 μL of Iodoacetamide (Sigma-Aldrich) was added, then thoroughly vortexed and heated at 70° C. for 10 minutes, followed by 5 minutes on ice. The samples were then briefly vortexed and spun down, 50 μL of sample mix was transferred to a 96-well plate and centrifuged at 1000×g for 10 minutes then placed in the Maurice instrument. For reducing conditions samples were run at 4600 Volts for 25 seconds for injection followed by 5750 Volts for 25 minutes for separation. For non-reducing conditions samples were run at 4600 Volts for 25 seconds for injection followed by 5750 Volts for 35 minutes for separation, data was analysed using Chromeleon software (Thermo Scientific).

### Stability Conditions

100 μl vials of 12.6 mg/ml mAb (ATL_5262) or 50 mg/ml (ATL_6178) were stored at 40° ° C. in a static incubator, 4° ° C. fridge or −80° ° C. freezer for a 2- or 4-week period. Triplicate vials were prepared for each condition. For freeze thaw conditions mAb samples at 12.6 mg/ml were freeze thawed from −80 C storage to room temperature (RT) over three cycles within an eight-hour period.

Samples at 50 mg/ml (ATL_6178) for freeze-thaw assessments were temperature cycled between −70° C. and room temperature, allowing the sample to completely thaw each time, for either 3 or 5 cycles. All stressed samples were compared to a −70° C. frozen control sample.

Samples requiring agitation were placed in a shaking incubator at 25° C. and 300 rpm.

Samples requiring low pH conditions were adjusted to pH3.5 using hydrochloric acid and placed at 25° C. for 6 h or 24 h, after which they were frozen at −70° C. until required for analysis.

### Solubility Conditions

Prior to performing solubility assessments, purified ATL_6178 was concentrated to 50 mg/mL, 100 mg/mL or to a maximum of 288.9 mg/mL by ultrafiltration centrifugation. The sample at 288.9 mg/mL was diluted to 200 mg/mL for analysis. Samples were then analysed before or after being incubated at 25° C. for 7 days.

### Thermal Shift Assay

Protein thermal shift measurements were performed on an Uncle (Unchained labs). Antibodies were diluted to 1 mg/ml or 5 mg/ml in 20 mM Histidine Acetate, 150 mM NaCl, pH 5.5 buffer and run through a temperature ramp of 25-95° C. increasing at a rate of 0.5° C./minute. Samples were run in triplicate, loading 8.8 μl in 3 different wells of a uni (Unchained Labs). Laser settings were set to achieve an initial fluorescence in the 300-350 nm range of 10000-50000 counts. Melting temperature (Tm1/Tm2) and aggregation temperature (Tagg/Tonset) were analysed using Uncle Analysis software v6 (Unchained Labs). Tm measurement was calculated using the 350/330 nm ratio, while Tonset and Tagg were obtained from SLS read at 266 nm.

### UNC5C PK

To determine the PK of UNC5C antibodies, an in vivo PK experiment was performed with ATL_0005262. Six-eight week-old male C57BL/6J mice were treated via IP with ATL_0005262 at 1, 10 and 60 mg/kg and serum was collected 1, 4, 8, 24, 72 and 144 hours post-dose. Brains and cerebrospinal fluid (CSF) were collected at 24, 72 and 144 hours post-dose. ATL_5338 was administered at 10 mg/kg as a control antibody. Antibody levels were assessed via ELISA.

### Netrin-1 Competition Assay

To determine if Netrin-1 could displace phage-derived antibodies, UNC5C recombinant human antigen (R&D Systems; #1005-UN) was directly absorbed to ELISA plates and incubated overnight at 4° C. The plates were washed with PBS. Plates were blocked with 1% BSA w/v in PBS blocking solution for 1 hour at room temperature. Following this, the phage-derived antibodies (ATL6530-ATL6539) or negative control antibody ATL_0005338 were added to the plates in a dilution curve ranging from 1 μM to 0.1 pM (final concentration; once Netrin-1 added). Plates were incubated for 30 minutes at room temperature. Netrin-1 (R&D Systems; #6419-N1) was then added to the wells at a final concentration of 75 nM, and incubated at room temperature for 1 hour. The assay was set up in replicate wells to be able to detect both antibody binding UNC5C in the presence and absence of Netrin-1, in addition to Netrin-1 binding UNC5C in the presence of antibody. The plate was then washed with PBS/0.1% Tween. Anti-human IgG HRP was added to the plates (Jackson ImmunoResearch; #109-035-097) and incubated for 1 hour at room temperature to detect antibody binding to UNC5C. Anti-His-HRP (Invitrogen #MA1-21315-HRP) was added to wells to detect Netrin-1 binding to UNC5C. The plates were washed with PBS/0.1% Tween and TMB solution (LifeTechnology; #002023) added. Plates were incubated for 5 minutes at room temperature prior to the addition of stopping solution (0.5 M sulphuric acid). Absorbance was read on a Molecular Devices FilterMaxF5 plate reader at 450 nm. Analysis was performed using a non-linear fit curve fitting algorithm on GraphPad prism and EC50 calculated.

### Immunohistochemistry

C57BL/6 naïve mouse brains were cryopreserved via snap freezing in OCT, sectioned using a cryostat onto glass slides and stored at −20° C. until use. Slides were thawed and air-dried prior to fixation in neutral buffered formalin (10%) for 10 minutes and washed in PBS-Triton X (0.5%) for 30 minutes. Hydrophobic barriers were drawn around each tissue section and blocked with appropriate blocking solution (1% BSA, 10% FBS, 5 mM EDTA, 0.3 M glycine in PBS-Tx 0.5%) with addition of either Clusterin (R&D Systems cat. #2937-HS-050), Netrin-4 (R&D Systems cat. #1254-N4-025/CF) or Netrin-1 (R&D Systems cat. #6419-N1-025/CF) [10 nM] for 1 hour. Blocking solution was removed and primary antibodies added in the same blocking buffer (ATLX-1282/ATL_5262 [10 μg/ml]) overnight at 4 degrees. Slides were washed 3 times with PBS-Tx (0.5%) 5 minutes each, followed by addition of secondary antibody anti-human IgG DyLight® 594 (Abcam cat. #ab97005) [2.5 μg/ml] and DAPI [0.1 μg/ml] (Thermo Scientific cat. #62248) in respective buffer for 90 minutes at room temperature, washed and mounted with coverslips using VectaShield anti-fade mounting medium (Vector Labs cat. #H-1200-10). Images of slides were acquired using Molecular Devices PICO and analysis performed using FIJI to threshold and measure images, GraphPad t-tests were performed for significance values.

### Flow Cytometry

The nucleotide sequences encoding canonical UNC5C (SEQ ID NO: 99) and mutated UNC5C T835M variant (SEQ ID NO: 110) were cloned into the lentiviral transfer vector pCDH-EF1α-MCS-(PGK-GFP-T2A-Puro) (System Biosciences; #CD813A-1) and confirmed with sanger sequencing. Lentiviral particles were produced in the 293T cell line (ATCC; #CRL-3216) using TransIT-VirusGEN Transfection Reagent (Mirus; #MIR 6700) and pPACKH1 HIV Lentivector Packaging Kit (System Biosciences; #LV500A-1). Stable cell lines were generated by transduction of HEK-293 cell line (ECACC; #CRL-1573) with lentiviral particles and subsequent selection of successfully transduced cells with puromycin (Gibco™; #A1113803) at 1 μg/ml. Overexpressing cell lines were monitored for GFP expression and cultured in the presence of selection antibiotic.

For the detection of surface antigens, HEK-293 cells as well as cell lines expressing UNC5C, mutated UNC5C T835M and empty vector (EV) control were harvested using non-enzymatic dissociation buffer (CORNING; cat #25-056-Cl). Cells were then labelled with viability dye eF780 (Invitrogen; #65-0865), washed with FACS buffer (DPBS, Gibco; #14190-169, 2% FBS, LSG; #S-001A-USDA) and incubated with the antibodies at 100 μg/ml (0.69 nM), in triplicate, for 1 h at 4° C. Cells were then washed twice with the FACS buffer and incubated with the secondary detection anti-IgG APC antibody (clone IS11-3B2.2.3, Miltenyi; #130-119-772) for 30 minutes at 4° C. Cells were then washed twice, resuspended in FACS buffer and analysed using NovoCyte Penteon Flow Cytometer (Agilent). Compensation was performed automatically using NovoExpress software (Agilent) and samples labelled with individual fluorochromes.

Detection parameters for each fluorochrome are set out in Table 3 below. Data analysis was performed using FlowJo software (BD).

TABLE 3
Flow cytometry detection parameters

| Fluorochrome | Detection Channel | Excitation | Detection |
|---|---|---|---|
| GFP | B525 | 488 | 525/45 |
| APC | R667 | 637 | 667/30 |
| eF780 | R780 | 637 | 780/60 |

The percentage of APC positive cells (IgG+ cells) as well as IgG-APC median fluorescence intensity (MFI) were measured in the population gated on single/viable/cells. The HEK-293 cell line was used as the negative control for APC (IgG+) gating and the background signal was subtracted from the results.

### Retrogenix Analysis

This was performed by Charles River (UK) using their proprietary Retrogenix Cell Microarray Technology (see www.criver.com/products-services/discovery-services/screening-and-profiling-assays/retrogenix-cell-microarray-technology).

### X-Ray Crystallography

Recombinant human UNC5C was produced in ExpiHEK293 by transient transfection of a construct corresponding to Ig domains 1 and 2 and purified via a C-terminal hexahistidine tag (SEQ ID NO: 111). The Fab fragment of ATL_5262 was produced by transient transfection of ExpiCHO and purified via the CH1 domain. Both components were purified by SEC, mixed together to form the complex and screened for crystallisation using commercial crystallisation screens. X-ray diffraction data was collected from crystals grown in 22% PEG Smear Broad, 0.1 M Bicine pH 9.3 (among many other formulations that had a similar composition and pH). Data was autoprocessed with Xia2/Dials and used for molecular replacement with the program Phaser using a truncated “Netrin receptor UNC5C” monomer (UniProt ID D6RE16 AlphaFold model) and a Fab heterodimer (PDB ID 6CNR) as search models. The original data from autoprocessing was reindexed to P6522 (space group of the protein crystal) and the model was taken through several rounds of interactive model rebuilding with the program Coot and refinement with Refmac, and finally manual and automatic solvent building with ARP/wARP, to produce the final model. Data collection and refinement statistics are set out in Table 4. The model contains one molecule each of the Fab heavy and light chains and one molecule of UNC5C in the asymmetric unit. The asymmetric unit is the area within the crystal lattice that contains the molecules that are repeated throughout the crystal. Finding only one copy of each the Fab and antigen, in combination with analysis of how the repeating units interact with each other here show that there are no other biologically relevant interactions e.g. dimerisation of the target.

TABLE 4
Data collection and refinement statistics. Statistics for
the highest-resolution shell are shown in parentheses.

| ATL_5262 Fab - UNC5C Ig1-2 | | |
|---|---|---|
| Wavelength (Å) | 0.9537 | |
| Resolution range (Å) | 63.51-2.27 | (2.351-2.27) |
| Space group | P 65 22 | |
| Unit cell | 83.9251 Å, 83.9251 Å, | |
| | 381.065 Å, 90°, 90°, 120° | |
| Total reflections | 1491857 | (142455) |
| Unique reflections | 38062 | (3670) |
| Multiplicity | 39.2 | (38.8) |
| Completeness (%) | 99.94 | (99.67) |
| Mean I/sigma(I) | 16.60 | (0.37) |
| Wilson B-factor | 71.71 | |
| R-merge | 0.1394 | (5.711) |
| R-meas | 0.1413 | (5.786) |
| R-pim | 0.02248 | (0.9228) |
| CC1/2 1 | −0.373 | |
| CC* 1 | −0.737 | |
| Reflections used in refinement | 38042 | (3658) |
| Reflections used for R-free | 1924 | (201) |
| R-work | 0.2694 | (0.4369) |
| R-free | 0.3332 | (0.4530) |
| CC(work) | 0.928 | (0.288) |
| CC(free) | 0.909 | (0.273) |

| | |
|---|---|
| Number of non-hydrogen atoms | 4821 |
| Macromolecules | 4812 |
| Ligands | 0 |
| Solvent | 9 |
| Protein residues | 622 |
| RMS(bonds) | 0.008 |
| RMS(angles) | 1.68 |
| Ramachandran favored (%) | 87.3 |
| Ramachandran allowed (%) | 8.96 |
| Ramachandran outliers (%) | 3.75 |
| Rotamer outliers (%) | 10.36 |
| Clashscore | 15.26 |
| Average B-factor | 82.2 |
| Macromolecules | 82.26 |
| Solvent | 52.57 |

### Phage Display

A phage library of scFv molecules displayed on an M13 phage was generated by cloning the heavy chain variable region (VH) repertoire of subjects SU_0000656 and SU_0000697 into a sub-library of phagemid vectors with light chain variable region (VL) sequences from healthy donors. A phage display library of a size 1.3e8 clones was generated.

The following reagents were used:
Hyper phage (1.5e12 cfu/ml) from Progen, Germany catalogue number PRHYPE, K07deltapIII (used in Phage display and phage ELISA);
Helper phage (1e11 cfu/ml) from Invitrogen, USA catalogue number 18311-019 (used in Phage display and phage ELISA);
UNC5C_HUMAN antigen (SEQ ID NO: 194) produced In-house (in house number UNC5C_HUMAN_008 (Alchemab) res41-380 Batch #004) (used in Phage Display and Phage ELISA)
UNC5C_human from R&D Systems, USA catalogue number 1005-UN-050 (used in Phage Display)
   Phage ELISA on scFv Derived from Phage Display

Phages were prepared by culturing each phagemid-carrying TG1 clone from a glycerol stock in 100 μl of 2TYAG (2TY media supplemented with 100 μg/ml ampicillin and 2% glucose) at 37° C. with aeration to optical density OD600=0.6 followed by rescue with helper or hyper phage added at MOI 10 (Invitrogen, cat: 18311-019 and Progen, cat: PRHYPE, K07deltapIII, respectively) for 1 hour and media change to 2TYAK (2TY media supplemented with 100 μg/ml ampicillin and 50 mg/ml kanamycin). The cultures were then incubated overnight at 25° C. with good aeration and next day the phages were separated from bacteria by centrifugation for 10 minutes at 3200 rpm. The phage-containing supernatant was transferred to a new plate and blocked with 3% milk w/v in PBS.

UNC5C recombinant human antigen (in-house, UNC5C_HUMAN_008 (Alchemab) res41-380 Batch #004) or negative control Lysozyme (MP Biomedicals #195303) were directly absorbed to ELISA plate at 3 μg/ml (50 μl per well) and incubated overnight at 4° C. Each antigen-coated plate was washed with PBS and blocked with 200 μl/well of blocking solution (3% milk w/v in PBS) for 1 hour at room temperature. Following this, the blocking solution was removed and blocked phage samples to be assessed were applied to the plates. Plates were incubated at room temperature for 1 hour. Each plate was washed with PBS/0.1% Tween and incubated with anti-M13 HRP (Sino Biological, #11973-MM05T-H) for 1 hour at room temperature to detect phage binding. The plates were washed with PBS/0.1% Tween and TMB solution (Life Technology; #002023) added. Plates were incubated for 5 minutes at room temperature prior to the addition of stopping solution (0.5 M sulphuric acid). Absorbance was read on a Molecular Devices FilterMaxF5 plate reader at 450 nm.

### Screening IgG Binding to UNC5C

To determine binding of the IgG antibodies to UNC5C, UNC5C_Fc (R&D Systems #1005-UN), or in-house production from HEK293F cells with N-terminal His-tag or negative control Lysozyme (MP Biomedicals #195303) were directly absorbed to an ELISA plate at 1 μg/ml and incubated overnight at 4° C. The plate was washed with PBS. Plates were blocked with 1% BSA w/v in PBS blocking solution for 1 hour at room temperature. Following this, the blocking solution was removed and antibodies to be assessed were diluted in dilution series (2 μM to 0.4 pM) in blocking solution (1% BSA w/v in PBS) and applied to the plate. Plates were incubated at room temperature for 1 hour. The plate was washed with PBS/0.1% Tween. Anti-Human IgG HRP (Jackson ImmunoResearch; #109-035-097) was added to the plate and incubated for 1 hour at room temperature to detect antibody binding. The plate was washed with PBS/0.1% Tween and TMB solution (Life Technology; #002023) added. Plates were incubated for 5 minutes at room temperature prior to the addition of stopping solution (0.5 M sulphuric acid). Absorbance was read on a Molecular Devices FilterMaxF5 plate reader at 450 nm. Analysis was performed using a non-linear fit curve fitting algorithm on GraphPad prism and EC50 calculated.

### Example 1—Convergence Analysis

Convergent sequence clusters derived from the antibody repertoire of resilient groups of individuals can be used to identify disease-specific antibody sequences. The present inventors sought to identify candidate protective antibodies from Frontotemporal dementia (FTD)-resilient individuals from a cohort of FTD patients (Genetic FTD Initiative (GENFI), www.genfi.org/). FTD encompasses several types of dementia impacting frontal and temporal lobes of the brain, and major genetic risk factors are GRN and C9orf72 mutations.

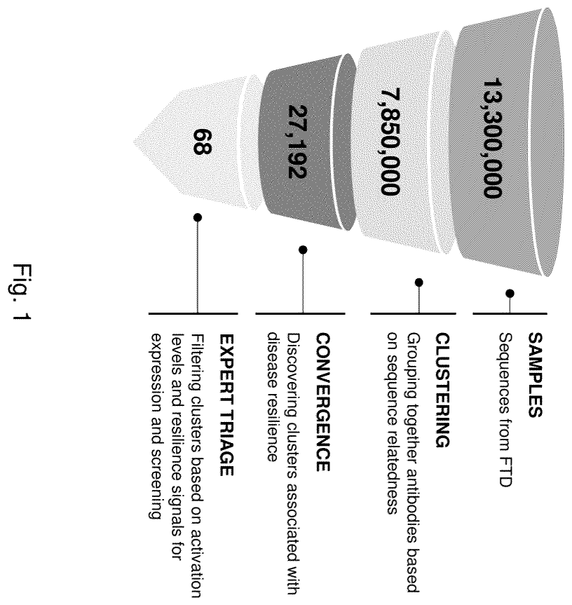

Resilience to FTD was defined as individuals not developing disease despite being within the top 33% of the cumulative age of onset of frontotemporal dementia and the presence of GRN or C9orf72 mutations. From the total cohort of over 1000 individuals, the 22 most highly resilient individuals were selected. Sequencing of the antibody repertoires of these resilient patients revealed a convergent heavy chain variable (VH) sequence among three highly resilient individuals (See FIG. 1 for workflow used to identify convergent VH sequences from the FTD dataset). The probability of all three individuals producing this antibody by chance is extremely low. It is likely that this particular sequence was highly selected for and thus advantageous to the individual.

The VH sequence was subsequently paired with a VL using a transformer-based model comprising an encoder-decoder model trained on a corpus of paired VH-VL sequences. Further details on how such a model may be trained and used is provided in WO2022/223451. The trained model takes a VH sequence as input and generates a single complementary VL sequence as output.

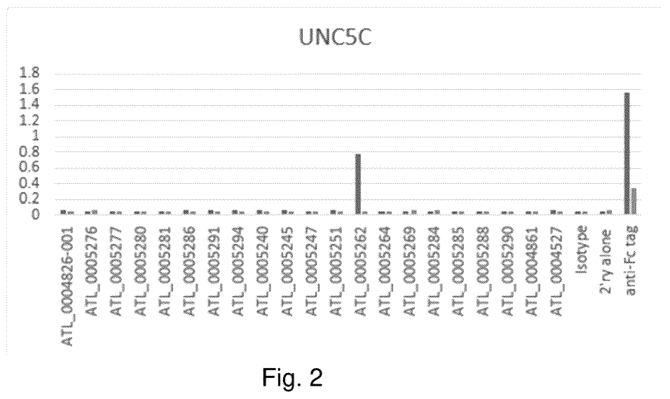

The resulting antibody, ATL_5262 (ATL_0005262) was included in a proteomics array for target deconvolution, identifying the Netrin receptor UNC5C as its target (data not shown). Binding of ATL_5262 to UNC5C was confirmed by ELISA (FIG. 2).

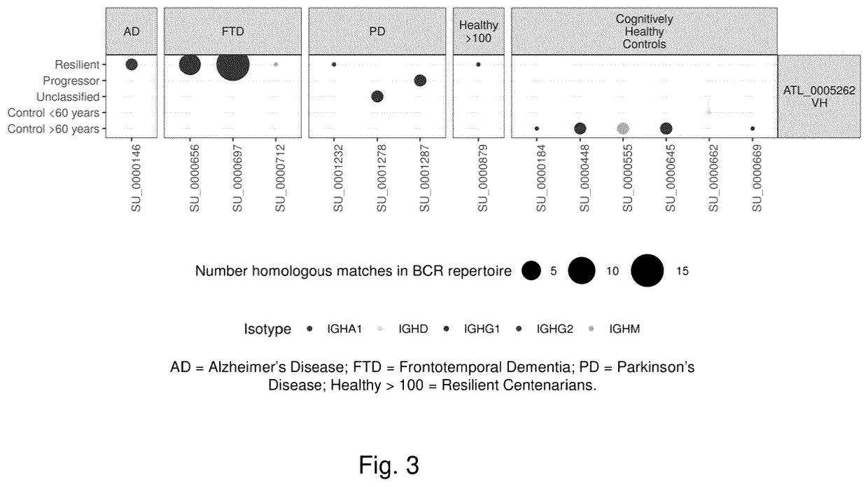

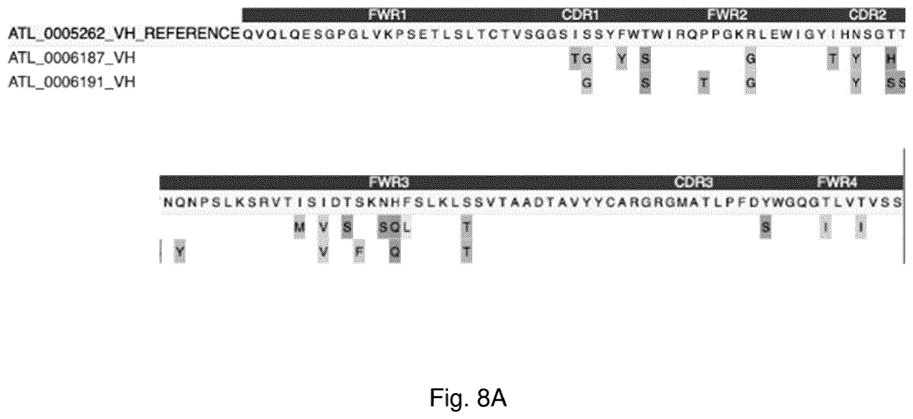

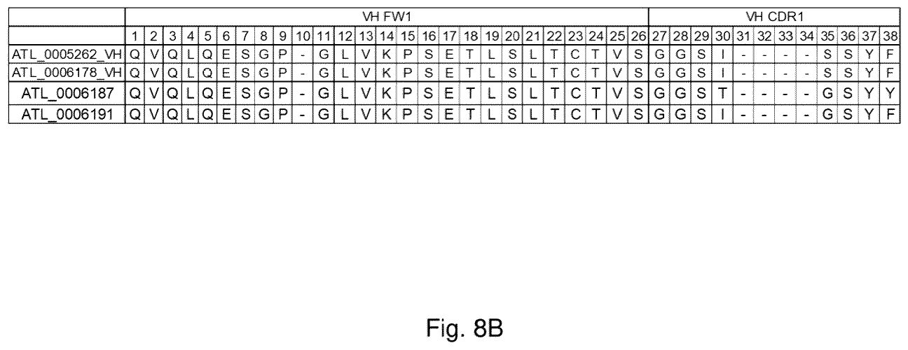

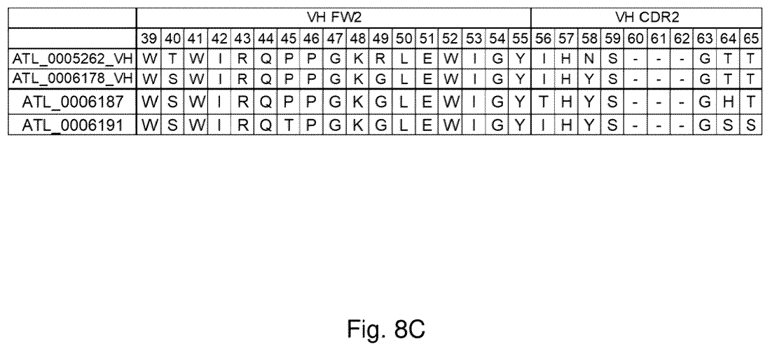

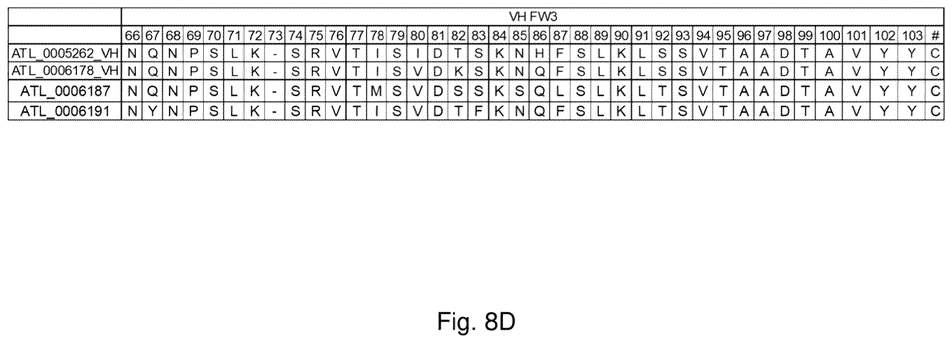

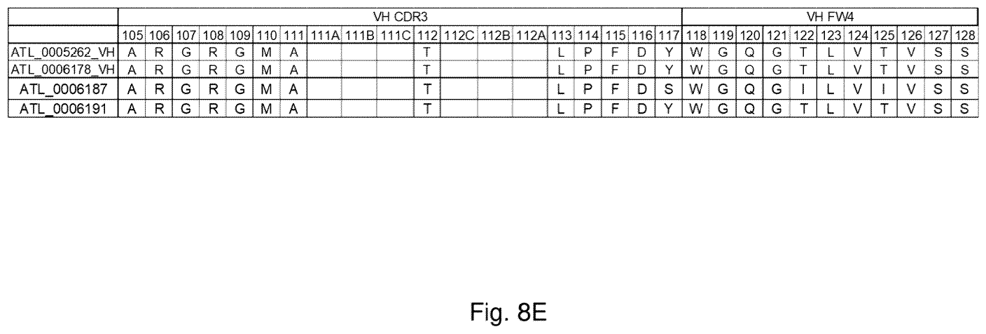

Interestingly, UNC5C antibodies were subsequently identified across repertoires derived from subjects who are resilient to neurodegeneration (FIG. 3). In particular, the heavy chain sequence of ATL_5262 was used as a probe to search similar sequences in an unpublished repertoire database derived from neurodegeneration. Sequences with high homology were found in a resilient Alzheimer's disease subject (individual showing slow cognitive progression); a subject with resilience for Parkinson's disease (prodromal REM sleep behaviour disorder (RBD) but no Parkinson's diagnosis); three subjects with resilience for FTD (carriers of high-risk mutations and older age but no progression to FTD); and resilient centenarians with no symptoms of neurodegeneration (FIG. 3). Sequences were also found in cognitively healthy controls—predominantly individuals over 60 years of age. Notably there is an absence of UNC5C antibodies in so-called “Progressors” from these cohorts, with exception to SU_0001278 who has a diagnosis of Parkinson's disease and Mild Cognitive Impairment. PD subject SU_0001278 is ‘Unclassified’ since they were initially included in the cohort as a healthy control, but subsequently declassified since they have suspected REM sleep behaviour disorder. Since they have no PD diagnosis they are ‘potentially resilient’. All the sequences identified, referred to herein as “ATL_5262 homologues”, have the same V and J gene as ATL5262 VH and at most 2AA mismatches across the junction region. FIG. 8 shows a VH sequence alignment of representative ATL_5262 homologous sequences ATL_6187 and ATL_6191 (SEQ ID NO: 112-113). The representative VH sequences of the ATL_5262 homologues were subsequently paired with the VL of ATL_5262 (SEQ ID NO: 9) and produced as IgG1 Fc NULL antibodies. The VH of these antibodies had at least 80% sequence identify with the sequence of the VH of ATL_5262 (in particular: 6187: 84.87%, 6191:89.92%—for reference the 5262 derivative antibody 6178 has 94.96% VH sequence identity with the VH of ATL_5262, all determined using Clustal). Antibodies 6187 and 6191 both had EC50 for binding to EC50 below 10⁻⁷ (determined by ELISA). These antibodies have 0 or 1 mutation in the VH CDR3 compared to ATL_5262 (at most 1 mutation at position 117 in IMGT numbering, Y117S) and up to 3 mutations in each of the VH CDR2 and CDR1 compared to ATL_5262. Both ATL_6178 and ATL_6191 had a mutated variable region and IGHG1 constant region, which indicates that class-switch recombination and affinity maturation through somatic hypermutation have occurred, and thus that most likely there has been a germinal centre-induced immune response to antigen (see Table 5). These highly mutated class switched sequences are likely from memory B cells, which are typically more specific to the antigen than a naïve B cell.

The fact that UNC5C antibodies could be found in resilient individuals in these cohorts highlights the relevance of UNC5C as a therapeutic target in various neurodegenerative conditions.

The identified homologues were subsequently tested for binding to UNC5C by ELISA and the results are shown in Table 14.

TABLE 5
Characteristics of ATL_5262 homologues.

| | | | | | AA |
|---|---|---|---|---|---|
| | | | | | mismatches |
| | | | | | (vs |
| Antibody | | Disease | Sex | Sequence | ATL5262 |
| ID | Subject | status | (age) | features | full VH) |
| ATL6187(*) | SU_0000448 | Cognitively | Female (62) | Highly mutated and | 18 |
| | | healthy control | | IGHG1-class switched | |
| ATL6191(*) | SU_0001278 | PD-unclassified | Male (63) | Mutated and IGHG1- | 12 |
| | | | | class switched | |

Highly mutated: >20 mutations (indicative of a response to antigen exposure).
(*) indicate antibodies with an ELISA binding profile similar to ATL5262.

### Example 2—Competition with Netrin 1

To determine whether ATL_5262 competes for the same binding site as the UNC5C ligand, Netrin 1, a competition assay was performed.

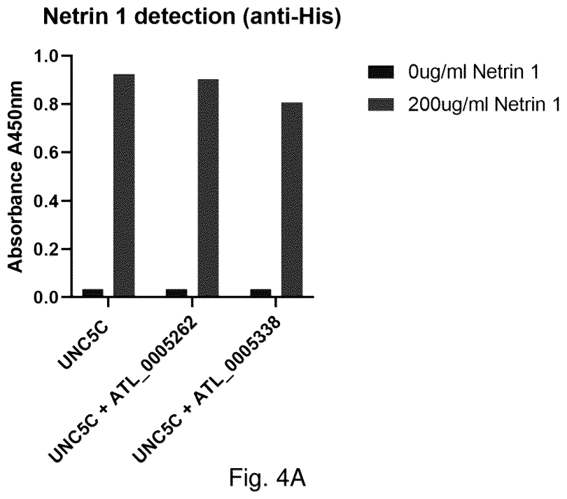

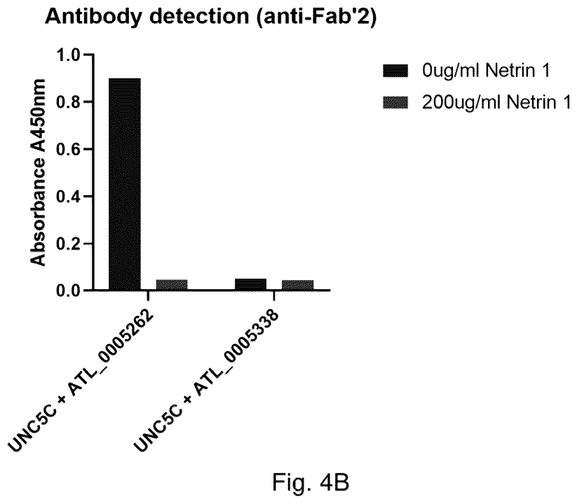

Briefly, ELISA plates were coated with 45 nM UNC5C and pre-incubated with 3 μM Netrin 1 for 30 minutes prior to adding 27 nM ATL_5262, or a negative control anti-fluorescein antibody labelled “ATL_5338”. FIG. 4A shows that Netrin 1 could be detected in wells containing UNC5C alone, UNC5C and ATL5262, and UNC5C and ATL_5338, indicating that Netrin 1 inhibits binding of ATL_5262 to UNC5C. FIG. 4B shows that ATL_5262 could only be detected in wells not preincubated with Netrin 1, confirming that ATL_5262 binds to UNC5C and this binding is inhibited by Netrin 1. FIG. 4B further shows that the control antibody ATL_5338 does not bind to UNC5C.

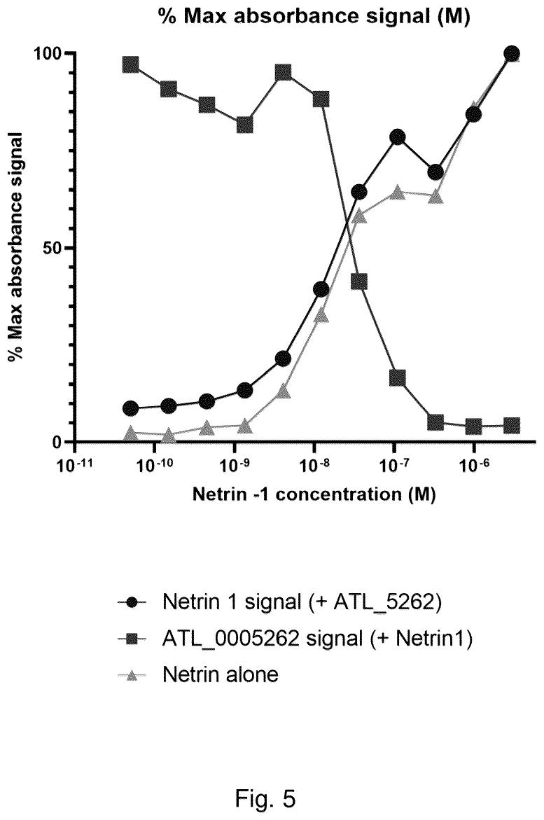

To determine whether Netrin 1 inhibits ATL_5262 binding to UNC5C in a concentration-dependent manner, a 12-point dilution series of Netrin 1, starting at 3 μM, was performed and binding of ATL_5262 to UNC5C was assessed using ELISA (FIG. 5). FIG. 5 shows that Netrin 1 inhibits binding of ATL_5262 to UNC5C in a concentration-dependent manner.

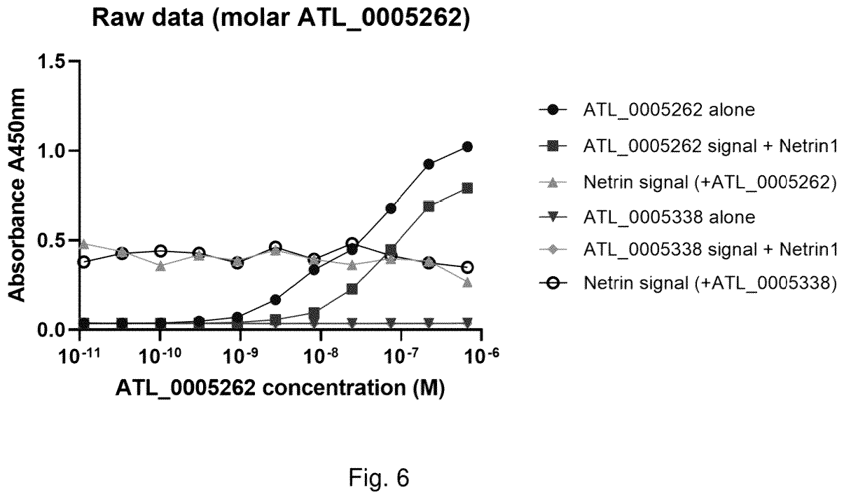

To test whether Netrin 1 is able to compete with ATL_5262 already bound to UNC5C, ELISA plates were coated with 45 nM UNC5C and preincubated with a 12-point dilution series of ATL_5262, starting at 666 nM for 30 minutes prior to adding 75 nM Netrin 1 (FIG. 6). FIG. 6 shows that Netrin 1 is able to outcompete ATL_5262 binding as indicated by the “ATL_0005262+Netrin 1 curve” being shifted to the right compared with “ATL_0005262 alone”, i.e. only at high concentrations of ATL_5262, can ATL_5262 partly outcompete Netrin 1. Similarly, only at the highest concentration of ATL_5262 was a small decrease in Netrin signal detected. As expected, no binding signal was observed for wells incubated with UNC5C and control antibody ATL_5338.

Together, these data show that Netrin 1 inhibits binding of ATL_5262 to UNC5C in a concentration-dependent manner, indicating that ATL_5262 competes for the same binding site in UNC5C as Netrin 1. ATL_5262 may thus act as a Netrin 1 mimic. To the best of the inventors' knowledge, the antibodies described herein are the first antibodies that bind UNC5C and compete with binding of netrin 1.

### Example 3—Murine Cross Reactivity

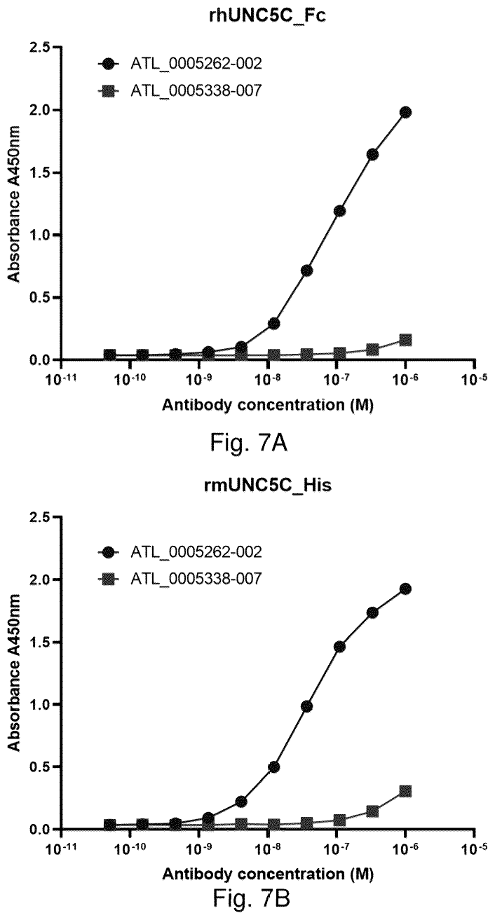

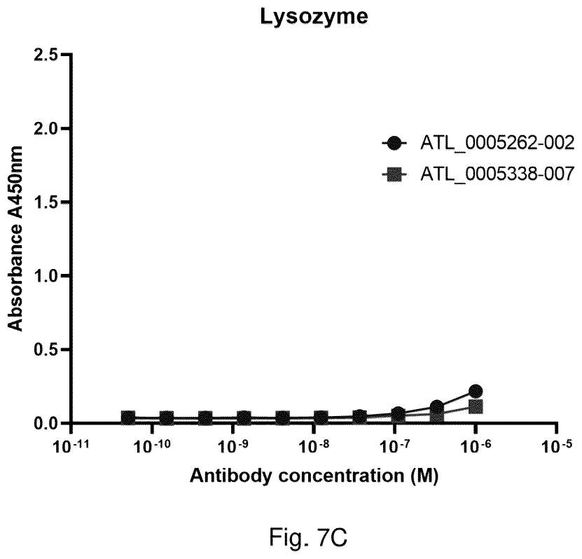

The ability of ATL_5262 to bind both murine and human recombinant UNC5C was tested using ELISA (FIG. 7).

Human and murine UNC5C have 97% homology, and ATL_5262 is able to bind both rhUNC5C (FIG. 7A) and rmUNC5C (FIG. 7B). Control antibody ATL_5338 shows negligible binding to rhUNC5C (FIG. 7A) and rmUNC5C (FIG. 7B). ATL_5262 does not bind to irrelevant protein lysozyme (FIG. 7C), indicating that the antibody does not exhibit non-specific binding.

In conclusion, ATL_5262 binds both human and mouse recombinant UNC5C.

### Example 4—Binding to Other Netrin Receptors and Related Proteins

The inventors subsequently sought to investigate the selectivity of ATL_5262 for UNC5C. To identify other potential antibody binding targets, a BLAST search of human protein sequences with >50-% sequence identity was performed using the reference UNC5C sequence (accession 095185) in the UniProtKB database (www.uniprot.org/). This returned the related proteins UNC5A, UNC5B, UNC5D. In addition, to account for the possibility of netrin structural or functional mimicry, the human netrin receptors neogenin, DCC, and DSCAM were considered as potential binding partners.

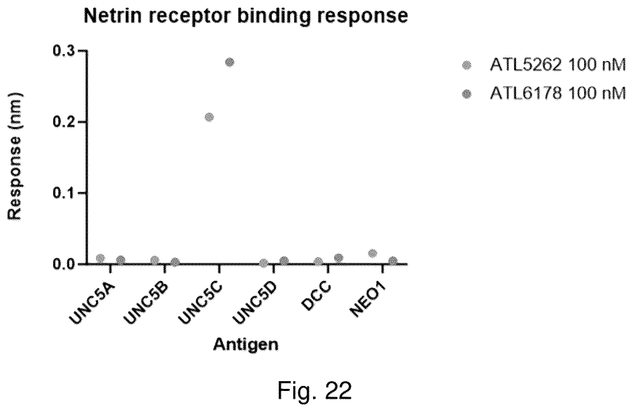

Binding of ATL_5262 and ATL_6178 (a variant of ATL_5262, see Example 6) to other netrin receptor family members and related proteins was tested using biolayer interferometry analysis (FIG. 22). FIG. 22 shows that ATL_5262 and ATL6178 bind to UNC5C but not to any other Netrin receptors or related proteins in this assay (UNC5A, B, D, DCC, and neogenin-1 ectodomains). No evidence of binding to any non-UNC5C antigens by either antibody at a concentration of up to 100 nM was observed, indicating selectivity of these ATL_5262 and ATL_6178 for UNC5C.

To further confirm the selectivity of ATL_6178 it was analysed in a binding assay against fixed HEK293 cells expressing 6105 individual full-length human plasma membrane proteins and cell surface-tethered secreted human proteins, as well as a further 400 human heterodimers, followed by a series of confirmatory screens, using the Retrogenix platform with the operators blinded to the antibody target. Other Netrin-1 receptors including UNC5A, UNC5B and UNC5D, NEO1, DCC and DSCAM were in the screening panel and displayed no binding to ATL_6178. UNC5C was the only confirmed target for ATL_6178.

These data demonstrate the specificity of ATL_5262 and ATL_6178 for UNC5C, showing they do not bind other netrin receptors or related proteins.

### Example 5—Stability Study

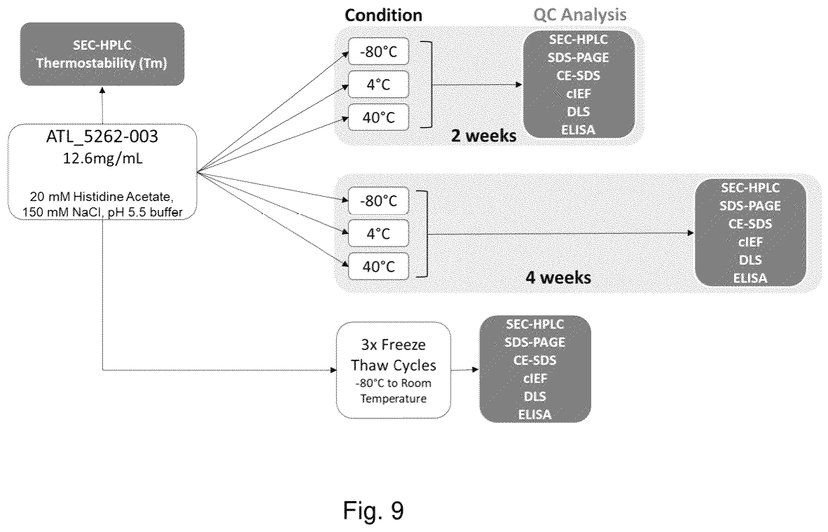

To ensure that ATL_5262 is stable and therefore suitable for further optimisation, a stability study as shown in FIG. 9 was carried out.

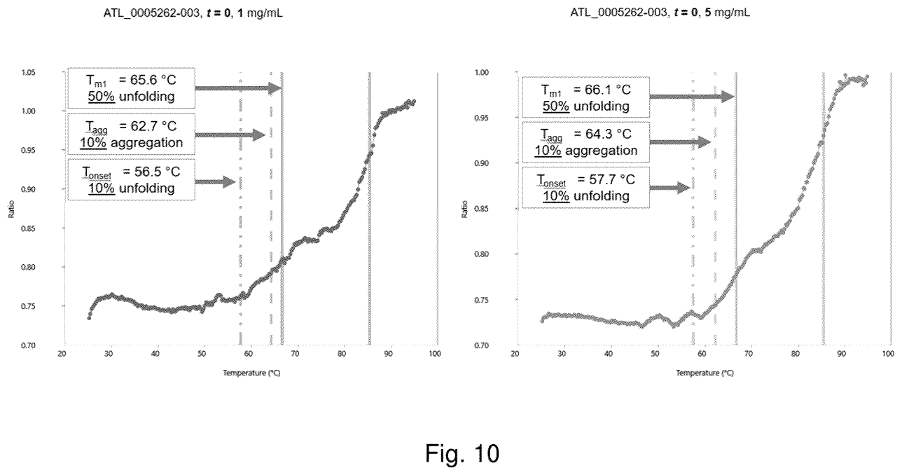

ATL_5262 was tested in a thermal shift assay at both 1 and 5 mg/ml. The results of this are shown in Table 6, and FIG. 10. This shows that Tm1 and Tm2 was within a normal range for monoclonal antibodies (mAbs) and Tagg occurred before Tonset, indicating that aggregation is induced by unfolding more strongly than by protein-protein or protein-buffer interaction. Tₘ₁ is after T_{agg} indicating that the protein begins to aggregate at <50% ratio of unfolded-to-folded protein.

TABLE 6
Tonset, Tm1, Tm2 and Tagg for
ATL_5262 in a thermal shift assay.

| | Concentration | Tₒₙₛₑₜ | Tₘ1 | Tₘ2 | T_{agg} |
|---|---|---|---|---|---|
| Antibody | (mg/mL) | (° C.) | (° C.) | (° C.) | (° C.) |
| ATL_5262 | 1 | 56.48 | 65.583 | 84.83 | 62.737 |
| | 5 | 57.7 | 66.143 | 88.623 | 64.343 |

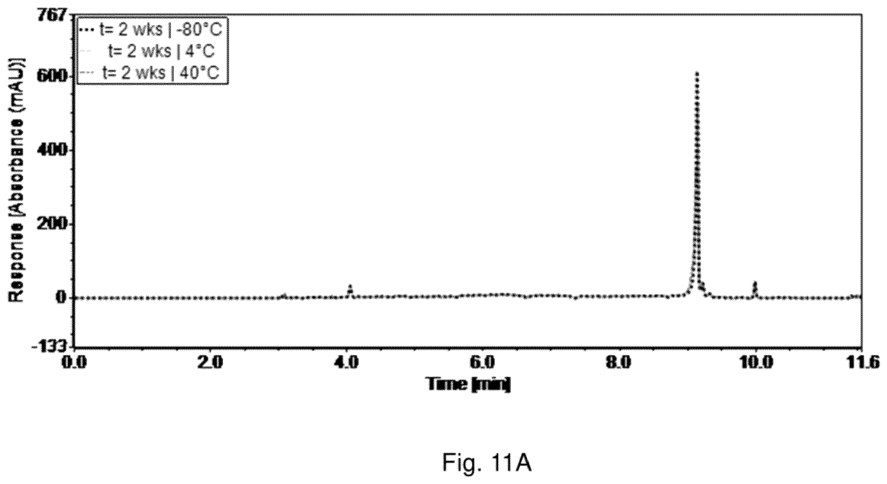

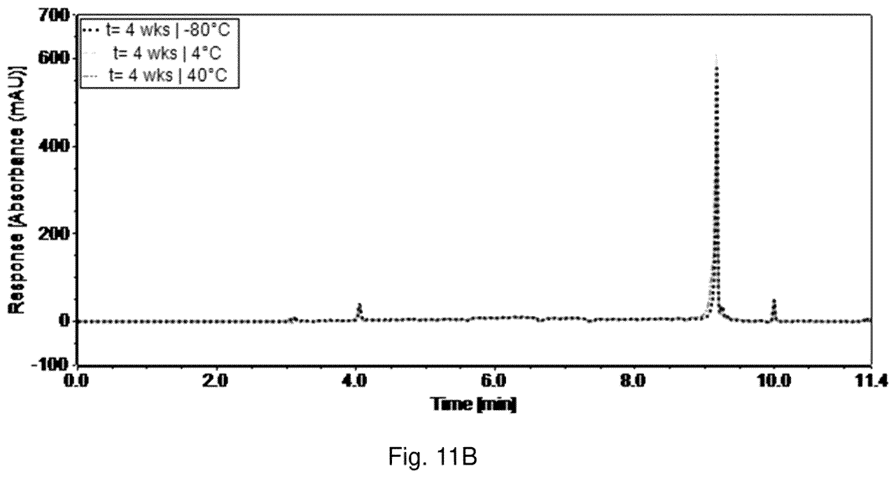

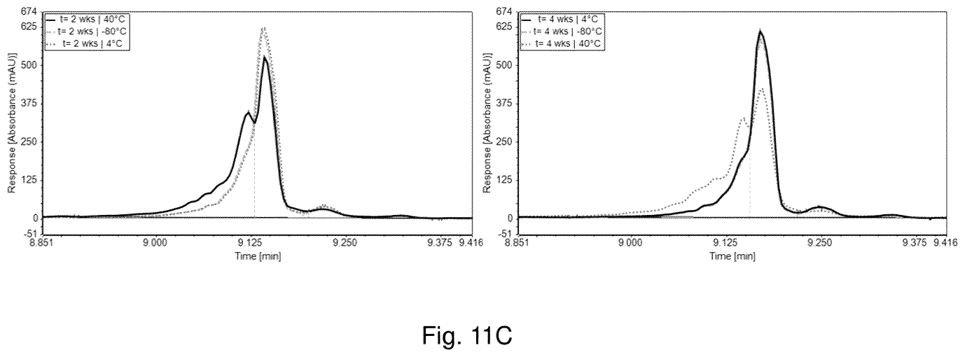

Capillary isoelectric focusing (cIEF) was used to analyse charge heterogeneity of ATL_5262 after 2 weeks (FIG. 11A) or 4 weeks (FIG. 11B) in storage at −80° C., 4° C., and 40° C. Whilst an increase in acidic charge variants was observed at 40° C. (49.27% at 2 weeks and 53.39% at 4 weeks) (FIG. 11C, Table 4), minimal changes in charge species present were observed in all other conditions (Table 7).

TABLE 7
Calculated percentage of acidic species area;
main peak area; and basic species area.

| | | | | Acidic | | Basic |
|---|---|---|---|---|---|---|
| | | | | Species | Main | Species |
| | Time- | | | Peak | Peak | Peak |
| Antibody | point | Condition | | Area (%) | Area (%) | Area (%) |
| ATL_0005262 | 2 weeks | −80° | C. | N.A | 92.86 | 7.14 |
| | | 4° | C. | N.A | 92.74 | 7.26 |
| | | 40° | C. | 49.27 | 45.59 | 5.14 |
| | 4 weeks | −80° | C. | N.A | 92.53 | 7.47 |
| | | 4° | C. | N.A | 92.26 | 7.38 |
| | | 40° | C. | 53.39 | 42.22 | 4.4 |

Protein aggregation during antibody storage must be kept to a minimum as it can cause immunogenic reactions. Size exclusion chromatography (SEC-HPLC) was used to evaluate the purity and aggregation of the antibodies in the 2/4-week stability study.

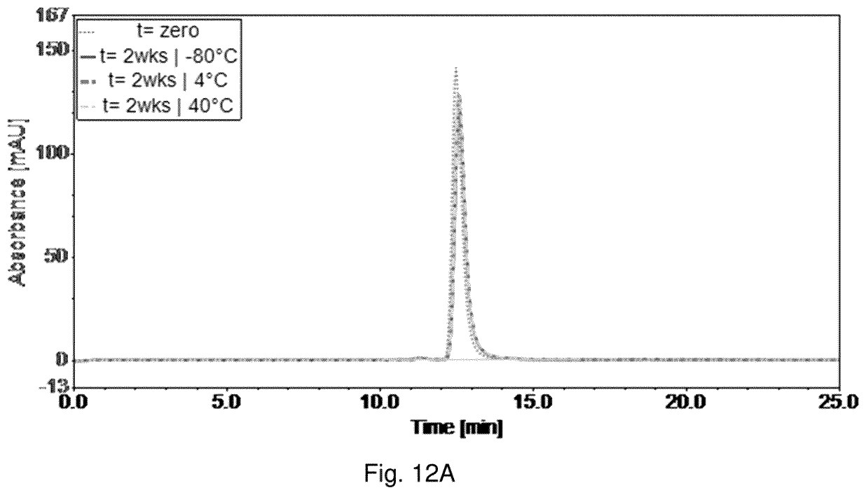

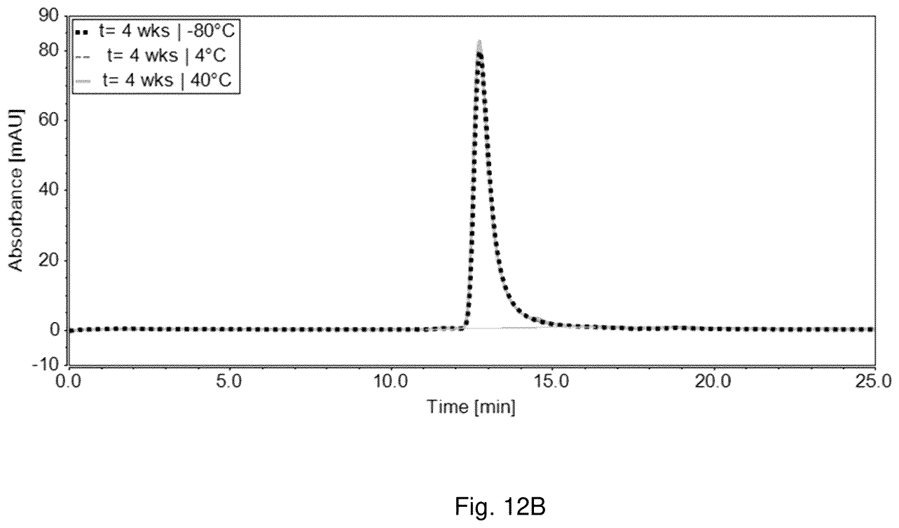

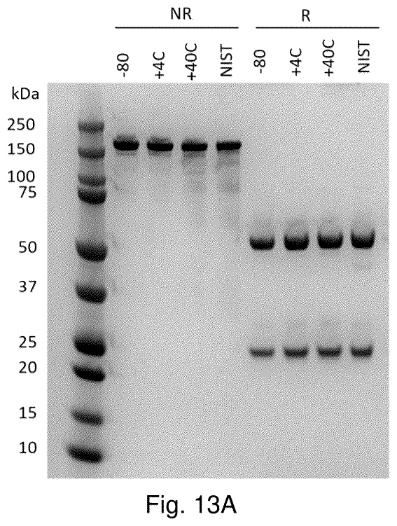

FIG. 12 shows that no soluble aggregates formed following incubation at −80° C., 4° C., 40° C. for 2 weeks (FIG. 12A) or 4 weeks (FIG. 12B).

Table 8 further supports the excellent stability of ATL_5262 and shows low levels of high molecular weight species (HMWS) and low levels of low molecular weight species (LMWS) indicating that there is low aggregation and degradation propensity of the antibody up to 40 C, which is further supported by the high percentage of monomers in all of the samples (>95%).

TABLE 8
Results from SEC-HPLC.

| Antibody | Timepoint | Condition | | % HMWS | % Monomer | % LMWS |
|---|---|---|---|---|---|---|
| ATL 5262 | Initial | N/A | | 1.21 | 98.79 | n.a |
| | 2 weeks | −80° | C. | 1.21 | 98.79 | n.a |
| | | 4° | C. | 0.92 | 99.08 | n.a |
| | | 40° | C. | 0.88 | 99.12 | 1.19 |
| | 4 weeks | −80° | C. | 0.44 | 99.56 | n.a |
| | | 4° | C. | 0.46 | 99.54 | n.a |
| | | 40° | C. | 0.49 | 96.76 | 2.75 |

HMWS = high molecular weight species; LWMS = low molecular weight species.

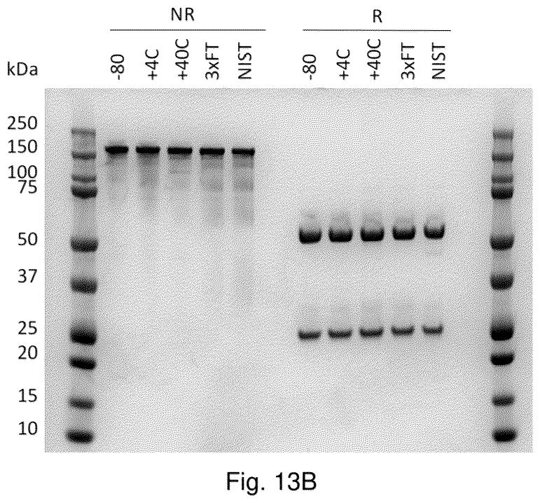

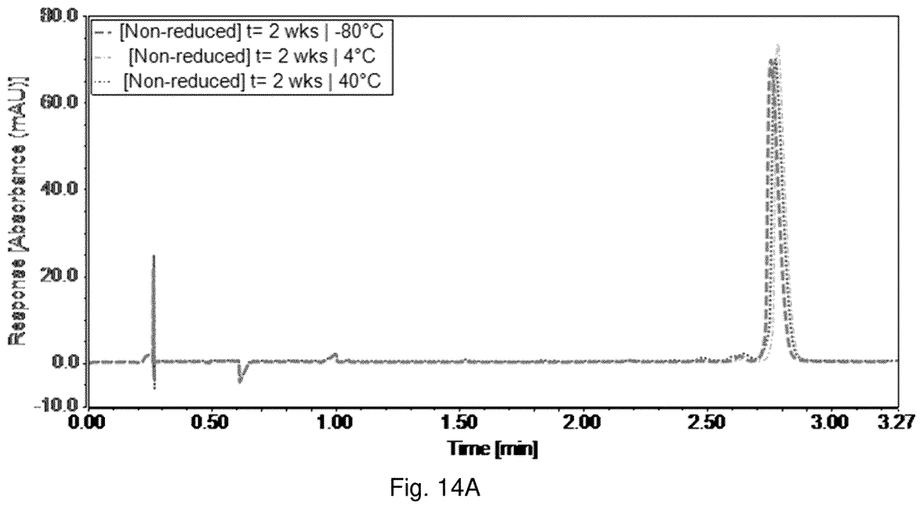

SDS-PAGE was used to analyse the purity of ATL_5262 after storage at −80° C., 4° C., 40° C. for two weeks (FIG. 13A) or 4 weeks (FIG. 13B), or after 3 FT cycles (FIG. 13B). Reduced IgG antibodies typically give rise to glycosylated heavy chains of approximately 50 kDa and light chains of approximately 25 kDa on SDS-PAGE (as indicated by “R” in FIG. 13) and 150 kDa bands for non-reduced antibodies (as indicated by NR in FIGS. 13A and B). Minimal changes to samples molecular weight separations are detected for both reduced and non-reduced samples following the temperature and freeze thaw stability time course. NIST mAb was run as a molecular weight control of a human IgG1 mAb.

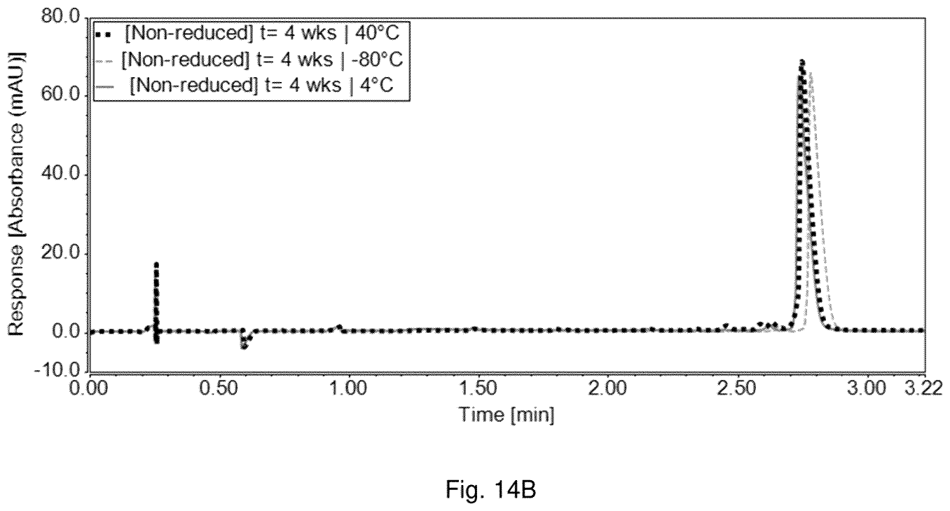

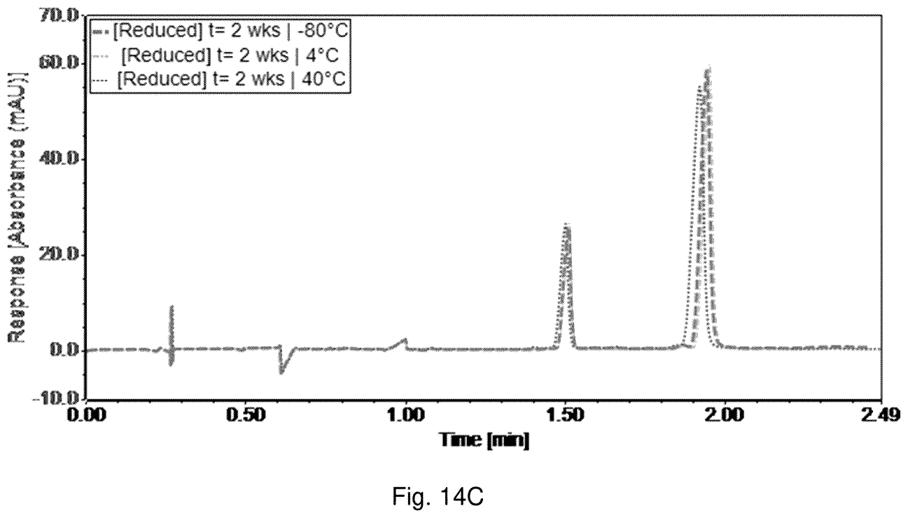

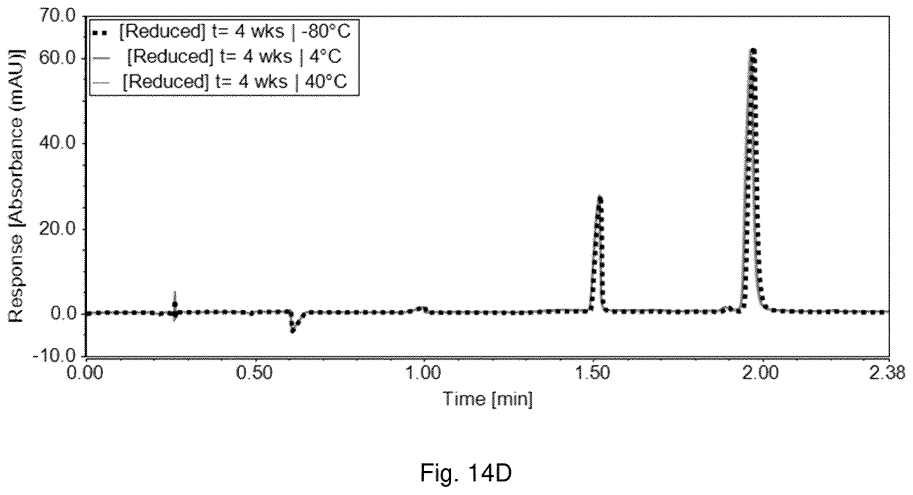

Further to running SDS-PAGE, CE-SDS was used to analyse the molecular weight and purity of species following the temperature time course stability samples. Non-reduced samples show a 3-5% decrease in main species at 40 C at both 2 weeks (FIG. 14A) and 4 weeks (FIG. 14B) but not at other storage temperatures (Table 9), under reducing conditions minimal changes were detected through the temperature time course stability study at both 2 weeks (FIG. 14C) and 4 weeks (FIG. 14D) (Table 10).

TABLE 9
CE-SDS results for non-reduced ATL_5262

| Antibody | Timepoint | Condition | | Main Peak Area % |
|---|---|---|---|---|
| ATL 5262 | 2 weeks | −80° | C. | 97.55 |
| | | 4° | C. | 97.58 |
| | | 40° | C. | 94.66 |
| | 4 weeks | −80° | C. | 97.68 |
| | | 4° | C. | 97.68 |
| | | 40° | C. | 92.89 |

TABLE 10
CE-SDS results for reduced ATL_5262

| Antibody | Timepoint | Condition | | LC | HC | NGHC |
|---|---|---|---|---|---|---|
| ATL_005262 | 2 weeks | −80° | C. | 24.39 | 74.7 | 0.74 |
| | | 4° | C. | 24.43 | 74.28 | 0.93 |
| | | 40° | C. | 24.45 | 71.48 | 0.92 |
| | 4 weeks | −80° | C. | 24.54 | 73.79 | 0.78 |
| | | 4° | C. | 24.26 | 74.01 | 1.04 |
| | | 40° | C. | 25.34 | 71.07 | 0.99 |

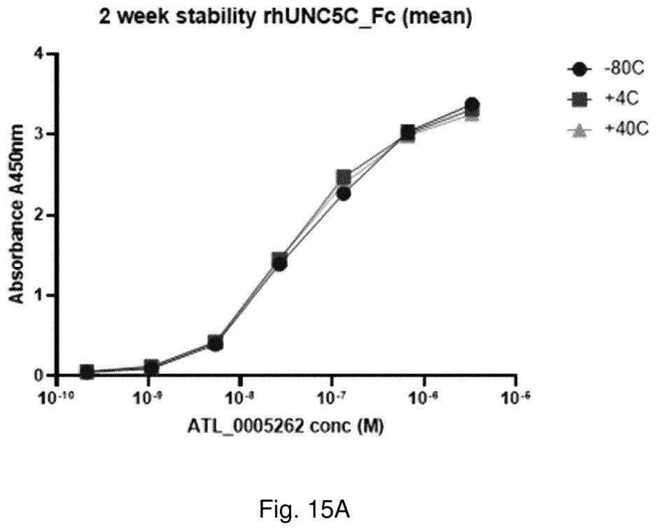

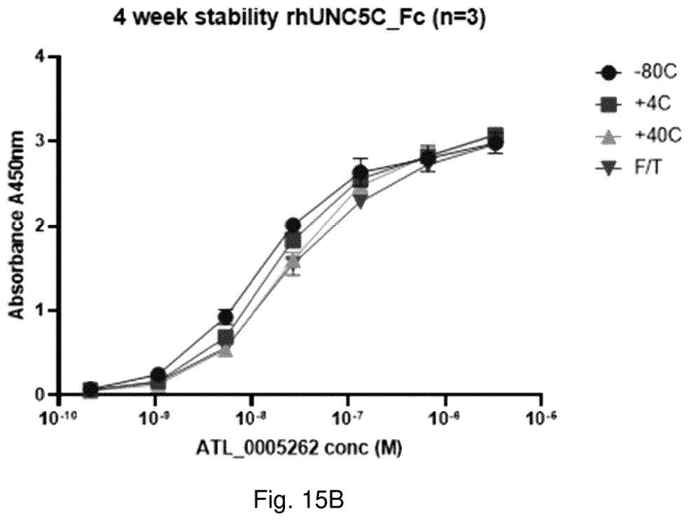

Finally, binding of ATL_5262 to rhUNC5C was tested after two weeks (FIG. 15A) or 4 weeks (FIG. 15B) at −80° C., 4° C., or 40° C., or after 3× FT cycles (FIG. 15B) using ELISA. ATL_5262 showed consistent binding to UNC5C under all conditions and this was reflected in consistent EC50 values (Table 11), indicating that the binding potency of ATL_5262 is unaffected by temperature changes or freeze thaw conditions.

TABLE 11
EC50 values for ATL_5262 two weeks or 4 weeks after storage
at the indicated temperatures or after 3× FT cycles.

| Antibody | Timepoint | Condition | | EC50 (M) |
|---|---|---|---|---|
| ATL 5262 | 2 weeks | −80° | C. | 5.06E−08 |
| | | 4° | C. | 4.29E−08 |
| | | 40° | C. | 4.54E−08 |
| | 4 weeks | −80° | C. | 1.55E−08 |
| | | 4° | C. | 2.1E−08 |
| | | 40° | C. | 2.81E−08 |

| | | |
|---|---|---|
| N.A | 3× FT | 3.35E-08 |

In conclusion, ATL_5262 is stable at −80° ° C. and 4° C. up to 4 weeks tested, and through −80° C. 3× Freeze Thaw cycles, and while some biophysical changes were observed at 40° C., ELISA shows that binding to UNC5C is not affected by these changes. ATL_5262 is therefore stable and suitable for further development.

### Example 6—Antibody Variants

To identify lead antibodies with optimal development characteristics, variants of the VH (SEQ ID NO: 1) and VL sequence (SEQ ID NO: 9) of ATL_5262 were generated and their binding potency to UNC5C was tested using direct ELISA for binding to human UNC5C and mouse UNC5C.

In order to generate VH variants, the ATL_5262 sequence was compared with the VH germline sequence and found to have five amino acid substitutions compared with the germline sequence. Reverting sequences to germline typically reduces immunogenicity and improves antibody expression and stability. Therefore, one VH variant tested was the germline reversed VH framework sequence, ATL_6033 (SEQ ID NO: 2). To identify if any specific reversion to germline was detrimental to antibody expression or binding activity partially germline reversed VH framework sequences were generated with 4 out of 5 germline mutations—that is all germline reversions minus one position remaining identical to the VH of ATL_5262 (ATL_6034 (SEQ ID NO:3); ATL_6035 (SEQ ID NO: 4); ATL_6036 (SEQ ID NO: 5); ATL_6037 (SEQ ID NO: 6); ATL_6038 (SEQ ID NO: 7). In addition, a VH sequence of ATL_5262 with liability removal mutation T82K (using IMGT numbering) (ATL_6039) (SEQ ID NO: 8) was also generated, (see Table 12). All antibodies were expressed using the ATL_5262 VL chain (SEQ ID NO: 9).

All antibodies tested were IgG1 variants L234A/L235A (LALA), i.e. IgG1 variants comprising L234A/L235A substitutions reducing binding to the IgG Fc receptors FcγRI, FcγRII and FcγRIII as well as to complement component C1q, reducing Fc-mediated toxicity (Lund J, et al. 1991).

TABLE 12
EC50 values for VH variant antibodies tested in ELISA.

| Antibodies (AA seq numbering) | EC50 (M) | IGMT numbering |
|---|---|---|
| ATL_0006033 (full germline) | 4.77E−08 | |
| ATL_0006034 (germline - T35) | 9.81E−08 | germline - T40 |
| ATL_0006035 (germline - R44) | 9.74E−08 | germline - R49 |
| ATL_0006036 (germline - Q59) | 5.38E−09 | germline - Q67 |
| ATL_0006037 (germline - 171) | 5.51E−08 | germline - I80 |
| ATL_0006038 (germline - H77) | 4.82E−08 | germline - H86 |
| ATL_0006039 (T73K) | 1.09E−09 | T82K |
| ATL_0005262 | 3.48E−09 | |

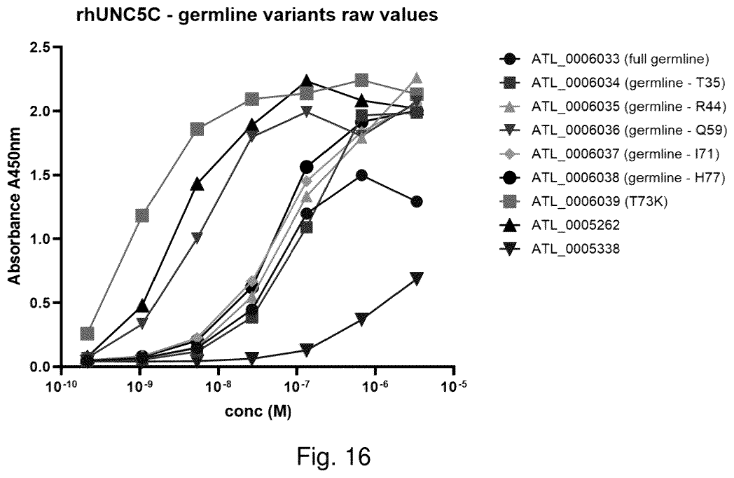

Reversing the entire ATL_5262 VH sequence to germline increases the EC50, and therefore reduces potency, over 10-fold compared with ATL_5262 (Table 12 and FIG. 16). All variants with germline reversion Q67Y (IMGT) (ATL_6033; 6034; 6035; 6037; and 6038) show reduced binding activity in ELISA (FIG. 16), reflected in an increased EC50 value. It is therefore desirable to keep glutamine (Q) at IMGT position 67 so as to maintain the degree of UNC5C binding observed for ATL_5262. The inventors further observed that mutating IMGT position 82 of the VH to a lysine (K) in order to reduce the risk of aspartate (T) isomerisation, enhances binding of the resulting antibody (ATL_6039) to UNC5C about 3-fold compared with ATL_5262 (FIG. 16). These data therefore indicate that IMGT residues 67 and 82 are important for UNC5C binding. These mutations were subsequently combined in the VHs of ATL_6177 (SEQ ID NO: 91) and ATL_6178 (SEQ ID NO: 93), with both of these mutating IMGT position 82 (T82K) and retaining Q67. In addition, ATL_6178 (SEQ ID NO: 93) also removes an additional potential sequence liability by mutating Asparagine (N) at position 58 IMGT, which is part of a predicted Asparagine deamidation site in CDR2 (NS motif), to Tyrosine (Y) (N58Y). By combining beneficial mutations, ATL_6177 and ATL_6178 are expected to have improved stability and binding potency compared with other ATL_5262 variants.

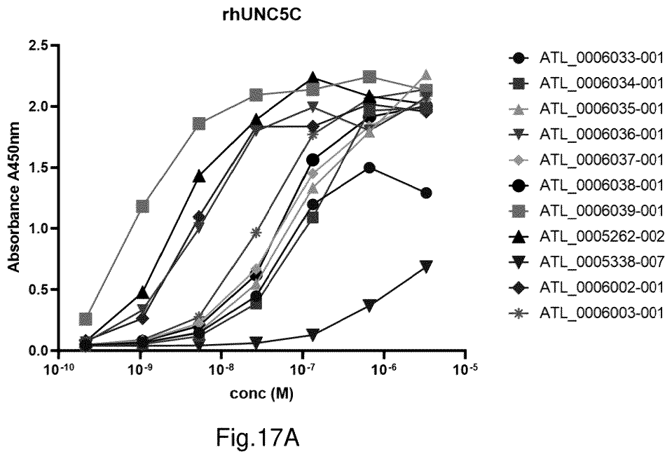

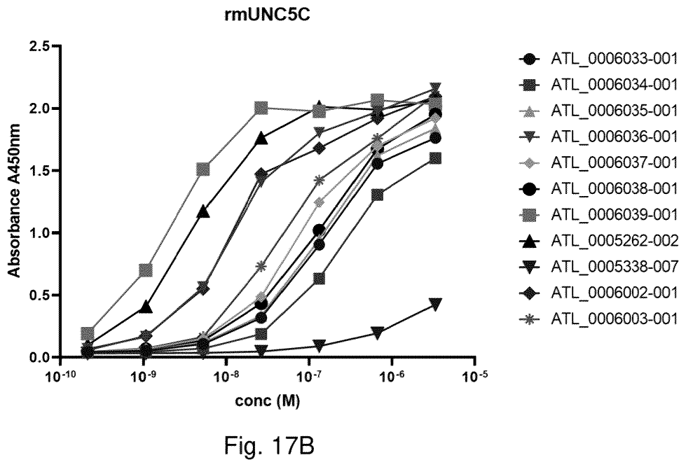

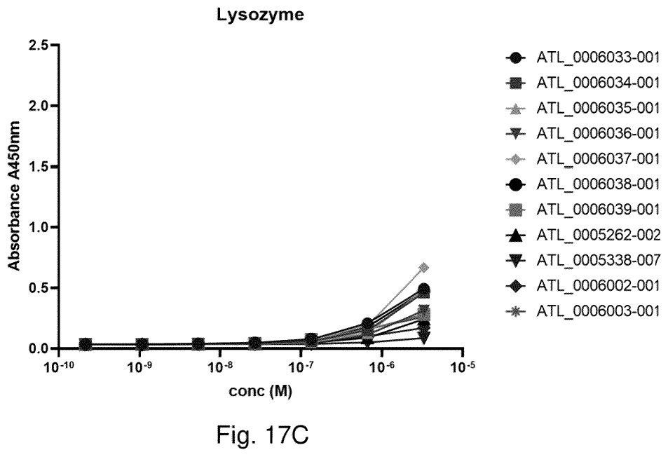

The tested VH variants show similar binding profiles for human (FIG. 17A) and murine UNC5C (FIG. 17B), whilst showing minimal binding to lysozyme at the highest concentration of antibody used (FIG. 17C).

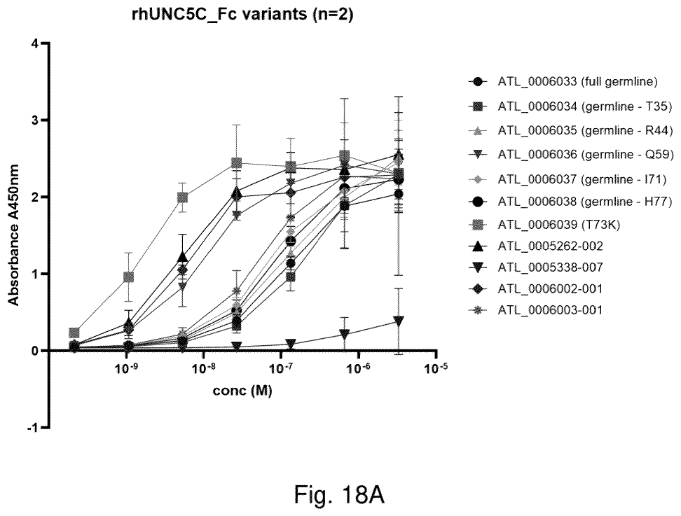

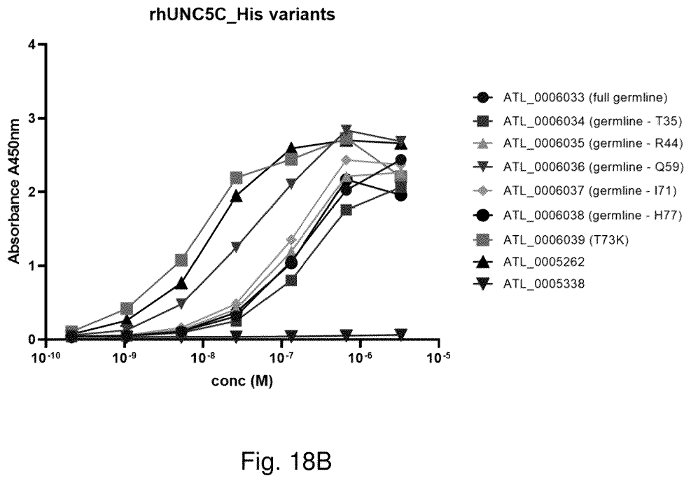

UNC5C exists in monomeric form in the absence of its ligand netrin 1, and in dimeric form when it binds to netrin-1. To test whether ATL_5262 binds both monomeric and dimeric UNC5C, an ELISA was performed using Fc-tagged dimeric rhUNC5C and His-tagged monomeric rhUNC5C. Similar binding profiles were observed for binding to the dimeric Fc-tagged rhUNC5 (FIG. 18A) and monomeric his-tagged rhUNC5C (FIG. 18B), and this was reflected in similar EC50 values (Table 13) indicating that ATL_5262 is capable of binding to both monomeric and dimeric UNC5C.

TABLE 13
EC50 values for VH variant antibodies tested
in ELISA using Fc-tagged or His-tagged UNC5C.

| | | rhUNC5C_Fc | rhUNC5C_His |
|---|---|---|---|
| EC50 (M) | IGMT numbering | (n = 2) | (n = 1) |
| ATL_0006033 (full germline) | | 1.90E−07 | 2.65E−07 |
| ATL_0006034 (germline - T35) | germline - T40 | 2.10E−07 | 5.08E−07 |
| ATL_0006035 (germline - R44) | germline - R49 | 1.28E−07 | 2.12E−07 |
| ATL_0006036 (germline - Q59) | germline - Q67 | 1.16E−08 | 3.70E−08 |
| ATL_0006037 (germline - 171) | germline - I80 | 8.71E−08 | 1.48E−07 |
| ATL_0006038 (germline - H77) | germline - H86 | 1.04E−07 | 3.02E−07 |
| ATL_0006039 (T73K) | T82K | 1.90E−09 | 9.78E−09 |
| ATL_0005262 parental | | 5.84E−09 | 1.40E−08 |
| ATL_0006002-001 (VL variant) | | 8.178E−09 | 2.126E−08 |

Antibody variants of ATL_5262 with different light chains were also generated and tested.

Further candidate VLs were obtained by a two-step process. First, 600 different VLs were computationally synthesised for the VH of ATL_5262 using a modified version of the model described in WO2022/223451, where encoder and decoder models were replaced with a pretrained AntiBERTa model as described in Leem at al., 2022. As a second step, the 600 different VH-VL pairs were prioritised using a separate AntiBERTa model, described in GB 2215218.5 filed 14 Oct. 2022. In brief, the model from GB2215218.5 takes a VH-VL pair as input (natural or synthetic) and computes the probability that the two chains would form a pairing.

10 VLs were selected for synthesis based on the predicted probability from the model (selecting candidate VLs with high predicted pairing probability with the VH of ATL_5262), low sequence liability content, and sequence diversity. VL variants tested included the VL of ATL_5996 (SEQ ID NO: 10); ATL_5997 (SEQ ID NO: 11); ATL_5998 (SEQ ID NO: 12); ATL_5999 (SEQ ID NO: 13); ATL_6000 (SEQ ID NO: 14); ATL_6001 (SEQ ID NO: 15); ATL_6002 (SEQ ID NO: 16); ATL_6003 (SEQ ID NO:17); ATL_6004 (SEQ ID NO: 18); ATL_6005 (SEQ ID NO:19). All of the above VL variants antibodies include the VH of ATL_5262.

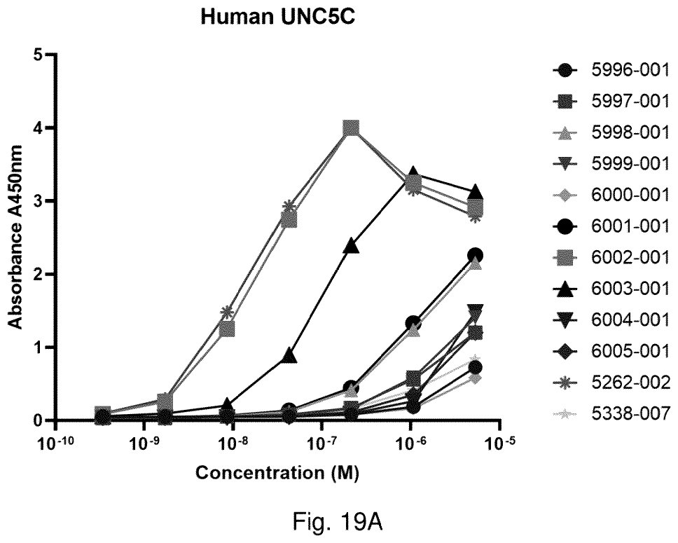

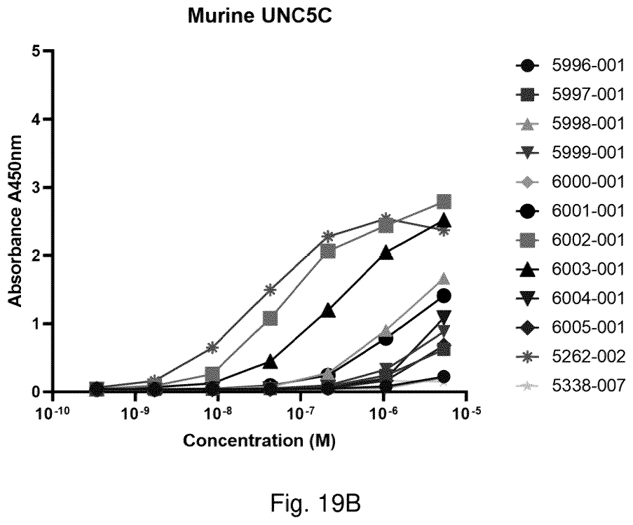

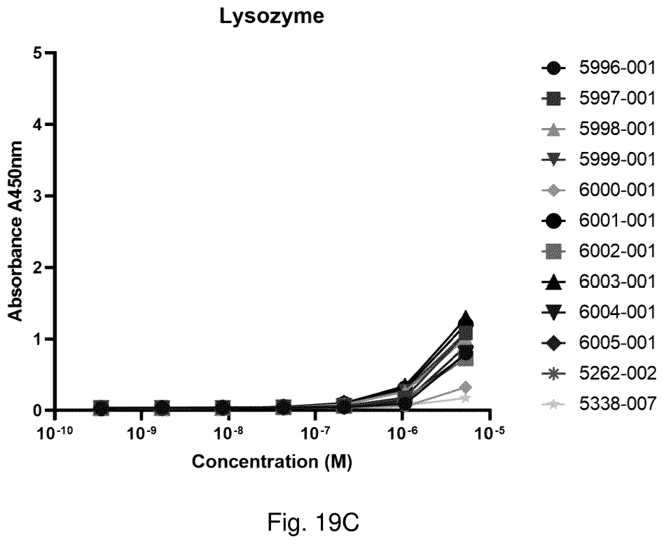

The different light chain variants were screened for binding to rhUNC5C and rmUNC5C by ELISA and compared with ATL_5262. The results show ATL_6002 (same VH as ATL_0005262 but different VL) binding to rhUNC5C in a very similar way to ATL_0005252 (FIG. 19A). Similar results were also observed for binding to rmUNC5C (FIG. 19B). Variant ATL_6003 demonstrated reduced binding (approximately 10-fold) to both rhUNC5C and rmUNC5C.

This data demonstrates that other VL pairings, for example using the VL of ATL_6002, are possible that maintain the functionality of the VH described herein. The data further demonstrates that the initial VH-VL pairing obtained (resulting in ATL_5262) was already very good.

To measure the binding potency of antibodies to UNC5C protein, antibodies were assessed in a direct ELISA format for binding to human UNC5C. In addition, a selection of these antibodies was also screened for binding to mouse, cyno, and rat UNC5C in a direct ELISA format. Lysozyme was run as a control antigen and showed no discernible binding to any of the antibodies assessed at a concentration below 1 μM. The anti-fluorescein antibody ATL-5338 was used as a negative isotype control and showed no binding to any of the UNC5C antigens assessed to antibody concentrations <1 μM. In addition, some of the antibodies were assessed in an additional assay to assess their ability to compete with Netrin 1 for binding to UNC5C. The results of these tests are shown in Table 14.

TABLE 14
Binding potency of listed antibodies to human, mouse, and cyno UNC5C and ability to compete with netrin-1.

| | | Human UNC5C | | | | Netrin 1 |
|---|---|---|---|---|---|---|
| | | EC50 (M) | Mouse UNC5C | Cyno UNC5C | Rat UNC5C | induced |
| | | UNC5C (R&D | EC50 (M) | EC50 (M) | ELISA EC50 | shift in |
| | | Systems; | (Inhouse | (Inhouse | (inhouse | binding |
| | Antibody | #1005-UN) | antigen) | antigen) | antigen) | curve |
| Alternative | ATL_0005996 | >0.000001 | >0.000001 | n/a | n/a | n/a |
| light | ATL_0005997 | >0.000001 | >0.000001 | n/a | n/a | n/a |
| chain | ATL_0005998 | Binding at | Binding at | n/a | n/a | n/a |
| | | 0.00000107 | 0.00000107 | | | |
| | | but top of | but top of | | | |
| | | curve not | curve not | | | |
| | | reached | reached | | | |
| | ATL_0005999 | >0.000001 | >0.000001 | n/a | n/a | n/a |
| | ATL_0006000 | >0.000001 | >0.000001 | n/a | n/a | n/a |
| | ATL_0006001 | Binding at | Binding at | n/a | n/a | n/a |
| | | 0.00000107 | 0.00000107 | | | |
| | | but top of | but top of | | | |
| | | curve not | curve not | | | |
| | | reached | reached | | | |
| | ATL_0006002 | 2.45E−08 | 6.537E−08 | n/a | n/a | Yes |
| | ATL_0006003 | 9.5e−8 | 3.072E−07 | n/a | n/a | n/a |
| | ATL_0006004 | >0.000001 | >0.000001 | n/a | n/a | n/a |
| | ATL_0006005 | >0.000001 | >0.000001 | n/a | n/a | n/a |
| Germline | ATL_0006033 | 4.77E−08 | 1.26E−07 | n/a | n/a | n/a |
| and | ATL_0006034 | 9.81E−08 | 1.94E−07 | n/a | n/a | n/a |
| development | ATL_0006035 | 9.74E−08 | 1.24E−07 | n/a | n/a | n/a |
| | ATL_0006036 | 5.38E−09 | 1.74E−08 | n/a | n/a | Yes |
| | ATL_0006037 | 5.51E−08 | 7.99E−08 | n/a | n/a | n/a |
| | ATL_0006038 | 4.82E−08 | 1.17E−07 | n/a | n/a | n/a |
| | ATL_0006039 | 1.09E−09 | 2.55E−09 | Binding at | Binding at | Yes |
| | | | | 0.00000000411523 | 0.00000000411523 | |
| | | | | but bottom of | but bottom of | |
| | | | | curve not | curve not | |
| | | | | reached | reached | |
| Combo | ATL_0006177 | 1.36E−08 | | n/a | | Yes |
| | ATL_0006178 | 9.62E−09 | 5.956E−09 | 1.578E−08 | 7.218E−09 | Yes |
| 5262 | ATL_0006187 | 2.24E−08 | n/a | n/a | n/a | n/a |
| homologues | ATL_0006191 | 4.52E−09 | Binding at | Binding at | Binding at | |
| | | | 0.00000000411523 | 0.00000000411523 | 0.00000000411523 | |
| | | | but bottom of | but bottom of | but bottom of | n/a |
| | | | curve not | curve not | curve not | |
| | | | reached | reached | reached | |

ATL_0005262, ATL_0005998, ATL_0006001, ATL_0006002, ATL_0006003, ATL_0006033, ATL_0006034, ATL_0006035, ATL_0006036, ATL_0006037, ATL_0006038, ATL_0006039, ATL_0006177, ATL_0006178, ATL_0006187, ATL_0006191 all demonstrated binding to human UNC5C with various potencies (see Table 14). For ATL_0006001 and ATL_6039, a signal at least 3× the level of lysosome control was observed, but a full dose response curve could not be obtained and so an EC50 cannot be reported. In both cases the data therefore shows that the antibodies do bind the target.

In the case of ATL_6039 an EC50 could not be obtained because the antibody was very potent and a lower concentration range would need to be tested to get an EC50. This data means that antibody certainly binds, just cannot report potency number.

ATL_0005262, ATL_0005998, ATL_0006001, ATL_0006002, ATL_0006003, ATL_0006178, ATL_0006191 showed binding to mouse UNC5C. ATL_0005262, ATL_0006178 and ATL_0006191 were demonstrated binding to cyno UNC5C. ATL_0005262, ATL_0006178 and ATL_0006191 were tested and demonstrated binding to rat UNC5C. In addition, Netrin 1 competed with ATL_0005262, ATL_0006002, ATL_0006036, ATL_0006039, ATL_0006177 and ATL_0006178 for binding to human UNC5C.

These data demonstrate that at least ATL_0006178 exhibits binding to human, mouse, rat, and cyno UNC5C, and is capable of competing with netrin-1 for binding to UNC5C.

### Example 7—PK Analysis

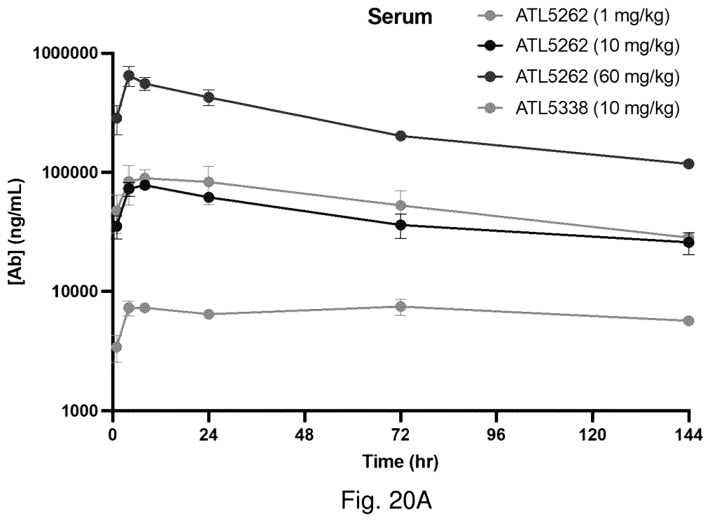

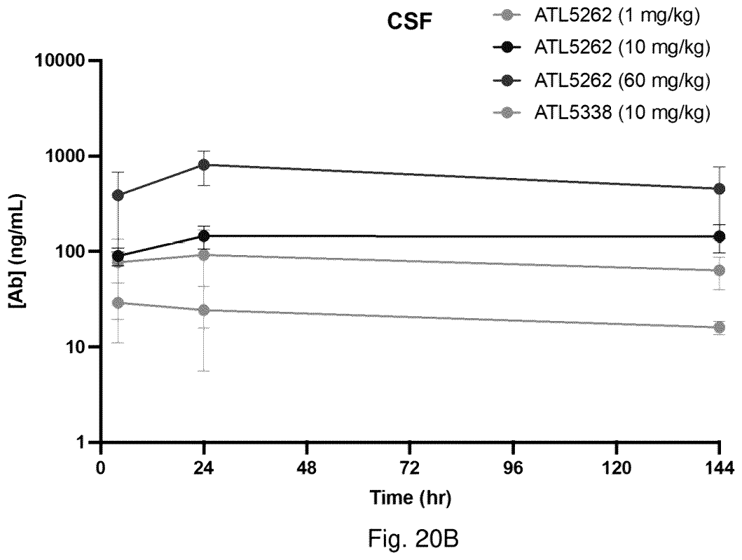

An in vivo pharmacokinetic study was conducted in mice to assess circulating levels of ATL_5262 and its ability for CNS-penetration compared with the control antibody, ATL_5338. Antibody levels were assessed via ELISA of serum samples collected at 1, 4, 8, 24, 72 and 144 hours and of CSF samples collected at 4, 24 and 144 hrs following intraperitoneal administration. ATL_5262 exhibits linear serum (FIG. 20A) and CSF PK (FIG. 20B) with a clearance profile that is comparable to the non-binding IgG1 control, ATL_5338. Importantly, ATL_5262 shows evidence of CNS-penetration with a serum:CSF ratio of ˜0.1-0.2%.

### Example 8—UNC5C Apoptosis

The antibodies described herein are expected to be able to enhance synaptic health (such as e.g. plasticity) by acting as a netrin-1 mimic. This can be tested using in vitro assays that directly assess measures of synaptic health which may include but are not limited to parameters such as the number of processes per cell, the length of neurite outgrowth, and branching (using for example the methods provided in Spijkers et al., Sci Rep, 2021), as would be expected from their binding profile.

Together, this data shows that UNC5C antibodies as described herein are likely to show therapeutic effects in the treatment and/or prevention of neurodegenerative diseases such as FTD and AD and provides antibodies that can achieve this by binding to the same binding site as Netrin 1.

### Example 9—Solubility and Stability of ATL 6178 (ATLX_1282)

The manufacturability of ATL_6178 (also referred to herein as ATXL_1282) was tested following exposure to different stress conditions. Such conditions included freeze thaw cycles, agitation, thermal stability and low pH. Stressed samples were evaluated for appearance, purity, aggregation, size and change in charge variants using MFI, SEC-HPLC, CE-SDS, cIEF and DLS. Samples were also concentrated to high densities (50 mg/mL, 100 mg/mL and 200 mg/mL) and analysed at time 0 and after 7 days incubation at room temperature. Solubility samples were analysed for appearance, viscosity and purity using SEC-HPLC.

ATL_6178 was expressed from a CHO-K1 stable pool transfection, following a 14 day fed batch the culture was harvested and the monoclonal antibody purified using protein A capture and a second stage cation exchange chromatography step followed by centrifugation using a 30 kDa cut off and buffer exchange into formulation buffer 20 mM histidine HCl, 8% sucrose, 0.04% tween 80, pH 6.0 followed by sterile filtration.

ATLX-1282 solubility was tested at 50 mg/mL, 100 mg/mL and 200 mg/mL. Samples were evaluated at time 0 and after 7 days at 25° C. The results are shown in Table 15 and Table 16. Visual evaluation showed the protein is free of particles and there is no change in appearance (Table 15), while SEC-HPLC analysis demonstrates no substantial change in monomeric form, small changes in low molecular weight species (LMWS) and high molecular weight species (HMWS) present after concentration and incubation at 25° C. for 7 days (Table 16). This indicates the molecule is stable at high concentrations. Viscosity reaches a maximum of 24 cP at 200 mg/mL indicating the molecule is manufacturable.

TABLE 15
Solubility analysis results for ATL_6178.

| | | | | Protein | |
|---|---|---|---|---|---|
| | Protein | Sampling | | Conc. | Viscosity |
| Antibody | Concentration | Points | Appearance | (mg/mL) | (cP, 20° C.) |
| ATL_6178 | 50 mg/mL | T0 | SY, SO, FP | 54.2 | 1.9 |
| | | 25-7 D | SY, SO, FP | 57 | NT |
| | 100 mg/mL | T0 | SY, SO, FP | 106.6 | 3.6 |
| | | 25-7 D | SY, SO, FP | 104.6 | NT |
| | 200 mg/mL | T0 | SY, SO, FP | 205.1 | 24 |
| | | 25-7 D | SY, SO, FP | 192.9 | NT |

D; day, SY; Slightly yellow; SO: Slightly opalescent liquid; FP: Free of visible particles; cP: centipoise.

TABLE 16
SEC-HPLC results for ATL_6178 solubility study.

| | | | Protein | SEC-HPLC | | |
|---|---|---|---|---|---|---|
| | Protein | Sampling | Conc. | Monomer | HMW | LMW |
| Antibody | Concentration | Points | (mg/mL) | (%) | (%) | (%) |
| ATL_6178 | 50 mg/mL | T0 | 54.2 | 98.9 | 1.1 | 0 |
| | | 25-7 D | 57 | 98.8 | 1.1 | 0 |
| | 100 mg/mL | T0 | 106.6 | 98.9 | 1.1 | 0 |
| | | 25-7 D | 104.6 | 98.4 | 1.5 | 0.1 |
| | 200 mg/mL | T0 | 205.1 | 98.6 | 1.4 | 0 |
| | | 25-7 D | 192.9 | 97.6 | 2.3 | 0.1 |

HMW: High molecular weight; LMW: Low molecular weight.

To further evaluate the stability of ATL_6178, the antibody was subjected to a panel of different stress conditions, including temperature, freeze-thaw cycles, agitation and low pH. Visual analysis of the samples before and after the stress conditions showed that samples were slightly yellow, slightly opalescent and free of visible particles, with no change in appearance following assessed conditions with the exception of thermal stress at 40° ° C. for 4 weeks showing a low level of visible particles. Table 17 shows the results of these stress studies. 10

TABLE 17
Visual analysis and sub-visible particle count of ATL_6178 stress studies.

| | | | | Protein | Sub-visible Particle | | |
|---|---|---|---|---|---|---|---|
| | | | | Conc. | Counts (counts/mL) | | |
| Sampling Points | | | Appearance | (mg/mL) | ≥2 μm | ≥10 μm | ≥25 μm |
| Time 0 | | | SY, SO, FP | 50 | 985 | 12 | 2 |
| Thermal | 2 | W | SY, SO, FP | 51.1 | 1687 | 17 | 0 |
| (40° C.) | | | | | | | |
| (40° C.) | 4 | W | SY, SO, | 49.6 | 4965 | 97 | 14 |
| | | | NEFP | | | | |
| Freeze-Thaw | 3 | C | SY, SO, FP | 51.7 | 1828 | 14 | 5 |
| (−70° C. to RT) | | | | | | | |
| (−70° C. to RT) | 5 | C | SY, SO, FP | 50.4 | 2858 | 61 | 15 |
| Agitation | 1 | D | SY, SO, FP | 50.3 | 1347 | 16 | 2 |
| (25° C., 300 rpm) | | | | | | | |
| (25° C., 300 rpm) | 3 | D | SY, SO, FP | 50.7 | 1532 | 33 | 2 |
| Low pH | 6 | h | SY, SO, FP | 49.1 | Not tested | | |
| (25° C., ~pH 3.5) | | | | | | | |
| (25° C., ~pH 3.5) | 24 | h | SY, SO, FP | 49.2 | | | |

SY: Slightly yellow; SO: Slightly opalescent liquid; FP: Free of visible particles; NEFP: Not essentially free of visible particles (more than 3 visible particles were observed); W: Week(s); C: Cycle(s); D: Day(s); h: hour(s).

Micro flow imaging (MFI) analysis shows an increase in subvisible particle detecting >2-25 um for the 40 C stress condition. Particle size and distribution were analysed using dynamic light scattering (DLS). The Z-average showed no significant change in any sample compared to the un-stressed stored at −70 C. The polydispersity index (PDI) indicates the samples were monodispersed (Table 18).

TABLE 18
DLS results of ATL_1678 stress studies.

| | | | | Protein | DLS (Size) | |
|---|---|---|---|---|---|---|
| | | | | Conc. | Z-Average | |
| Sampling Points | | | Appearance | (mg/mL) | (d · nm) | PDI |
| Time 0 | | | SY, SO, FP | 50 | 6.2 | 0.15 |
| Thermal | 2 | W | SY, SO, FP | 51.1 | 6.3 | 0.14 |
| (40° C.) | | | | | | |
| (40° C.) | 4 | W | SY, SO, | 49.6 | 6.6 | 0.15 |
| | | | NEFP | | | |
| Freeze-thaw | 3 | C | SY, SO, FP | 51.7 | 6.3 | 0.15 |
| (−70° C. to RT) | | | | | | |
| (−70° C. to RT) | 5 | C | SY, SO, FP | 50.4 | 6.3 | 0.15 |
| Agitation | 1 | D | SY, SO, FP | 50.3 | 6.2 | 0.15 |
| (25° C., 300 rpm) | | | | | | |
| (25° C., 300 rpm) | 3 | D | SY, SO, FP | 50.7 | 6.2 | 0.14 |
| Low pH | 6 | h | SY, SO, FP | 49.1 | Not tested | |
| (25° C., ~pH 3.5) | | | | | | |
| (25° C., ~pH 3.5) | 24 | h | SY, SO, FP | 49.2 | | |

W: Week(s); C: Cycle(s); D: Day(s); h: hour(s); SY: Slightly yellow; SO: Slightly opalescent liquid; FP: Free of visible particles.

SEC-HPLC analysis shows no change after 3 or 5 freeze-thaw cycles or agitation for 1 or 3 days. A small decline in monomer main peak % was detected after incubation at 40° ° C. for 4 weeks with increase in HMWS and LMWS present, 0.7% and 0.5% respectively. Low pH hold at 25° C. at pH3.4 resulted in 1.3% increase in HMWS (Table 19).

TABLE 19
SEC-HPLC results of ATLX-1282 stress studies.

SEC-HPLC

| | | | Monomer | HMW | LMW |
|---|---|---|---|---|---|
| Sampling Points | | | (%) | (%) | (%) |
| T0 | | | 99 | 1 | 0 |
| Thermal | 2 | W | 98.3 | 1.4 | 0.3 |
| (40° C.) | 4 | W | 97.8 | 1.7 | 0.5 |
| Freeze-thaw | 3 | C | 99 | 1 | 0 |
| (−70° C. to RT) | 5 | C | 99 | 1 | 0 |
| Agitation | 1 | D | 99 | 1 | 0 |
| (25° C., 300 rpm) | 3 | D | 98.8 | 1.2 | 0.1 |
| Low pH | 6 | h | 97.7 | 2.3 | 0 |
| (25° C., ~pH 3.5) | 24 | h | 97.9 | 2 | 0 |

HMW: High molecular weight, LMW: Low molecular weight, W: Week(s), C: Cycle(s), D: Day(s), h: hour(s).

Charge heterogeneity of ATL_1678 was analysed using cIEF. A decrease in the main charge variants was observed at 40° C. (−22.6 pp at 2 weeks and 34.3 pp at 4 weeks) with both an increase in acidic and basic species, however minimal changes in charge species present were observed in all other conditions (Table 20).

TABLE 20
iCIEF results of ATL_1678 stress studies.

| | | | | iCIEF | | |
|---|---|---|---|---|---|---|
| | Sampling Points | | | MP (%) | AP (%) | BP (%) |
| T0 | | | | 65.4 | 32.6 | 2 |
| | Thermal | 2 | W | 42.8 | 41.8 | 15.4 |
| | (40° C.) | 4 | W | 31.1 | 54.6 | 14.3 |
| | Freeze-Thaw | 3 | C | 64.1 | 34 | 1.9 |
| | (−70° C. to RT) | 5 | C | 64.2 | 33.7 | 2.1 |
| | Agitation | 1 | D | 64 | 33.9 | 2.1 |
| | (25° C., 300 rpm) | 3 | D | 63.5 | 34.7 | 1.8 |
| | Low pH | 6 | h | 63.4 | 33.9 | 2.7 |
| | (25° C., ~pH 3.5) | 24 | h | 64.6 | 33.1 | 2.4 |

MP: Main peak; AP: Acidic Peak; BP: Basic peak; W: Week(s); C: Cycle(s); D: Day(s); h: hour(s).

Molecular weight species and purity were analysed following exposure of the samples to stress conditions using CE-SDS. Samples incubated at 40° C. for 4 weeks demonstrated a decrease in purity of 3.5 pp for the non-reduced samples and 1 pp for the reduced samples. Minimal changes were observed for samples experiencing other stress conditions (Table 21).

TABLE 21
iCIEF results of ATL_6178 stress studies.

| | | | SDS-Caliper-NR | SDS-Caliper-R |
|---|---|---|---|---|
| Sampling Points | | | Purity (%) | Purity (%) |
| T0 | | | 96.5 | 98.4 |
| Thermal | 2 | W | 94.4 | 97.9 |
| (40° C.) | 4 | W | 93 | 97.4 |
| Freeze-Thaw | 3 | C | 96.6 | 98.5 |
| (−70° C. to RT) | 5 | C | 96.5 | 98.4 |
| Agitation | 1 | D | 96.4 | 98.5 |
| (25° C., 300 rpm) | 3 | D | 96.4 | 98.5 |
| Low pH | 6 | h | 96.4 | 98.6 |
| (25° C., ~pH 3.5) | 24 | h | 95.9 | 98.3 |

W: Week(s), C: Cycle(s), D: Day(s), h: hour(s).

The above data show that ATL_6178 is highly stable under various stress conditions.

### Example 10—Binding to Cellular UNC5C

To assess the ability of ATL_6178 (also referred to herein as ATLX-1282) and ATL5262 to bind to UNC5C in its natural central nervous system (CNS) context, antibody binding to frozen mouse brain sections was assessed.

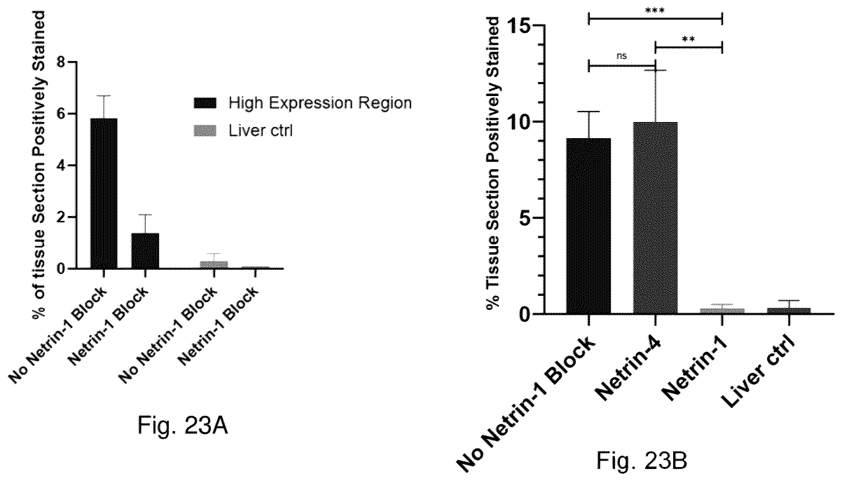

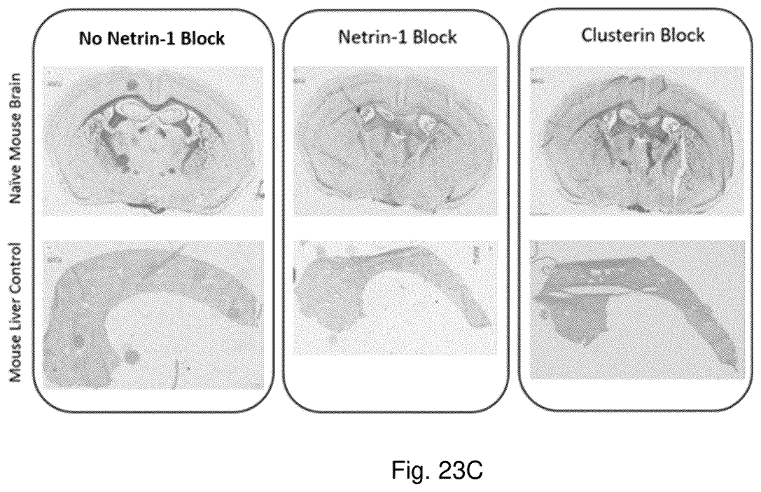

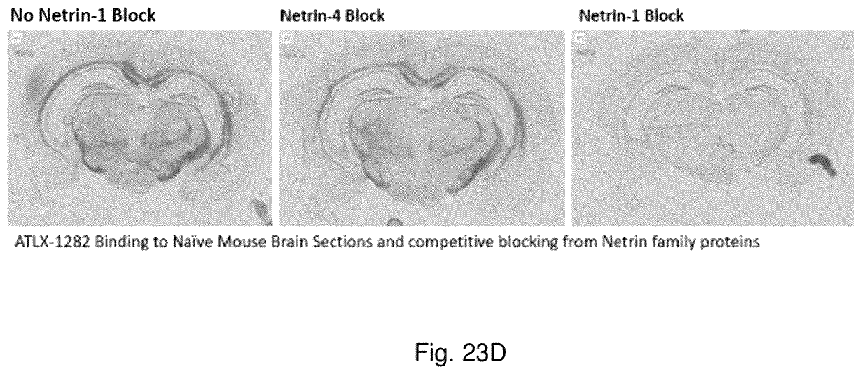

ATLX-1282 (ATL_6178) and ATL_5262 demonstrated a similar binding pattern, and this was blocked by 10 nM Netrin-1, but not by similar sized proteins such as clusterin and Netrin-4 (which do not bind to UNC5C), as shown in FIGS. 23A-D. This is in keeping with the in vitro data described above showing the ability of ATLX-1282 and ATL_5262 to bind to UNC5C selectively.

To verify binding of antibodies to UNC5C expressed on the cell surface, antibodies were assessed in a flow cytometry assay. The canonical human UNC5C isoform (095185) and UNC5C T835M variant linked to AD (VAR_081368) were constitutively overexpressed in a HEK-293 cell line using a lentiviral system. Antibodies were then tested for binding using flow cytometry. The cell line transduced with the empty vector (EV) and an antibody specific for an unrelated target expressed in relevant IgG format (control ATL) were used as negative controls.

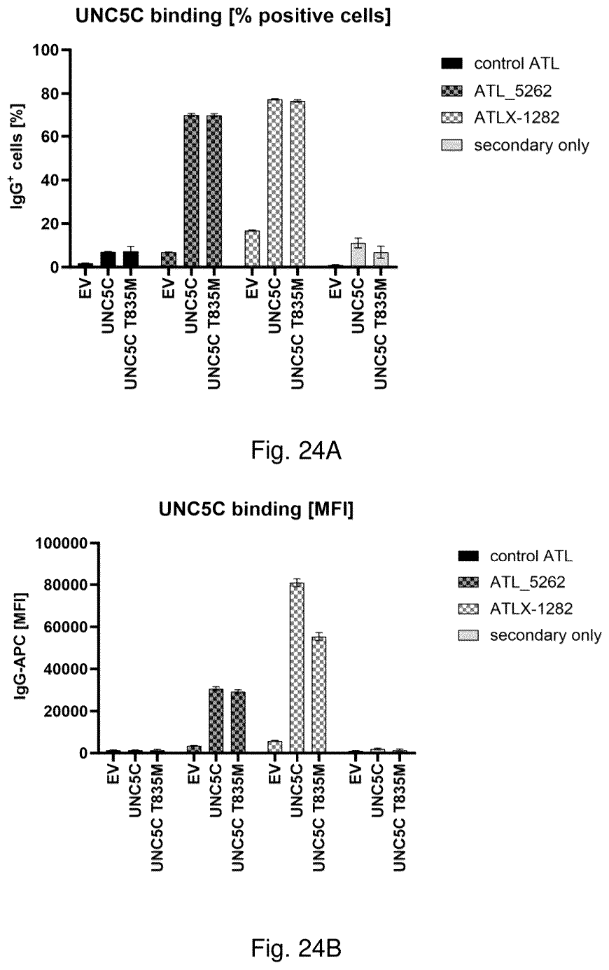

ATL5262 and ATLX-1282 showed increased binding to HEK-293 cells overexpressing UNC5C and HEK-293 cells overexpressing UNC5C T835M relative to cells transfected with an empty vector control (FIGS. 24A and B).

These data demonstrate that ATL5262 and ATLX-1282 can bind to UNC5C and the UNC5C T835M variant linked to AD (VAR_081368) when they are in a natural cell membrane context. This data shows that the antibodies bind to the Alzheimer's disease associated form of UNC5C. The antibodies described herein were shown to bind to the extracellular region of UNC5C and this mutation affects the intracellular region. Therefore, all antibodies described are expected to bind to the AD relevant form of UNC5C.

### Example 11—Determination of Crystal Structure of ATL 5262 Fab Fragment Bound to Human UNC5C Iq Domains

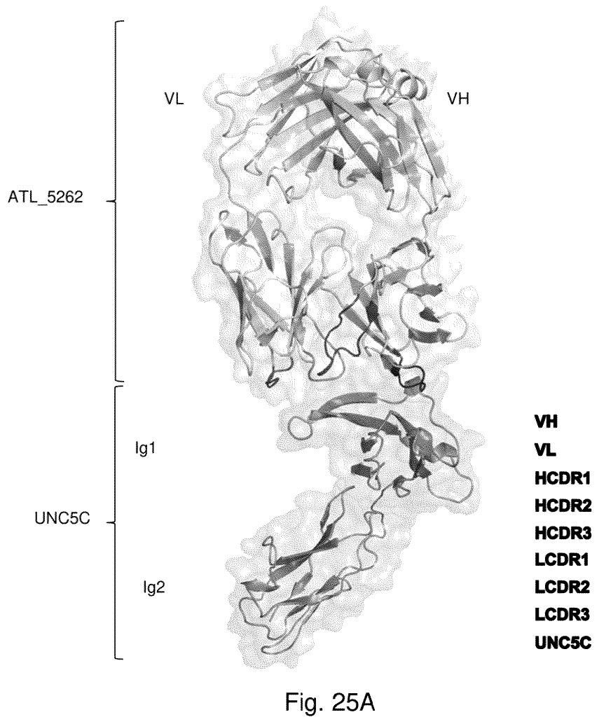

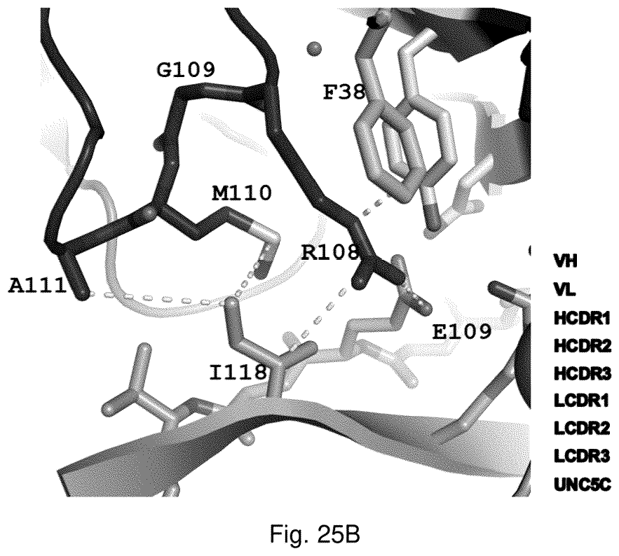

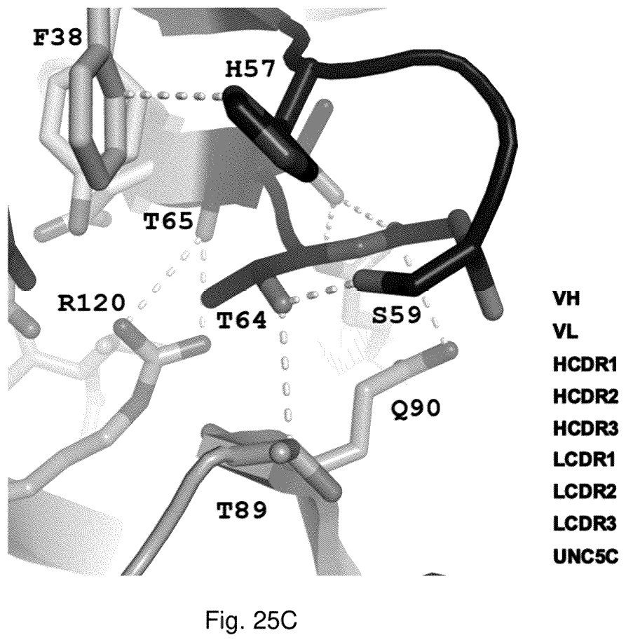

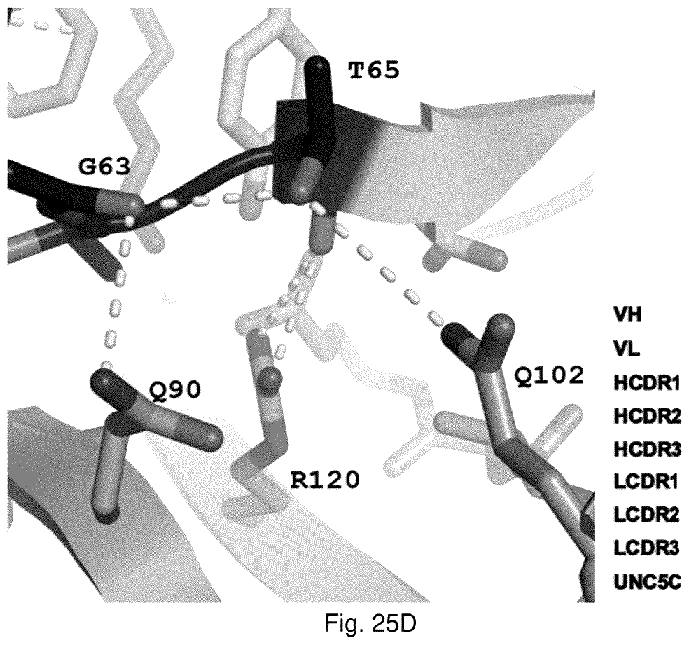

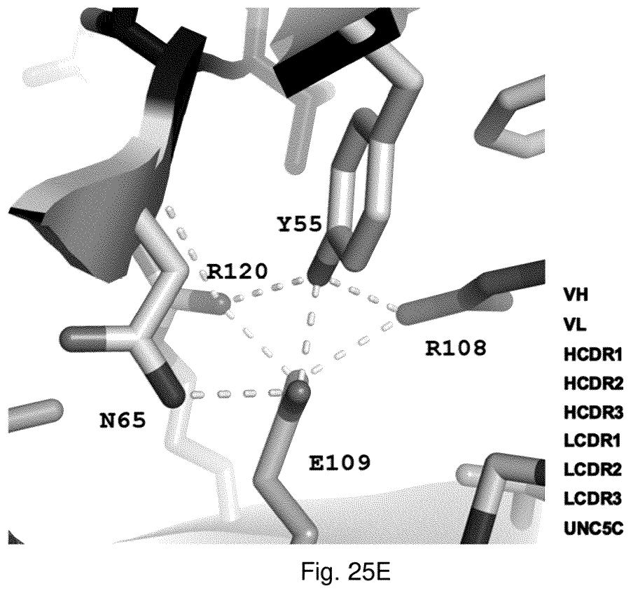

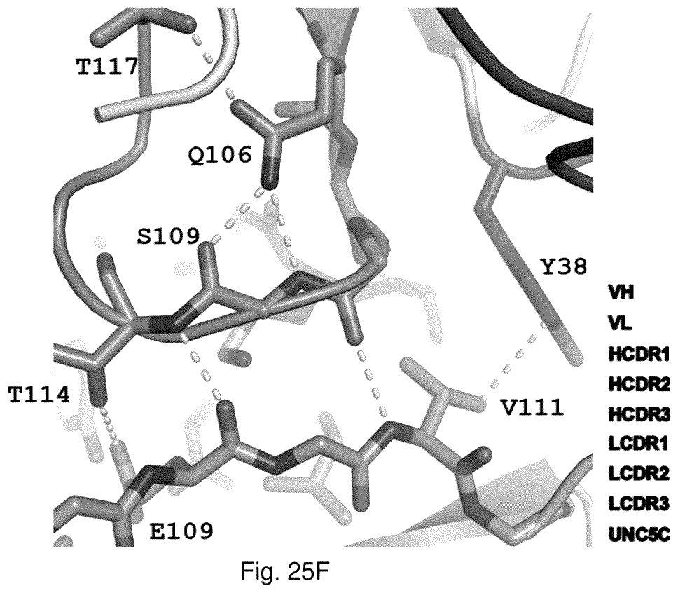

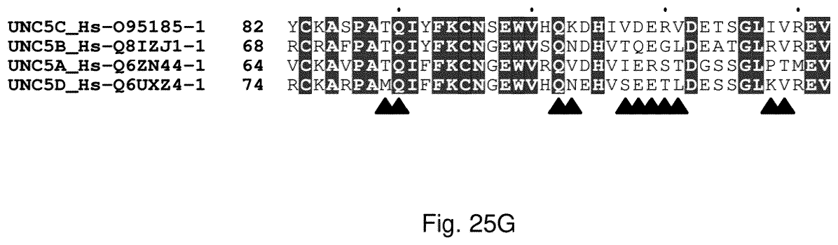

To better understand the binding of ATL_5262 to UNC5C, an X-ray crystal structure was obtained for a complex containing a Fab fragment of ATL_5262 bound to the immunoglobulin (Ig) domains of human UNC5C. A cartoon view of the full Fab-UNC5C complex is shown in FIG. 25A, and close-up views of the specific interactions between the proteins in FIGS. 25B-E. A summary of the inter-chain contacts is set out in Table 22 below, with details of the specific amino acid interactions in Table 23.

TABLE 22
ATL_5262 Fab:UNC5C interchain contacts summary

| Inter-chain | | | |
|---|---|---|---|
| interaction | Buried surface area, Å2 | H-Bonds | Salt Bridges |
| VH:UNC5C | 397.9 | 5 | 2 |
| VL:UNC5C | 238.5 | 4 | 0 |
| VH:VL | 1816.9 | 17 | 2 |

TABLE 23
Summary of inter-chain interactions within 4.0 A, including
polar contacts and hydrophobic interactions.

| VH | UNC5C | VL |
|---|---|---|
| Thr64 | Thr89 | |
| Gly63 | Gln90 | |
| Thr65 | Gln102 | |
| Ser74 | Lys103 | |
| Asn66 | Val107 | Thr114 |
| | Asp108 | Thr114 |
| Tyr55, Asn66, Arg108, Met110 | Glu109 | Ser109, T114 |
| | Arg110 | Tyr108, Ser109 |
| | Val111 | Tyr38, Ser107, Tyr108 |
| Arg108, Met110 | Ile118 | |
| Tyr55, Thr64, Thr65 | Arg120 | |

FIG. 24F shows a protein alignment of human UNC5 family members B, A and D to region of human UNC5C Ig-like domain 1 that interacts with ATL_5262. Identical sequences highlighted in red. Amino acids of UNC5C involved in the interaction to ATL_5262 and summarised in Table 23 are indicated by arrows. These data indicate that specificity of ATL_5262 to UNC5C over the other UNC5 family members may be driven by amino acids that are different between UNC5C and the other UNC5 proteins.

### Example 12—Further Antibodies

Phage display selections were carried out to discover additional functional and resilient patient-derived antibodies for UNC5C. Phage display libraries were generated from VH repertoires of patients who carried the original antibody ATL_0005262, and a VL repertoire from healthy donors. Phage display selections were done by panning on two UNC5C antigens. A subset of enriched antibodies was screened by phage ELISA on UNC5C antigen and 10 antibodies were chosen for IgG conversion.

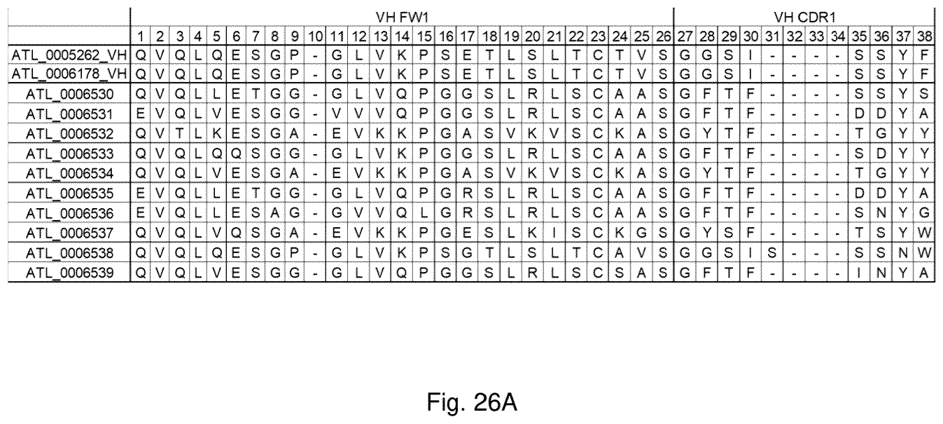

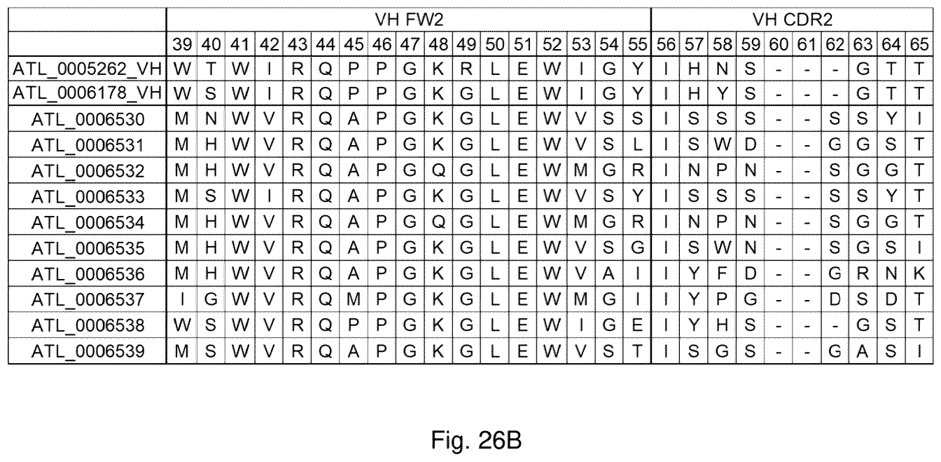

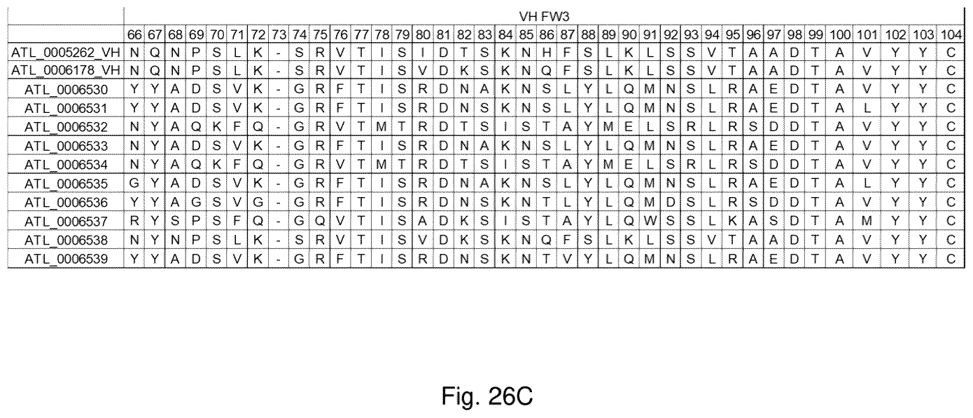

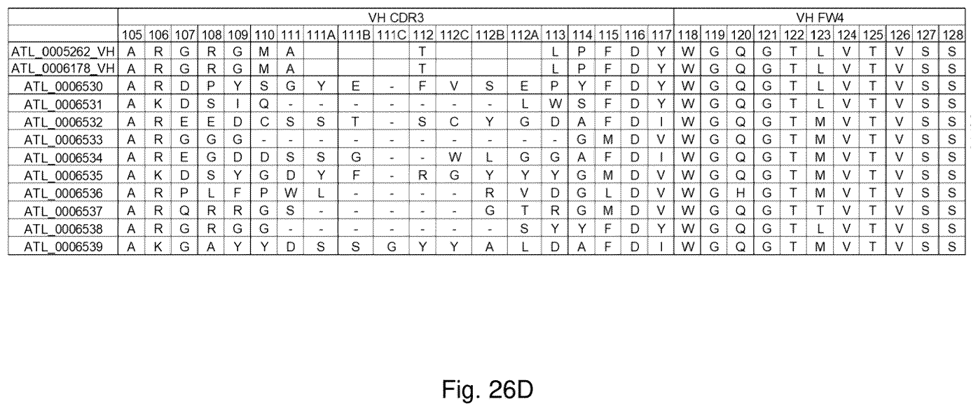

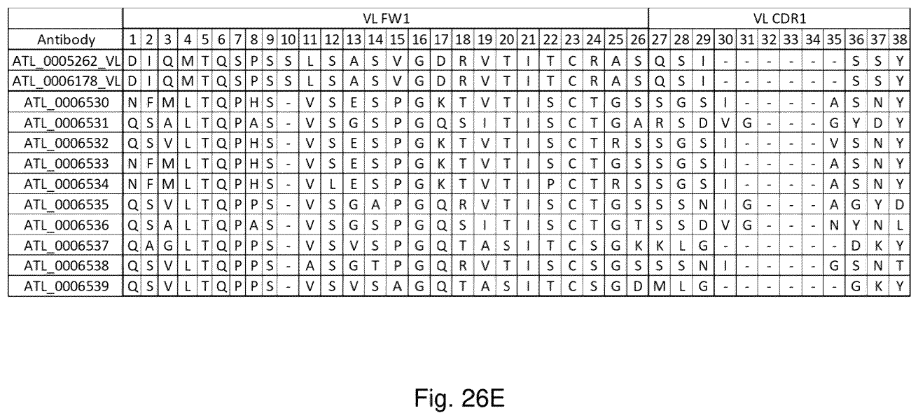

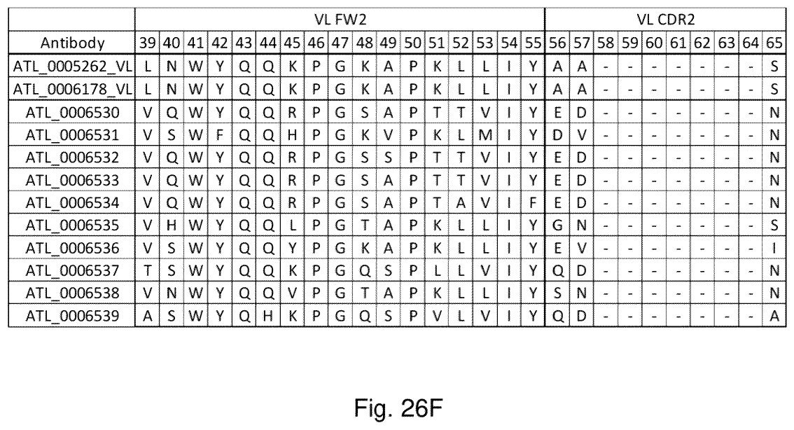

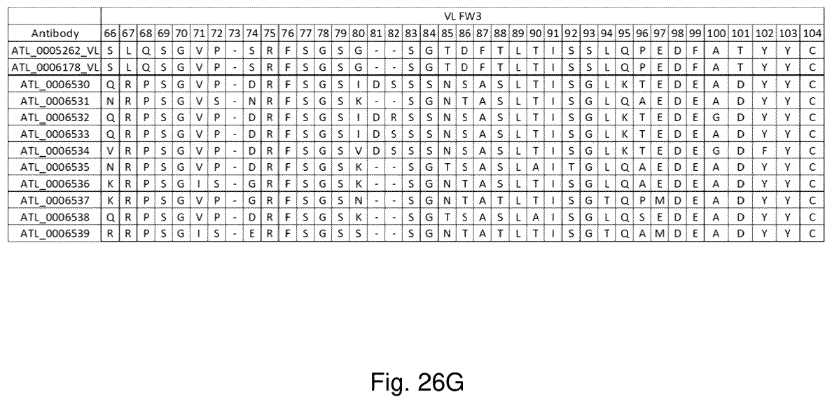

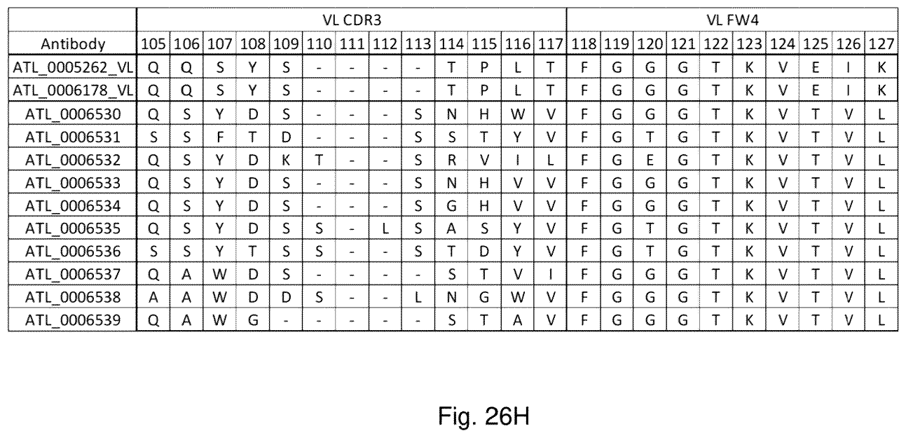

Ten phage-derived IgGs (ATL_6530 to ATL_6539) were screened for binding to UNC5C by ELISA and the EC50 values estimated from UNC5C binding curves are listed in Table 24 below. All antibodies showed binding to UNC5C with high affinity. ATL_6178 was also included for reference. FIG. 26 shows an alignment of the VH and VL sequences of these antibodies.

TABLE 24
EC50 values from UNC5C binding curves.

| | | | Assay | |
|---|---|---|---|---|
| | | Assay | Orien- | EC50 |
| Antibody Batch | Antigen | Type | tation | (M) |
| Y_0001389-001 | UNC5C (R&D | ELISA | Antigen | 3.90E−11 |
| (ATL_6530) | Systems; #1005-UN) | | Down | |
| Y_0001390-001 | UNC5C (R&D | ELISA | Antigen | 1.30E−11 |
| (ATL_6531) | Systems; #1005-UN) | | Down | |
| Y_0001391-001 | UNC5C (R&D | ELISA | Antigen | 2.80E−12 |
| (ATL_6532) | Systems; #1005-UN) | | Down | |
| Y_0001392-001 | UNC5C (R&D | ELISA | Antigen | 9.20E−12 |
| (ATL_6533) | Systems; #1005-UN) | | Down | |
| Y_0001393-001 | UNC5C (R&D | ELISA | Antigen | 1.00E−11 |
| (ATL_6534) | Systems; #1005-UN) | | Down | |
| Y_0001394-001 | UNC5C (R&D | ELISA | Antigen | 2.40E−08 |
| (ATL_6535) | Systems; #1005-UN) | | Down | |
| Y_0001395-001 | UNC5C (R&D | ELISA | Antigen | 1.10E−09 |
| (ATL_6536) | Systems; #1005-UN) | | Down | |
| Y_0001396-001 | UNC5C (R&D | ELISA | Antigen | 7.10E−10 |
| (ATL_6537) | Systems; #1005-UN) | | Down | |
| Y_0001397-001 | UNC5C (R&D | ELISA | Antigen | 5.50E−10 |
| (ATL_6538) | Systems; #1005-UN) | | Down | |
| Y_0001398-001 | UNC5C (R&D | ELISA | Antigen | 1.10E−08 |
| (ATL_6539) | Systems; #1005-UN) | | Down | |
| Y_0001282-003 | UNC5C (R&D | ELISA | Antigen | 8.00E−09 |
| (ATL_6178) | Systems; #1005-UN) | | Down | |

Next, these antibodies were screened to determine if they were able to compete for binding to UNC5C with recombinant Netrin 1. UNC5C was coated down on ELISA plates and a titration of each antibody individually was pre-incubated with UNC5C protein prior to the addition of netrin 1 at a set concentration. In each case the binding of each antibody and netrin 1 was determined using secondary detection antibodies specific for each reagent. If the presence of netrin 1 was able to shift the antibody binding curve to the right (i.e. require a higher concentration of antibody to see a similar level of signal for binding to UNC5C), this was observed as competition in the assay. Furthermore, if presence of antibody was able to reduce the signal of Netrin1 binding detected in the assay, this was also observed as competition (see Table 25). All antibodies displayed competition with Netrin-1.

TABLE 25
Competition of antibodies with Netrin 1 binding.

| | Antibody | | | Ab Top | Ab Bottom | Competitor |
|---|---|---|---|---|---|---|
| | (Antigen | Target | Competitor | Concentration | Conc | Conc |
| | Binding) | Antigen | (Antigen) | (M) | (M) | (M) |
| 1 | Y_0001389-001 | UNC5C (R&D | Netrin 1 (R&D | 1e−6 | 1e−13 | 7.5e−8 |
| | (ATL_6530) | Systems; | Systems; | | | |
| | | #1005-UN) | #6419-N1) | | | |
| 2 | Y_0001390-001 | UNC5C (R&D | Netrin 1 (R&D | 1e−6 | 1e−13 | 7.5e−8 |
| | (ATL_6531) | Systems; | Systems; | | | |
| | | #1005-UN) | #6419-N1) | | | |
| 3 | Y_0001391-001 | UNC5C (R&D | Netrin 1 (R&D | 1e−6 | 1e−13 | 7.5e−8 |
| | (ATL_6532) | Systems; | Systems; | | | |
| | | #1005-UN) | #6419-N1) | | | |
| 4 | Y_0001392-001 | UNC5C (R&D | Netrin 1 (R&D | 1e−6 | 1e−13 | 7.5e−8 |
| | (ATL_6533) | Systems; | Systems; | | | |
| | | #1005-UN) | #6419-N1) | | | |
| 5 | Y_0001393-001 | UNC5C (R&D | Netrin 1 (R&D | 1e−6 | 1e−13 | 7.5e−8 |
| | (ATL_6534) | Systems; | Systems; | | | |
| | | #1005-UN) | #6419-N1) | | | |
| 6 | Y_0001394-001 | UNC5C (R&D | Netrin 1 (R&D | 1e−6 | 1e−13 | 7.5e−8 |
| | (ATL_6535) | Systems; | Systems; | | | |
| | | #1005-UN) | #6419-N1) | | | |
| 7 | Y_0001395-001 | UNC5C (R&D | Netrin 1 (R&D | 1e−6 | 1e−13 | 7.5e−8 |
| | (ATL_6536) | Systems; | Systems; | | | |
| | | #1005-UN) | #6419-N1) | | | |
| 8 | Y_0001396-001 | UNC5C (R&D | Netrin 1 (R&D | 1e−6 | 1e−13 | 7.5e−8 |
| | (ATL_6537) | Systems; | Systems; | | | |
| | | #1005-UN) | #6419-N1) | | | |
| 9 | Y_0001397-001 | UNC5C (R&D | Netrin 1 (R&D | 1e−6 | 1e−13 | 7.5e−8 |
| | (ATL_6538) | Systems; | Systems; | | | |
| | | #1005-UN) | #6419-N1) | | | |
| 10 | Y_0001398-001 | UNC5C (R&D | Netrin 1 (R&D | 1e−6 | 1e−13 | 7.5e−8 |
| | (ATL_6539) | Systems; | Systems; | | | |
| | | #1005-UN) | #6419-N1) | | | |
| 11 | Y_0001282-003 | UNC5C (R&D | Netrin 1 (R&D | 1e−6 | 1e−13 | 7.5e−8 |
| | (ATL_6178) | Systems; | Systems; | | | |
| | | #1005-UN) | #6419-N1) | | | |

| | Antibody | Hit | |
|---|---|---|---|
| | (Antigen | (Competition | |
| | Binding) | Observed) | Notes |
| 1 | Y_0001389-001 | Yes | shift in bind curve profile vs what is |
| | (ATL_6530) | | seen in absence of Netrin 1 |
| 2 | Y_0001390-001 | Yes | shift in bind curve profile vs what is |
| | (ATL_6531) | | seen in absence of Netrin 1 |
| 3 | Y_0001391-001 | Yes | No shift in bind curve profile vs |
| | (ATL_6532) | | what is seen in absence of Netrin 1; |
| | | | Netrin 1 binding signal is reduced, |
| | | | suggesting ATL6532 is particularly |
| | | | potent and out competes Netrin 1 |
| | | | for binding in this assay setup |
| 4 | Y_0001392-001 | Yes | shift in bind curve profile vs what is |
| | (ATL_6533) | | seen in absence of Netrin 1 |
| 5 | Y_0001393-001 | Yes | shift in bind curve profile vs what is |
| | (ATL_6534) | | seen in absence of Netrin 1 |
| 6 | Y_0001394-001 | Yes | Shift in bind curve profile vs what is |
| | (ATL_6535) | | seen in absence of Netrin 1 |
| 7 | Y_0001395-001 | Yes | Shift in bind curve profile vs what is |
| | (ATL_6536) | | seen in absence of Netrin 1 |
| 8 | Y_0001396-001 | Yes | shift in bind curve profile vs what is |
| | (ATL_6537) | | seen in absence of Netrin 1 |
| 9 | Y_0001397-001 | Yes | shift in bind curve profile vs what is |
| | (ATL_6538) | | seen in absence of Netrin 1 |
| 10 | Y_0001398-001 | Yes | Shift in bind curve profile vs what is |
| | (ATL_6539) | | seen in absence of Netrin 1 |
| 11 | Y_0001282-003 | Yes | Shift in bind curve profile vs what is |
| | (ATL_6178) | | seen in absence of Netrin 1 |

Ab = antibody, Conc = concentration.

This example thus identifies further antibodies that bind UNC5C with high affinity and are able to compete with Netrin-1 for UNC5C binding and are therefore expected to have similar properties to ATL_5262 and ATLX-1282, including reducing UNC5C mediated apoptosis, reducing beta-amyloid toxicity, increasing neuron activity, viability and burst frequency, and enhancing synaptic health in vitro and in vivo.

The data above show that these antibodies all bind to an area of UNC5C that is shared for Netrin binding (as is the case for the other UNC5C-binding antibodies described herein, including ATL_5262 and ATLX-1282). The binding site of Netrin on UNC5C was not previously known precisely, but was believed to be somewhere in the first two immunoglobulin domains of UNC5C. This data provides the latest information to suggest that the binding site is within the first immunoglobulin domain of UNC5C, and likely to be at least partially overlapping with where the antibodies described herein bind to UNC5C.

Together, the data described herein show that UNC5C antibodies as described herein show therapeutic effects in the treatment and/or prevention of neurodegenerative diseases such as PD, FTD, AD and ALS and provides antibodies that can achieve this by binding to the same binding site as Netrin 1.

### REFERENCES

All documents cited herein are incorporated by reference in their entirety.
Ackerman, SL, and B B Knowles. “Cloning and mapping of the UNC5C gene to human chromosome 4q21-q23.” Genomics vol. 52, 2 (1998): 205-8. doi:10.1006/geno. 1998.5425
Bang, Jee et al. “Frontotemporal dementia.” Lancet (London, England) vol. 386, 10004 (2015): 1672-82. doi:10.1016/S0140-6736(15)00461-4
Bang, Jee et al. “Frontotemporal dementia.” Lancet (London, England) vol. 386, 10004 (2015): 1672-82. doi:10.1016/S0140-6736(15)00461-4
Boyer, Nicholas P, and Stephanie L Gupton. “Revisiting Netrin-1: One Who Guides (Axons).” Frontiers in cellular neuroscience vol. 12 221. 31 Jul. 2018, doi:10.3389/fncel.2018.00221
Burrell, James R et al. “Motor neuron dysfunction in frontotemporal dementia.” Brain: a journal of neurology vol. 134, Pt 9 (2011): 2582-94. doi:10.1093/brain/awr195
Chen, Guiqin et al. “Netrin-1 receptor UNC5C cleavage by active δ-secretase enhances neurodegeneration, promoting Alzheimer's disease pathologies.” Science advances vol. 7, 16 eabe4499. 16 Apr. 2021, doi:10.1126/sciadv.abe4499
Chen, Guiqin et al. “UNC5C Receptor Proteolytic Cleavage by Active AEP Promotes Dopaminergic Neuronal Degeneration in Parkinson's Disease.” Advanced science (Weinheim, Baden-Wurttemberg, Germany) vol. 9, 7 (2022): e2103396. doi:10.1002/advs.202103396
Cunningham, B C, and J A Wells. “High-resolution epitope mapping of hGH-receptor interactions by alanine-scanning mutagenesis.” Science (New York, N.Y.) vol. 244, 4908 (1989): 1081-5. doi:10.1126/science.2471267
Dun, Xin-Peng, and David B Parkinson. “Role of Netrin-1 Signaling in Nerve Regeneration.” International journal of molecular sciences vol. 18, 3 491. 24 Feb. 2017, doi:10.3390/ijms18030491
Glasgow, Stephen D et al. “Guiding synaptic plasticity: Novel roles for netrin-1 in synaptic plasticity and memory formation in the adult brain.” The Journal of physiology vol. 599, 2 (2021): 493-505. doi:10.1113/JP278704
Golde, Todd E. “Disease-Modifying Therapies for Alzheimer's Disease: More Questions than Answers.” Neurotherapeutics: the journal of the American Society for Experimental Neuro Therapeutics vol. 19, 1 (2022): 209-227. doi:10.1007/s13311-022-01201-2
Hashimoto, Yuichi et al. “An Alzheimer Disease-linked Rare Mutation Potentiates Netrin Receptor Uncoordinated-5C-induced Signaling That Merges with Amyloid B Precursor Protein Signaling.” The Journal of biological chemistry vol. 291, 23 (2016): 12282-93. doi:10.1074/jbc.M115.698092
Hardiman, Orla et al. “Amyotrophic lateral sclerosis.” Nature reviews. Disease primers vol. 3 17071. 5 Oct. 2017, doi:10.1038/nrdp.2017.71
Jasmin, Melissa et al. “Netrin-1 and its receptor DCC modulate survival and death of dopamine neurons and Parkinson's disease features.” The EMBO journal vol. 40, 3 (2021): e105537. doi:10.15252/embj.2020105537
Ju, Ting et al. “Decreased Netrin-1 in Mild Cognitive Impairment and Alzheimer's Disease Patients.” Frontiers in aging neuroscience vol. 13 762649. 16 Feb. 2022, doi:10.3389/fnagi.2021.762649
Knopman, David S et al. “Alzheimer disease.” Nature reviews. Disease primers vol. 7, 1 33. 13 May. 2021, doi:10.1038/s41572-021-00269-y
Llambi, F et al. “Netrin-1 acts as a survival factor via its receptors UNC5H and DCC.” The EMBO journal vol. 20, 11 (2001): 2715-22. doi:10.1093/emboj/20.11.2715
Leem, Jinwoo et al. “Deciphering the language of antibodies using self-supervised learning.” Patterns (New York, N.Y.) vol. 3, 7 100513. 18 May. 2022, doi:10.1016/j.patter.2022.100513
Leonardo, E D et al. “Vertebrate homologues of C. elegans UNC-5 are candidate netrin receptors.” Nature vol. 386, 6627 (1997): 833-8. doi:10.1038/386833a0
Lund, J et al. “Human Fc gamma RI and Fc gamma RII interact with distinct but overlapping sites on human IgG.” Journal of immunology (Baltimore, Md.: 1950) vol. 147, 8 (1991): 2657-62
Li, Quan et al. “The role of UNC5C in Alzheimer's disease.” Annals of translational medicine vol. 6, 10 (2018): 178. doi:10.21037/atm.2018.04.43
Obien, Marie Engelene J et al. “Revealing neuronal function through microelectrode array recordings.” Frontiers in neuroscience vol. 8 423. 6 Jan. 2015, doi:10.3389/fnins.2014.00423
Panza, Francesco et al. “Development of disease-modifying drugs for frontotemporal dementia spectrum disorders.” Nature reviews. Neurology vol. 16, 4 (2020): 213-228. doi:10.1038/s41582-020-0330-x
Phukan, Julie et al. “Cognitive impairment in amyotrophic lateral sclerosis.” The Lancet. Neurology vol. 6, 11 (2007): 994-1003. doi:10.1016/S1474-4422(07)70265-X
Poliak, Sebastian et al. “Synergistic integration of Netrin and ephrin axon guidance signals by spinal motor neurons.” eLife vol. 4 e10841. 3 Dec. 2015, doi: 10.7554/eLife. 10841
Pokinko, Matthew et al. “Resilience to amphetamine in mouse models of netrin-1 haploinsufficiency: role of mesocortical dopamine.” Psychopharmacology vol. 232, 20 (2015): 3719-29. doi:10.1007/s00213-015-4032-9
Prince, M et al (2014) Dementia UK: Update Second Edition report produced by King's College London and the London School of Economics for the Alzheimer's Society.
Rajasekharan, Sathyanath, and Timothy E Kennedy. “The netrin protein family.” Genome biology vol. 10, 9 (2009): 239. doi:10.1186/gb-2009-10-9-239
Renaud, Jean-Paul et al. “Cryo-EM in drug discovery: achievements, limitations and prospects.” Nature reviews. Drug discovery vol. 17, 7 (2018): 471-492. doi:10.1038/nrd.2018.77
Rosso, Sonia M et al. “Frontotemporal dementia in The Netherlands: patient characteristics and prevalence estimates from a population-based study.” Brain: a journal of neurology vol. 126, Pt 9 (2003): 2016-22. doi:10.1093/brain/awg204
Smith, Alec S T et al. “Human Induced Pluripotent Stem Cell-Derived TDP-43 Mutant Neurons Exhibit Consistent Functional Phenotypes Across Multiple Gene Edited Lines Despite Transcriptomic and Splicing Discrepancies.” Frontiers in cell and developmental biology vol. 9 728707. 29 Sep. 2021, doi:10.3389/fcell.2021.728707
Spijkers, Xandor M et al. “A directional 3D neurite outgrowth model for studying motor axon biology and disease.” Scientific reports vol. 11, 1 2080. 22 Jan. 2021, doi:10.1038/s41598-021-81335-z
Zamani, Elham et al. “Netrin-1 protects the SH-SY5Y cells against amyloid beta neurotoxicity through NF-κB/Nrf2 dependent mechanism.” Molecular biology reports vol. 47, 12 (2020): 9271-9277. doi:10.1007/s11033-020-05996-1

1. An isolated antibody or antibody fragment thereof which specifically binds to UNC5C protein or a fragment thereof, wherein the antibody mimics and/or competes with Netrin-1 for binding to UNC5C, optionally wherein competition with Netrin-1 is determined by ELISA.

2. The isolated antibody of claim 1, wherein the antibody binds human and/or mouse UNC5C, wherein the antibody binds human UNC5C with an EC50 of at most 9.81E-08 M, or at most 1E-08M, or at most 5.5E-09M or at most 9.7E-09M as assessed by ELISA (such as binding of plated rhUNC5C), wherein the antibody selectively binds to UNC5C over other one or more netrin receptors, wherein the antibody selectively binds to UNC5C over one or more (or all of): neogenin, DCC, and DSCAM, UNC5A, UNC5B, UNC5, and/or wherein the antibody binds to UNC5C in accordance with SEQ ID NO: 99 and to an UNC5C T835M variant in accordance with SEQ ID NO: 110.

3. The isolated antibody of claim 1, wherein the antibody reduces UNC5C-mediated apoptosis, optionally wherein the antibody reduces UNC5C-mediated neuronal apoptosis and/or in induced pluripotent stem cell (iPSC)-derived neurons, optionally wherein apoptosis is measured by measuring caspase 3/7.

4. The isolated antibody according to claim 1, wherein the antibody binds to the extracellular region of UNC5C, and/or wherein the antibody binds to one or more residues of UNC5C that are involved in binding of Netrin-1 to UNC5C, and/or wherein the antibody binds to an epitope in the N terminal immunoglobulin domain of UNC5C.

5. The isolated antibody according to claim 4, wherein the antibody binds to an epitope in UNC5C comprising one or more of, or all of, the following residues: Thr89, Gln90, Gln102, Lys 103, Val107, Asp108, Glu109, Arg110, Val111, Ile118, Arg120 of UNC5C when numbered in accordance with SEQ ID NO: 99, optionally wherein the antibody binds to residues Thr89, Gln90, Gin 102, Lys 103, Val107, Glu109, Ile118, and Arg120 of UNC5C when numbered in accordance with SEQ ID NO: 99.

6. The isolated antibody according to claim 1, wherein the antibody comprises a heavy chain variable domain (VH) with the following CDRs:
CDRH1 comprising amino acid sequence SEQ ID NO:20,
CDRH2 comprising amino acid sequence SEQ ID NO:21 or SEQ ID NO:22, and
CDRH3 comprising amino acid sequence SEQ ID NO:23, or
a set of CDRs which contains one or two amino acid mutations, optionally substitutions, compared with the above set of CDRs.

7. The isolated antibody according to claim 6, wherein the antibody has a heavy chain variable domain (VH) with the following framework sequences:
HFWR1 of SEQ ID NO:50,
HFWR2 of SEQ ID NO:51,
HFWR3 of SEQ ID NO:55, and
HFWR4 of SEQ ID NO:62, or
framework sequences with one to five mutations, optionally substitutions, compared to the framework sequences above, optionally wherein the mutations in the framework sequences do not include a mutation at position 67 in standard IMGT numbering and/or are selected from the following positions in standard IMGT numbering:
HFWR2: positions 40, 49,
HFWR3: positions 80, 82, 86, and/or wherein the substitutions in the framework sequences are selected from the following positions in standard IMGT numbering:
HFWR2: at positions 40. T40S, at position 49: R49G,
HFWR3: at position 80: 180V, at position 82: T82K, at position 86: H86Q.

8. The isolated antibody according to claim 1, wherein the antibody has a heavy chain variable domain (VH) with the following framework sequences:
HFWR1 of SEQ ID NO:50;
HFWR2 of SEQ ID NO:51, 52, 53 or 54;
HFWR3 of SEQ ID NO:55, 56, 57, 58, or 61;
HFWR4 of SEQ ID NO:62,
optionally wherein the antibody has a heavy chain variable domain (VH) with the following framework sequences:
HFWR1 of SEQ ID NO:50;
HFWR2 of SEQ ID NO: 52;
HFWR3 of SEQ ID NO: 61; and
HFWR4 of SEQ ID NO:62.

9. The isolated antibody according to claim 1, wherein the antibody has a heavy chain variable domain (VH) comprising a sequence selected that has at least 80%, at least 85%, at least 90%, or at least 95% sequence identity with a sequence selected from SEQ ID Nos: 1 (ATL_5262 VH), 5 (ATL_0006036_VH), 8 (ATL_0006039_VH), 91 (ATL_0006177_VH), and 93 (ATL_0006178_VH), optionally wherein the antibody has a heavy chain variable domain (VH) comprising a sequence that has at least 80%, at least 85%, at least 90%, or a least 95% sequence identity with a sequence selected from: SEQ ID NO: 91 (ATL_6177 VH); and SEQ ID NO:93 (ATL_6178 VH).

10. The isolated antibody according to claim 1, wherein the antibody comprises a heavy chain variable domain (VH) with the following CDRs:
CDRH1 comprising amino acid sequence SEQ ID NO: 115 or 116,
CDRH2 comprising amino acid sequence SEQ ID NO: 117 or 118, and
CDRH3 comprising amino acid sequence SEQ ID NO: 119 or 120,
optionally wherein the antibody comprises a heavy chain variable domain (VH) with the CDRs of ATL_6187 VH (SEQ ID NO. 115, 117 and 119) or the CDRs of ATL_6191 VH (SEQ ID NO: 116, 118 and 120).

11. The isolated antibody according to claim 10, wherein the antibody has a heavy chain variable domain (VH) with the following framework sequences:
HFWR1 of SEQ ID NO: 50;
HFWR2 of SEQ ID NO: 52 or 195;
HFWR3 of SEQ ID NO: 196 or 197;
HFWR4 of SEQ ID NO: 198 or 62;
Or framework sequences comprising 1 to 5 mutations, such as substitutions, compared to these framework sequences,
optionally wherein the antibody comprises a heavy chain variable domain (VH) with the HFWRs of ATL_6187 VH (SEQ ID NO.50, 52, 196, 198) or the HFWRs of ATL_6191 VH (SEQ ID NO: 50, 195, 197, 62).

12. The isolated antibody according to claim 11, wherein the antibody has a heavy chain variable domain (VH) comprising a sequence selected that has at least 80%, at least 85%, at least 90%, or at least 95% sequence identity with a sequence selected from SEQ ID NOs: 112 (ATL_0006187 VH), and 113 (ATL_0006191 VH).

13. The isolated antibody according to claim 1, wherein the antibody comprises a light chain variable domain (VL) with the following CDRs:
CDRL1 comprising amino acid sequence SEQ ID NO:24, 27, 30, 31 or 32,
CDRL2 comprising amino acid sequence SEQ ID NO:34, and
CDRL3 comprising amino acid sequence SEQ ID NO:40, 43, 46, 47, or
a set of CDRs which contains one, two or three amino acid mutations, optionally substitutions compared with the above set of CDRs.

14. The isolated antibody according to claim 13, wherein the antibody has a light chain variable domain (VL) with the following framework sequences:
LFWR1 of SEQ ID NO:63; 66;
LFWR2 of SEQ ID NO:71; 73; 75,
LFWR3 of SEQ ID NO:77; 80; and
LFWR4 of SEQ ID NO:85; 87; 89; or
a set of FWRs which contains one to five amino acid substitutions compared with the above set of FWR.

14. The isolated antibody according to claim 14, wherein the antibody has a light chain variable domain (VL) comprising a sequence selected that has at least 80%, at least 85%, at least 90%, or a least 95% sequence identity with a sequence selected from SEQ ID Nos: 9 (ATL_5262 VL), 12 (ATL 5998 VL): 15 (ATL_6001 VL), 16 (ATL_6002 VL), 17 (ATL_6003 VL),
optionally wherein the antibody has a light chain variable domain (VL) comprising a sequence that has at least 80%, at least 85%, at least 90%, or at least 95% sequence identity with a sequence selected from: SEQ ID: 9 (ATL_5262 VL), SEQ ID NO: 16 (ATL_6002 VL); and SEQ ID NO: 17 (ATL_6003 VL).

15. The isolated antibody according to claim 1, wherein one or more of (or all of) VH residues Tyr55, Gly63, Thr64, Thr65, Asn66, Ser74, Arg108, Met110 interact with one or more residues of UNC5C;
and/or wherein one or more of (or all of) VL residues Tyr38, Ser107, Tyr108, Ser109, Thr114, interact with one or more residues of UNC5C, optionally wherein the antibody comprises a VH with:
at position 55: Tyr, at position 63: Gly, at position 64: Thr, at position 65: Thr, at position 66; Asn, at position 74: Ser, at position 108: Arg, and at position 110: Met, or a sequence with conservative amino acid substitutions at one or more of these positions; and/or
wherein the antibody comprises a VL with:
at position 38: Tyr, at position 107: Ser, at position 108: Tyr, at position 109: Ser, at position 114: Thr, or a sequence with conservative amino acid substitutions at one or more of these positions.

16. The isolated antibody according to claim 15, wherein the antibody has a VH with at least 80% sequence identity with the VH of SEQ ID NO: 1 (ATL_5262 VH), and a VL with at least 80% sequence identity with the VL of SEQ ID: 9 (ATL_5262 VL), wherein:
(a) VH residue 66 is Asn, optionally wherein Asn66 interacts with Val 107 of UNC5C; and/or
(b) VH residues 55, 66, 108 and 110 are respectively Tyr, Asn, Arg and Met, optionally wherein Tyr55, Asn66, Arg108, and/or Met110 interact with Glu109 of UNC5C; and/or
(c) VH residues 108 is Arg and/or residue 110 is Met, optionally wherein Arg108, and/or Met110 interact with Ile118 of UNC5C; and/or
(d) VH residues 55, 64, 65 are respectively Tyr, Thr and Thr, optionally wherein Tyr55, Thr64, and/or Thr65 interacts with Arg120 of UNC5C, and/or
(e) VH residue 64 is Thr, optionally wherein Thr64 interacts with Thr89 and/or Arg120 of UNC5C; and/or
(f) VH residue 63 is Gly, optionally wherein Gly63 interacts with Gln90 of UNC5C; and/or
(g) VH residue 65 is Thr, optionally wherein Thr65 interacts with Gln102 and/or Arg120 of UNC5C; and/or
(h) VH residue 74 is Ser, optionally wherein Ser74 interacts with Lys103 of UNC5C; and/or
(i) VL residue 114 is Thr, optionally wherein Thr114 interacts with Val107 and/or Glu109, and/or Arg110 of UNC5C, and/or
(j) VL residue 109 is Ser, optionally wherein Ser109 interacts with Glu109 and/or Arg110 of UNC5C; and/or
(k) VL residue 108 is Tyr, optionally wherein Tyr108 interacts with Arg110 and/or Val111 of UNC5C; and/or
(l) VL residue 107 is Ser, optionally wherein Ser107 interacts with Val111 of UNC5C; and/or
(m) VL residue 38 is Tyr, optionally wherein Tyr38 interacts with Val111 of UNC5C;
wherein the UNC5C residues are numbered in accordance with SEQ ID NO: 99.

17. The isolated antibody according to claim 1, wherein the antibody comprises:
a heavy chain variable domain (VH) with the following CDRs:
CDRH1 comprising amino acid sequence SEQ ID NO: 142-149;
CDRH2 comprising amino acid sequence SEQ ID NO: 150-158, and
CDRH3 comprising amino acid sequence SEQ ID NO: 159-168,
optionally wherein the antibody comprises a VH with the CDRs of any of the antibodies ATL_6530 (SEQ ID NO: 142, 150, 159); ATL_6531 (SEQ ID NO:143, 151, 160); ATL_6532 (SEQ ID NO: 144, 152, 161); ATL_6533 (SEQ ID NO: 145, 153, 162); ATL_6534 (SEQ ID NO:144, 152, 163); ATL 6535 (SEQ ID NO: 143, 154, 164); ATL_6536 (SEQ ID NO: 146, 155, 165); ATL 6537 (SEQ ID NO:147, 156, 166); ATL_6538 (SEQ ID NO:148, 157, 167); ATL 6539 (SEQ ID NO: 149, 158, 168); and/or
a light chain variable domain (VL) with the following CDRs:
CDRL1 comprising amino acid sequence SEQ ID NO: 169-176;
CDRL2 comprising amino acid sequence SEQ ID NO: 177-183;
CDRL3 comprising amino acid sequence SEQ ID NO: 184-193,
optionally wherein the antibody comprises a VL with the CDRs of any of the antibodies ATL_6530 (SEQ ID NO:169, 177, 184); ATL_6531 (SEQ ID NO:170, 178, 185); ATL_6532 (SEQ ID NO:171, 177, 186); ATL_6533 (SEQ ID NO:169, 177, 187); ATL_6534 (SEQ ID NO:169, 177, 188); ATL_6535 (SEQ ID NO:172, 179, 189); ATL_6536 (SEQ ID NO: 173, 180, 190); ATL 6537 (SEQ ID NO:174, 181, 191): ATL 6538 (SEQ ID NO:175, 182, 192); ATL_6539 (SEQ ID NO:176, 183, 193).

18. The isolated antibody according to claim 17, wherein the antibody has:
a heavy chain variable domain (VH) with the following framework sequences:
HFWR1 of SEQ ID NO: 199, 202, 205, 209, 212, 213, 216, 220, 224, 227;
HFWR2 of SEQ ID NO: 200, 203, 206, 210, 214, 217, 221, 225, 228;
HFWR3 of SEQ ID NO: 201, 204, 207, 211, 215, 218, 222, 226, 229;
HFWR4 of SEQ ID NO: 62, 208, 219, 223,
Optionally wherein the antibody has a heavy chain variable domain (VH) with the framework sequences of any of the antibodies ATL 6530 (SEQ ID NO: 199, 200, 201, 62); ATL_6531 (SEQ ID NO:202, 203, 204, 62): ATL 6532 (SEQ ID NO:205, 206, 207, 208); ATL_6533 (SEQ ID NO:209, 210, 211, 208); ATL_6534 (SEQ ID NO:212, 206, 207, 208); ATL_6535 (SEQ ID NO:213, 214, 215, 208); ATL_6536 (SEQ ID NO:216, 217, 218, 219); ATL_6537 (SEQ ID NO:220, 221, 222, 223); ATL_6538 (SEQ ID NO: 224, 225, 226, 208): ATL_6539 (SEQ ID NO:227, 228, 229, 208); and/or a light chain variable domain (VL) with the following framework sequences:
LFWR1 of SEQ ID NO: 230 to 237 and 68;
LFWR2 of SEQ ID NO: 239 to 246;
LFWR3 of SEQ ID NO: 247 to 255; and
LFWR4 of SEQ ID NO: 256 to 258,
optionally wherein the antibody comprises a VL with the FWRs of any of the antibodies ATL 6530 (SEQ ID NO:230, 238, 247, 256); ATL_6531 (SEQ ID NO:231, 239, 248, 257); ATL_6532 (SEQ ID NO:232, 240, 249, 258); ATL_6533 (SEQ ID NO:230, 241, 247, 256); ATL 6534 (SEQ ID NO:233, 238, 250, 256); ATL_6535 (SEQ ID NO:234, 242, 251, 257); ATL_6536 (SEQ ID NO:68, 243, 252, 257); ATL_6537 (SEQ ID NO: 235, 244, 253, 256); ATL_6538 (SEQ ID NO:236, 245, 254, 256); ATL_6539 (SEQ ID NO:237, 246, 255, 256), and/or wherein the antibody has a heavy chain variable domain (VH) with the CDRs and/or the framework sequences of ATL_6532 or ATL6533 and/or wherein the antibody has a heavy chain variable domain (VH) comprising a sequence selected that has at least 80%, at least 85%, at least 90%, or at least 95% sequence identity with a sequence selected from SEQ ID NOs: 122 (ATL_0006530 VH): 123 (ATL_0006531 VH); 124 (ATL_0006532 VH): 125 (ATL_0006533 VH); 126 (ATL_0006534 VH); 127 (ATL_0006535 VH); 128 (ATL_0006536 VH); 129 (ATL_0006537 VH); 130 (ATL_0006538 VH).
optionally wherein the antibody has a heavy chain variable domain (VH) comprising a sequence that has at least 70%, at least 75%, at least 80%, at least 85%, at least 90%, or at least 95% sequence identity with a sequence selected from: SEQ ID NO: 124 (ATL_6532 VH); SEQ ID NO: 125 (ATL 6533 VH); and/or
wherein the antibody has a light chain variable domain (VL) comprising a sequence selected that has at least 80%, at least 85%, at least 90%, or at least 95% sequence identity with a sequence selected from SEQ ID NO: 132-141 (ATL6530-6539 VL), optionally wherein the antibody has a light chain variable domain (VL) comprising a sequence that has at least 80%, at least 85%, at least 90%, or at least 95% sequence identity with a sequence selected from: SEQ ID NO: 134 (ATL_0006532); or SEQ ID NO: 135 (ATL_0006533).

19. The isolated antibody according to claim 1, wherein the antibody is a monoclonal antibody and/or wherein the antibody is a whole antibody and/or wherein the antibody is an IgG1 or variant thereof, optionally wherein the antibody is an IgG1 variant L234A/L235A (LALA).

20. A method of treating a neurodegenerative disease or disorder in a subject, wherein the method comprises administering to the subject a therapeutically effective amount of an antibody or fragment thereof that binds UNC5C, wherein the antibody is as described in claim 1, optionally wherein the neurodegenerative disorder is selected from: frontotemporal dementia (FTD), Alzheimer's disease (AD), Huntington's Disease (HD), Parkinson's disease (PD), amyotrophic lateral sclerosis (ALS), human immunodeficiency virus (HIV)-induced encephalitis, Chronic traumatic encephalopathy (CTE), vascular dementia, prion diseases, Lewy body disease, Spinal muscular atrophy (SMA), Motor Neuron Disease (MND), such as amyotrophic lateral sclerosis (ALS), progressive supranuclear palsy (PSP), spinocerebellar ataxias (SCA) types 1, 2, 6, 7 and 17, Machado-Joseph disease (MJD/SCA3), dentatorubral pallidoluysian atrophy (DRPLA), spinal bulbar muscular atrophy X-linked type 1 (SMAX1/SBMA), Anderson-Fabry (X-linked Fabry Disease), and DNAJB6 Myopathies, optionally wherein the neurodegenerative disease is selected from FTD, AD, HD, and PD.
